(19) United States
(12) Patent Application Publication
(10) Pub. No.: US 2022/0362351 A1
(43) Pub. Date: Nov. 17, 2022

(54) RECOMBINANT SIALIDASES AND METHODS OF USING THE SAME

(71) Applicant: Palleon Pharmaceuticals Inc., Waltham, MA (US)

(72) Inventors: Li Peng, Lexington, MA (US); Lizhi Cao, Stoneham, MA (US); Sandip A. Shelke, Belmont, MA (US); Andrew S. Turner, Waltham, MA (US); Lihui Xu, Chestnut Hill, MA (US); Wayne C. Gatlin, Wakefield, MA (US); James W. Broderick, Wellesley, MA (US); Karl D. Normington, Prides Crossing, MA (US); Sujata B. Nerle, Somerville, MA (US); Zakir B. Siddiquee, Cambridge, MA (US); Abhishek Das, Wayland, MA (US)

(21) Appl. No.: 17/624,123

(22) PCT Filed: Jul. 3, 2020
(86) PCT No.: PCT/US2020/040827

§ 371 Date: Dec. 30, 2021

Related U.S. Application Data
(60) Provisional application No. 62/957,027, filed on Jan. 3, 2020.
(60) Provisional application No. 62/870,336, filed on Jul. 3, 2019.

(51) Int. Cl. A61K 38/47; A61K 47/68; C12N 9/24

(52) U.S. Cl. CPC A61K 38/47; A61K 47/68; C12N 9/2402; C12Y 302/01018; C07K 2317/76; C07K 2319/31

(57) ABSTRACT

The invention relates generally to recombinant sialidases, methods and compositions for extending the serum half-life of the recombinant sialidases, and use of the same in the treatment of a sialic acid-related disorder.

### CROSS REFERENCE TO RELATED APPLICATIONS

This application claims the benefit of, and priority to, U.S. Provisional Patent Application Ser. No. 62/870,336, filed Jul. 3, 2019 and U.S. Provisional Patent Application Ser. No. 62/957,027, filed Jan. 3, 2020, the entire disclosure of each of which is hereby incorporated by reference in its entirety.

### FIELD OF THE INVENTION

The invention relates generally to recombinant sialidases, methods and compositions for extending the serum half-life of recombinant sialidases, and use of the same in the treatment of a sialic acid-related disorder.

### BACKGROUND

A growing body of evidence supports roles for glycans, and sialoglycans in particular, at various pathophysiological steps of tumor progression. Glycans regulate tumor proliferation, invasion, hematogenous metastasis and angiogenesis (Fuster et al. (2005) NAT. REV. CANCER 5(7): 526-42). The sialylation of cell surface glycoconjugates is frequently altered in cancers, resulting in the expression of sialylated tumor-associated carbohydrate antigens. The expression of sialylated glycans by tumor cells is often associated with increased aggressiveness and metastatic potential of a tumor.

It has recently become apparent that Siglecs (sialic acid-binding immunoglobulin-like lectins), a family of sialic acid binding lectins, play a role in cancer immune suppression by binding to hypersialylated cancer cells and mediating the suppression of signals from activating NK cell receptors, thereby inhibiting NK cell-mediated killing of tumor cells (Jandus et al. (2014) J. CLIN. INVEST. 124: 1810-1820; Läubli et al. (2014) PROC. NATL. ACAD. SCI. USA 111: 14211-14216; Hudak et al. (2014) NAT. CHEM. BIOL. 10: 69-75). Likewise, enzymatic removal of sialic acids by treatment with sialidase can enhance NK cell-mediated killing of tumor cells (Jandus, supra; Hudak, supra; Xiao et al. (2016) PROC. NATL. ACAD. SCI. USA 113(37): 10304-9.)

Cancer immunotherapy with immune checkpoint inhibitors, including antibodies blocking the PD-1/PD-L1 pathway, has improved the outcome of many cancer patients. However, despite advances that have been made to date, many patients do not respond to currently available immune checkpoint inhibitors. Accordingly, there is still a need for effective interventions that overcome the immune suppressive tumor microenvironment and for treating cancers associated with hypersialylated cancer cells.

### SUMMARY OF THE INVENTION

The invention is based, in part, upon the discovery that it is possible to treat a sialic acid-mediated disorder by administering a sialidase enzyme or a sialidase enzyme conjugated to a serum half-life enhancer. Surprisingly, it has been discovered that a sialidase or a sialidase enzyme conjugated to a serum half-life enhancer that lacks a targeting moiety (e.g., an antibody binding domain directed to a tumor antigen) can effectively treat a sialic acid-mediated disorder (e.g., cancer, e.g., a solid tumor) in vivo.

The invention further relates to recombinant forms of sialidase enzymes, sialidase enzymes conjugated to a serum half-life enhancer, and pharmaceutical compositions thereof, that have suitable substrate specificities and activities to be useful in removing sialic acid and/or sialic acid containing molecules from the surface of cancer cells and/or removing sialic acid and/or sialic acid containing molecules from the tumor microenvironment, and/or reducing the concentration of sialic acid and/or sialic acid containing molecules in the tumor microenvironment.

Thus, in certain aspects, the invention provides a pharmaceutical composition comprising or consisting essentially of a sialidase conjugated to a serum half-life enhancer that increases the serum half-life of the sialidase when administered to a subject.

In another aspect, the invention provides a method of treating a sialic acid-related disorder in a subject in need thereof. The method includes administering to the subject an effective amount of a pharmaceutical composition comprising or consisting essentially of a sialidase and a serum half-life enhancer that increases the serum half-life of the sialidase when administered to the subject, thereby to treat the disorder.

In certain embodiments, the sialidase is not conjugated to a cancer antigen targeting agent that binds a cancer antigen associated with a cancerous cell.

In certain embodiments, the sialidase is a functional fragment of a full-length sialidase or a variant that exhibits at least 50% of the activity of the full-length sialidase.

In certain embodiments, the sialidase and the serum half-life enhancer are covalently linked together in a fusion protein or are chemically conjugated together.

In certain embodiments, the serum half-life enhancer is selected from the group consisting of an Fc domain, transferrin, albumin, XTEN, a homo-amino acid polymer (HAP), a proline-alanine-serine polymer (PAS), an elastin-like peptide (ELP), albumin binding domain, CTP fusion, GLK fusion, and a polyethylene glycol.

In certain embodiments, the serum half-life enhancer is an Fc domain.

In certain embodiments, the serum half-life enhancer is not an Fc domain or polyethylene glycol.

In certain embodiments, the sialidase comprises one or more mutations relative to a template, wild-type sialidase.

In certain embodiments, the sialidase comprises a substitution or deletion of a methionine residue at a position corresponding to position 1 of wild-type human Neu2 (M1); a substitution of a valine residue at a position corresponding to position 6 of wild-type human Neu2 (V6); a substitution of an isoleucine residue at a position corresponding to position 187 of wild-type human Neu2 (I187); or a substitution of a cysteine residue at a position corresponding to position 332 of wild-type human Neu2 (C332); or a combination of any of the foregoing substitutions. In certain embodiments, in the sialidase, (a) the methionine residue at a position corresponding to position 1 of wild-type human Neu2 is deleted (ΔM1), is substituted by alanine (M1A), or is substituted by aspartic acid (M1D); (b) the valine residue at a position corresponding to position 6 of wild-type human Neu2 is substituted by tyrosine (V6Y); (c) the isoleucine residue at a position corresponding to position 187 of wild-type human Neu2 is substituted by lysine (I187K); (d) or the cysteine residue at a position corresponding to position 332 of wild-type human Neu2 is substituted by alanine (C332A); or the sialidase comprises a combination of any of the foregoing substitutions.

In certain embodiments, the sialidase comprises a substitution or deletion of a methionine residue at a position corresponding to position 1 of wild-type human Neu2 (M1); a substitution of a valine residue at a position corresponding to position 6 of wild-type human Neu2 (V6); a substitution of an proline residue at a position corresponding to position 62 of wild-type human Neu2 (P62); a substitution of an alanine residue at a position corresponding to position 93 of wild-type human Neu2 (A93); a substitution of an isoleucine residue at a position corresponding to position 187 of wild-type human Neu2 (I187); a substitution of a glutamine residue at a position corresponding to position 126 of wild-type human Neu2 (Q126); a substitution of an alanine residue at a position corresponding to position 242 of wild-type human Neu2 (A242); a substitution of a glutamine residue at a position corresponding to position 270 of wild-type human Neu2 (Q270); a substitution of a serine residue at a position corresponding to position 301 of wild-type human Neu2 (S301); a substitution of a tryptophan residue at a position corresponding to position 302 of wild-type human Neu2 (W302); a substitution of a cysteine residue at a position corresponding to position 332 of wild-type human Neu2 (C332); or a combination of any of the foregoing substitutions.

In certain embodiments, the sialidase comprises a combination of substitutions selected from the group consisting of:

(a) M1D, V6Y, P62G, A93E, I187K, C332A;

(b) M1D, V6Y, P62G, A93E, I187K, S301A, W302R, C332A;

(c) M1D, V6Y, P62G, A93E, Q126Y, I187K, A242F, Q270T, C332A;

(d) M1D, V6Y, P62G, A93E, Q126Y, I187K, C332A; and

(e) A93E, Q126Y, I187K, A242F, Q270T, C332A.

In certain embodiments, the sialidase conjugated to a serum half-life enhancer comprises an amino acid sequence selected from the group consisting of SEQ ID NOs: 115, 152, 180, 184, and 188, or an amino acid sequence that has at least 85%, 90%, 95%, 96%, 97%, 98%, or 99% to an amino acid sequence selected from the group consisting of SEQ ID NOs: 115, 152, 180, 184, and 188.

In certain embodiments, the sialidase comprises a substitution of a proline residue at a position corresponding to position 5 of wild-type human Neu2 (P5); a substitution of a lysine residue at a position corresponding to position 9 of wild-type human Neu2 (K9); a substitution of a lysine residue at a position corresponding to position 44 of wild-type human Neu2 (K44); a substitution of a lysine residue at a position corresponding to position 45 of wild-type human Neu2 (K45); a substitution of a leucine residue at a position corresponding to position 54 of wild-type human Neu2 (L54); a substitution of a proline residue at a position corresponding to position 62 of wild-type human Neu2 (P62); a substitution of a glutamine residue at a position corresponding to position 69 of wild-type human Neu2 (Q69); a substitution of an arginine residue at a position corresponding to position 78 of wild-type human Neu2 (R78); a substitution of an aspartic acid residue at a position corresponding to position 80 of wild-type human Neu2 (D80); a substitution of an alanine residue at a position corresponding to position 93 of wild-type human Neu2 (A93); a substitution of a glycine residue at a position corresponding to position 107 of wild-type human Neu2 (G107); a substitution of a glutamine residue at a position corresponding to position 108 of wild-type human Neu2 (Q108); a substitution of a glutamine residue at a position corresponding to position 112 of wild-type human Neu2 (Q112); a substitution of a cysteine residue at a position corresponding to position 125 of wild-type human Neu2 (C125); a substitution of a glutamine residue at a position corresponding to position 126 of wild-type human Neu2 (Q126); a substitution of an alanine residue at a position corresponding to position 150 of wild-type human Neu2 (A150); a substitution of a cysteine residue at a position corresponding to position 164 of wild-type human Neu2 (C164); a substitution of an arginine residue at a position corresponding to position 170 of wild-type human Neu2 (R170); a substitution of an alanine residue at a position corresponding to position 171 of wild-type human Neu2 (A171); a substitution of a glutamine residue at a position corresponding to position 188 of wild-type human Neu2 (Q188); a substitution of an arginine residue at a position corresponding to position 189 of wild-type human Neu2 (R189); a substitution of an alanine residue at a position corresponding to position 213 of wild-type human Neu2 (A213); a substitution of a leucine residue at a position corresponding to position 217 of wild-type human Neu2 (L217); a substitution of a glutamic acid residue at a position corresponding to position 225 of wild-type human Neu2 (E225); a substitution of a histidine residue at a position corresponding to position 239 of wild-type human Neu2 (H239); a substitution of a leucine residue at a position corresponding to position 240 of wild-type human Neu2 (L240); a substitution of an arginine residue at a position corresponding to position 241 of wild-type human Neu2 (R241); a substitution of an alanine residue at a position corresponding to position 242 of wild-type human Neu2 (A242); a substitution of a valine residue at a position corresponding to position 244 of wild-type human Neu2 (V244); a substitution of a threonine residue at a position corresponding to position 249 of wild-type human Neu2 (T249); a substitution of an aspartic acid residue at a position corresponding to position 251 of wild-type human Neu2 (D251); a substitution of a glutamic acid residue at a position corresponding to position 257 of wild-type human Neu2 (E257); a substitution of a serine residue at a position corresponding to position 258 of wild-type human Neu2 (S258); a substitution of a leucine residue at a position corresponding to position 260 of wild-type human Neu2 (L260); a substitution of a valine residue at a position corresponding to position 265 of wild-type human Neu2 (V265); a substitution of a glutamine residue at a position corresponding to position 270 of wild-type human Neu2 (Q270); a substitution of a tryptophan residue at a position corresponding to position 292 of wild-type human Neu2 (W292); a substitution of a serine residue at a position corresponding to position 301 of wild-type human Neu2 (S301); a substitution of a tryptophan residue at a position corresponding to position 302 of wild-type human Neu2 (W302); a substitution of a cysteine residue at a position corresponding to position 332 of wild-type human Neu2 (C332); a substitution of a valine residue at a position corresponding to position 363 of wild-type human Neu2 (V363); or a substitution of a leucine residue at a position corresponding to position 365 of wild-type human Neu2 (L365); or a combination of any of the foregoing substitutions.

In certain embodiments, the sialidase is selected from the group consisting of a bacterial sialidase, a viral sialidase, and a mammalian sialidase. In certain embodiments, the sialidase is a human sialidase. In certain embodiments, the human sialidase is selected from the group consisting of neu1, neu2, neu3, and neu4. In certain embodiments, the human sialidase is neu2.

In certain embodiments, the pharmaceutical comprises from about 0.01 mg/kg to about 100 mg/kg of the sialidase.

In certain embodiments, the pharmaceutical composition comprises a second therapeutic agent. In certain embodiments, the second therapeutic agent is selected from the group consisting of an anti-inflammatory agent, anti-angiogenic agent, anti-fibrotic agent, or an anti-proliferative compound (e.g., a cytotoxic agent or a checkpoint inhibitor).

In certain embodiments, the pharmaceutical composition further comprises a stabilizing amount of a sialidase stabilizing agent. In certain embodiments, the sialidase stabilizing agent is a cation. In certain embodiments, the cation is selected from the group consisting of calcium and magnesium.

In certain embodiments, the pharmaceutical composition is disposed in a sterile container (e.g., bottle or vial). In certain embodiments, the pharmaceutical composition is lyophilized in the sterile container. In certain embodiments, the pharmaceutical composition is present as a solution in the sterile container. In certain embodiments, sterile container is sealed with a septum. In certain embodiments, sterile container has a label disposed thereon identifying the pharmaceutical composition contained in the container.

In another aspect, the disclosure relates to a method of treating a sialic acid-related disorder in a subject in need thereof, the method comprising administering to the subject a pharmaceutical composition comprising an effective amount of a sialidase and a serum half-life enhancer that increases the serum half-life of the sialidase when administered to a subject, thereby to treat the disorder.

In certain embodiments, the sialic acid-related disorder is cancer. In certain embodiments, the sialidase is not conjugated to a cancer antigen targeting agent that binds a cancer antigen associated with a cancerous cell.

In certain embodiments, the sialidase is a functional fragment of a full-length sialidase that exhibits at least 50% of the activity of the full-length sialidase. In certain embodiments, the sialidase is a variant that exhibits at least 50% of the activity of the wild-type sialidase.

In certain embodiments, the sialidase and the serum half-life enhancer are covalently linked together in a fusion protein. In certain embodiments, the sialidase and serum half-life enhancer are chemically conjugated together.

In certain embodiments, the serum half-life enhancer is selected from the group consisting of an Fc domain, transferrin, albumin, XTEN, a homo-amino acid polymer (HAP), a proline-alanine-serine polymer (PAS), an elastin-like peptide (ELP), and a polyethylene glycol. In certain embodiments, the serum half-life enhancer is an Fc domain. In certain embodiments, the serum half-life enhancer is not an Fc domain or polyethylene glycol.

In certain embodiments, the sialidase comprises one or more mutations relative to a template, wild-type sialidase. In certain embodiments, the sialidase comprises a substitution or deletion of a methionine residue at a position corresponding to position 1 of wild-type human Neu2 (M1); a substitution of a valine residue at a position corresponding to position 6 of wild-type human Neu2 (V6); a substitution of an isoleucine residue at a position corresponding to position 187 of wild-type human Neu2 (I187); or a substitution of a cysteine residue at a position corresponding to position 332 of wild-type human Neu2 (C332); or a combination of any of the foregoing substitutions.

In certain embodiments, in the sialidase, the methionine residue at a position corresponding to position 1 of wild-type human Neu2 is deleted (ΔM1), is substituted by alanine (M1A), or is substituted by aspartic acid (M1D); the valine residue at a position corresponding to position 6 of wild-type human Neu2 is substituted by tyrosine (V6Y); the isoleucine residue at a position corresponding to position 187 of wild-type human Neu2 is substituted by lysine (I187K); or the cysteine residue at a position corresponding to position 332 of wild-type human Neu2 is substituted by alanine (C332A); or the sialidase comprises a combination of any of the foregoing substitutions.

In certain embodiments, the sialidase comprises a substitution or deletion of a methionine residue at a position corresponding to position 1 of wild-type human Neu2 (M1); a substitution of a valine residue at a position corresponding to position 6 of wild-type human Neu2 (V6); a substitution of an proline residue at a position corresponding to position 62 of wild-type human Neu2 (P62); a substitution of an alanine residue at a position corresponding to position 93 of wild-type human Neu2 (A93); a substitution of an isoleucine residue at a position corresponding to position 187 of wild-type human Neu2 (I187); a substitution of a glutamine residue at a position corresponding to position 126 of wild-type human Neu2 (Q126); a substitution of an alanine residue at a position corresponding to position 242 of wild-type human Neu2 (A242); a substitution of a glutamine residue at a position corresponding to position 270 of wild-type human Neu2 (Q270); a substitution of a serine residue at a position corresponding to position 301 of wild-type human Neu2 (S301); a substitution of a tryptophan residue at a position corresponding to position 302 of wild-type human Neu2 (W302); a substitution of a cysteine residue at a position corresponding to position 332 of wild-type human Neu2 (C332); or a combination of any of the foregoing substitutions.

In certain embodiments, the sialidase comprises a combination of substitutions selected from the group consisting of:

(a) M1D, V6Y, P62G, A93E, I187K, C332A;

(b) M1D, V6Y, P62G, A93E, I187K, S301A, W302R, C332A;

(c) M1D, V6Y, P62G, A93E, Q126Y, I187K, A242F, Q270T, C332A;

(d) M1D, V6Y, P62G, A93E, Q126Y, I187K, C332A; and

(e) A93E, Q126Y, I187K, A242F, Q270T, C332A.

In certain embodiments, the sialidase conjugated to a serum half-life enhancer comprises an amino acid sequence selected from the group consisting of SEQ ID NOs: 115, 152, 180, 184, and 188, or an amino acid sequence that has at least 85%, 90%, 95%, 96%, 97%, 98%, or 99% to an amino acid sequence selected from the group consisting of SEQ ID NOs: 115, 152, 180, 184, and 188.

In certain embodiments, the sialidase comprises a substitution of a proline residue at a position corresponding to position 5 of wild-type human Neu2 (P5); a substitution of a lysine residue at a position corresponding to position 9 of wild-type human Neu2 (K9); a substitution of a lysine residue at a position corresponding to position 44 of wild-type human Neu2 (K44); a substitution of a lysine residue at a position corresponding to position 45 of wild-type human Neu2 (K45); a substitution of a leucine residue at a position corresponding to position 54 of wild-type human Neu2 (L54); a substitution of a proline residue at a position corresponding to position 62 of wild-type human Neu2 (P62); a substitution of a glutamine residue at a position corresponding to position 69 of wild-type human Neu2 (Q69); a substitution of an arginine residue at a position corresponding to position 78 of wild-type human Neu2 (R78); a substitution of an aspartic acid residue at a position corresponding to position 80 of wild-type human Neu2 (D80); a substitution of an alanine residue at a position corresponding to position 93 of wild-type human Neu2 (A93); a substitution of a glycine residue at a position corresponding to position 107 of wild-type human Neu2 (G107); a substitution of a glutamine residue at a position corresponding to position 108 of wild-type human Neu2 (Q108); a substitution of a glutamine residue at a position corresponding to position 112 of wild-type human Neu2 (Q112); a substitution of a cysteine residue at a position corresponding to position 125 of wild-type human Neu2 (C125); a substitution of a glutamine residue at a position corresponding to position 126 of wild-type human Neu2 (Q126); a substitution of an alanine residue at a position corresponding to position 150 of wild-type human Neu2 (A150); a substitution of a cysteine residue at a position corresponding to position 164 of wild-type human Neu2 (C164); a substitution of an arginine residue at a position corresponding to position 170 of wild-type human Neu2 (R170); a substitution of an alanine residue at a position corresponding to position 171 of wild-type human Neu2 (A171); a substitution of a glutamine residue at a position corresponding to position 188 of wild-type human Neu2 (Q188); a substitution of an arginine residue at a position corresponding to position 189 of wild-type human Neu2 (R189); a substitution of an alanine residue at a position corresponding to position 213 of wild-type human Neu2 (A213); a substitution of a leucine residue at a position corresponding to position 217 of wild-type human Neu2 (L217); a substitution of a glutamic acid residue at a position corresponding to position 225 of wild-type human Neu2 (E225); a substitution of a histidine residue at a position corresponding to position 239 of wild-type human Neu2 (H239); a substitution of a leucine residue at a position corresponding to position 240 of wild-type human Neu2 (L240); a substitution of an arginine residue at a position corresponding to position 241 of wild-type human Neu2 (R241); a substitution of an alanine residue at a position corresponding to position 242 of wild-type human Neu2 (A242); a substitution of a valine residue at a position corresponding to position 244 of wild-type human Neu2 (V244); a substitution of a threonine residue at a position corresponding to position 249 of wild-type human Neu2 (T249); a substitution of an aspartic acid residue at a position corresponding to position 251 of wild-type human Neu2 (D251); a substitution of a glutamic acid residue at a position corresponding to position 257 of wild-type human Neu2 (E257); a substitution of a serine residue at a position corresponding to position 258 of wild-type human Neu2 (S258); a substitution of a leucine residue at a position corresponding to position 260 of wild-type human Neu2 (L260); a substitution of a valine residue at a position corresponding to position 265 of wild-type human Neu2 (V265); a substitution of a glutamine residue at a position corresponding to position 270 of wild-type human Neu2 (Q270); a substitution of a tryptophan residue at a position corresponding to position 292 of wild-type human Neu2 (W292); a substitution of a serine residue at a position corresponding to position 301 of wild-type human Neu2 (S301); a substitution of a tryptophan residue at a position corresponding to position 302 of wild-type human Neu2 (W302); a substitution of a cysteine residue at a position corresponding to position 332 of wild-type human Neu2 (C332); a substitution of a valine residue at a position corresponding to position 363 of wild-type human Neu2 (V363); or a substitution of a leucine residue at a position corresponding to position 365 of wild-type human Neu2 (L365); or a combination of any of the foregoing substitutions.

In certain embodiments, the sialidase is selected from the group consisting of a bacterial sialidase, a viral sialidase, and a mammalian sialidase. In certain embodiments, the mammalian sialidase is a human sialidase. In certain embodiments, the human sialidase is selected from the group consisting of neu1, neu2, neu3, and neu4. In certain embodiments, the human sialidase is neu2.

In certain embodiments, from about 0.01 mg/kg to about 100 mg/kg of the sialidase is administered to the subject.

In certain embodiments, the cancer is a solid tumor, soft tissue tumor, hematopoietic tumor or metastatic lesion. In certain embodiments, the solid tumor is a sarcoma, adenocarcinoma, or carcinoma. In certain embodiments, the solid tumor is a head and neck (e.g., pharynx), thyroid, lung (e.g., small cell or non-small cell lung carcinoma (NSCLC)), breast, lymphoid, gastrointestinal (e.g., oral, esophageal, stomach, liver, pancreas, small intestine, colon and rectum, anal canal), genital or genitourinary tract (e.g., renal, urothelial, bladder, ovarian, uterine, cervical, endometrial, prostate, testicular), CNS (e.g., neural or glial cell, e.g., neuroblastoma or glioma), or skin (e.g., melanoma) tumor. In certain embodiments, the cancer is breast cancer.

In certain embodiments, the hematopoietic tumor is a leukemia, acute leukemia, acute lymphoblastic leukemia (ALL), B-cell, T-cell or FAB ALL, acute myeloid leukemia (AML), chronic myelocytic leukemia (CML), chronic lymphocytic leukemia (CLL), e.g., transformed CLL, diffuse large B-cell lymphoma (DLBCL), follicular lymphoma, hairy cell leukemia, myelodyplastic syndrome (MDS), lymphoma, Hodgkin's disease, malignant lymphoma, non-Hodgkin's lymphoma, Burkitt's lymphoma, multiple myeloma, or Richter's Syndrome (Richter's Transformation). In certain embodiments, the cancer is lymphoma.

In certain embodiments, administration of the pharmaceutical composition increases expression of granzyme B, IFNγ, IL-10, IL-6, or IL-17A in the subject.

In certain embodiments, the pharmaceutical composition is administered to the subject in combination with another therapeutic agent. In certain embodiments, the therapeutic agent is selected from the group consisting of an anti-inflammatory agent, anti-angiogenic agent, anti-fibrotic agent, or an anti-proliferative compound (e.g., a cytotoxic agent or a checkpoint inhibitor).

In certain embodiments, the pharmaceutical composition further comprises a stabilizing amount of a sialidase stabilizing agent. In certain embodiments, the sialidase stabilizing agent is a cation. In certain embodiments, the cation is selected from the group consisting of calcium and magnesium.

In certain embodiments, the pharmaceutical composition, prior to administration, is disposed in a sterile container (e.g., bottle or vial).

In certain embodiments, the method comprises administering an effective amount of the pharmaceutical composition to the subject.

In certain embodiments, the disclosure relates to a method of removing sialic acid from a cell in a subject, the method comprising administering to the subject an effective amount of the pharmaceutical composition thereby to remove sialic acid from the cell.

In certain embodiments, the cell is a tumor cell, dendritic cell (DC) or monocyte. In certain embodiments, the cell is a monocyte, and the method results in increased expression of an MHC-II molecule on the monocyte.

In certain embodiments, the disclosure relates to a method of increasing phagocytosis of a tumor cell in a subject, the method comprising administering to the subject an effective amount of the pharmaceutical composition in an amount effective to remove sialic acid from the tumor cell, thereby increasing phagocytosis of the tumor cell.

In certain embodiments, the disclosure relates to a method of activating a dendritic cell (DC) in a subject, the method comprising administering to the subject an amount of the pharmaceutical composition effective to remove sialic acid from a tumor cell in the subject, thereby to activate the DC in the subject.

In certain embodiments, the disclosure relates to a method of reducing Siglec-15 binding activity, thereby increasing anti-tumor activity in a tumor microenvironment of a patient, the method comprising administering to the subject an effective amount of the pharmaceutical composition, thereby increasing anti-tumor activity (e.g., T cell activity) in the subject.

In another aspect, the invention provides a method of expressing a recombinant sialidase. The method can include (a) providing a cell comprising a nucleic acid encoding the recombinant sialidase and (b) expressing the recombinant sialidase in the presence of a stabilizing agent. In certain embodiments, the method further includes purifying the recombinant sialidase produced in step (b). The purification can be performed in the presence of a stabilizing agent, such as a cation (e.g., calcium or magnesium).

These and other aspects and features of the invention are described in the following detailed description and claims.

### DESCRIPTION OF THE DRAWINGS

The invention can be more completely understood with reference to the following drawings.

FIG. 1 depicts different configurations for sialidase-Fc fusion constructs. Sialidase-Fc fusion constructs can comprise a first polypeptide comprising a first immunoglobulin Fc domain (“Fc domain”), and a second polypeptide comprising a second immunoglobulin Fc domain. The first and second polypeptides can be covalently linked together, e.g., by disulfide bond(s). FIG. 1A shows a construct having two Fc domains and a sialidase enzyme conjugated to the N-terminus of each Fc domain. FIG. 1B shows a construct having two Fc domains and a sialidase enzyme conjugated to the C-terminus of the first Fc domain and the N-terminus of the second Fc domain. FIG. 1C shows a construct having two Fc domains and a sialidase enzyme conjugated to the N-terminus of the second Fc domain. FIG. 1D shows a construct having two Fc domains and a sialidase enzyme conjugated to the C-terminus of the first Fc domain. FIG. 1E shows a construct having two Fc domains and a sialidase enzyme conjugated to the C-terminus of the each Fc domain. It is understood that the Fc domains can be naturally occurring Fc domains or engineered Fc domains containing modifications, such as, point mutations in each polypeptide chain that facilitates a knob into hole configuration, or to provide a modified Fc domain functionality.

FIG. 2 depicts an SDS-PAGE gel showing recombinant human Neu1, Neu2, Neu3, and Salmonella typhimurium (ST-sialidase) under non-reducing and reducing conditions. Monomer and dimer species are indicated.

FIG. 3 is a bar graph showing the enzymatic activity of recombinant human Neu1, Neu2, and Neu3.

FIG. 4 is a line graph showing enzymatic activity as a function of substrate concentration for recombinant human Neu2 and Neu3 at the indicated pH.

FIG. 5A depicts an SDS-PAGE gel showing recombinant wildtype human Neu2-Fc and the Neu2-Fc variant M106 (“M106”) under non-reducing and reducing conditions.

FIGS. 5B and 5C show SEC-HPLC traces comparing wildtype Neu2-Fc versus M106, wherein the monomer species has a retention time of 21 minutes.

FIG. 6 is a line graph showing the enzymatic activity as a function of substrate concentration for M106.

FIG. 7 is a bar graph showing the enzymatic activity of Neu3-Fc in the supernatant (“Supernatant”) or membrane-bound (“Washed Cells”) Expi293 cells.

FIG. 8 is an SEC-HPLC trace of Fc-ST Sialidase, wherein the monomer species has a retention time of 21 minutes.

FIGS. 9A-D are a series of line graphs showing tumor volume in a mouse A20 (lymphoma) syngeneic tumor model. Mice were administered a negative control (“Isotype Control,” FIG. 9A), Fc-ST Sialidase (FIG. 9B), Avelumab (anti-mouse PD-L1 antibody, FIG. 9C), or the combination of Fc-ST Sialidase and Avelumab (FIG. 9D) at 10 mg/kg twice a week for 15 days and tumor volume was measured over time. Administration of FC-ST Sialidase alone or in combination with Avelumab reduces tumor volume.

FIGS. 10A-D is a series of line graphs showing tumor volume in a mouse syngeneic tumor model utilizing EMT6 cells engineered for human Her2 expression. Mice were administered isotype control (Vehicle Control, FIG. 10A), Fc-ST Sialidase (FC-ST, FIG. 10B), trastuzumab (anti human Her2 antibody, FIG. 10C), or Fc human Sialidase (M106, FIG. 10D) for at 10 mg/kg twice a week for 15 days, as indicated by the triangles, and tumor volume was measured over time. Administration of Fc human Sialidase or Fc-ST Sialidase reduces tumor volume.

FIG. 11 is a bar graph showing that neuraminidase activity following incubation at 37° C. for up to 14 days is stabilized by the addition of CaCl₂.

FIG. 12A is a bar graph showing neuraminidase activity in conditioned media of cells expressing a human neuraminidase Fc construct at the indicated days following transfection in the presence or absence of 4 mM CaCl₂. As shown, the presence of CaCl₂ stabilizes activity. FIG. 12B is a bar graph depicting cell viability at the indicated days following transfection in the presence or absence of 4 mM CaCl₂.

FIG. 13A is a bar graph showing that neuraminidase activity is stabilized by CaCl₂ at different concentrations in conditioned media of cells expressing a human neuraminidase Fc construct. Enzyme activity at the indicated days following transfection in the presence of 0, 0.05, 0.5, 1, 2 and 4 mM CaCl₂ is shown. FIG. 13B shows total protein yield at day 6 in the presence of 0, 0.05, 0.5, 1, 2 and 4 mM CaCl₂.

FIG. 14 provides bar graphs depicting geometric mean fluorescence intensity (gMFIs) resulting from staining with Hydra-3 (FIG. 14A), Hydra-7 (FIG. 14B), and Hydra-9 (FIG. 14C) of different immune subset populations.

FIG. 15 provides bar graphs depicting geometric mean fluorescence intensity (gMFIs) resulting from staining with PNA (FIG. 15A), MAL-II (FIG. 15B), and SNA (FIG. 15C) of different immune subset populations.

FIG. 16 provides line graphs depicting the degree of desialylation of dendritic cells (DCs) by increasing concentrations of M106. FIG. 16A depicts mean fluorescence intensity (MFI) and FIG. 16B provides bar graphs depicting fold increase in desialylation compared to untreated DCs.

FIG. 17 provides line graphs the degree of desialylation of BT-20 (breast cancer) tumor cells following treatment with increasing concentrations of M106 (triangles) compared to LOF control (squares) as determined by Hydra 9 binding (FIG. 17A) or PNA binding (FIG. 17B), measured by gMFI.

FIG. 18 provides line graphs depicting the degree of desialylation of HT-29 tumor cells following treatment with increasing concentrations of M106 (triangles) compared to LOF control (squares) as determined by Hydra 9 binding (FIG. 18A) or PNA binding (FIG. 18B) and measured as gMFI.

FIG. 19 provides line graphs depicting the degree of desialylation of SK-BR-3 tumor cells following treatment with increasing concentrations of M106 (triangles) compared to LOF control (squares) as determined by Hydra 9 binding (FIG. 19A), MAL-II binding (FIG. 19B) or PNA binding (FIG. 19C) and measured as gMFI.

FIG. 20 provides bar graphs depicting the percent increase in CD83hi expression (FIG. 20A) and CD86hi expression (FIG. 20B) on DCs following incubation with SKBR3 tumor cells treated with or without M106 in the presence or absence of lipopolysaccharide (LPS) treatment (open bars versus filled bars).

FIG. 21 depicts the dose-dependent enhancement of phagocytosis by M2-like macrophages of HT-29 tumor cells that were desialylated by M106 or LOF, as indicated. Tumor cells were derived from two different healthy donors (FIG. 21A and FIG. 21B). A similar increase in phagocytosis of desialylated BT20 and SKBR-3 tumor cells by M2 like macrophages is depicted in FIG. 21C and FIG. 21D respectively.

FIG. 22 provides bar graphs depicting the dose-dependent enhancement of HLA-DR expression following desialylation of monocytes by M106 or LOF control. Monocytes were obtained from two different healthy donors (FIG. 22A and FIG. 22B).

FIG. 23 provides tumor growth curves depicting the in vivo activity of sialidases of the current disclosure in a mouse MC38 syngeneic tumor model. Tumor growth curves for individual mice are shown for isotype control treated mice (FIG. 23A), M106-treated mice (FIG. 23B), anti-PD-1 treated mice (FIG. 23C) or mice treated with a combination of M106 and anti-PD-1 (FIG. 23D). Triangles indicate administration times of test articles.

FIG. 24 provides tumor growth curves depicting the in vivo activity of sialidases of the current invention in a mouse B16F10 syngeneic tumor model. Tumor growth curves for individual mice are shown for isotype control treated mice (FIG. 24A), M106 treated mice (FIG. 24B) or anti-PD-1 treated mice (FIG. 24C). FIG. 24D is an overlay of the tumor growth curves for isotype control group and the M106 group. Triangles indicate administration times of test articles.

FIG. 25 provides tumor growth curves depicting the in vivo activity of sialidases of the current invention in a mouse EMT6 syngeneic tumor model. Tumor growth curves for each individual mouse is shown for Isotype control treated mice (FIG. 25A) or M106 treated mice (FIG. 25B). Triangles indicate administration times of test articles.

FIG. 26 depicts the in vivo efficacy of M106 alone or in combination with avelumab (“Ave”) at the indicated dose in a mouse A20 syngeneic subcutaneous tumor model. Tumor growth curves for each mouse are depicted. Observed partial responses (PR) and complete responses (CR) are also indicated.

FIG. 27 depicts the in vivo efficacy of M106 alone or in combination with avelumab at the indicated dose in a mouse A20 syngeneic subcutaneous tumor model. Tumor growth curves for each mouse are depicted. Triangles indicate dosing.

FIG. 28 depicts the in vivo activity of ofatumumab, a combination of ofatumumab and Neu2-M106-Fc (“M106 FC”), and an isotype control in a syngeneic EL4-CD20 lymphoma intravenous dissemination model as of day 28 (FIG. 28A) or at the end of in-life as of day 41 (FIG. 28B). Triangles indicate dosing of various test articles. P-value was calculated by Log-rank (Mantle-Cox) test.

FIG. 29 depicts the results of Siglec-15-Fc staining of CD4+ cells (FIG. 29A) and CD8+ cells (FIG. 29B) following no treatment (“none”), treatment with a loss of function sialidase (“LOF FC”), or treatment with a sialidase (M106 (“M106 FC”) or BiNaNH2 (positive control)). As a negative control, Isotype IgG1 staining is also shown. As shown, treatment of activated CD4 and CD8 cells with M106 or BiNaNH2 decreased Siglec-15-Fc staining as compared to no treatment or treatment with a loss of function sialidase. Bar graphs showing levels of fluorescence (gMFI) and the underlying flow cytometry histogram data are provided in each figure.

FIG. 30 depicts the results of Siglec-15-Fc staining of CD4+ cells (FIG. 30A) and CD8+ cells (FIG. 30B) using the same methods as in FIG. 30A-B, with PBMCs from a second healthy donor.

### DETAILED DESCRIPTION

The invention is based, in part, upon the discovery that it is possible to treat a sialic acid-mediated disorder by administering a sialidase enzyme or a sialidase enzyme conjugated to a serum half-life enhancer. Surprisingly, it has been discovered that a sialidase or a sialidase enzyme conjugated to a serum half-life enhancer that lacks a targeting moiety (e.g., an antibody binding domain directed to a tumor antigen) can effectively treat a sialic acid-mediated disorder (e.g., cancer, e.g., a solid tumor) in vivo. As a result, the constructs described herein can be used on their own to treat a sialic acid-medicated disorder, e.g., cancer, or they can be used in combination with another agent, e.g., an anti-cancer agent, to treat the disorder, e.g., cancer. For example, when used in combination with another anti-cancer agent, the constructs can enhance the activity of the anti-cancer agent, for example, by making the cancer more susceptible to treatment with the anti-cancer agent.

The invention further relates to recombinant forms of sialidase enzymes, sialidase enzymes conjugated to a serum half-life enhancer, and pharmaceutical compositions thereof, that have suitable substrate specificities and activities to be useful in removing sialic acid and/or sialic acid containing molecules from the surface of cancer cells and/or removing sialic acid and/or sialic acid containing molecules from the tumor microenvironment, and/or reducing the concentration of sialic acid and/or sialic acid containing molecules in the tumor microenvironment.

The invention further relates to pharmaceutical compositions and methods of using sialidase or sialidase conjugated to a half-life extender to treat cancer, e.g., a solid tumor, soft tissue tumor, hematopoietic tumor, metastatic lesion, or an epithelial cell cancer.

Various features and aspects of the invention are discussed in more detail below.

### I. Recombinant Sialidases

As used herein, the term “sialidase” refers to any enzyme, or a functional fragment or variant thereof, that cleaves a terminal sialic acid residue from a substrate, for example, a glycoprotein or a glycolipid. The term sialidase includes variants having one or more amino acid substitutions, deletions, or insertions relative to a wild-type sialidase sequence, and/or fusion proteins or conjugates including a sialidase. Sialidases are also called neuraminidases, and, unless indicated otherwise, the two terms are used interchangeably herein. As used herein, the term “functional fragment” of a sialidase refers to fragment of a full-length sialidase that retains, for example, at least 10%, at least 20%, at least 30%, at least 40%, at least 50%, at least 60%, at least 70%, at least 80%, at least 90%, at least 95%, or 100% of the enzymatic activity of the corresponding full-length, naturally occurring sialidase. Sialidase enzymatic activity may be assayed by any method known in the art, including, for example, by measuring the release of sialic acid from the fluorogenic substrate 4-methylumbelliferyl-N-acetylneuraminic acid (4MU-NeuAc). In certain embodiments, the functional fragment comprises at least 100, 150, 200, 250, 300, 310, 320, 330, 340, 350, 360, or 370 consecutive amino acids present in a full-length, naturally occurring sialidase.

The sialidase described herein can be any sialidase, e.g., a viral, fungal, bacterial, non-human mammalian or human sialidase. In certain embodiments, the sialidase is a recombinant human sialidase comprising at least one mutation relative to a wild-type human sialidase, e.g., a substitution, deletion, or addition of at least one amino acid, as described above.

In certain embodiments, the sialidase is any recombinant mutant human sialidase disclosed herein, or a functional fragment thereof.

In certain embodiments, the sialidase comprises a C332A and C352L mutation. In certain embodiments, the sialidase comprises an N-terminal addition of MEDLRP (SEQ ID NO: 4) or EDLRP (SEQ ID NO: 3). In certain embodiments, the sialidase comprises a LSHSLST (SEQ ID NO: 22) peptide on the N-terminus. In certain embodiments, the sialidase comprises an N-terminal addition of MEDLRP (SEQ ID NO: 4) and an A2K substitution. In certain embodiments, the sialidase comprises an N-terminal addition of MEDLRP (SEQ ID NO: 4) and a C332A substitution. In certain embodiments, the sialidase comprises an N-terminal addition of MEDLRP (SEQ ID NO: 4), a C332A substitution, and a C352L substitution.

In certain embodiments, the sialidase portion comprises an M1 deletion (ΔM1), M1A substitution, M1D substitution, V6Y substitution, K9D substitution, P62G substitution, P62N substitution, P62S substitution, P62T substitution, A93E substitution, Q126Y substitution, I187K substitution, A242T substitution, Q270A substitution, Q270T substitution, S301R substitution, S301R substitution, W302K substitution, W302R substitution, C332A substitution, V363R substitution, L365I substitution, or a combination of any of the foregoing.

In certain embodiments, the sialidase comprises the amino acid sequence of any one of SEQ ID NOs: 48-62, 169-171, or 196, or an amino acid sequence that has at least 85%, 90%, 95%, 96%, 97%, 98%, or 99% sequence identity to any one of 48-62, 169-171, or 196.

a. Viral Sialidases

Exemplary viral sialidases include Influenza A virus surface glycoprotein neuraminidase (e.g., NCBI accession no. ACY01419.1, SEQ ID NO: 63), Influenza B virus surface glycoprotein neuraminidase (e.g., NCBI accession no. AIX94926.1, SEQ ID NO: 64), or an Influenza C virus surface glycoprotein neuraminidase, or a variant or functional fragment thereof. Other exemplary viral sialidases include Paramyxoviridae Respirovirus Parainfluenzavirus type 1 & 3 (e.g., NCBI accession no. BAD89145.1, SEQ ID NO: 65), Bovine Parainfluenza virus type 3 (e.g., NCBI accession no. ADQ43755, SEQ ID NO: 66), Sendai virus (e.g., UniProtKB accession P04853.1, SEQ ID NO: 67), Rubulavirus, Mumps virus, Simian virus 5, and Parainfluenza virus type 2 & 4a, 4b.

b. Prokaryotic Sialidases

Exemplary prokaryotic sialidases include sialidases from Salmonella typhimurium and Vibrio cholera. The amino acid sequence of Salmonella typhimurium sialidase (St-sialidase) is depicted in SEQ ID NO: 30, and a nucleotide sequence encoding Salmonella typhimurium sialidase is depicted in SEQ ID NO: 6. The amino acid sequence of Vibrio cholera sialidase is depicted in SEQ ID NO: 36, and a nucleotide sequence encoding Vibrio cholera sialidase is depicted in SEQ ID NO: 37.

Other exemplary prokaryotic sialidases include sialidases from Actinomyces viscosus (Avis_NanH; Uniprot accession no. AAA21932, SEQ ID NO:68); Arthrobacter nicotianae NA1 and NA2; sialidases from Arthrobacter sialophilus; Arthrobacter ureafaciens L, M1, M2 and S (GenBank accession no. BAD66680, SEQ ID NO:69); sialidases from Bacteroides fragilis; sialidases from Clostridium chauvoei; i A99 NanH (GenBank accession no. CAA50436, SEQ ID NO:70), NanI (GenBank accession no. ABG83208, SEQ ID NO:71), NanJ (GenBank accession no. ABG84247, SEQ ID NO:72); sialidases from Clostridium septicum (e.g., GenBank accession no. CAA44916.1, SEQ ID NO: 107); sialidases from Clostridium sordellii; sialidases from Clostridium tertium (e.g., GenBank accession no. CAA69951, SEQ ID NO: 73); sialidases from Corynebacterium diphtheriae (e.g., GenBank accession no. ACS34893, SEQ ID NO: 74); sialidases from Haemophilus parasuis; sialidases from Micromonospora viridifaciens (e.g., GenBank accession no. BAA00852, SEQ ID NO: 75); Pasteurella multocida NanH (GenBank accession no. AAG35310.1, SEQ ID NO: 76) and NanB (AAG35309, SEQ ID NO: 77); sialidases from Pseudomonas Aeruginosa (e.g., GenBank accession no. AAG06182, SEQ ID NO: 78); sialidases from Salmonella Typhimurium (e.g., GenBank accession no. NP 459905, SEQ ID NO: 79); Streptococcus pneumoniae NanA (GenBank accession no. P62575, SEQ ID NO: 108), NanB (GenBank accession no. AAC44396, SEQ ID NO: 80) and NanC; sialidases from Tannerella forsythia (e.g., GenBank accession no. TF0035, SEQ ID NO: 81; sialidases from Vibrio cholerae (e.g., GenBank accession no. YP_001217324, SEQ ID NO: 82), sialidases from C. diphtheriae (C. diphtheriae KCTC3075 NanH, designated as Cdip_NanH (GenBank accession number ACS34893, SEQ ID NO: 83) and its homologues; Corynebacterium glutamicum R hypothetical protein (Cglu_hypP; YP_001138502, SEQ ID NO: 84); C. perfringens NCTC 8239 sialidase I (Cper_NanI; ZP_02643014, SEQ ID NO: 85); B. fragilis YCH46 sialidase (Bfra_NanH; Uniprot accession no. BAA05853, SEQ ID NO:86); M. viridifaciens sialidase (Mvir_NanH; Uniprot accession no. BAA0085, SEQ ID NO: 87); S. pneumoniae NanA sialidase (Spne_NanA; P62575, SEQ ID NO: 88); Streptomyces coelicolor A3(2) sialidase (Scoe_NanH; NP_630638, SEQ ID NO: 89); Streptomyces griseus NBRC 13350 sialidase (Sgri_NanH; YP_001827941, SEQ ID NO: 90); Propionibacterium acnes SK137 sialidase (Pacn_NanH; ZP_03389398, SEQ ID NO: 91); Macrobdella decora trans-sialidase (Mdec_NanL; AAC47263, SEQ ID NO: 92); T. cruzi trans-sialidase (Tcru_TS; GenBank accession no. AAA99442, SEQ ID NO:93); Akkermansia muciniphila (ATCC BAA-835/DSM 22959) Amuc_0625/Am0707 (Uniprot accession no. B2UPI5, SEQ ID NO: 94); B. fragilis TAL2480 YCH46 sialidase (GenBank accession no. BF1729, SEQ ID NO: 95) (P31206); B. fragilis SBT3182; B. fragilis 4852; B. fragilis YM4000; B. thetaiotaomicron VPI-5482 sialidase (BtsA;BTSA;BT0455) (GenBank accession no. Q8AAK9, SEQ ID NO: 96); B. vulgatus ATCC 8482/DSM 1447/NCTC 11154 BVU 4143 (Uniprot accession no. A6L7T1, SEQ ID NO:97); B. bifidum JCM 1254 exo-α-sialidase (SiaBb2;BBP 0054) (GenBank accession no. BAK26854.1, SEQ ID NO: 98); C. perfringens A99 sialidase 1 ‘small’ (P10481, SEQ ID NO: 99); C. perfringens ATCC 10543 sialidase 2 (NanH) (Uniprot accession no. Q59311, SEQ ID NO: 100); C. perfringens ATCC 13124 sialidase (CPF 0721) (Uniprot accession no. QOTT67, SEQ ID NO: 101); C. perfringens str 13 exo-α-sialidase (NanI;CPSA;CPE0725) (Uniprot accession no. Q8XMG4, SEQ ID NO: 102); C. perfringens str 13/ATCC 13124 exo-α-sialidase (NanJ;CPE0553 (Uniprot accession no. Q8XMY5, SEQ ID NO: 103); Clostridium tertium ATCC 14573 sialidase (NanH;SiaH) (Uniprot accession no. P77848, SEQ ID NO: 104); R. gnavus ATCC 29149 RgNanH (Uniprot accession no. A7B557, SEQ ID NO: 105); S. typhimurium TA262/LT2 sialidase (NanH;STSA) (P29768, SEQ ID NO: 106).

Other exemplary sialidases include Sialidases or neuraminidases from A. castellani, A. polyphaga, A. culbertsoni, A. astronyxis, A. hatchetti, A. palestinensis, A. rhysodes, E. tenella, E. maxima, E. necatrix, E. Spec, T. brucei, and T. rangeli.

c. Mouse Sialidases

Four sialidases have also been found in the mouse genome and are referred to as Neu1, Neu2, Neu3 and Neu4. The amino acid sequence of mouse Neu1 is depicted in SEQ ID NO: 38, and a nucleotide sequence encoding mouse Neu1 is depicted in SEQ ID NO: 42. The amino acid sequence of mouse Neu2 is depicted in SEQ ID NO: 39 and a nucleotide sequence encoding mouse Neu2 is depicted in SEQ ID NO: 43. The amino acid sequence of mouse Neu3 is depicted in SEQ ID NO: 40, and a nucleotide sequence encoding mouse Neu3 is depicted in SEQ ID NO: 44. The amino acid sequence of mouse Neu4 is depicted in SEQ ID NO: 41, and a nucleotide sequence encoding mouse Neu4 is depicted in SEQ ID NO: 45.

d. Human Sialidases

Four sialidases have also been found in the human genome and are referred to as Neu1, Neu2, Neu3 and Neu4.

Human Neu1 is a lysosomal neuraminidase enzyme which functions in a complex with beta-galactosidase and cathepsin A. The amino acid sequence of human Neu1 is depicted in SEQ ID NO: 7, and a nucleotide sequence encoding human Neu1 is depicted in SEQ ID NO: 23.

Human Neu2 is a cytosolic sialidase enzyme. The amino acid sequence of human Neu2 is depicted in SEQ ID NO: 1, and a nucleotide sequence encoding human Neu2 is depicted in SEQ ID NO: 24.

Human Neu3 is a plasma membrane sialidase with an activity specific for gangliosides. Human Neu3 has two isoforms: isoform 1 and isoform 2. The amino acid sequence of human Neu3, isoform 1 is depicted in SEQ ID NO: 8, and a nucleotide sequence encoding human Neu3, isoform 1 is depicted in SEQ ID NO: 25. The amino acid sequence of human Neu3, isoform 2 is depicted in SEQ ID NO: 9, and a nucleotide sequence encoding human Neu3, isoform 2 is depicted in SEQ ID NO: 34.

Human Neu4 has two isoforms: isoform 1 is a peripheral membrane protein and isoform 2 localizes to the lysosome lumen. The amino acid sequence of human Neu4, isoform 1 is depicted in SEQ ID NO: 10, and a nucleotide sequence encoding human Neu4, isoform 1 is depicted in SEQ ID NO: 26. The amino acid sequence of human Neu4, isoform 2 is depicted in SEQ ID NO: 11, and a nucleotide sequence encoding human Neu4, isoform 2 is depicted in SEQ ID NO: 35.

In certain embodiments, a recombinant mutant human sialidase has about 5%, about 10%, about 15%, about 20%, about 25%, about 30%, about 35%, about 40%, about 45%, about 50%, about 60%, about 65%, about 70%, about 75%, about 80%, about 85%, about 90%, about 95%, about 100%, or more than 100% of the enzymatic activity of a corresponding (or template) wild-type human sialidase.

In certain embodiments, the recombinant mutant human sialidase has the same substrate specificity as the corresponding wild-type human sialidase. In other embodiments, the recombinant mutant human sialidase has a different substrate specificity than the corresponding wild-type human sialidase. For example, in certain embodiments the recombinant mutant human sialidase can cleave α2,3, α2,6, and/or α2,8 linkages. In certain embodiments the sialidase can cleave α2,3 and α2,8 linkages.

In certain embodiments, the expression yield of the recombinant mutant human sialidase in mammalian cells, e.g., HEK293 cells, CHO cells, murine myeloma cells (NS0, Sp2/0), or human fibrosarcoma cells (HT-1080), e.g., HEK293 cells, is greater than about 10%, about 20%, about 50%, about 75%, about 100%, about 150%, about 200%, about 250%, about 300%, about 400%, about 500%, about 600%, about 700%, about 800%, about 900%, or about 1,000% of the expression yield of the corresponding wild-type human sialidase.

In certain embodiments, the recombinant mutant human sialidase has about 5%, about 10%, about 15%, about 20%, about 25%, about 30%, about 35%, about 40%, about 45%, about 50%, about 60%, about 65%, about 70%, about 75%, about 80%, about 85%, about 90%, about 95%, about 100%, or more than 100% of the enzymatic activity of a corresponding wild-type human sialidase, and the expression yield of the recombinant mutant human sialidase in mammalian cells, e.g., HEK293 cells, is greater than about 10%, about 20%, about 50%, about 75%, about 100%, about 150%, about 200%, about 250%, about 300%, about 400%, about 500%, about 600%, about 700%, about 800%, about 900%, or about 1,000% of the expression yield of a corresponding wild-type human sialidase.

In certain embodiments, the amino acid sequence of the recombinant mutant human sialidase has at least 60%, 65%, 70%, 75%, 80%, 85%, 90%, 95%, 96%, 97%, 98%, or 99% sequence identity to the amino acid sequence of a corresponding wild-type human sialidase.

It is understood that the sialidases described herein, for example, the human sialidases, can be modified to enhance one or more properties of the enzyme, e.g., to improve expression, activity, stability (e.g., improve resistance to protease degradation). Some of these properties are applicable to the various sialidases described herein, e.g., the improved resistance to protease degradation.

i. Substitution of Cysteine Residues

In certain embodiments, the recombinant mutant human sialidase comprises a substitution of at least one cysteine (cys, C) residue. It has been discovered that certain cysteine residues in sialidases may inhibit expression of functional protein as a result of protein aggregation. Accordingly, in certain embodiments, the recombinant mutant human sialidase contains at least one mutation to remove a free cysteine (e.g., for Neu1 (SEQ ID NO: 7), a mutation of one or more of C111, C117, C171, C183, C218, C240, C242, and C252; for Neu2 (SEQ ID NO: 1), a mutation of one or more of C125, C196, C219, C272, C332, and C352; for Neu3 (SEQ ID NO: 8), a mutation of one or more of C7, C90, C99, C106, C127, C136, C189, C194, C226, C242, C250, C273, C279, C295, C356, C365, C368, C384, C383, C394, and C415; and for Neu4 (SEQ ID NO: 10), a mutation of one or more of C88, C125, C126, C186, C191, C211, C223, C239, C276, C437, C453, C480, and C481). Free cysteines can be substituted with any amino acid. In certain embodiments, the free cysteine is substituted with serine (ser, S), isoleucine (iso, I), valine (val, V), phenylalanine (phe, F), leucine (leu, L), or alanine (ala, A). Exemplary cysteine substitutions in Neu2 include C125A, C1251, C125S, C125V, C196A, C196L, C196V, C272S, C272V, C332A, C332S, C332V, C352L, and C352V.

In certain embodiments, the recombinant mutant human sialidase comprises two or more cysteine substitutions. Exemplary double or triple substitutions in Neu2 include: C125S and C332S; C272V and C332A; C272V and C332S; C332A and C352L; C125S and C196L; C196L and C352L; C196L and C332A; C332A and C352L; and C196L, C332A and C352L.

In certain embodiments, the recombinant mutant human sialidase is a Neu2 sialidase and comprises the substitutions C322A and C352L (SEQ ID NO: 5).

In certain embodiments, the sialidase contains an amino acid substitution at 2, 3, 4, 5, or 6 cysteines typically present in a human sialidase, e.g., Neu2 or Neu3.

In certain embodiments, the recombinant mutant human sialidase comprises a substitution or combination of substitutions corresponding to a substitution or combination of substitutions listed in TABLE 1 (amino acid positions corresponding to wild-type human Neu2 (SEQ ID NO: 1)).

TABLE 1
Substitution(s)

C125A
C125I
C125S
C125V
C196A
C196L
C196V
C272S
C272V
C332A
C332S
C332V
C352L
C352V
C125S + C332S
C272V + C332A
C272V + C332S
C332A + C352L
C125S + C196L
C196L + C352L
C196L + C332A
C196L + C332A + C352L

ii. Substitutions of Residues to Increase pI and/or Decrease Hydrophobicity

The isoelectric point (pI) of a protein is the pH at which the net charge is zero. The pI also indicates the pH at which the protein is least soluble, which affects the ability to express and purify the protein. Generally, a protein has good solubility if its pI is greater than 2 units above the pH of the solution. Human Neu2 has a predicted pI of 7.5. Thus, human Neu2 is least soluble around neutral pH, which is undesirable because expression and physiological systems are at neutral pH. In contrast, the sialidase from Salmonella typhimurium (St-sialidase), which exhibits good solubility and recombinant expression, has a pI of 9.6. Accordingly, to increase expression of human Neu2 or the other human sialidases, a recombinant mutant human sialidase may be designed to contain one or more amino acid substitution(s) wherein the substitution(s) increase(s) the pI of the sialidase relative to a sialidase without the substitution. Additionally, decreasing the number of hydrophobic amino acids on the surface of a sialidase may improve expression of sialidase by, for example, reducing aggregation. Accordingly, to increase expression of human Neu2 or the other human sialidases, a recombinant mutant human sialidase may be designed to contain one or more amino acid substitution(s) wherein the substitution(s) decrease(s) the hydrophobicity of a surface of the sialidase relative to a sialidase without the substitution(s).

Accordingly, in certain embodiments, the recombinant mutant human sialidase comprises at least one amino acid substitution, wherein the substitution increases the isoelectric point (pI) of the sialidase and/or decreases the hydrophobicity of the sialidase relative to a sialidase without the substitution. This may be achieved by introducing one or more charged amino acids, for example, positively or negatively charged amino acids, into the recombinant sialidase. In certain embodiments, the amino acid substitution is to a charged amino acid, for example, a positively charged amino acid such as lysine (lys, K), histidine (his, H), or arginine (arg, R), or a negatively charged amino acid such as aspartic acid (asp, D) or glutamic acid (glu, E). In certain embodiments, the amino acid substitution is to a lysine residue. In certain embodiments, the substitution increases the pI of the sialidase to about 7.75, about 8, about 8.25, about 8.5, about 8.75, about 9, about 9.25, about 9.5, or about 9.75.

In certain embodiments, the amino acid substitution occurs at a surface exposed D or E amino acid, in a helix or loop, or in a position that has a K or R in the corresponding position of St-sialidase. In certain embodiments, the amino acid substitution occurs at an amino acid that is remote from the catalytic site or otherwise not involved in catalysis, an amino acid that is not conserved with the other human Neu proteins or with an St-Sialidase or Clostridium NanH, or an amino acid that is not located in a domain important for function (e.g., an Asp-box or beta strand).

Exemplary amino acid substitutions in Neu2 that increase the isoelectric point (pI) of the sialidase and/or decrease the hydrophobicity of the sialidase relative to a sialidase without the substitution include A2E, A2K, D215K, V325E, V325K, E257K, and E319K. In certain embodiments, the recombinant mutant human sialidase comprises two or more amino acid substitutions, including, for example, A2K and V325E, A2K and V325K, E257K and V325K, A2K and E257K, and E257K and A2K and V325K.

In certain embodiments, the recombinant mutant human sialidase comprises a substitution or combination of substitutions corresponding to a substitution or combination of substitutions listed in TABLE 2 (amino acid positions corresponding to wild-type human Neu2 (SEQ ID NO: 1)).

TABLE 2
Substitution(s)
A2K
E72K
D215K
E257K
V325K
A2K + E257K
A2K + V325E
A2K + V325K
E257K + V325K

iii. Addition of N-terminal Peptides and N- or C-terminal Substitutions

It has been discovered that the addition of a peptide sequence of two or more amino acids to the N-terminus of a human sialidase can improve expression and/or activity of the sialidase. In certain embodiments, the peptide is at least 2 amino acids in length, for example, from 2 to 20, from 2 to 10, from 2 to 5, or 2, 3, 4, 5, 6, 7, 8, 9, 10, 11, 12, 13, 14, 15, 16, 17, 18, 19, or 20 amino acids in length. In certain embodiments, the peptide may form, or have a propensity to form, an α-helix.

In mice, a Neu2 isoform (type B) found in thymus contains six amino acids not present in the canonical isoform of Neu2 found in skeletal muscle. In certain embodiments herein, the N-terminal six amino acids of the mouse thymus Neu2 isoform, MEDLRP (SEQ ID NO: 4), or variations thereof, can be added onto a human Neu, e.g., human Neu2. In certain embodiments, the recombinant mutant human sialidase comprises a peptide at least two amino acid residues in length covalently associated with an N-terminal amino acid of the sialidase. In certain embodiments the recombinant mutant human sialidase comprises the peptide MEDLRP (SEQ ID NO: 4) or EDLRP (SEQ ID NO: 3) covalently associated with an N-terminal amino acid of the sialidase. In certain embodiments, the sialidase may further comprise a cleavage site, e.g., a proteolytic cleavage site, located between the peptide, e.g., MEDLRP (SEQ ID NO: 4) or EDLRP (SEQ ID NO: 3), and the remainder of the sialidase. In certain embodiments, the peptide, e.g., MEDLRP (SEQ ID NO: 4) or EDLRP (SEQ ID NO: 3), may be post-translationally cleaved from the remainder of the sialidase.

Alternatively to, or in combination with, the N-terminal addition, 1-5 amino acids of the 12 amino acid N-terminal region of the recombinant mutant human sialidase may be removed, e.g., the N-terminal methionine can be removed. In certain embodiments, if the recombinant mutant human sialidase is Neu2, the N-terminal methionine can be removed, the first five amino acids (MASLP; SEQ ID NO: 12) can be removed, or the second through fourth amino acids (ASLP; SEQ ID NO: 13) can be removed.

In certain embodiments, 1-5 amino acids of the 12 amino acid N-terminal region of the recombinant mutant human sialidase are substituted with MEDLRP (SEQ ID NO: 4), EDLRP (SEQ ID NO: 3), or TVEKSVVF (SEQ ID NO: 14). For example, in certain embodiments, if the recombinant mutant human sialidase is Neu2, the amino acids MASLP (SEQ ID NO: 12), ASLP (SEQ ID NO: 13) or M are substituted with MEDLRP (SEQ ID NO: 4), EDLRP (SEQ ID NO: 3) or TVEKSVVF (SEQ ID NO: 14).

Human sialidases have a β-propeller structure, characterized by 6 blade-shaped β-sheets arranged toroidally around a central axis. Generally, hydrophobic interactions between the blades of a β-propeller, including between the N- and C-terminal blades, enhance stability. Accordingly, in order to increase expression of human Neu2 or the other human sialidases, a recombinant mutant human sialidase can be designed comprising an amino acid substitution that increases hydrophobic interactions and/or hydrogen bonding between the N- and C-terminal β-propeller blades of the sialidase.

Accordingly, in certain embodiments, the recombinant mutant human sialidase comprises a substitution of at least one wild-type amino acid residue, wherein the substitution increases hydrophobic interactions and/or hydrogen bonding between the N- and C-termini of the sialidase relative to a sialidase without the substitution. In certain embodiments, the wild-type amino acid is substituted with asparagine (asn, N), lysine (lys, K), tyrosine (tyr, Y), phenylalanine (phe, F), or tryptophan (trp, W). Exemplary substitutions in Neu2 that increase hydrophobic interactions and/or hydrogen bonding between the N- and C-termini include L4N, L4K, V6Y, L7N, L4N and L7N, L4N and V6Y and L7N, V12N, V12Y, V12L, V6Y, V6F, or V6W. In certain embodiments, the sialidase comprises the V6Y substitution.

In certain embodiments, the recombinant mutant human sialidase comprises a combination of the above substitutions. For example, a recombinant mutant human Neu2 sialidase can comprise the additional amino acids MEDLRP (SEQ ID NO: 4), EDLRP (SEQ ID NO: 3), or TVEKSVVF (SEQ ID NO: 14) at the N-terminus and, in combination, can comprise at least one L4N, L4K, V6Y, L7N, L4N and L7N, L4N and V6Y and L7N, V12N, V12Y, V12L, V6Y, V6F, or V6W substitution. In certain embodiments, the amino acids MASLP (SEQ ID NO: 12), ASLP (SEQ ID NO: 13) or M of a recombinant mutant human Neu2 sialidase are replaced with MEDLRP (SEQ ID NO: 4), EDLRP (SEQ ID NO: 3) or TVEKSVVF (SEQ ID NO: 14) and the recombinant mutant human Neu2 sialidase also comprises at least one L4N, L4K, V6Y, L7N, L4N and L7N, L4N and V6Y and L7N, V12N, V12Y, V12L, V6Y, V6F, or V6W substitution.

In certain embodiments, the recombinant mutant human sialidase comprises a mutation or combination of mutations corresponding to a mutation or combination of mutations listed in TABLE 3 (amino acid positions corresponding to wild-type human Neu2 (SEQ ID NO: 1)).

TABLE 3
Mutation(s)
Substitute M at the N-terminus with EDLRP (SEQ ID NO: 3)
Substitute M at the N-terminus with MEDLRP (SEQ ID NO: 4)
Insert MEDLRP (SEQ ID NO: 4) at the N-terminus
Substitute MASLP (SEQ ID NO: 12) at the N-terminus with
MEDLRP (SEQ ID NO: 4)
L4N
V6Y
L7N
V6F
V6W

Additionally, in certain embodiments, the sialidase comprises a substitution or deletion of an N-terminal methionine at the N-terminus of the sialidase. For example, in certain embodiments, the sialidase comprises a substitution of a methionine residue at a position corresponding to position 1 of wild-type human Neu2 (SEQ ID NO: 1), e.g., the methionine at a position corresponding to position 1 of wild-type human Neu2 is substituted by alanine (M1A) or aspartic acid (M1D). In other embodiments, the sialidase comprises a deletion of a methionine residue at a position corresponding to position 1 (ΔM1) of wild-type human Neu2 (SEQ ID NO: 1).

In certain embodiments, the recombinant mutant human sialidase comprises a substitution or combination of substitutions corresponding to a substitution or combination of substitutions listed in TABLE 4 (amino acid positions corresponding to wild-type human Neu2 (SEQ ID NO: 1)).

TABLE 4
Mutation(s)
Deletion of M1, V6Y, I187K
M1R, V6Y, I187K
M1H, V6Y, I187K
M1K, V6Y, I187K
M1D, V6Y, I187K
M1T, V6Y, I187K
M1N, V6Y, I187K
M1Q, V6Y, I187K
M1G, V6Y, I187K
M1A, V6Y, I187K
M1V, V6Y, I187K
M1L, V6Y, I187K
M1F, V6Y, I187K
M1Y, V6Y, I187K

d. Substitutions of Residues to Decrease Proteolytic Cleavage

It has been discovered that certain sialidases (e.g., human Neu2) are susceptible to cleavage by a protease (e.g., trypsin). As a result, proteolytic cleavage of the sialidase may occur during recombinant protein production, harvesting, purification, formulation, during administration to a subject, or after administration to a subject, or any combination of the foregoing. Accordingly, in certain embodiments, the recombinant mutant human sialidase comprises a substitution of at least one wild-type amino acid residue, wherein the substitution decreases cleavage of the sialidase by a protease (e.g., trypsin) relative to a sialidase without the substitution.

In certain embodiments, incubation of the recombinant mutant human sialidase with a protease (e.g., trypsin) results in from about 1% to about 50%, from about 1% to about 40%, from about 1%, to about 30%, from about 1% to about 20%, from about 1% to about 10%, from about 1% to about 5%, from about 5% to about 50%, from about 5% to about 40%, from about 5% to about 30%, from about 5% to about 20%, from about 5% to about 10%, from about 10% to about 50%, from about 10% to about 40%, from about 10% to about 30%, from about 10% to about 20%, from about 20% to about 50%, from about 20% to about 40%, from about 20% to about 30%, from about 30% to about 50%, from about 30% to about 40%, or from about 40% to about 50% of the proteolytic cleavage of a corresponding wild-type sialidase when incubated with the protease under the same conditions. In certain embodiments, incubation of the recombinant mutant human sialidase with a protease (e.g., trypsin) results in less than 50%, less than 40%, less than 30%, less than 10%, less than 5%, less than 3%, less than 1%, or less than 0.5% of the proteolytic cleavage of a corresponding wild-type sialidase when incubated with the protease under the same conditions. Proteolytic cleavage can be assayed by any method known in the art, including for example, by SDS-PAGE as described in Example 5 herein.

Exemplary substitutions that increase resistance to proteolytic cleavage include: (i) a substitution of an alanine residue at a position corresponding to position 242 of wild-type human Neu2 (SEQ ID NO: 1), e.g., a substitution by cysteine (A242C), phenylalanine (A242F), glycine (A242G), histidine (A242H), isoleucine (A242I), lysine (A242K), leucine (A242L), methionine (A242M), asparagine (A242N), glutamine (A242Q), arginine (A242R), serine (A242S), valine (A242V), tryptophan (A242W), or tyrosine (A242Y); (ii) a substitution of an arginine residue at a position corresponding to position 243 of wild-type human Neu2 (SEQ ID NO: 1), e.g., a substitution by glutamic acid (R243E), histidine (R243H), asparagine (R243N), glutamine (R243Q), or lysine (R243K); (iii) a substitution of a valine residue at a position corresponding to position 244 of wild-type human Neu2 (SEQ ID NO: 1), e.g., a substitution by isoleucine (V244I), lysine (V244K), or proline (V244P); or (iv) a combination of any of the foregoing. In certain embodiments, the recombinant mutant human sialidase comprises a substitution selected from A242C, A242F, A242Y, and A242W. In certain embodiments, the recombinant mutant human sialidase comprises a substitution or combination of substitutions corresponding to a substitution or combination of substitutions listed in TABLE 5 (amino acid positions corresponding to wild-type human Neu2 (SEQ ID NO: 1)).

TABLE 5

| Wild Type | |
|---|---|
| Human Neu2 | |
| (SEQ ID NO: 1) | |
| Amino Acid | Exemplary Substitution(s) at Specified Position(s) |
| A242 | C, F, G, H, I, K, L, M, N, P, Q, R, S, V, W, Y |
| R243 | E, H, N, Q, K |
| V244 | I, K, P |

Additional exemplary substitutions that increase resistance to proteolytic cleavage (and/or increase expression yield and/or enzymatic activity) include: (i) a substitution of a leucine residue at a position corresponding to position 240 of wild-type human Neu2 (SEQ ID NO: 1), e.g., a substitution by aspartic acid (L240D), asparagine (L240N), or tyrosine (L240Y); (ii) a substitution of an alanine residue at a position corresponding to position 213 of wild-type human Neu2 (SEQ ID NO: 1), e.g., a substitution by cysteine (A213C), asparagine (A213N), serine (A213S), or threonine (A213T); (iii) a substitution of an arginine residue at a position corresponding to position 241 of wild-type human Neu2 (SEQ ID NO: 1), e.g., a substitution by alanine (R241A), aspartic acid (R241D), leucine (R241L), glutamine (R241Q). or tyrosine (R241Y); (iv) a substitution of a serine residue at a position corresponding to position 258 of wild-type human Neu2 (SEQ ID NO: 1), e.g., a substitution by cysteine (S258C); (v) a substitution of a leucine residue at a position corresponding to position 260 of wild-type human Neu2 (SEQ ID NO: 1), e.g., a substitution by aspartic acid (L260D), phenylalanine (L260F), glutamine (L260Q), or threonine (L260T); (vi) a substitution of a valine residue at a position corresponding to position 265 of wild-type human Neu2 (SEQ ID NO: 1), e.g., a substitution by phenylalanine (V265F); or (vii) a combination of any of the foregoing. It is contemplated that, in certain embodiments, a substitution or a combination of substitutions at these positions may improve hydrophobic and/or aromatic interaction between secondary structure elements in the sialidase (e.g., between an α-helix and the nearest β-sheet) thereby stabilizing the structure and improving resistance to proteolytic cleavage.

In certain embodiments, the recombinant mutant sialidase comprises a mutation at position L240. In certain embodiments, the recombinant mutant sialidase comprises a combination of mutations at positions (i) A213 and A242, (ii) A213, A242, and S258, (iii) L240 and L260, (iv) R241 and A242, (v) A242 and L260, (vi) A242 and V265, and (vii) L240 and A242. In certain embodiments, the recombinant mutant human sialidase comprises a combination of substitutions selected from (i) A213C, A242F, and S258C, (ii) A213C and A242F, (iii) A213T and A242F, (iv) R241Y and A242F, or (v) L240Y and A242F. In certain embodiments, the recombinant mutant human sialidase comprises a substitution or combination of substitutions corresponding to a substitution or combination of substitutions listed in TABLE 6 (amino acid positions corresponding to wild-type human Neu2 (SEQ ID NO: 1)).

TABLE 6
Substitution(s)
A242C, V244P
A242R, V244R
A242R, V244H
A242Y, V244P
A242T, V244P
A242N, V244P
A213C, A242F
A213S, A242F
A213T, A242F
A213N, A242F
A213C, A242F, S258C
A242F, L260F
A242F, V265F
L240Y
L240Y, L260F
L240D, L260T
L240N, L260T
L240N, L260D
L240N, L260Q
L240Y, A242F
R241A, A242F
R241Y, A242F

iv. Other Substitutions

The invention further provides a recombinant mutant human sialidase comprising at least one of the following substitutions: I187K, A328E, K370N, or H210N. In certain embodiments, a recombinant mutant human Neu2 comprises the substitution of the amino acids GDYDAPTHQVQW (SEQ ID NO: 15) with the amino acids SMDQGSTW (SEQ ID NO: 16) or STDGGKTW (SEQ ID NO: 17). In certain embodiments, a recombinant mutant human Neu2 comprises the substitution of the amino acids PRPPAPEA (SEQ ID NO: 18) with the amino acids QTPLEAAC (SEQ ID NO: 19). In certain embodiments, a recombinant mutant human Neu2 comprises the substitution of the amino acids NPRPPAPEA (SEQ ID NO: 20) with the amino acids SQNDGES (SEQ ID NO: 21).

The invention further provides a recombinant mutant human sialidase comprising at least one substitution at a position corresponding to V212, A213, Q214, D215, T216, L217, E218, C219, Q220, V221, A222, E223, V224, E225, or T225.

The invention further provides a recombinant mutant human sialidase comprising an amino acid substitution at a position identified in TABLE 7 (amino acid positions corresponding to wild-type human Neu2 (SEQ ID NO: 1). In certain embodiments, the sialidase comprises an amino acid substitution identified in TABLE 7. In certain embodiments, the sialidase comprises a combination of any amino acid substitution identified in TABLE 7.

TABLE 7

| Wild Type | |
|---|---|
| Human Neu2 | |
| (SEQ ID NO: 1) | |
| Amino Acid | Substitution at Specified Position |
| M1 | D |
| L4 | S, T, Y, L, F, A, P, V, I, N, D, H |
| P5 | G |
| V6 | Y |
| L7 | F, Y, S, I, T, N |
| K9 | D |
| V12 | L, A, P, V, N, D, H |
| F13 | S, N, R, K, T, G, D, E, A |
| I22 | S, N, R, K, T, G, D, E, A, Y, L, F, P, V, I, H |
| A24 | S, N, R, K, T, G, D, E, A, Y, L, F, P, V, I, H |
| L34 | S, T, Y, L, F, A, P, V, I, N, D, H |
| A36 | S, T, Y, L, F, A, P, V, I, N, D, H |
| K44 | R, E |
| K45 | A, E, R |
| L54 | M |
| P62 | H, G, N, T, S, F, I, D, E |
| H64 | F, Y, S, I, T, N |
| Q69 | H |
| R78 | K |
| D80 | P |
| P89 | S, T, Y, L, F, A, P, V, I, N, D, H, M |
| A93 | E,K |
| G107 | D |
| Q108 | H |
| Q112 | R, K |
| C125 | Y, F, L |
| Q126 | E, F, H, I, L, or Y |
| A150 | V |
| T156 | R, N, D, C, G, H, I, L, F, S, Y, V, A, P, T |
| F157 | R, N, D, C, G, H, I, L, F, S, Y, V, A, P |
| A158 | R, N, D, C, G, H, I, L, F, S, Y, V, A, P, T |
| V159 | R, N, D, C, G, H, I, L, F, S, Y, V, A, P |
| G160 | R, N, D, C, G, H, I, L, F, S, Y, V, A, P, T |
| P161 | R, N, D, C, G, H, I, L, F, S, Y, V, A, P |
| G162 | R, N, D, C, G, H, I, L, F, S, Y, V, A, P, T |
| H163 | R, N, D, C, G, H, I, L, F, S, Y, V, A, P |
| C164 | R, N, D, C, G, H, I, L, F, S, Y, V, A, P, T |
| L165 | R, N, D, C, G, H, I, L, F, S, Y, V, A, P |
| R170 | P |
| A171 | G |
| V176 | R, N, D, C, G, H, I, L, F, S, Y, V, P, A |
| P177 | S, T, Y, L, F, A, P, V, I, N, D, H |
| A178 | S, T, Y, L, F, A, P, V, I, N, D, H |
| L184 | S, N, R, K, T, G, D, E, A, F, H, I, L, P, V, Y |
| H185 | S, N, R, K, T, G, D, E, A |
| P186 | S, N, R, K, T, G, D, E, A, F, H, I, L, P, V, Y, |
| I187 | S, N, R, K, T, G, D, E, A |
| Q188 | P, S, N, R, K, T, G, D, E, A |
| R189 | P |
| P190 | F, M, A, D, G, H, N, P, R, S, T |
| I191 | M, A, D, F, H, I, L, N, P, S, T, V, Y, E, G, K, R |
| A194 | S, T, Y, L, F, A, P, V, I, N, D, H |
| A213 | C, N, S, or T |
| L217 | R, N, D, C, G, H, I, L, F, S, Y, V |
| C219 | R, N, D, C, G, H, I, L, F, S, Y, V |
| A222 | D |
| E225 | P |
| T249 | A |
| D251 | G |
| E257 | P |
| S258 | C |
| L260 | D, F,Q, or T |
| V265 | F |
| Q270 | S, T, A, H, P, F |
| G271 | S, N, R, K, T, G, D, E, A |
| C272 | S, N, R, K, T, G, D, E, A, C, H, Y, F, H, L, P, V |
| W292 | R |
| S301 | A, D, E, F, G, H, I, K, L, M, N, P, Q, T, V, W, Y, |
| | C, or R |
| W302 | A, D, E, F, G, H, I, L, M, N, P, Q, R, S, T, V, Y, or |
| | K |
| E319 | D |
| V325 | F, Y, S, I, T, N, A, D, H, L, P, V |
| L326 | F, Y, S, I, T, N, A, D, H, L, P, V |
| L327 | F, Y, S, I, T, N, A, D, H, L, P, V |
| C332 | A, D, G, H, N, P, R, S, T |
| Y359 | A, S |
| V363 | R, S, T, Y, L, F, A, P, V, I, N, D, H |
| L365 | K, Q, F, Y, S, I, T, N, A, D, H, L, P, V |

For example, in certain embodiments, the recombinant mutant human sialidase comprises: (a) a substitution of a proline residue at a position corresponding to position 5 of wild-type human Neu2 (P5); (b) a substitution of a lysine residue at a position corresponding to position 9 of wild-type human Neu2 (K9); (c) a substitution of a lysine residue at a position corresponding to position 44 of wild-type human Neu2 (K44); (d) a substitution of a lysine residue at a position corresponding to position 45 of wild-type human Neu2 (K45); (e) a substitution of a leucine residue at a position corresponding to position 54 of wild-type human Neu2 (L54); (f) a substitution of a proline residue at a position corresponding to position 62 of wild-type human Neu2 (P62); (g) a substitution of a glutamine residue at a position corresponding to position 69 of wild-type human Neu2 (Q69); (h) a substitution of an arginine residue at a position corresponding to position 78 of wild-type human Neu2 (R78); (i) a substitution of an aspartic acid residue at a position corresponding to position 80 of wild-type human Neu2 (D80); (j) a substitution of an alanine residue at a position corresponding to position 93 of wild-type human Neu2 (A93); (k) a substitution of a glycine residue at a position corresponding to position 107 of wild-type human Neu2 (G107); (1) a substitution of a glutamine residue at a position corresponding to position 108 of wild-type human Neu2 (Q108); (m) a substitution of a glutamine residue at a position corresponding to position 112 of wild-type human Neu2 (Q112); (n) a substitution of a cysteine residue at a position corresponding to position 125 of wild-type human Neu2 (C125); (o) a substitution of a glutamine residue at a position corresponding to position 126 of wild-type human Neu2 (Q126); (p) a substitution of an alanine residue at a position corresponding to position 150 of wild-type human Neu2 (A150); (q) a substitution of a cysteine residue at a position corresponding to position 164 of wild-type human Neu2 (C164); (r) a substitution of an arginine residue at a position corresponding to position 170 of wild-type human Neu2 (R170); (s) a substitution of an alanine residue at a position corresponding to position 171 of wild-type human Neu2 (A171); (t) a substitution of a glutamine residue at a position corresponding to position 188 of wild-type human Neu2 (Q188); (u) a substitution of an arginine residue at a position corresponding to position 189 of wild-type human Neu2 (R189); (v) a substitution of an alanine residue at a position corresponding to position 213 of wild-type human Neu2 (A213); (w) a substitution of a leucine residue at a position corresponding to position 217 of wild-type human Neu2 (L217); (x) a substitution of a glutamic acid residue at a position corresponding to position 225 of wild-type human Neu2 (E225); (y) a substitution of a histidine residue at a position corresponding to position 239 of wild-type human Neu2 (H239); (z) a substitution of a leucine residue at a position corresponding to position 240 of wild-type human Neu2 (L240); (aa) a substitution of an arginine residue at a position corresponding to position 241 of wild-type human Neu2 (R241); (bb) a substitution of an alanine residue at a position corresponding to position 242 of wild-type human Neu2 (A242); (cc) a substitution of a valine residue at a position corresponding to position 244 of wild-type human Neu2 (V244); (dd) a substitution of a threonine residue at a position corresponding to position 249 of wild-type human Neu2 (T249); (ee) a substitution of an aspartic acid residue at a position corresponding to position 251 of wild-type human Neu2 (D251); (ff) a substitution of a glutamic acid residue at a position corresponding to position 257 of wild-type human Neu2 (E257); (gg) a substitution of a serine residue at a position corresponding to position 258 of wild-type human Neu2 (S258); (hh) a substitution of a leucine residue at a position corresponding to position 260 of wild-type human Neu2 (L260); (ii) a substitution of a valine residue at a position corresponding to position 265 of wild-type human Neu2 (V265); (jj) a substitution of a glutamine residue at a position corresponding to position 270 of wild-type human Neu2 (Q270); (kk) a substitution of a tryptophan residue at a position corresponding to position 292 of wild-type human Neu2 (W292); (ll) a substitution of a serine residue at a position corresponding to position 301 of wild-type human Neu2 (S301); (mm) a substitution of a tryptophan residue at a position corresponding to position 302 of wild-type human Neu2 (W302); (nn) a substitution of a valine residue at a position corresponding to position 363 of wild-type human Neu2 (V363); or (oo) a substitution of a leucine residue at a position corresponding to position 365 of wild-type human Neu2 (L365); or a combination of any of the foregoing substitutions. For example, the sialidase may comprise a substitution of K9, P62, A93, Q216, A242, Q270, 5301, W302, V363, or L365, or a combination of any of the foregoing substitutions.

In certain embodiments, in the sialidase: (a) the proline residue at a position corresponding to position 5 of wild-type human Neu2 is substituted by histidine (P5H); (b) the lysine residue at a position corresponding to position 9 of wild-type human Neu2 is substituted by aspartic acid (K9D); (c) the lysine residue at a position corresponding to position 44 of wild-type human Neu2 is substituted by arginine (K44R) or glutamic acid (K44E); (d) the lysine residue at a position corresponding to position 45 of wild-type human Neu2 is substituted by alanine (K45A), arginine (K45R), or glutamic acid (K45E); (e) the leucine residue at a position corresponding to position 54 of wild-type human Neu2 is substituted by methionine (L54M); (f) the proline residue at a position corresponding to position 62 of wild-type human Neu2 is substituted by asparagine (P62N), aspartic acid (P62D), histidine (P62H), glutamic acid (P62E), glycine (P62G), serine (P62S), or threonine (P62T); (g) the glutamine residue at a position corresponding to position 69 of wild-type human Neu2 is substituted by histidine (Q69H); (h) the arginine residue at a position corresponding to position 78 of wild-type human Neu2 is substituted by lysine (R78K); (i) the aspartic acid residue at a position corresponding to position 80 of wild-type human Neu2 is substituted by proline (D80P); (j) the alanine residue at a position corresponding to position 93 of wild-type human Neu2 is substituted by glutamic acid (A93E) or lysine (A93K); (k) the glycine residue at a position corresponding to position 107 of wild-type human Neu2 is substituted by aspartic acid (G107D); (1) the glutamine residue at a position corresponding to position 108 of wild-type human Neu2 is substituted by histidine (Q108H); (m) the glutamine residue at a position corresponding to position 112 of wild-type human Neu2 is substituted by arginine (Q112R) or lysine (Q112K); (n) the cysteine residue at a position corresponding to position 125 of wild-type human Neu2 is substituted by leucine (C125L); (o) the glutamine residue at a position corresponding to position 126 of wild-type human Neu2 is substituted by leucine (Q126L), glutamic acid (Q126E), phenylalanine (Q126F), histidine (Q126H), isoleucine (Q126I), or tyrosine (Q126Y); (p) the alanine residue at a position corresponding to position 150 of wild-type human Neu2 is substituted by valine (A150V); (q) the cysteine residue at a position corresponding to position 164 of wild-type human Neu2 is substituted by glycine (C164G); (r) the arginine residue at a position corresponding to position 170 of wild-type human Neu2 is substituted by proline (R170P); (s) the alanine residue at a position corresponding to position 171 of wild-type human Neu2 is substituted by glycine (A171G); (t) the glutamine residue at a position corresponding to position 188 of wild-type human Neu2 is substituted by proline (Q188P); (u) the arginine residue at a position corresponding to position 189 of wild-type human Neu2 is substituted by proline (R189P); (v) the alanine residue at a position corresponding to position 213 of wild-type human Neu2 is substituted by cysteine (A213C), asparagine (A213N), serine (A213S), or threonine (A213T); (w) the leucine residue at a position corresponding to position 217 of wild-type human Neu2 is substituted by alanine (L217A) or valine (L217V); (x) the threonine residue at a position corresponding to position 249 of wild-type human Neu2 is substituted by alanine (T249A); (y) the aspartic acid residue at a position corresponding to position 251 of wild-type human Neu2 is substituted by glycine (D251G); (z) the glutamic acid residue at a position corresponding to position 225 of wild-type human Neu2 is substituted by proline (E225P); (aa) the histidine residue at a position corresponding to position 239 of wild-type human Neu2 is substituted by proline (H239P); (bb) the leucine residue at a position corresponding to position 240 of wild-type human Neu2 is substituted by aspartic acid (L240D), asparagine (L240N), or tyrosine (L240Y); (cc) the arginine residue at a position corresponding to position 241 of wild-type human Neu2 is substituted by alanine (R241A), aspartic acid (R241D), leucine (R241L), glutamine (R241Q). or tyrosine (R241Y); (dd) the alanine residue at a position corresponding to position 242 of wild-type human Neu2 is substituted by cysteine (A242C), phenylalanine (A242F), glycine (A242G), histidine (A242H), isoleucine (A242I), lysine (A242K), leucine (A242L), methionine (A242M), asparagine (A242N), glutamine (A242Q), arginine (A242R), serine (A242S), valine (A242V), tryptophan (A242W), or tyrosine (A242Y); (ee) the valine residue at a position corresponding to position 244 of wild-type human Neu2 is substituted by isoleucine (V244I), lysine (V244K), or proline (V244P); (ff) the glutamic acid residue at a position corresponding to position 257 of wild-type human Neu2 is substituted by proline (E257P); (gg) the serine residue at a position corresponding to position 258 is substituted by cysteine (S258C); (hh) the leucine residue at a position corresponding to position 260 of wild-type human Neu2 is substituted by aspartic acid (L260D), phenylalanine (L260F), glutamine (L260Q), or threonine (L260T); (ii) the valine residue at a position corresponding to position 265 of wild-type human Neu2 is substituted by phenylalanine (V265F); (jj) the glutamine residue at a position corresponding to position 270 of wild-type human Neu2 is substituted by alanine (Q270A), histidine (Q270H), phenylalanine (Q270F), proline (Q270P), serine (Q270S), or threonine (Q270T); (kk) the tryptophan residue at a position corresponding to position 292 of wild-type human Neu2 is substituted by arginine (W292R); (ll) the serine residue at a position corresponding to position 301 of wild-type human Neu2 is substituted by alanine (S301A), aspartic acid (S301D), glutamic acid (S301E), phenylalanine (S301F), glycine (S301G), histidine (S301H), isoleucine (S301I), lysine (S301K), leucine (S301L), methionine (S301M), asparagine (S301N), proline (S301P), glutamine (S301Q), arginine (S301R), threonine (S301T), valine (S301V), tryptophan (S301W), or tyrosine (S301Y)); (mm) the tryptophan residue at a position corresponding to position 302 of wild-type human Neu2 is substituted by alanine (W302A), aspartic acid (W302D), glutamic acid (W302E), phenylalanine (W302F), glycine (W302G), histidine (W302H), isoleucine (W3021), lysine (W302K), leucine (W302L), methionine (W302M), asparagine (W302N), proline (W302P), glutamine (W302Q), arginine (W302R), serine (W302S), threonine (W302T), valine (W302V), or tyrosine (W302Y); (nn) the valine residue at a position corresponding to position 363 of wild-type human Neu2 is substituted by arginine (V363R); or (oo) the leucine residue at a position corresponding to position 365 of wild-type human Neu2 is substituted by glutamine (L365Q), histidine (L365H), isoleucine (L365I), lysine (L365K) or serine (L365S); or the sialidase comprises a combination of any of the foregoing substitutions. For example, the sialidase may comprise a substitution selected from K9D, P62G, P62N, P62S, P62T, D80P, A93E, Q126H, Q126Y, R189P, H239P, A242T, Q270A, Q270S, Q270T, S301A, S301R, W302K, W302R, V363R, and L365I, or a combination of any of the foregoing substitutions.

In certain embodiments, the recombinant mutant human sialidase comprises a deletion of a leucine residue at a position corresponding to position 184 of wild-type human Neu2 (ΔL184), a deletion of a histidine residue at a position corresponding to position 185 of wild-type human Neu2 (ΔH185), a deletion of a proline residue at a position corresponding to position 186 of wild-type human Neu2 (ΔP186), a deletion of an isoleucine residue at a position corresponding to position 187 of wild-type human Neu2 (ΔI187), and a deletion of a glutamine residue at a position corresponding to position 184 of wild-type human Neu2 (ΔQ188), or a combination of any of the foregoing deletions.

In certain embodiments, the recombinant mutant human sialidase comprises an insertion between a threonine residue at a position corresponding to position 216 of wild-type human Neu2 and a leucine residue at a position corresponding to position 217 of wild-type human Neu2, for example, an insertion of an amino acid selected from S, T, Y, L, F, A, P, V, I, N, D, and H.

Additional exemplary sialidase mutations, and combinations of sialidase mutations, are described in International (PCT) Patent Application No. PCT/US2019/012207, filed Jan. 3, 2019, including in the Detailed Description in the section entitled “I. Recombinant Human Sialidases,” and in the Examples in Examples 1, 2, 3, 4, 5, and 6.

v. Combinations of Substitutions

The invention further provides a recombinant mutant human sialidase comprising a combination of any of the mutations contemplated herein. For example, the recombinant mutant sialidase enzyme may comprise a combination of 2, 3, 4, 5, 6, 7, 8, 9, 10, 11, 12, 13, 14, 15 or more of the mutations contemplated herein. It is contemplated that the recombinant mutant sialidase enzyme may comprise 1-15, 1-10, 1-7, 1-6, 1-5, 1-4, 1-3, 1-2, 2-15, 2-10, 2-7, 2-6, 2-5, 2-4, 2-3, 3-15, 3-10, 3-7, 3-6, 3-5, or 3-4 of the mutations contemplated herein.

For example, the recombinant mutant sialidase enzyme may comprise a M1 deletion (ΔM1), M1A substitution, M1D substitution, V6Y substitution, K9D substitution, P62G substitution, P62N substitution, P62S substitution, P62T substitution, A93E substitution, I187K substitution, Q270A substitution, S301R substitution, W302K substitution, C332A substitution, V363R substitution, L365I substitution, or a combination of any of the foregoing.

In certain embodiments, the recombinant mutant sialidase enzyme comprises a M1 deletion (ΔM1), M1A substitution, M1D substitution, V6Y substitution, I187K substitution, C332A substitution, or a combination of any of the foregoing. For example, the recombinant mutant sialidase enzyme may comprise a combination of mutations selected from: M1A and V6Y; M1A and I187K; M1A and C332A; M1D and V6Y; M1D and I187K; M1D and C332A; ΔM1 and V6Y; ΔM1 and I187K; ΔM1 and C332A; V6Y and I187K; V6Y and C332A; I187K and C332A; M1A, V6Y, and I187K; M1A, V6Y, and C332A; M1A, I187K, and C332A; M1D, V6Y, and I187K; M1D, V6Y, and C332A; M1D, I187K, and C332A; ΔM1, V6Y, and I187K; ΔM1, V6Y, and C332A; ΔM1, I187K, and C332A; V6Y, I187K, and C332A; M1A, V6Y, I187K, and C332A; M1D, V6Y, I187K, and C332A; and ΔM1, V6Y, I187K, and C332A.

In certain embodiments, the recombinant mutant sialidase enzyme comprises (i) an amino acid substitution identified in TABLE 8, or a combination of any amino acid substitution identified in TABLE 8, and (ii) a substitution a M1 deletion (ΔM1), M1A substitution, M1D substitution, V6Y substitution, I187K substitution, C332A substitution, or a combination of any of the foregoing. For example, the recombinant mutant sialidase enzyme may comprise (i) an amino acid substitution identified in TABLE 8, or a combination of any amino acid substitution identified in TABLE 8, and (ii) a combination of mutations selected from: M1A and V6Y; M1A and I187K; M1A and C332A; M1D and V6Y; M1D and I187K; M1D and C332A; ΔM1 and V6Y; ΔM1 and I187K; ΔM1 and C332A; V6Y and I187K; V6Y and C332A; I187K and C332A; M1A, V6Y, and I187K; M1A, V6Y, and C332A; M1A, I187K, and C332A; M1D, V6Y, and I187K; M1D, V6Y, and C332A; M1D, I187K, and C332A; ΔM1, V6Y, and I187K; ΔM1, V6Y, and C332A; ΔM1, I187K, and C332A; V6Y, I187K, and C332A; M1A, V6Y, I187K, and C332A; M1D, V6Y, I187K, and C332A; and ΔM1, V6Y, I187K, and C332A.

In certain embodiments, the recombinant mutant sialidase enzyme comprises: (a) the M1D, V6Y, P62G, A93E, I187K, and C332A substitutions; (b) the M1D, V6Y, K9D, A93E, I187K, C332A, V363R, and L365I substitutions; (c) the M1D, V6Y, P62N, I187K, and C332A substitutions; (d) the M1D, V6Y, I187K, Q270A, S301R, W302K, and C332A substitutions; (e) the M1D, V6Y, P62S, I187K, Q270A, S301R, W302K, and C332A substitutions; (f) the M1D, V6Y, P62T, I187K, Q270A, S301R, W302K, and C332A substitutions; (g) the M1D, V6Y, P62N, I187K, Q270A, S301R, W302K, and C332A substitutions; (h) the M1D, V6Y, P62G, A93E, I187K, S301A, W302R, and C332A substitutions; (i) the M1D, V6Y, P62G, A93E, Q126Y, I187K, Q270T, and C332A substitutions; or (j) the M1D, V6Y, P62G, A93E, Q126Y, I187K, and C332A substitutions; or (k) the M1D, V6Y, P62G, A93E, Q126Y, I187K, A242F, Q270T, and C332A substitutions.

In certain embodiments, the recombinant mutant human sialidase comprises a substitution of a serine residue at a position corresponding to position 301 of wild-type human Neu2 (S301) in combination with a substitution of a tryptophan residue at a position corresponding to position 302 of wild-type human Neu2 (W302). For example, the recombinant mutant human sialidase may comprise a combination of substitutions corresponding to a combination of substitutions listed in a row of TABLE 8 (amino acid positions corresponding to wild-type human Neu2 (SEQ ID NO: 1)). For example, the recombinant mutant human sialidase may comprise: the S301K and W302R substitutions; the S301K and W302K substitutions; or the S301A and W302S substitutions.

TABLE 8
Substitutions
S301 A, W302R
S301A, W302S
S301A, W302T
S301K, W302S
S301N, W302S
S301T, W302S
S301T, W302T
S301T, W302R
S301A, W302A
S301K, W302R
S301K, W302T
S301N, W302T
S301K, W302K
S301P, W302R
S301P, W302S
S301P, W302T

In certain embodiments, the recombinant mutant human sialidase comprises a combination of substitutions corresponding to a combination of substitutions listed in a row of TABLE 9 (amino acid positions corresponding to wild-type human Neu2 (SEQ ID NO: 1)).

TABLE 9
Substitutions
M1D, V6Y, P62G, I187K, C332A
M1D, V6Y, K9D, I187K, C332A, V363R, L365I
M1D, V6Y, P62G, A93E, I187K, C332A
M1D, V6Y, K9D, I187K, C332A, V363R, L365K
M1D, V6Y, K9D, I187K, C332A, V363R, L365S
M1D, V6Y, K9D, I187K, C332A, V363R, L365Q
M1D, V6Y, K9D, I187K, C332A, V363R, L365H
M1D, V6Y, A93K, I187K, C332A
M1D, V6Y, A93E, I187K, C332A
V6Y, I187K, W292R
V6Y, G107D, I187K
V6Y, C125L
C125L, I187K
V6Y, C125L, I187K
M1D, V6Y, K45A, I187K, C332A
M1D, V6Y, Q270A, I187K, C332A
M1D, V6Y, K44R, K45R, I187K, C332A
M1D, V6Y, Q112R, I187K, C332A
M1D, V6Y, Q270F, I187K, C332A
M1D, V6Y, I187K, S301R, W302K, C332A
M1D, V6Y, K44E, K45E, I187K, C332A
M1D, V6Y, I187K, L217V, C332A
M1D, V6Y, I187K, L217A, C332A
M1D, V6Y, K44E, K45E, I187K, S301R, W302K, C332A
M1D, V6Y, Q112R, I187K, S301R, W302K, C332A
M1D, V6Y, I187K, Q270A, S301R, W302K, C332A
M1D, V6Y, K44E, K45E, Q112R, I187K, C332A
M1D, V6Y, K44E, K45E, I187K, Q270A, C332A
M1D, V6Y, K45A, I187K, Q270A, C332A
M1D, V6Y, I187K, Q270H, C332A
M1D, V6Y, I187K, Q270P, C332A
M1D, V6Y, Q112K, I187K, C332A
M1D, V6Y, P62S, I187K, Q270A, S301R, W302K, C332A
M1D, V6Y, P62T, I187K, Q270A, S301R, W302K, C332A
M1D, V6Y, P62N, I187K, Q270A, S301R, W302K, C332A
V6Y, P62H, I187K
V6Y, Q108H, I187K
M1D, V6Y, P62H, I187K, C332A
M1D, V6Y, P62G, I187K, C332A
V6Y, P62G, I187K
M1D, V6Y, P62H, I187K
M1D, V6Y, Q108H, I187K
M1D, V6Y, P62N, I187K, C332A
M1D, V6Y, P62D, I187K, C332A
M1D, V6Y, P62E, I187K, C332A
V6Y, C164G, I187K, T248A
V6Y, C164G, I187K
V6Y, Q126L, I187K D251G
V6Y, L54M, Q69H, R78K, A171G, I187K
V6Y, P62T, I187K
V6Y, Al 50V, I187K
P5H, V6Y, P62S, I187K
V6Y, C164G, I187K
Q126Y, Q170T
Q126Y, A242F, Q270T
M1D, V6Y, P62G, A93E, Q126E, I187K, C332A
M1D, V6Y, P62G, A93E, Q126I, I187K, C332A
M1D, V6Y, P62G, A93E, Q126L, I187K, C332A
M1D, V6Y, P62G, A93E, Q126Y, I187K, C332A
M1D, V6Y, P62G, A93E, Q126F, I187K, C332A
M1D, V6Y, P62G, A93E, Q126H, I187K, C332A
M1D, V6Y, P62G, A93E, I187K, Q270S, C332A
M1D, V6Y, P62G, A93E, I187K, Q270T, C332A
M1D, V6Y, P62G, A93E, Q126Y, I187K, Q270T, C332A
M1D, V6Y, P62G, A93E, Q126Y, I187K, A242F, Q270T, C332A
M1D, V6Y, P62G, D80P, A93E, I187K, C332A
M1D, V6Y, P62G, A93E, R170P, I187K, C332A
M1D, V6Y, P62G, A93E, I187K, Q188P, C332A
M1D, V6Y, P62G, A93E, I187K, R189P, C332A
M1D, V6Y, P62G, A93E, I187K, E225P, C332A
M1D, V6Y, P62G, A93E, I187K, H239P, C332A
M1D, V6Y, P62G, A93E, I187K, E257P, C332A
M1D, V6Y, P62G, A93E, I187K, S301A, C332A
M1D, V6Y, P62G, A93E, I187K, S301D, C332A
M1D, V6Y, P62G, A93E, I187K, S301E, C332A
M1D, V6Y, P62G, A93E, I187K, S301F, C332A
M1D, V6Y, P62G, A93E, I187K, S301H, C332A
M1D, V6Y, P62G, A93E, I187K, S301K, C332A
M1D, V6Y, P62G, A93E, I187K, S301L, C332A
M1D, V6Y, P62G, A93E, I187K, S301M, C332A
M1D, V6Y, P62G, A93E, I187K, S301N, C332A
M1D, V6Y, P62G, A93E, I187K, S301P, C332A
M1D, V6Y, P62G, A93E, I187K, S301Q, C332A
M1D, V6Y, P62G, A93E, I187K, S301R, C332A
M1D, V6Y, P62G, A93E, I187K, S301T, C332A
M1D, V6Y, P62G, A93E, I187K, S301V, C332A
M1D, V6Y, P62G, A93E, I187K, S301W, C332A
M1D, V6Y, P62G, A93E, I187K, S301Y, C332A
M1D, V6Y, P62G, A93E, I187K, W302A, C332A
M1D, V6Y, P62G, A93E, I187K, W302D, C332A
M1D, V6Y, P62G, A93E, I187K, W302F, C332A
M1D, V6Y, P62G, A93E, I187K, W302G, C332A
M1D, V6Y, P62G, A93E, I187K, W302H, C332A
M1D, V6Y, P62G, A93E, I187K, W302I, C332A
M1D, V6Y, P62G, A93E, I187K, W302L, C332A
M1D, V6Y, P62G, A93E, I187K, W302M, C332A
M1D, V6Y, P62G, A93E, I187K, W302N, C332A
M1D, V6Y, P62G, A93E, I187K, W302P, C332A
M1D, V6Y, P62G, A93E, I187K, W302Q, C332A
M1D, V6Y, P62G, A93E, I187K, W302R, C332A
M1D, V6Y, P62G, A93E, I187K, W302S, C332A
M1D, V6Y, P62G, A93E, I187K, W302T, C332A
M1D, V6Y, P62G, A93E, I187K, W302V, C332A
M1D, V6Y, P62G, A93E, I187K, W302Y, C332A
M1D, V6Y, P62G, A93E, I187K, S301A, W302A, C332A
M1D, V6Y, P62G, A93E, I187K, S301A, W302R, C332A
M1D, V6Y, P62G, A93E, I187K, S301A, W302S, C332A
M1D, V6Y, P62G, A93E, I187K, S301A, W302T, C332A
M1D, V6Y, P62G, A93E, I187K, S301K, W302S, C332A
M1D, V6Y, P62G, A93E, I187K, S301K, W302R, C332A
M1D, V6Y, P62G, A93E, I187K, S301K, W302T, C332A
M1D, V6Y, P62G, A93E, I187K, S301N, W302S, C332A
M1D, V6Y, P62G, A93E, I187K, S301N, W302T, C332A
M1D, V6Y, P62G, A93E, I187K, S301T, W302R, C332A
Q126Y, Q270T
Q126Y, A242F, Q270T

In certain embodiments, the recombinant mutant human sialidase comprises the amino acid sequence of any one of SEQ ID NOs: 48-62, 169-171, or 196 or an amino acid sequence that has at least 85%, 90%, 95%, 96%, 97%, 98%, or 99% sequence identity to any one of SEQ ID NOs: 48-62, 169-171, or 196.

In certain embodiments, the recombinant mutant human sialidase comprises the amino acid sequence of

(SEQ ID NO: 47)
X₁X₂SX₃X₄X₅LQX₆ESVFQSGAHAYRIPALLYLPGQQSLLAFAEQRAS
X₇X₈DEHAELIVX₉RRGDYDAX₁₀THQVQWX₁₁AQEVVAQAX₁₂LDGHR
SMNPCPLYDX₁₃QTGTLFLFFIAIPX₁₄X₁₅VTEX₁₆QQLQTRANVTRL
X₁₇X₁₈VTSTDHGRTWSSPRDLTDAAIGPX₁₉YREWSTFAVGPGHX₂₀L
QLHDRX₂₁RSLVVPAYAYRKLHPX₂₂QRPIPSAFX₂₃FLSHDHGRTWAR
GHFVAQDTX₂₄ECQVAEVETGEQRVVTLNARSHLRARVQAQSX₂₅NX₂₆
GLDFQX₂₇SQLVKKLVEPPPX₂₈GX₂₉QGSVISFPSPRSGPGSPAQX₃₀
LLYTHPTHX₃₁X₃₂QRADLGAYLNPRPPAPEAWSEPX₃₃LLAKGSX₃₄A
YSDLQSMGTGPDGSPLFGX₃₅LYEANDYEEIX₃₆FX₃₇MFTLKOAFPAE
YLPQ,

wherein X₁ is Ala, Arg, Asn, Asp, Gln, Glu, Gly, His, Leu, Lys, Met, Phe, Thr, Val, or not present, X₂ is Ala or Lys, X₃ is Asn or Leu, X₄ is Pro or His, X₅ is Phe, Trp, Tyr or Val, X₆ is Lys or Asp. X₇ is Lys, Arg, or Glu. X₈ is Lys, Ala, Arg, or Glu, X₉ is Leu or Met, X₁₀ is Pro, Asn, Asp, His, Glu, Gly, Ser or Thr, X₁₁ is Gln or His, X₁₂ is Arg or Lys, X₁₃ is Ala, Glu or Lys, X₁₄ is Gly or Asp, X₁₅ is Gln or His, X₁₆ is Gln, Arg, or Lys, X₁₇ is Ala, Cys, Ile, Ser, Val, or Leu, X₁₈ is Gln or Leu, X₁₉ is Ala or Val, X₂₀ is Cys or Gly, X₂₁ is Ala or Gly, X₂₂ is Arg, Ile, or Lys, X₂₃ is Ala, Cys, Leu, or Val, X₂₄ is Leu, Ala, or Val, X₂₅ is Thr or Ala, X₂₆ is Asp or Gly, X₂₇ is Glu or Lys, X₂₈ is Gln, Ala, His, Phe, or Pro, X₂₉ is Cys or Val, X₃₀ is Trp or Arg, X₃₁ is Ser or Arg, X₃₂ is Trp or Lys, X₃₃ is Lys or Val, X₃₄ is Ala, Cys, Ser, or Val, X₃₅ is Cys, Leu, or Val, X₃₆ is Val or Arg, and X₃₇ is Leu, Gln, His, Ile, Lys, or Ser, and the sialidase comprises at least one mutation relative to wild-type human Neu2 (SEQ ID NO: 1).

In certain embodiments, the recombinant mutant human sialidase comprises the amino acid sequence of

(SEQ ID NO: 46)
X₁ASLPX₂LQX₃ESVFQSGAHAYRIPALLYLPGQQSLLAFAEQRASKKD
EHAELIVLRRGDYDAX₄THQVQWQAQEVVAQARLDGHRSMNPCPLYDX₅
QTGTLFLFFIAIPGQVTEQQQLQTRANVTRLCQVTSTDHGRTWSSPRDL
TDAAIGPAYREWSTFAVGPGHCLQLHDRARSLWPAYAYRKLHPX₆QRPI
PSAFCFLSHDHGRTWARGHFVAQDTLECQVAEVETGEQRWTLNARSHLR
ARVQAQSTNDGLDFQESQLVKKLVEPPPX₇GCQGSVISFPSPRSGPGSP
AQWLLYTHPTHX₈X₉QRADLGAYLNPRPPAPEAWSEPVLLAKGSX₁₀AY
SDLQSMGTGPDGSPLFGCLYEANDYEEIX₁₁FX₁₂MFTLKQAFPAEYLP
Q,

wherein X₁ is Ala, Arg, Asn, Asp, Gln, Glu, Gly, His, Leu, Lys, Met, Phe, Thr, Val, or not present, X₂ is Phe, Trp, Tyr or Val, X₃ is Lys or Asp, X₄ is Pro, Asn, Asp, His, Glu, Gly, Ser or Thr, X₅ is Ala, Glu, or Lys, X₆ is Arg, Ile, or Lys, X₇ is Gln, Ala, His, Phe, or Pro, X₈ is Ser or Arg, X₉ is Trp or Lys, X₁₀ is Ala, Cys, Ser, or Val, X₁₁ is Val or Arg, and X₁₂ is Leu, Gln, His, Ile, Lys, or Ser, and the sialidase comprises at least one mutation relative to wild-type human Neu2 (SEQ ID NO: 1). In certain embodiments, X₁ is Ala, Asp, Met, or not present, X₂ is Tyr or Val, X₃ is Lys or Asp, X₄ is Pro, Asn, Gly, Ser or Thr, X₅ is Ala or Glu, X₆ is Ile or Lys, X₇ is Gln or Ala, X₈ is Ser or Arg, X₉ is Trp or Lys, X₁₀ is Ala or Cys, X₁₁ is Val or Arg, and X₁₂ is Leu or Ile.

In certain embodiments, the recombinant mutant human sialidase comprises the amino acid sequence of

(SEQ ID NO: 172)
X₁X₂SX₃X₄X₅LQX₆ESVFQSGAHAYRIPALLYLPGQQSLLAFAEQRAS
X₇X₈DEHAELIVX₉RRGDYDAX₁₀THQVQWX₁₁AQEVVAQAX₁₂LX₁₃G
HRSMNPCPLYDX₁₄QTGTLFLFFIAIPX₁₅X₁₆VTEX₁₇QQLQTRANVT
RLX₁₈X_{I9}VTSTDHGRTWSSPRDLTDAAIGPX₂₀YREWSTFAVGPGHX₂₁
LQLHDX₂₂X₂₃RSLVVPAYAYRKLHPX₂₄X₂₅X₂₆PIPSAFX₂₇FLSHD
HGRTWARGHFVX₂₈QDTX₂₉ECQVAEVX₃₀TGEQRVVTLNARSX₃₁X₃₂
X₃₃X₃₄RX₃₅QAQSX₃₆NX₃₇GLDFQX₃₈X₃₉QX₄₀VKKLX₄₁EPPPX₄₂
GX₄₃QGSVISFPSPRSGPGSPAQX₄₄LLYTHPTHX₄₅X₄₆QRADLGAYLN
PRPPAPEAWSEPX₄₇LLAKGSX₄₈AYSDLQSMGTGPDGSPLFGX₄₉LYEA
NDYEEIX₅₀FX₅₁MFTLKQAFPAEYLPQ,

wherein X₁ is Ala, Arg, Asn, Asp, Gln, Glu, Gly, His, Leu, Lys, Met, Phe, Thr, Val, or not present, X₂ is Ala or Lys, X₃ is Asn or Leu, X₄ is Pro or His, X₅ is Phe, Trp, Tyr or Val, X₆ is Lys or Asp, X₇ is Lys, Arg, or Glu, X₈ is Lys, Ala, Arg, or Glu, X₉ is Leu or Met, X₁₀ is Pro, Asn, Asp, His, Glu, Gly, Ser or Thr, X₁₁ is Gln or His, X₁₂ is Arg or Lys, X₁₃ is Asp or Pro, X₁₄ is Ala, Glu or Lys, X₁₅ is Gly or Asp, X₁₆ is Gln or His, X₁₇ is Gln, Arg, or Lys, X₁₈ is Ala, Cys, Ile, Ser, Val, or Leu, X₁₉ is Gln, Leu, Glu, Phe, His, Ile, Leu, or Tyr, X₂₀ is Ala or Val, X₂₁ is Cys or Gly, X₂₂ is Arg or Pro, X₂₃ is Ala or Gly, X₂₄ is Arg, Ile, or Lys, X₂₅ is Gln or Pro, X₂₆ is Arg or Pro, X₂₇ is Ala, Cys, Leu, or Val, X₂₈ is Ala, Cys, Asn, Ser, or Thr, X₂₉ is Leu, Ala, or Val, X₃₀ is Glu or Pro, X₃₁ is His or Pro, X₃₂ is Leu, Asp, Asn, or Tyr, X₃₃ is Arg, Ala, Asp, Leu, Gln, or Tyr, X₃₄ is Ala, Cys, Phe, Gly, His, Ile, Lys, Leu, Met, Asn, Gln, Arg, Ser, Val, Trp, or Tyr, X₃₅ is Val, Ile, or Lys, X₃₆ is Thr or Ala, X₃₇ is Asp or Gly, X₃₈ is Glu, Lys, or Pro, X₃₉ is Ser or Cys, X₄₀ is Leu, Asp, Phe, Gln, or Thr, X₄₁ is Val or Phe, X₄₂ is Gln, Ala, His, Phe, Pro, Ser, or Thr, X₄₃ is Cys or Val, X₄₄ is Trp or Arg, X₄₅ is Ser, Arg, Ala, Asp, Glu, Phe, Gly, His, Ile, Lys, Leu, Met, Asn, Pro, Gln, Thr, Val, Trp, or Tyr, X₄₆ is Trp, Lys, Ala, Asp, Glu, Phe, Gly, His, Ile, Lys, Leu, Met, Asn, Pro, Gln, Arg, Ser, Thr, Val, or Tyr, X₄₇ is Lys or Val, X₄₈ is Ala, Cys, Ser, or Val, X₄₉ is Cys, Leu, or Val, X₅₀ is Val or Arg, and X₅₁ is Leu, Gln, His, Ile, Lys, or Ser, and the sialidase comprises at least one mutation relative to wild-type human Neu2 (SEQ ID NO: 1).

In certain embodiments, the recombinant mutant human sialidase comprises the amino acid sequence of

(SEQ ID NO: 173)
X₁ASLPX₂LQX₃ESVFQSGAHAYRIPALLYLPGQQSLLAFAEQRASKKDE
HAELIVLRRGDYDAX₄THQVQWQAQEVVAQARLDGHRSMNPCPLYDX₅QT
GTLFLFFIAIPGQVTEQQQLQTRANVTRLCX₆VTSTDHGRTWSSPRDLTD
AAIGPAYREWSTFAVGPGHCLQLHDRARSLWPAYAYRKLHPX₇QRPIPSA
FCFLSHDHGRTWARGHFVAQDTLECQVAEVETGEQRWTLNARSHLRX₈RV
QAQSTNDGLDFQESQLVKKLVEPPPX₉GCQGSVISFPSPRSGPGSPAQWL
LYTHPTHX₁₀X₁₁QRADLGAYLNPRPPAPEAWSEPVLLAKGSX₁₂AYSDL
QSMGTGPDGSPLFGCLYEANDYEEIX₁₃FX₁₄MFTLKQAFPAEYLPQ,

wherein X₁ is Ala, Arg, Asn, Asp, Gln, Glu, Gly, His, Leu, Lys, Met, Phe, Thr, Val, or not present, X₂ is Phe, Trp, Tyr or Val, X₃ is Lys or Asp, X₄ is Pro, Asn, Asp, His, Glu, Gly, Ser or Thr, X₅ is Ala, Glu, or Lys, X₆ is Gln, Leu, Glu, Phe, His, Ile, Leu, or Tyr, X₇ is Arg, Ile, or Lys, X₈ is Ala, Cys, Phe, Gly, His, Ile, Lys, Leu, Met, Asn, Gln, Arg, Ser, Val, Trp, or Tyr, X₉ is Gln, Ala, His, Phe, Pro, Ser, or Thr, X₁₀ is Ser, Arg, Ala, Asp, Glu, Phe, Gly, His, Ile, Lys, Leu, Met, Asn, Pro, Gln, Thr, Val, Trp, or Tyr, X₁₁ is Trp, Lys, Ala, Asp, Glu, Phe, Gly, His, Ile, Lys, Leu, Met, Asn, Pro, Gln, Arg, Ser, Thr, Val, or Tyr, X₁₂ is Ala, Cys, Ser, or Val, X₁₃ is Val or Arg, and X₁₄ is Leu, Gln, His, Ile, Lys, or Ser, and the sialidase comprises at least one mutation relative to wild-type human Neu2 (SEQ ID NO: 1). In certain embodiments, X₁ is Ala, Asp, Met, or not present, X₂ is Tyr or Val, X₃ is Lys or Asp, X₄ is Pro, Asn, Gly, Ser or Thr, X₅ is Ala or Glu, X₆ is Gln or Tyr, X₇ is Ile or Lys, X₈ is Ala or Thr, X₉ is Gln, Ala, or Thr, X₁₀ is Ser, Arg, or Ala, X₁₁ is Trp, Lys, or Arg, X₁₂ is Ala or Cys, X₁₃ is Val or Arg, and X₁₄ is Leu or Ile.

In certain embodiments, the recombinant mutant human sialidase comprises a conservative substitution relative to a recombinant mutant human sialidase sequence disclosed herein. As used herein, the term “conservative substitution” refers to a substitution with a structurally similar amino acid. For example, conservative substitutions may include those within the following groups: Ser and Cys; Leu, Ile, and Val; Glu and Asp; Lys and Arg; Phe, Tyr, and Trp; and Gln, Asn, Glu, Asp, and His. Conservative substitutions may also be defined by the BLAST (Basic Local Alignment Search Tool) algorithm, the BLOSUM substitution matrix (e.g., BLOSUM 62 matrix), or the PAM substitution:p matrix (e.g., the PAM 250 matrix).

Sequence identity may be determined in various ways that are within the skill of a person skilled in the art, e.g., using publicly available computer software such as BLAST, BLAST-2, ALIGN or Megalign (DNASTAR) software. BLAST (Basic Local Alignment Search Tool) analysis using the algorithm employed by the programs blastp, blastn, blastx, tblastn and tblastx (Karlin et al., (1990) PROC. NATL. ACAD. SCI. USA 87:2264-2268; Altschul, (1993) J. MOL. EVOL. 36:290-300; Altschul et al., (1997) NUCLEIC ACIDS RES. 25:3389-3402, incorporated by reference herein) are tailored for sequence similarity searching. For a discussion of basic issues in searching sequence databases see Altschul et al., (1994) NATURE GENETICS 6:119-129, which is fully incorporated by reference herein. Those skilled in the art can determine appropriate parameters for measuring alignment, including any algorithms needed to achieve maximal alignment over the full length of the sequences being compared. The search parameters for histogram, descriptions, alignments, expect (i.e., the statistical significance threshold for reporting matches against database sequences), cutoff, matrix and filter are at the default settings. The default scoring matrix used by blastp, blastx, tblastn, and tblastx is the BLOSUM62 matrix (Henikoff et al., (1992) PROC. NATL. ACAD. SCI. USA 89:10915-10919, fully incorporated by reference herein). Four blastn parameters may be adjusted as follows: Q=10 (gap creation penalty); R=10 (gap extension penalty); wink=1 (generates word hits at every wink.sup.th position along the query); and gapw=16 (sets the window width within which gapped alignments are generated). The equivalent blastp parameter settings may be Q=9; R=2; wink=1; and gapw=32. Searches may also be conducted using the NCBI (National Center for Biotechnology Information) BLAST Advanced Option parameter (e.g.: -G, Cost to open gap [Integer]: default=5 for nucleotides/11 for proteins; -E, Cost to extend gap [Integer]: default=2 for nucleotides/1 for proteins; -q, Penalty for nucleotide mismatch [Integer]: default=−3; -r, reward for nucleotide match [Integer]: default=1; -e, expect value [Real]: default=10; —W, wordsize [Integer]: default=11 for nucleotides/28 for megablast/3 for proteins; -y, Dropoff (X) for blast extensions in bits: default=20 for blastn/7 for others; -X, X dropoff value for gapped alignment (in bits): default=15 for all programs, not applicable to blastn; and —Z, final X dropoff value for gapped alignment (in bits): 50 for blastn, 25 for others). ClustalW for pairwise protein alignments may also be used (default parameters may include, e.g., Blosum62 matrix and Gap Opening Penalty=10 and Gap Extension Penalty=0.1). A Bestfit comparison between sequences, available in the GCG package version 10.0, uses DNA parameters GAP=50 (gap creation penalty) and LEN=3 (gap extension penalty). The equivalent settings in Bestfit protein comparisons are GAP=8 and LEN=2.

### II. Serum Half-Life Extenders

As used herein, a “serum half-life extender” refers to a moiety that can be associated with a sialidase to extend its circulating half-life in the serum of a subject. In certain embodiments, a serum half-life extender can be selected from an Fc domain (see, e.g., Beck et al. (2011) MABS 4:1015-28), albumin (e.g., human serum albumin (HSA), see, Weimer et al. (2013) Recombinant albumin fusion proteins. In: Schmidt S, editor. Fusion protein technologies for biopharmaceuticals: applications and challenges. Hoboken: Wiley; 2013, p. 297-323), albumin binding domain (e.g., an HSA binder, see Walker et al. (2013) Albumin-binding fusion proteins in the development of novel long-acting therapeutics. In: Schmidt S, editor. Fusion protein technologies for biopharmaceuticals: applications and challenges. Hoboken: Wiley; 2013, p. 325-43), transferrin (see Kim et al. (2010) J PHARMACOL EXP THER 334:682-92), XTEN (also called recombinant PEG or “rPEG”, see Schellenberger et al. (2009) NAT. BIO IECHNOL. 27:1186-90), a homo-amino acid polymer (HAP, see Schlapschy et al. (2007) PROTEIN ENG DES SEL. 20:273-84)), a proline-alanine-serine polymer (PAS, see Schlapschy et al. (2013) PROTEIN ENG DES SEL. 26:489-501), an elastin-like peptide (ELP, see Floss et al. (2013) Fusion protein technologies for biopharmaceuticals: applications and challenges, p. 372-98), carboxy-terminal peptide (CTP, Duijkers et al. (2002) HUM REPROD. 17:1987-93)), gelatin-like protein (GLK, Huang et al. (2010) EUR J PHARM BIOPHARM 72:435-41), and a polyethylene glycol (PEG).

Suitable serum half-life extenders also include a variety of polymers, such as those described in U.S. Pat. No. 7,842,789. For example, block copolymers of polyoxyethylene and polyoxypropylene (Pluronics); polymethacrylates; carbomers; and branched or unbranched polysaccharides which comprise the saccharide monomers such as D-mannose, D- and L-galactose, fucose, fructose, D-xylose, L-arabinose, and D-glucuronic acid can be used. In other embodiments, the serum half-life extender can be a hydrophilic polyvinyl polymer such as polyvinyl alcohol and polyvinylpyrrolidone (PVP)-type polymers. The serum half-life extender can be a functionalized polyvinylpyrrolidone, for example, carboxy or amine functionalized on one (or both) ends of the polymer (as available from PolymerSource). Alternatively, the serum half-life extender can include Poly N-(2-hydroxypropyl)methacrylamide (HPMA), or functionalized HPMA (amine, carboxy, etc.), Poly(N-isopropylacrylamide) or functionalized poly(N-isopropylacrylamide).

In one embodiment, a sialidase is covalently attached to a naturally long-half-life polypeptide or protein such as an Fc domain (Beck et al., supra), transferrin (Kim et al., supra), or albumin (Weimer et al., supra) to form a fusion protein, either by genetic fusion (i.e., production of recombinant fusion protein) or by chemical conjugation.

In another embodiment, a sialidase is covalently attached to an inert polypeptide such as an XTEN (also called recombinant PEG or “rPEG”, see Schellenberger, supra), a homo amino acid polymer (HAP, see Schlapschy et al. (2007), supra), a proline-alanine-serine-polymer (PAS, see Schlapschy et al., (2013), supra), an elastin-like peptide (ELP, see Floss et al., supra), or gelatin-like protein (GLK, Huang et al., supra) to form a fusion protein, either by genetic fusion (i.e., production of recombinant fusion protein) or by chemical conjugation. Inert polypeptides function, among other things, to increase the size and hydrodynamic radius of the sialidase, thereby to extend half-life. In certain embodiments, an XTEN polypeptide has a length from about 25 amino acids to about 1500 amino acids (e.g., from about 25 amino acids to about 100 amino acids, from about 25 amino acids to about 250 amino acids, from about 25 amino acids to about 500 amino acids, from about 25 amino acids to about 750 amino acids, from about 25 amino acids to about 1000 amino acids, from about 25 amino acids to about 1250 amino acids, from about 100 amino acids to about 250 amino acids, from about 100 amino acids to about 250 amino acids, from about 100 amino acids to about 500 amino acids, from about 100 amino acids to about 750 amino acids, from about 100 amino acids to about 1000 amino acids, from about 100 amino acids to about 1250 amino acids, from about 100 amino acids to about 1500 amino acids, from about 250 amino acids to about 1250 amino acids, from about 250 amino acids to about 1000 amino acids, from about 250 amino acids to about 750 amino acids, from about 250 amino acids to about 500 amino acids, from about 500 amino acids to about 750 amino acids, from about 500 amino acids to about 1000 amino acids, from about 500 amino acids to about 1250 amino acids, from about 500 amino acids to about 1500 amino acids, from about 750 amino acids to about 1000 amino acids, from about 750 amino acids to about 1250 amino acids, from about 750 amino acids to about 1500 amino acids, from about 1000 amino acids to about 1250 amino acids, from about 1000 amino acids to about 1500 amino acids, or from about 1250 amino acids to about 1500 amino acids.

In certain embodiments, a sialidase is chemically conjugated to a repeat chemical moiety such as PEG or hyaluronic acid (see, Mero et al. (2013) CARB POLYMERS 92:2163-70), which increases the hydrodynamic radius of the sialidase thereby to extend half-life.

In another embodiment, a sialidase is itself polysialylated or covalently attached to a negatively charged, highly sialylated protein (e.g., carboxy-terminal peptide (CTP), of chorionic gonadotropin (CG) β-chain, see, Duijkers et al. (2002) HUM REPROD 17:1987-93).

Methods for making and using the foregoing serum half-life extenders are known in the art. See also, e.g., Strohl (2015) BIODRUGS 29:215-239.

In certain embodiments, the sialidase is conjugated to a serum half-life extender that is not and Fc domain and/or is not PEG.

It is contemplated that one or more sialidases may be covalently bound to one or more (for example, 2, 3, 4, 5, 6, 8, 9, 10 or more) serum half-life extenders.

In certain embodiments, the serum half-life of the sialidase enzyme conjugated to a serum half-life enhancer is at least 24, 36, 48, or 60 hours.

In general, the serum half-life extender may have a molecular weight from about 2 kDa to about 5 kDa, from about 2 kDa to about 10 kDa, from about 2 kDa to about 20 kDa, from about 2 kDa to about 30 kDa, from about 2 kDa to about 40 kDa, from about 2 kDa to about 50 kDa, from about 2 kDa to about 60 kDa, from about 2 kDa to about 70 kDa, from about 2 kDa to about 80 kDa, from about 2 kDa to about 90 kDa, from about 2 kDa to about 100 kDa, from about 2 kDa to about 150 kDa, from about 5 kDa to about 10 kDa, from about 5 kDa to about 20 kDa, from about 5 kDa to about 30 kDa, from about 5 kDa to about 40 kDa, from about 5 kDa to about 50 kDa, from about 5 kDa to about 60 kDa, from about 5 kDa to about 70 kDa, from about 5 kDa to about 80 kDa, from about 5 kDa to about 90 kDa, from about 5 kDa to about 100 kDa, from about 5 kDa to about 150 kDa, from about 10 kDa to about 20 kDa, from about 10 kDa to about 30 kDa, from about 10 kDa to about 40 kDa, from about 10 kDa to about 50 kDa, from about 10 kDa to about 60 kDa, from about 10 kDa to about 70 kDa, from about 10 kDa to about 80 kDa, from about 10 kDa to about 90 kDa, from about 10 kDa to about 100 kDa, from about 10 kDa to about 150 kDa, from about 20 kDa to about 30 kDa, from about 20 kDa to about 40 kDa, from about 20 kDa to about 50 kDa, from about 20 kDa to about 60 kDa, from about 20 kDa to about 70 kDa, from about 20 kDa to about 80 kDa, from about 20 kDa to about 90 kDa, from about 20 kDa to about 100 kDa, from about 20 kDa to about 150 kDa, from about 30 kDa to about 40 kDa, from about 30 kDa to about 50 kDa, from about 30 kDa to about 60 kDa, from about 30 kDa to about 70 kDa, from about 30 kDa to about 80 kDa, from about 30 kDa to about 90 kDa, from about 30 kDa to about 100 kDa, from about 30 kDa to about 150 kDa, from about 40 kDa to about 50 kDa, from about 40 kDa to about 60 kDa, from about 40 kDa to about 70 kDa, from about 40 kDa to about 80 kDa, from about 40 kDa to about 90 kDa, from about 40 kDa to about 100 kDa, from about 40 kDa to about 150 kDa, from about 50 kDa to about 60 kDa, from about 50 kDa to about 70 kDa, from about 50 kDa to about 80 kDa, from about 50 kDa to about 90 kDa, from about 50 kDa to about 100 kDa, from about 50 kDa to about 150 kDa, from about 60 kDa to about 70 kDa, from about 60 kDa to about 80 kDa, from about 60 kDa to about 90 kDa, from about 60 kDa to about 100 kDa, from about 60 kDa to about 150 kDa, from about 70 kDa to about 80 kDa, from about 70 kDa to about 90 kDa, from about 70 kDa to about 100 kDa, from about 70 kDa to about 150 kDa, from about 80 kDa to about 90 kDa, from about 80 kDa to about 100 kDa, from about 80 kDa to about 150 kDa, from about 90 kDa to about 100 kDa, from about 90 kDa to about 150 kDa, or from about 100 kDa to about 150 kDa.

a. Fc Domains

In certain embodiments, the fusion protein comprises an immunoglobulin Fc domain. As used herein, unless otherwise indicated, the term “immunoglobulin Fc domain” or “Fe domain” or “Fe” refers to a fragment of an immunoglobulin heavy chain constant region which, either alone or in combination with a second immunoglobulin Fc domain, or unconjugated or conjugated to a sialidase, is capable of binding to an Fc receptor. An immunoglobulin Fc domain may include, e.g., immunoglobulin CH2 and CH3 domains. An immunoglobulin Fc domain may include, e.g., immunoglobulin CH2 and CH3 domains and an immunoglobulin hinge region. Boundaries between immunoglobulin hinge regions, CH2, and CH3 domains are well known in the art, and can be found, e.g., in the PROSITE database (available on the world wide web at prosite.expasy.org).

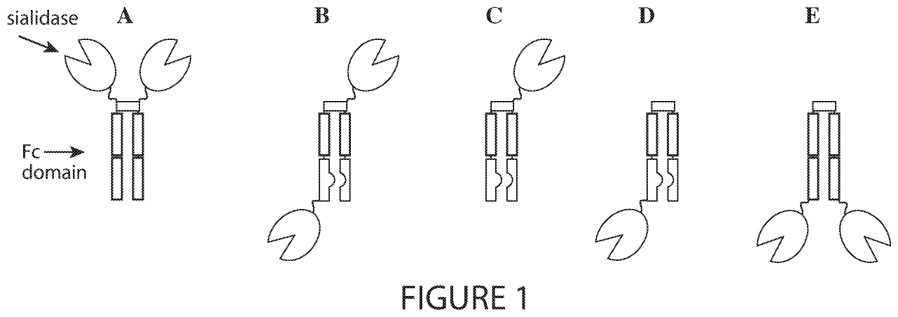

FIGS. 1A-E depict certain embodiments of sialidase-Fc fusion constructs comprising a first polypeptide comprising a first immunoglobulin Fc domain, and a second polypeptide comprising a second immunoglobulin Fc domain. The first and second polypeptides can be covalently linked together. The covalent linkages can be disulfide bonds. A sialidase enzyme can be conjugated to the N- or C-terminus of the first immunoglobulin Fc domain or to the N- or C-terminus of the second immunoglobulin Fc domain. An optional second sialidase enzyme can be conjugated to the N- or C-terminus of the first immunoglobulin Fc domain or to the N- or C-terminus of the second immunoglobulin Fc domain.

FIG. 1A shows a construct having two Fc domains and a sialidase enzyme conjugated to the N-terminus of each Fc domain. FIG. 1B shows a construct having two Fc domains and a sialidase enzyme conjugated to the C-terminus of the first Fc domain and the N-terminus of the second Fc domain. FIG. 1C shows a construct having two Fc domains and a sialidase enzyme conjugated to the N-terminus of the second Fc domain. FIG. 1D shows a construct having two Fc domains and a sialidase enzyme conjugated to the C-terminus of the first Fc domain. FIG. 1E shows a construct having two Fc domains and a sialidase enzyme conjugated to the C-terminus of the each Fc domain. It is understood that the Fc domains can be naturally occurring Fc domains or engineered Fc domains containing modifications, such as, point mutations in each polypeptide chain that facilitates a knob into hole configuration, or to provide a modified Fc domain functionality.

In certain embodiments, the immunoglobulin Fc domain is derived from a human IgG1, IgG2, IgG3, IgG4, IgA1, IgA2, IgD, IgE, and IgM Fc domain. A single amino acid substitution (S228P according to Kabat numbering; designated IgG4Pro) may be introduced to abolish the heterogeneity observed in recombinant IgG4 antibody. See Angal, S. et al. (1993) MOL. IMMUNOL. 30:105-108.

In certain embodiments, the immunoglobulin Fc domain is derived from a human IgG1 isotype or another isotype that elicits antibody-dependent cell-mediated cytotoxicity (ADCC) and/or complement mediated cytotoxicity (CDC). In certain embodiments, the immunoglobulin Fc domain is derived from a human IgG1 isotype (e.g., SEQ ID NO: 31 or SEQ ID NO: 69).

In certain embodiments, the immunoglobulin Fc domain is derived from a human IgG4 isotype or another isotype that elicits little or no antibody-dependent cell-mediated cytotoxicity (ADCC) and/or complement mediated cytotoxicity (CDC). In certain embodiments, the immunoglobulin Fc domain is derived from a human IgG4 isotype.

In certain embodiments, the immunoglobulin Fc domain comprises either a “knob” mutation, e.g., T366Y or a “hole” mutation, e.g., Y407T for heterodimerization with a second polypeptide (residue numbers according to EU numbering, Kabat, E. A., et al. (1991) SEQUENCES OF PROTEINS OF IMMUNOLOGICAL INTEREST, FIFTH EDITION, U.S. Department of Health and Human Services, NIH Publication No. 91-3242). In certain embodiments comprising a sialidase-Fc fusion with two Fc domains, the first Fc domain can comprises a “knob” mutation (such as SEQ ID NO: 33 and SEQ ID NO: 148) and the second Fc domain can comprise a “hole” mutation (such as SEQ ID NO: 32 and SEQ ID NO: 147).

In certain embodiments, a sialidase-Fc fusion protein comprises the amino acid sequence of any one of SEQ ID NOs: 129-158, 177-192, and 197-200, or an amino acid sequence that has at least 85%, 90%, 95%, 96%, 97%, 98%, or 99% sequence identity to any one of SEQ ID NOs: 129-158, 177-192, and 197-200.

In certain embodiments, the sialidase-Fc fusion protein comprises the amino acid sequence of

(SEQ ID NO: 159)
X₁X₂SX₃X₄X₅LQX₆ESVFQSGAHAYRIPALLYLPGQQSLLAFAEQRAS
X₇X₈DEHAELIVX₉RRGDYDAX₁₀THQVQWX₁₁AQEVVAQAX₁₂LDGHRS
MNPCPLYDX₁₃QTGTLFLFFIAIPX₁₄X₁₅VTEX₁₆QQLQTRANVTRL
X₁₇X₁₈VTSTDHGRTWSSPRDLTDAAIGPX₁₉YREWSTFAVGPGHX₂₀LQ
LHDRX₂₁RSLVVPAYAYRKLHPX₂₂QRPIPSAFX₂₃FLSHDHGRTWARGH
FVAQDTX₂₄ECQVAEVETGEQRVVTLNARSHLRARVQAQSX₂₅NX₂₆GLD
FQX₂₇SQLVKKLVEPPPX₂₈GX₂₉QGSVISFPSPRSGPGSPAQX₃₀LLYT
HPTHX₃₁X₃₂QRADLGAYLNPRPPAPEAWSEPX₃₃LLAKGSX₃₄AYSDLQ
SMGTGPDGSPLFGX₃₅LYEANDYEEIX₃₆FX₃₇MFTLKQAFPAEYLPQGG
GGSGGGGSDKTHTCPPCPAPELLGGPSVFLFPPKPKDTLMISRTPEVTCV
VVDVSHEDPEVKFNWYVDGVEVHNAKTKPREEQYNSTYRVVSVLTVLHQD
WLNGKEYKCKVSNKALPAPIEKTISKAKGQPREPQVYTLPPSREEMTKNQ
VSLTCLVKGFYPSDIAVEWESNGQPENNYKTTPPVLDSDGSFFLTSKLTV
DKSRWQQGNVFSCSVMHEALHNHYTQKSLSLSPGK,

wherein X₁ is Ala, Arg, Asn, Asp, Gln, Glu, Gly, His, Leu, Lys, Met, Phe, Thr, Val, or not present, X₂ is Ala or Lys, X₃ is Asn or Leu, X₄ is Pro or His, X₅ is Phe, Trp, Tyr or Val, X₆ is Lys or Asp. X₇ is Lys, Arg, or Glu. X₈ is Lys, Ala, Arg, or Glu, X₉ is Leu or Met, X₁₀ is Pro, Asn, Asp, His, Glu, Gly, Ser or Thr, X₁₁ is Gln or His, X₁₂ is Arg or Lys, X₁₃ is Ala, Glu or Lys, X₁₄ is Gly or Asp, X₁₅ is Gln or His, X₁₆ is Gln, Arg, or Lys, X₁₇ is Ala, Cys, Ile, Ser, Val, or Leu, X₁₈ is Gln or Leu, X₁₉ is Ala or Val, X₂₀ is Cys or Gly, X₂₁ is Ala or Gly, X₂₂ is Arg, Ile, or Lys, X₂₃ is Ala, Cys, Leu, or Val, X₂₄ is Leu, Ala, or Val, X₂₅ is Thr or Ala, X₂₆ is Asp or Gly, X₂₇ is Glu or Lys, X₂₈ is Gln, Ala, His, Phe, or Pro, X₂₉ is Cys or Val, X₃₀ is Trp or Arg, X₃₁ is Ser or Arg, X₃₂ is Trp or Lys, X₃₃ is Lys or Val, X₃₄ is Ala, Cys, Ser, or Val, X₃₅ is Cys, Leu, or Val, X₃₆ is Val or Arg, and X₃₇ is Leu, Gln, His, Ile, Lys, or Ser, and the sialidase comprises at least one mutation relative to wild-type human Neu2 (SEQ ID NO: 1).

In certain embodiments, the sialidase-Fc fusion protein comprises the amino acid sequence of

(SEQ ID NO: 160)
X₁ASLPX₂LQX₃ESVFQSGAHAYRIPALLYLPGQQSLLAFAEQRASKKDE
HAELIVLRRGDYDAX₄THQVQWQAQEVVAQARLDGHRSMNPCPLYDX₅QT
GTLFLFFIAIPGQVTEQQQLQTRANVTRLCQVTSTDHGRTWSSPRDLTDA
AIGPAYREWSTFAVGPGHCLQLHDRARSLVVPAYAYRKLHPX₆QRPIPSA
CFLSHDHGRTWARGHFVAQDTLECQVAEVETGEQRVVTLNARSHLRARVQ
FAQSTNDGLDFQESQLVKKLVEPPPX₇GCQGSVISFPSPRSGPGSPAQWL
LYTHPTHX₈X₉QRADLGAYLNPRPPAPEAWSEPVLLAKGSX₁₀AYSDLQS
MGTGPDGSPLFGCLYEANDYEEIX₁₁FX₁₂MFTLKQAFPAEYLPQGGGGS
GGGSDKTHTCPPCPAPELLGGPSVFLFPPKPKDTLMISRTPEVTCVVVD
VSHEDPEVKFNWYVDGVEVHNAKTKPREEQYNSTYRVVSVLTVLHQDWLN
GKEYKCKVSNKALPAPIEKTISKAKGQPREPQVYTLPPSREEMTKNQVSL
TCLVKGFYPSDIAVEWESNGQPENNYKTTPPVLDSDGSFFLTSKLTVDKS
RWQQGNVFSCSVMHEALHNHYTQKSLSLSPGK,

wherein X₁ is Ala, Arg, Asn, Asp, Gln, Glu, Gly, His, Leu, Lys, Met, Phe, Thr, Val, or not present, X₂ is Phe, Trp, Tyr or Val, X₃ is Lys or Asp, X₄ is Pro, Asn, Asp, His, Glu, Gly, Ser or Thr, X₅ is Ala, Glu, or Lys, X₆ is Arg, Ile, or Lys, X₇ is Gln, Ala, His, Phe, or Pro, X₈ is Ser or Arg, X₉ is Trp or Lys, X₁₀ is Ala, Cys, Ser, or Val, X₁₁ is Val or Arg, and X₁₂ is Leu, Gln, His, Ile, Lys, or Ser, and the sialidase comprises at least one mutation relative to wild-type human Neu2 (SEQ ID NO: 1). In certain embodiments, X₁ is Ala, Asp, Met, or not present, X₂ is Tyr or Val, X₃ is Lys or Asp, X₄ is Pro, Asn, Gly, Ser or Thr, X₅ is Ala or Glu, X₆ is Ile or Lys, X₇ is Gln or Ala, X₈ is Ser or Arg, X₉ is Trp or Lys, X₁₀ is Ala or Cys, X₁₁ is Val or Arg, and X₁₂ is Leu or Ile.

In certain embodiments, the sialidase-Fc fusion protein comprises the amino acid sequence of

(SEQ ID NO: 161)
X₁X₂SX₃X₄X₅LQX₆ESVFQSGAHAYRIPALLYLPGQQSLLAFAEQRAS
X₇X₈DEHAELIVX₉RRGDYDAX₁₀THQVQWX₁₁AQEVVAQAX₁₂LDGHRS
MNPCPLYDX₁₃QTGTLFLFFIAIPX₁₄X₁₅VTEX₁₆QQLQTRANVTRL
X₁₇X₁₈VTSTDHGRTWSSPRDLTDAAIGPX₁₉YREWSTFAVGPGHX₂₀LQ
LHDRX₂₁RSLVVPAYAYRKLHPX₂₂QRPIPSAFX₂₃FLSHDHGRTWARGH
FVAQDTX₂₄ECQVAEVETGEQRVVTLNARSHLRARVQAQSX₂₅NX₂₆GLD
FQX₂₇SQLVKKLVEPPPX₂₈GX₂₉QGSVISFPSPRSGPGSPAQX₃₀LLYTH
PTHX₃₁X₃₂QRADLGAYLNPRPPAPEAWSEPX₃₃LLAKGSX₃₄AYSDLQS
MGTGPDGSPLFGX₃₅LYEANDYEEIX₃₆FX₃₇MFTLKQAFPAEYLPQX₃₈
DKTHTCPPCPAPELLGGPSVFLFPPKPKDTLMISRTPEVTCVVVDVSHED
PEVKFNWYVDGVEVHNAKTKPREEQYNSTYRVVSVLTVLHQDWLNGKEYK
CKVSNKALPAPIEKTISKAKGQPREPQVYTLPPSREEMTKNQVSLTCLVK
GFYPSDIAVEWESNGQPENNYKTTPPVLDSDGSFFLTSKLTVDKSRWQQG
NVFSCSVMHEALHNHYTQKSLSLSPGK,

wherein X₁ is Ala, Arg, Asn, Asp, Gln, Glu, Gly, His, Leu, Lys, Met, Phe, Thr, Val, or not present, X₂ is Ala or Lys, X₃ is Asn or Leu, X₄ is Pro or His, X₅ is Phe, Trp, Tyr or Val, X₆ is Lys or Asp. X₇ is Lys, Arg, or Glu. X₈ is Lys, Ala, Arg, or Glu, X₉ is Leu or Met, X₁₀ is Pro, Asn, Asp, His, Glu, Gly, Ser or Thr, X₁₁ is Gln or His, X₁₂ is Arg or Lys, X₁₃ is Ala, Glu or Lys, X₁₄ is Gly or Asp, X₁₅ is Gln or His, X₁₆ is Gln, Arg, or Lys, X₁₇ is Ala, Cys, Ile, Ser, Val, or Leu, X₁₈ is Gln or Leu, X₁₉ is Ala or Val, X₂₀ is Cys or Gly, X₂₁ is Ala or Gly, X₂₂ is Arg, Ile, or Lys, X₂₃ is Ala, Cys, Leu, or Val, X₂₄ is Leu, Ala, or Val, X₂₅ is Thr or Ala, X₂₆ is Asp or Gly, X₂₇ is Glu or Lys, X₂₈ is Gln, Ala, His, Phe, or Pro, X₂₉ is Cys or Val, X₃₀ is Trp or Arg, X₃₁ is Ser or Arg, X₃₂ is Trp or Lys, X₃₃ is Lys or Val, X₃₄ is Ala, Cys, Ser, or Val, X₃₅ is Cys, Leu, or Val, X₃₆ is Val or Arg, X₃₇ is Leu, Gln, His, Ile, Lys, or Ser, X₃₈ is GGGGSGGGGS (SEQ ID NO: 162) or EPKSS (SEQ ID NO: 163), and the sialidase comprises at least one mutation relative to wild-type human Neu2 (SEQ ID NO: 1).

In certain embodiments, the sialidase-Fc fusion protein comprises the amino acid sequence of

(SEQ ID NO: 164)
X₁ASLPX₂LQX₃ESVFQSGAHAYRIPALLYLPGQQSLLAFAEQRASKKDE
HAELIVLRRGDYDAX₄THQVQWQAQEVVAQARLDGHRSMNPCPLYDX₅QT
GTLFLFFIAIPGQVTEQQQLQTRANVTRLCQVTSTDHGRTWSSPRDLTDA
AIGPAYREWSTFAVGPGHCLQLHDRARSLVVPAYAYRKLHPX₆QRPIPSA
FCFLSHDHGRTWARGHFVAQDTLECQVAEVETGEQRVVTLNARSHLRARV
QAQSTNDGLDFQESQLVKKLVEPPPX₇GCQGSVISFPSPRSGPGSPAQWL
LYTHPTHX₈X₉QRADLGAYLNPRPPAPEAWSEPVLLAKGSX₁₀AYSDLQS
MGTGPDGSPLFGCLYEANDYEEIX₁₁FX₁₂MFTLKQAFPAEYLPQX₁₃DK
THTCPPCPAPELLGGPSVFLFPPKPKDTLMISRTPEVTCVVVDVSHEDPE
VKFNWYVDGVEVHNAKTKPREEQYNSTYRVVSVLTVLHQDWLNGKEYKCK
VSNKALPAPIEKTISKAKGQPREPQVYTLPPSREEMTKNQVSLTCLVKGF
YPSDIAVEWESNGQPENNYKTTPPVLDSDGSFFLTSKLTVDKSRWQQGNV
FSCSVMHEALHNHYTQKSLSLSPGK,

wherein X₁ is Ala, Arg, Asn, Asp, Gln, Glu, Gly, His, Leu, Lys, Met, Phe, Thr, Val, or not present, X₂ is Phe, Trp, Tyr or Val, X₃ is Lys or Asp, X₄ is Pro, Asn, Asp, His, Glu, Gly, Ser or Thr, X₅ is Ala, Glu, or Lys, X₆ is Arg, Ile, or Lys, X₇ is Gln, Ala, His, Phe, or Pro, X₈ is Ser or Arg, X₉ is Trp or Lys, X₁₀ is Ala, Cys, Ser, or Val, X₁₁ is Val or Arg, X₁₂ is Leu, Gln, His, Ile, Lys, or Ser, and X₁₃ is GGGGSGGGGS (SEQ ID NO: 162) or EPKSS (SEQ ID NO: 163), and the sialidase comprises at least one mutation relative to wild-type human Neu2 (SEQ ID NO: 1). In certain embodiments, X₁ is Ala, Asp, Met, or not present, X₂ is Tyr or Val, X₃ is Lys or Asp, X₄ is Pro, Asn, Gly, Ser or Thr, X₅ is Ala or Glu, X₆ is Ile or Lys, X₇ is Gln or Ala, X₈ is Ser or Arg, X₉ is Trp or Lys, X₁₀ is Ala or Cys, X₁₁ is Val or Arg, and X₁₂ is Leu or Ile.

In certain embodiments, the sialidase-Fc fusion protein comprises the amino acid sequence of

(SEQ ID NO: 165)
X₁X₂SX₃X₄X₅LQX₆ESVFQSGAHAYRIPALLYLPGQQSLLAFAEQRAS
X₇X₈DEHAELIVX₉RRGDYDAX₁₀THQVQWX₁₁AQEVVAQAX₁₂LX₁₃GH
RSMNPCPLYDX₁₄QTGTLFLFFIAIPX₁₅X₁₆VTEX₁₇QQLQTRANVTRL
X₁₈X₁₉VTSTDHGRTWSSPRDLTDAAIGPX₂₀YREWSTFAVGPGHX₂₁LQ
LHDX₂₂X₂₃RSLVVPAYAYRKLHPX₂₄X₂₅X₂₆PIPSAFX₂₇FLSHDHGR
TWARGHFVX₂₈QDTX₂₉ECQVAEVX₃₀TGEQRVVTLNARSX₃₁X₃₂X₃₃
X₃₄RX₃₅QAQSX₃₆NX₃₇GLDFQX₃₈X₃₉QX₄₀VKKLX₄₁EPPPX₄₂G
X₄₃QGSVISFPSPRSGPGSPAQX₄₄LLYTHPTHX₄₅X₄₆QRADLGAYLNP
RPPAPEAWSEPX₄₇LLAKGSX₄₈AYSDLQSMGTGPDGSPLFGX₄₉LYEA
NDYEEIX₅₀FX₅₁MFTLKQAFPAEYLPQX₅₂DKTHTCPPCPAPELLGGPS
VFLFPPKPKDTLMISRTPEVTCVVVDVSHEDPEVKFNWYVDGVEVHNAKT
KPREEQYNSTYRVVSVLTVLHQDWLNGKEYKCKVSNKALPAPIEKTISKA
KGQPREPQVYTLPPSREEMTKNQVSLTCLVKGFYPSDIAVEWESNGQPEN
NYKTTPPVLDSDGSFFLTSKLTVDKSRWQQGNVFSCSVMHEALHNHYTQK
SLSLSPGK,

wherein X₁ is Ala, Arg, Asn, Asp, Gln, Glu, Gly, His, Leu, Lys, Met, Phe, Thr, Val, or not present, X₂ is Ala or Lys, X₃ is Asn or Leu, X₄ is Pro or His, X₅ is Phe, Trp, Tyr or Val, X₆ is Lys or Asp, X₇ is Lys, Arg, or Glu, X₈ is Lys, Ala, Arg, or Glu, X₉ is Leu or Met, X₁₀ is Pro, Asn, Asp, His, Glu, Gly, Ser or Thr, X₁₁ is Gln or His, X₁₂ is Arg or Lys, X₁₃ is Asp or Pro, X₁₄ is Ala, Glu or Lys, X₁₅ is Gly or Asp, X₁₆ is Gln or His, X₁₇ is Gln, Arg, or Lys, X₁₈ is Ala, Cys, Ile, Ser, Val, or Leu, X₁₉ is Gln, Leu, Glu, Phe, His, Ile, Leu, or Tyr, X₂₀ is Ala or Val, X₂₁ is Cys or Gly, X₂₂ is Arg or Pro, X₂₃ is Ala or Gly, X₂₄ is Arg, Ile, or Lys, X₂₅ is Gln or Pro, X₂₆ is Arg or Pro, X₂₇ is Ala, Cys, Leu, or Val, X₂₈ is Ala, Cys, Asn, Ser, or Thr, X₂₉ is Leu, Ala, or Val, X₃₀ is Glu or Pro, X₃₁ is His or Pro, X₃₂ is Leu, Asp, Asn, or Tyr, X₃₃ is Arg, Ala, Asp, Leu, Gln, or Tyr, X₃₄ is Ala, Cys, Phe, Gly, His, Ile, Lys, Leu, Met, Asn, Gln, Arg, Ser, Val, Trp, or Tyr, X₃₅ is Val, Ile, or Lys, X₃₆ is Thr or Ala, X₃₇ is Asp or Gly, X₃₈ is Glu, Lys, or Pro, X₃₉ is Ser or Cys, X₄₀ is Leu, Asp, Phe, Gln, or Thr, X₄₁ is Val or Phe, X₄₂ is Gln, Ala, His, Phe, Pro, Ser, or Thr, X₄₃ is Cys or Val, X₄₄ is Trp or Arg, X₄₅ is Ser, Arg, Ala, Asp, Glu, Phe, Gly, His, Ile, Lys, Leu, Met, Asn, Pro, Gln, Thr, Val, Trp, or Tyr, X₄₆ is Trp, Lys, Ala, Asp, Glu, Phe, Gly, His, Ile, Lys, Leu, Met, Asn, Pro, Gln, Arg, Ser, Thr, Val, or Tyr, X₄₇ is Lys or Val, X₄₈ is Ala, Cys, Ser, or Val, X₄₉ is Cys, Leu, or Val, X₅₀ is Val or Arg, X₅₁ is Leu, Gln, His, Ile, Lys, or Ser, X₅₂ is GGGGS (SEQ ID NO: 174), GGGGSGGGGS (SEQ ID NO: 162), or EPKSS (SEQ ID NO: 163), and the sialidase comprises at least one mutation relative to wild-type human Neu2 (SEQ ID NO: 1).

In certain embodiments, the sialidase-Fc fusion protein comprises the amino acid sequence of

(SEQ ID NO: 166)
X₁ASLPX₂LQX₃ESVFQSGAHAYRIPALLYLPGQQSLLAFAEQRASKKDE
HAELIVLRRGDYDAX₄THQVQWQAQEVVAQARLDGHRSMNPCPLYDX₅QT
GTLFLFFIAIPGQVTEQQQLQTRANVTRLCX₆VTSTDHGRTWSSPRDLTD
AAIGPAYREWSTFAVGPGHCLQLHDRARSLVVPAYAYRKLHPX₇QRPIPS
AFCFLSHDHGRTWARGHFVAQDTLECQVAEVETGEQRVVTLNARSHLRX₈
RVQAQSTNDGLDFQESQLVKKLVEPPPX₉GCQGSVISFPSPRSGPGSPAQ
WLLYTHPTHX₁₀X₁₁QRADLGAYLNPRPPAPEAWSEPVLLAKGSX₁₂AYS
DLQSMGTGPDGSPLFGCLYEANDYEEIX₁₃FX₁₄MFTLKQAFPAEYLPQ
X₁₅DKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPSREEMTKNQVSLTC
LVKGFYPSDIAVEWESNGQPENNYKTTPPVLDSDGSFFLTSKLTVDKSRW
QQGNVFSCSVMHEALHNHYTQKSLSLSPGK,

wherein X₁ is Ala, Arg, Asn, Asp, Gln, Glu, Gly, His, Leu, Lys, Met, Phe, Thr, Val, or not present, X₂ is Phe, Trp, Tyr or Val, X₃ is Lys or Asp, X₄ is Pro, Asn, Asp, His, Glu, Gly, Ser or Thr, X₅ is Ala, Glu, or Lys, X₆ is Gln, Leu, Glu, Phe, His, Ile, Leu, or Tyr, X₇ is Arg, Ile, or Lys, X₈ is Ala, Cys, Phe, Gly, His, Ile, Lys, Leu, Met, Asn, Gln, Arg, Ser, Val, Trp, or Tyr, X₉ is Gln, Ala, His, Phe, Pro, Ser, or Thr, X₁₀ is Ser, Arg, Ala, Asp, Glu, Phe, Gly, His, Ile, Lys, Leu, Met, Asn, Pro, Gln, Thr, Val, Trp, or Tyr, X₁₁ is Trp, Lys, Ala, Asp, Glu, Phe, Gly, His, Ile, Lys, Leu, Met, Asn, Pro, Gln, Arg, Ser, Thr, Val, or Tyr, X₁₂ is Ala, Cys, Ser, or Val, X₁₃ is Val or Arg, X₁₄ is Leu, Gln, His, Ile, Lys, or Ser, X₁₅ is GGGGS (SEQ ID NO: 184), GGGGSGGGGS (SEQ ID NO: 162), or EPKSS (SEQ ID NO: 163), and the sialidase comprises at least one mutation relative to wild-type human Neu2 (SEQ ID NO: 1). In certain embodiments, X₁ is Ala, Asp, Met, or not present, X₂ is Tyr or Val, X₃ is Lys or Asp, X₄ is Pro, Asn, Gly, Ser or Thr, X₅ is Ala or Glu, X₆ is Gln or Tyr, X₇ is Ile or Lys, X₈ is Ala or Thr, X₉ is Gln, Ala, or Thr, X₁₀ is Ser, Arg, or Ala, X₁₁ is Trp, Lys, or Arg, X₁₂ is Ala or Cys, X₁₃ is Val or Arg, and X₁₄ is Leu or Ile.
b. Polyethylene Glycol (PEG)

In one embodiment, the serum half-life extender is polyethylene glycol (PEG) and derivatives thereof (for example, alkoxy polyethylene glycol, for example, methoxypolyethylene glycol, ethoxypolyethylene glycol and the like). In one embodiment, the sialidase as described herein is covalently attached to at least one PEG having an actual MW of at least about 20,000 D. In another embodiment, the sialidase is covalently attached to at least one PEG having an actual MW of at least about 30,000 D. In another embodiment, the sialidase is covalently attached to at least one PEG having an actual MW of at least about 40,000 D. In certain embodiments, the PEG is methoxyPEG(5000)-succinimidylpropionate (mPEG-SPA), methoxyPEG(5000)-succinimidylsuccinate (mPEG-SS). Such PEGS are commercially available from Nektar Therapeutics or SunBiowest or LaysanBio or NOF. In one embodiment, the PEG may be branched, or Y-shaped, as available from JenKem USA or NOF, or comb-shaped, or synthesized by coupling two or more PEGs to a small molecule such as glutamic acid.

The omega position of PEG may include a hydroxyl group or a methoxy group and the PEG may also contain an amino group in the omega position. Such an amino group can in turn be coupled to a variety of agents. In another embodiment of the present invention, the biological modifier can be a pegylated poly-L-lysine or a pegylated poly-D-lysine.

Attachment sites on a sialidase for a PEG or a derivative thereof include the N-terminal amino group and epsilon amino groups found on lysine residues, as well as other amino, imino, carboxyl, sulfhydryl, hydroxyl or other hydrophilic groups. PEG may be covalently bonded directly to the sialidase with or without the known use of a multifunctional (ordinarily bifunctional) crosslinking agent using chemistries and used in the art. For example, the PEG modifier can be conjugated to the sialidase by using a thiol reactive cross linker and then reacting with a thiol group on the PEG. In certain embodiments, sulfhydryl groups can be derivatized by coupling to maleimido-substituted PEG (e.g. alkoxy-PEG amine plus sulfosuccinimidyl 4-(N-maleimidomethyl)cyclohexane-1-carboxylate), or PEG-maleimide commercially available from Shearwater Polymers, Inc., Huntsville, Ala.).

c. Human Serum Albumin (HSA) and HSA Binders

Human serum albumin (HSA) (molecular mass ˜67 kDa) is the most abundant protein in plasma, present at about 50 mg/mL (600 μM), and has a half-life of around 20 days is humans. HSA serves to maintain plasma pH, contributes to colloidal blood pressure, functions as carrier of many metabolites and fatty acids, and serves as a major drug transport protein in plasma.

In certain embodiments, the serum half-life extender is human serum albumin (HSA) or an HSA-binding peptide (see, e.g., PCT Publication Nos. WO2013128027A1 and WO2014140358A1). The neonatal Fc receptor (FcRn) appears to be involved in prolonging the life-span of albumin in circulation (see, Chaudhury et al. (2003) J. EXP. MED., 3: 315-22). Albumin and IgG bind noncooperatively to distinct sites of FcRn and form a tri-molecular (see id.). Binding of human FcRn to HSA and to human IgG is pH dependent, stronger at acidic pH and weaker at neutral or physiological pH (see id.). This observation suggests that proteins and protein complexes containing albumin, similar to those containing IgG (particularly Fc), are protected from degradation through pH-sensitive interaction with FcRn (see id.). Using surface plasmon resonance (SPR) to measure the capacity of individual HSA domains to bind immobilized soluble human FcRn, it has been shown that FcRn and albumin interact via the D-III domain of albumin in a pH-dependent manner, on a site distinct from the IgG binding site (see, Chaudhury et al. (2006) BIOCHEM. 45:4983-90 and PCT Publication No. WO2008068280A1).

Exemplary HSA-binding proteins are known in the art. For example, U.S. Patent Application Publication No. US20130316952A1 discloses a polypeptide that binds serum albumin having the amino acid sequence of

(SEQ ID NO: 109)
LKEAKEKAIEELKKAGITSDYYFDLINKAKTVEGVNALKDEILKA.

Additional exemplary polypeptides that bind HSA are described in Dennis et al. (2002) J. BIOL. CHEM., 277: 35035-43; Jacobs et al. (2015) PROTEIN ENG. DES. SEL., 28: 385-93; and Zorzi et al. (2017) NAT. COMMUN., 8: 16092.

### III. Linkers

In certain embodiments, the sialidase can be linked or fused directly to the serum half-life extender. In other embodiments, the sialidase can be covalently bound to the serum half-life extender by a linker.

The linker may couple, with one or more natural amino acids, the sialidase, or functional fragment thereof, and the serum half-life extender, where the one or more natural amino acids (for example, a cysteine amino acid) may be introduced by site-directed mutagenesis. The linker may include one or more unnatural amino acids. It is contemplated that, in certain circumstances, a linker containing for example, one or more sulfhydryl reactive groups (e.g., a maleimide) may covalently link a cysteine in the sialidase or the serum half-life extender that is a naturally occurring cysteine residue or is the product of site-specific mutagenesis.

The linker may be a cleavable linker or a non-cleavable linker. Optionally or in addition, the linker may be a flexible linker or an inflexible linker.

The linker should be a length sufficiently long to allow the sialidase and the serum half-life extender to be linked without steric hindrance from one another and sufficiently short to retain the intended activity of the fusion protein. The linker preferably is sufficiently hydrophilic to avoid or minimize instability of the fusion protein. The linker preferably is sufficiently hydrophilic to avoid or minimize insolubility of the fusion protein. The linker should be sufficiently stable in vivo (e.g., it is not cleaved by serum, enzymes, etc.) to permit the fusion protein to be operative in vivo.

The linker may be from about 1 angstroms (Å) to about 150 Å in length, or from about 1 Å to about 120 Å in length, or from about 5 Å to about 110 Å in length, or from about 10 Å to about 100 Å in length. The linker may be greater than about 2, 3, 4, 5, 6, 7, 8, 9, 10, 11, 12, 13, 14, 15, 16, 17, 18, 19, 20, 25, 27, 30 or greater angstroms in length and/or less than about 110, 100, 90, 85, 80, 75, 70, 65, 60, 55, 50, 45, 43, 42, 41, 40, 39, 38, 37, 36, 35, 34, 33, 32, 31, or fewer A in length. Furthermore, the linker may be about 5, 10, 15, 20, 25, 30, 35, 40, 45, 50, 55, 60, 65, 70, 75, 80, 85, 90, 95, 100, 110, and 120 Å in length.

In certain embodiments, the linker comprises a polypeptide linker that connects or fuses the sialidase to the serum half-life extender (e.g., Fc domain) of the fusion protein. For example, it is contemplated that a gene encoding a sialidase linked directly or indirectly (for example, via an amino acid containing linker) to a serum half-life extender can be created and expressed using conventional recombinant DNA technologies. For example, the amino terminus of a sialidase can be linked to the carboxy terminus of a serum half-life extender. When a linker is employed, the linker may comprise hydrophilic amino acid residues, such as Gln, Ser, Gly, Glu, Pro, His and Arg. In certain embodiments, the linker is a peptide containing 1-25 amino acid residues, 1-20 amino acid residues, 2-15 amino acid residues, 3-10 amino acid residues, 3-7 amino acid residues, 4-25 amino acid residues, 4-20 amino acid residues, 4-15 amino acid residues, 4-10 amino acid residues, 5-25 amino acid residues, 5-20 amino acid residues, 5-15 amino acid residues, or 5-10 amino acid residues. Exemplary linkers include glycine and serine-rich linkers, e.g., (GlyGlyPro)ₙ, (SEQ ID NO: 110) or (GlyGlyGlyGlySer)ₙ, (SEQ ID NO: 111) where n is 1-5. In certain embodiments, the linker comprises, consists, or consists essentially of GGGGS (SEQ ID NO: 174). In certain embodiments, the linker comprises, consists, or consists essentially of GGGGSGGGGS (SEQ ID NO: 162). In certain embodiments, the linker comprises, consists, or consists essentially of EPKSS (SEQ ID NO: 163). Additional exemplary linker sequences are disclosed, e.g., in George et al. (2003) PROTEIN ENGINEERING 15:871-879, and U.S. Pat. Nos. 5,482,858 and 5,525,491.

IV. Methods of Making a Sialidase and/or a Sialidase Conjugated to a Serum Half-life Enhancer

Methods for producing a sialidase or a sialidase conjugated to a serum half-life enhancer e.g., those disclosed herein, are known in the art. For example, DNA molecules encoding a serum half-life enhancer (e.g., an Fc domain) can be synthesized chemically or by recombinant DNA methodologies. For example, the sequences of the serum half-life enhancer can be cloned by conventional hybridization techniques or polymerase chain reaction (PCR) techniques, using the appropriate synthetic nucleic acid primers. The resulting DNA molecules encoding the protein of interest can be ligated to other appropriate nucleotide sequences, including, for example, expression control sequences, to produce conventional gene expression constructs (i.e., expression vectors) encoding the desired serum half-life enhancer. Production of defined gene constructs is within routine skill in the art.

Nucleic acids encoding desired sialidases can be incorporated (ligated) into expression vectors, which can be introduced into host cells through conventional transfection or transformation techniques. Exemplary host cells are E. coli cells, Chinese hamster ovary (CHO) cells, human embryonic kidney 293 (HEK 293) cells, HeLa cells, baby hamster kidney (BHK) cells, monkey kidney cells (COS), human hepatocellular carcinoma cells (e.g., Hep G2), and myeloma cells that do not otherwise produce IgG protein. Transformed host cells can be grown under conditions that permit the host cells to express the sialidase.

Specific expression and purification conditions will vary depending upon the expression system employed. For example, if a gene is to be expressed in E. coli, it is first cloned into an expression vector by positioning the engineered gene downstream from a suitable bacterial promoter, e.g., Trp or Tac, and a prokaryotic signal sequence. The expressed protein may be secreted. The expressed protein may accumulate in refractile or inclusion bodies, which can be harvested after disruption of the cells by French press or sonication. The refractile bodies then are solubilized, and the protein may be refolded and/or cleaved by methods known in the art.

If the engineered gene is to be expressed in eukaryotic host cells, e.g., CHO cells, it is first inserted into an expression vector containing a suitable eukaryotic promoter, a secretion signal, a poly A sequence, and a stop codon. Optionally, the vector or gene construct may contain enhancers and introns. The gene construct can be introduced into eukaryotic host cells using conventional techniques.

A polypeptide comprising a sialidase or a fusion protein, e.g., a fusion protein comprising an immunoglobulin heavy chain variable region or light chain variable region, can be produced by growing (culturing) a host cell transfected with an expression vector encoding such a variable region, under conditions that permit expression of the polypeptide. Following expression, the polypeptide can be harvested and purified or isolated using techniques known in the art, e.g., affinity tags such as glutathione-S-transferase (GST) or histidine tags.

In embodiments in which a sialidase or sialidase conjugated to an Fc region, can be produced by growing (culturing) a host cell transfected with: (a) an expression vector that encodes a one Fc polypeptide, and a separate expression vector that encodes another Fc polypeptide; or (b) a single expression vector that encodes both Fc polypeptides, under conditions that permit expression of both polypeptides. The sialidase will be fused to one or more of the polypeptides. The intact sialidase-Fc domain fusion protein can be harvested and purified or isolated using techniques known in the art, e.g., Protein A, Protein G, affinity tags such as glutathione-S-transferase (GST) or histidine tags.

In certain embodiments, a sialidase or a sialidase conjugated to a serum half-life extender is expressed and/or purified in the presence of a stabilizing agent. The stabilizing agent prevents one or more of protein unfolding, protein misfolding, protein aggregation, protein inhibition, enzymatic loss and/or protein degradation of the sialidase or the sialidase conjugated to a serum half-life extender during expression, purification and/or storage. In certain embodiments, the stabilizing agent is a cation, such as a divalent cation. In certain embodiments, the cation is calcium or magnesium. The cation can be in the form of a salt, such as calcium chloride (CaCl₂) or magnesium chloride (MgCl₂).

In certain embodiments, the stabilizing agent is present in an amount from about 0.05 mM to about 5 mM during expression and/or purification. For example, the stabilizing agent may be present in an amount of from about 0.05 mM to about 4 mM, from about 0.05 mM to about 3 mM, from about 0.05 mM to about 2 mM, from about 0.05 mM to about 1 mM, from about 0.05 mM to about 0.5 mM, from about 0.5 mM to about 4 mM, from about 0.5 mM to about 3 mM, from about 0.5 mM to about 2 mM, from about 0.5 mM to about 1 mM, from about 1 mM to about 4 mM, from about 1 mM to about 3 mM, of from about 1 mM to about 2 mM.

In certain embodiments, in order to express a protein, e.g., a sialidase, as a secreted protein, a native N-terminal signal sequence of the protein is replaced, e.g., with MDMRVPAQLLGLLLLWLPGARC (SEQ ID NO: 28). In certain embodiments, to express a protein, e.g., a recombinant human sialidase, as a secreted protein, an N-terminal signal sequence, e.g., MDMRVPAQLLGLLLLWLPGARC (SEQ ID NO: 28), is added. Additional exemplary N-terminal signal sequences include signal sequences from interleukin-2, CD-5, IgG kappa light chain, trypsinogen, serum albumin, and prolactin. In certain embodiments, in order to express a protein, e.g., a recombinant human sialidase, as a secreted protein, a C terminal lysosomal signal motif, e.g., YGTL (SEQ ID NO: 29) is removed.

In certain embodiments, when a sialidase is chemically conjugated to a serum half-life extender, the chemical conjugation can be performed using methods known in the art. Attachment sites on a sialidase and/or a serum half-life extender include the N-terminal amino group and epsilon amino groups found on lysine residues, as well as other amino, imino, carboxyl, sulfhydryl, hydroxyl or other hydrophilic groups. A serum half-life extender may be covalently bonded directly to the sialidase with or without the known use of a multifunctional (ordinarily bifunctional) crosslinking agent using chemistries and used in the art. For example, in the case of PEG, sulfhydryl groups can be derivatized by coupling to maleimido-substituted PEG (e.g. alkoxy-PEG amine plus sulfosuccinimidyl 4-(N-maleimidomethyl)cyclohexane-1-carboxylate), or PEG-maleimide commercially available from Shearwater Polymers, Inc., Huntsville, Ala.).

### V. Pharmaceutical Compositions

For therapeutic use, a sialidase or sialidase conjugated to a half-life extender preferably is combined with a pharmaceutically acceptable carrier. The term “pharmaceutically acceptable” as used herein refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The term “pharmaceutically acceptable carrier” as used herein refers to buffers, carriers, and excipients suitable for use in contact with the tissues of human beings and animals without excessive toxicity, irritation, allergic response, or other problem or complication, commensurate with a reasonable benefit/risk ratio. Pharmaceutically acceptable carriers include any of the standard pharmaceutical carriers, such as a phosphate buffered saline solution, water, emulsions (e.g., such as an oil/water or water/oil emulsions), and various types of wetting agents. The compositions also can include stabilizers and preservatives. For examples of carriers, stabilizers and adjuvants, see, e.g., Martin, Remington's Pharmaceutical Sciences, 15th Ed., Mack Publ. Co., Easton, Pa. [1975]. Pharmaceutically acceptable carriers include buffers, solvents, dispersion media, coatings, isotonic and absorption delaying agents, and the like, that are compatible with pharmaceutical administration. The use of such media and agents for pharmaceutically active substances is known in the art.

In certain embodiments, a pharmaceutical composition may contain formulation materials for modifying, maintaining or preserving, for example, the pH, osmolarity, viscosity, clarity, color, isotonicity, odor, sterility, stability, rate of dissolution or release, adsorption or penetration of the composition. In such embodiments, suitable formulation materials include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates or other organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides; disaccharides; and other carbohydrates (such as glucose, mannose or dextrins); proteins (such as serum albumin, gelatin or immunoglobulins); coloring, flavoring and diluting agents; emulsifying agents; hydrophilic polymers (such as polyvinylpyrrolidone); low molecular weight polypeptides; salt-forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate, triton, tromethamine, lecithin, cholesterol, tyloxapal); stability enhancing/stabilizing agents (such as sucrose, sorbitol, or a cation); tonicity enhancing agents (such as alkali metal halides, preferably sodium or potassium chloride, mannitol sorbitol); delivery vehicles; diluents; excipients and/or pharmaceutical adjuvants (see, Remington's Pharmaceutical Sciences, 18th ed. (Mack Publishing Company, 1990).

In certain embodiments, a pharmaceutical composition may contain a stabilizing agent. In certain embodiments, the stabilizing agent is a cation, such as a divalent cation. In certain embodiments, the cation is calcium or magnesium. The cation can be in the form of a salt, such as calcium chloride (CaCl₂) or magnesium chloride (MgCl₂).

In certain embodiments, the stabilizing agent is present in an amount from about 0.05 mM to about 5 mM. For example, the stabilizing agent may be present in an amount of from about 0.05 mM to about 4 mM, from about 0.05 mM to about 3 mM, from about 0.05 mM to about 2 mM, from about 0.05 mM to about 1 mM, from about 0.05 mM to about 0.5 mM, from about 0.5 mM to about 4 mM, from about 0.5 mM to about 3 mM, from about 0.5 mM to about 2 mM, from about 0.5 mM to about 1 mM, from about 1 mM to about 4 mM, from about 1 mM to about 3 mM, of from about 1 mM to about 2 mM.

In certain embodiments, a pharmaceutical composition may contain nanoparticles, e.g., polymeric nanoparticles, liposomes, or micelles (See Anselmo et al. (2016) BIOENG. TRANSL. MED. 1: 10-29).

In certain embodiments, a pharmaceutical composition may contain a sustained- or controlled-delivery formulation. Techniques for formulating sustained- or controlled-delivery means, such as liposome carriers, bio-erodible microparticles or porous beads and depot injections, are also known to those skilled in the art. Sustained-release preparations may include, e.g., porous polymeric microparticles or semipermeable polymer matrices in the form of shaped articles, e.g., films, or microcapsules. Sustained release matrices may include polyesters, hydrogels, polylactides, copolymers of L-glutamic acid and gamma ethyl-L-glutamate, poly (2-hydroxyethyl-methacrylate), ethylene vinyl acetate, or poly-D(−)-3-hydroxybutyric acid. Sustained release compositions may also include liposomes that can be prepared by any of several methods known in the art.

Pharmaceutical compositions containing a sialidase or sialidase conjugated to a half-life extender can be presented in a dosage unit form and can be prepared by any suitable method. A pharmaceutical composition should be formulated to be compatible with its intended route of administration. Examples of routes of administration are intravenous (IV), intradermal, inhalation, transdermal, topical, transmucosal, intrathecal and rectal administration. In certain embodiments, a sialidase or sialidase conjugated to a half-life extender is administered by IV infusion. In certain embodiments, a sialidase or sialidase conjugated to a half-life extender is administered by intratumoral injection. Useful formulations can be prepared by methods known in the pharmaceutical art. For example, see Remington's Pharmaceutical Sciences, 18th ed. (Mack Publishing Company, 1990). Formulation components suitable for parenteral administration include a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as EDTA; buffers such as acetates, citrates or phosphates; and agents for the adjustment of tonicity such as sodium chloride or dextrose.

For intravenous administration, suitable carriers include physiological saline, bacteriostatic water, Cremophor ELTM (BASF, Parsippany, N.J.) or phosphate buffered saline (PBS). The carrier should be stable under the conditions of manufacture and storage, and should be preserved against microorganisms. The carrier can be a solvent or dispersion medium containing, for example, water, ethanol, polyol (for example, glycerol, propylene glycol, and liquid polyethylene glycol), and suitable mixtures thereof.

Pharmaceutical formulations preferably are sterile. Sterilization can be accomplished by any suitable method, e.g., filtration through sterile filtration membranes. Where the composition is lyophilized, filter sterilization can be conducted prior to or following lyophilization and reconstitution.

In certain embodiments, the pharmaceutical composition is disposed in a sterile container (e.g., bottle or vial). The pharmaceutical composition can be, for example, lyophilized or present as a solution in the sterile container. The sterile container can be sealed with a septum and can have a label disposed thereon identifying the pharmaceutical composition contained in the container.

The compositions described herein may be administered locally or systemically. Administration will generally be parenteral administration. In a preferred embodiment, the pharmaceutical composition is administered subcutaneously and in an even more preferred embodiment intravenously. Preparations for parenteral administration include sterile aqueous or non-aqueous solutions, suspensions, and emulsions.

Generally, a therapeutically effective amount of active component, for example, a sialidase or sialidase conjugated to a half-life extender, is in the range of 0.1 mg/kg to 100 mg/kg, e.g., 1 mg/kg to 100 mg/kg, 1 mg/kg to 10 mg/kg. The amount administered will depend on variables such as the type and extent of disease or indication to be treated, the overall health of the patient, the in vivo potency of the active component, the pharmaceutical formulation, and the route of administration. The initial dosage can be increased beyond the upper level in order to rapidly achieve the desired blood-level or tissue-level. Alternatively, the initial dosage can be smaller than the optimum, and the daily dosage may be progressively increased during the course of treatment. Human dosage can be optimized, e.g., in a conventional Phase I dose escalation study designed to run from 0.5 mg/kg to 20 mg/kg. Dosing frequency can vary, depending on factors such as route of administration, dosage amount, serum half-life of the sialidase or sialidase conjugated to a half-life extender, and the disease being treated. Exemplary dosing frequencies are once per day, once per week and once every two weeks. A preferred route of administration is parenteral, e.g., intravenous infusion. In certain embodiments, a sialidase or sialidase conjugated to a half-life extender is lyophilized, and then reconstituted in buffered saline, at the time of administration.

### VI. Therapeutic Uses

The compositions and methods disclosed herein can be used to treat various forms of cancer in a subject or inhibit cancer growth in a subject. The invention provides a method of treating a cancer in a subject. The method comprises administering to the subject an effective amount of a sialidase or sialidase conjugated to a half-life extender either alone or in a combination with another therapeutic agent to treat the cancer in the subject. The term “effective amount” as used herein refers to the amount of an active agent (e.g., sialidase or sialidase conjugated to a half-life extender according to the present invention) sufficient to effect beneficial or desired results. An effective amount can be administered in one or more administrations, applications or dosages and is not intended to be limited to a particular formulation or administration route.

As used herein, “treat”, “treating” and “treatment” mean the treatment of a disease in a subject, e.g., in a human. This includes: (a) inhibiting the disease, i.e., arresting its development; and (b) relieving the disease, i.e., causing regression of the disease state. As used herein, the terms “subject” and “patient” refer to an organism to be treated by the methods and compositions described herein. Such organisms preferably include, but are not limited to, mammals (e.g., murines, simians, equines, bovines, porcines, canines, felines, and the like), and more preferably includes humans.

Examples of cancers include solid tumors, soft tissue tumors, hematopoietic tumors and metastatic lesions. Examples of hematopoietic tumors include, leukemia, acute leukemia, acute lymphoblastic leukemia (ALL), B-cell, T-cell or FAB ALL, acute myeloid leukemia (AML), chronic myelocytic leukemia (CML), chronic lymphocytic leukemia (CLL), e.g., transformed CLL, diffuse large B-cell lymphomas (DLBCL), follicular lymphoma, hairy cell leukemia, myelodyplastic syndrome (MDS), a lymphoma, Hodgkin's disease, a malignant lymphoma, non-Hodgkin's lymphoma, Burkitt's lymphoma, multiple myeloma, or Richter's Syndrome (Richter's Transformation). Examples of solid tumors include malignancies, e.g., sarcomas, adenocarcinomas, and carcinomas, of the various organ systems, such as those affecting head and neck (including pharynx), thyroid, lung (small cell or non-small cell lung carcinoma (NSCLC)), breast, lymphoid, gastrointestinal (e.g., oral, esophageal, stomach, liver, pancreas, small intestine, colon and rectum, anal canal), genitals and genitourinary tract (e.g., renal, urothelial, bladder, ovarian, uterine, cervical, endometrial, prostate, testicular), CNS (e.g., neural or glial cells, e.g., neuroblastoma or glioma), or skin (e.g., melanoma).

In certain embodiments the cancer is an epithelial cancer, e.g., an epithelial cancer that upregulates the expression of sialylated glycans. Exemplary epithelial cancers include, but are not limited to, endometrial cancer, colon cancer, ovarian cancer, cervical cancer, vulvar cancer, uterine cancer or fallopian tube cancer, breast cancer, prostate cancer, lung cancer, pancreatic cancer, urinary cancer, bladder cancer, head and neck cancer, oral cancer and liver cancer. Epithelial cancers also include carcinomas, for example, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, baso squamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiermoid carcinoma, carcinoma epitheliale adenoides, exophytic carcinoma, carcinoma ex ulcere, carcinoma fibrosum, gelatiniforni carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypemephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, oat cell carcinoma, carcinoma ossifi cans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, and carcinoma villosum.

In certain embodiments, the cancer is breast cancer. In certain embodiments, the cancer is an adenocarcinoma. In certain embodiments, the cancer is a metastatic cancer. In certain embodiments, the cancer is a refractory cancer.

In certain embodiments, the cancer is resistant to or non-responsive to treatment with an antibody, e.g., an antibody with ADCC activity, e.g., trastuzumab.

The methods and compositions described herein can be used alone or in combination with other therapeutic agents and/or modalities. The term administered “in combination,” as used herein, is understood to mean that two (or more) different treatments are delivered to the subject during the course of the subject's affliction with the disorder, such that the effects of the treatments on the patient overlap at a point in time. In certain embodiments, the delivery of one treatment is still occurring when the delivery of the second begins, so that there is overlap in terms of administration. This is sometimes referred to herein as “simultaneous” or “concurrent delivery.” In other embodiments, the delivery of one treatment ends before the delivery of the other treatment begins. In certain embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In certain embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered.

In certain embodiments, a method or composition described herein, is administered in combination with one or more additional therapies, e.g., surgery, radiation therapy, or administration of another therapeutic preparation. In certain embodiments, the additional therapy may include chemotherapy, e.g., a cytotoxic agent. In certain embodiments the additional therapy may include a targeted therapy, e.g. a tyrosine kinase inhibitor, a proteasome inhibitor, or a protease inhibitor. In certain embodiments, the additional therapy may include an anti-inflammatory, anti-angiogenic, anti-fibrotic, or anti-proliferative compound, e.g., a steroid, a biologic immunomodulator, a monoclonal antibody, an antibody fragment, an aptamer, an siRNA, an antisense molecule, a fusion protein, a cytokine, a cytokine receptor, a bronchodilator, a statin, an anti-inflammatory agent (e.g. methotrexate), or an NSAID. In certain embodiments, the additional therapy may include a combination of therapeutics of different classes.

In certain embodiments, a method or composition described herein is administered in combination with a checkpoint inhibitor. The checkpoint inhibitor may, for example, be selected from a PD-1 antagonist, PD-L1 antagonist, CTLA-4 antagonist, adenosine A2A receptor antagonist, B7-H3 antagonist, B7-H4 antagonist, BTLA antagonist, KIR antagonist, LAG3 antagonist, TIM-3 antagonist, VISTA antagonist or TIGIT antagonist.

In certain embodiments, the checkpoint inhibitor is a PD-1 or PD-L1 inhibitor. PD-1 is a receptor present on the surface of T-cells that serves as an immune system checkpoint that inhibits or otherwise modulates T-cell activity at the appropriate time to prevent an overactive immune response. Cancer cells, however, can take advantage of this checkpoint by expressing ligands, for example, PD-L1, that interact with PD-1 on the surface of T-cells to shut down or modulate T-cell activity. Exemplary PD-1/PD-L1 based immune checkpoint inhibitors include antibody based therapeutics. Exemplary treatment methods that employ PD-1/PD-L1 based immune checkpoint inhibition are described in U.S. Pat. Nos. 8,728,474 and 9,073,994, and EP Patent No. 1537878B1, and, for example, include the use of anti-PD-1 antibodies. Exemplary anti-PD-1 antibodies are described, for example, in U.S. Pat. Nos. 8,952,136, 8,779,105, 8,008,449, 8,741,295, 9,205,148, 9,181,342, 9,102,728, 9,102,727, 8,952,136, 8,927,697, 8,900,587, 8,735,553, and 7,488,802. Exemplary anti-PD-1 antibodies include, for example, nivolumab (Opdivo®, Bristol-Myers Squibb Co.), pembrolizumab (Keytruda®, Merck Sharp & Dohme Corp.), PDR001 (Novartis Pharmaceuticals), and pidilizumab (CT-011, Cure Tech). Exemplary anti-PD-L1 antibodies are described, for example, in U.S. Pat. Nos. 9,273,135, 7,943,743, 9,175,082, 8,741,295, 8,552,154, and 8,217,149. Exemplary anti-PD-L1 antibodies include, for example, atezolizumab (Tecentriq®, Genentech), durvalumab (AstraZeneca), MEDI4736, avelumab, and BMS 936559 (Bristol Myers Squibb Co.).

In certain embodiments, a method or composition described herein is administered in combination with a CTLA-4 inhibitor. In the CTLA-4 pathway, the interaction of CTLA-4 on a T-cell with its ligands (e.g., CD80, also known as B7-1, and CD86) on the surface of an antigen presenting cells (rather than cancer cells) leads to T-cell inhibition. Exemplary CTLA-4 based immune checkpoint inhibition methods are described in U.S. Pat. Nos. 5,811,097, 5,855,887, 6,051,227. Exemplary anti-CTLA-4 antibodies are described in U.S. Pat. Nos. 6,984,720, 6,682,736, 7,311,910; 7,307,064, 7,109,003, 7,132,281, 6,207,156, 7,807,797, 7,824,679, 8,143,379, 8,263,073, 8,318,916, 8,017,114, 8,784,815, and 8,883,984, International (PCT) Publication Nos. WO98/42752, WO00/37504, and WO01/14424, and European Patent No. EP 1212422 B1. Exemplary CTLA-4 antibodies include ipilimumab or tremelimumab.

In certain embodiments, a method or composition described herein is administered in combination with (i) a PD-1 or PD-L1 inhibitor, e.g., a PD-1 or PD-L1 inhibitor disclosed herein, and (ii) CTLA-4 inhibitor, e.g., a CTLA-4 inhibitor disclosed herein.

In certain embodiments, a method or composition described herein is administered in combination with a CD20 inhibitor. In certain embodiments, the CD20 inhibitor is an anti-CD20 antibody. In certain embodiments, the anti-CD20 antibody is selected from the group consisting of ofatumumab, rituximab, ocrelizumab, iodine I 131 tositumomab, obinutuzumab, ibritumomab, and hyaluronidase ritixumab.

In certain embodiments, a method or composition described herein is administered in combination with an IDO inhibitor. Exemplary IDO inhibitors include 1-methyl-D-tryptophan (known as indoximod), epacadostat (INCB24360), navoximod (GDC-0919), and BMS-986205.

Exemplary cytotoxic agents that can be administered in combination with a method or composition described herein include, for example, antimicrotubule agents, topoisomerase inhibitors, antimetabolites, protein synthesis and degradation inhibitors, mitotic inhibitors, alkylating agents, platinating agents, inhibitors of nucleic acid synthesis, histone deacetylase inhibitors (HDAC inhibitors, e.g., vorinostat (SAHA, MK0683), entinostat (MS-275), panobinostat (LBH589), trichostatin A (TSA), mocetinostat (MGCD0103), belinostat (PXD101), romidepsin (FK228, depsipeptide)), DNA methyltransferase inhibitors, nitrogen mustards, nitrosoureas, ethylenimines, alkyl sulfonates, triazenes, folate analogs, nucleoside analogs, ribonucleotide reductase inhibitors, vinca alkaloids, taxanes, epothilones, intercalating agents, agents capable of interfering with a signal transduction pathway, agents that promote apoptosis and radiation, or antibody molecule conjugates that bind surface proteins to deliver a toxic agent. In one embodiment, the cytotoxic agent that can be administered with a method or composition described herein is a platinum-based agent (such as cisplatin), cyclophosphamide, dacarbazine, methotrexate, fluorouracil, gemcitabine, capecitabine, hydroxyurea, topotecan, irinotecan, azacytidine, vorinostat, ixabepilone, bortezomib, taxanes (e.g., paclitaxel or docetaxel), cytochalasin B, gramicidin D, ethidium bromide, emetine, mitomycin, etoposide, tenoposide, vincristine, vinblastine, vinorelbine, colchicin, anthracyclines (e.g., doxorubicin or epirubicin) daunorubicin, dihydroxy anthracin dione, mitoxantrone, mithramycin, actinomycin D, adriamycin, 1-dehydrotestosterone, glucocorticoids, procaine, tetracaine, lidocaine, propranolol, puromycin, ricin, or maytansinoids.

The invention also provides a method of increasing the expression of granzyme B, IL-1b, IL-2, IL-6, IL-10, IL-17A, HLA-DR, CD86, CD83, IFNγ, or TNFα in a cell, tissue, or subject. The method comprises contacting the cell, tissue, or subject with an effective amount of a sialidase or sialidase conjugated to a half-life extender so as to increase the expression of granzyme B, IL-1b, IL-2, IL-6, IL-10, IL-17A, HLA-DR, CD86, CD83, IFNγ, or TNFα in a cell, tissue, or subject relative the corresponding expression level prior to contact with the sialidase or sialidase conjugated to the half-life extender. In certain embodiments, the cell is selected from a dendritic cell and a peripheral blood mononuclear cell (PBMC, e.g., a monocyte).

In certain embodiments, expression of granzyme B, IL-1b, IL-2, IL-6, IL-10, IL-17A, HLA-DR, CD86, CD83, IFNγ, or TNFα in the cell, tissue, or subject is increased by at least about 10%, at least about 20%, at least about 50%, at least about 75%, at least about 100%, at least about 150%, at least about 200%, at least about 250%, at least about 300%, at least about 400%, at least about 500%, at least about 600%, at least about 700%, at least about 800%, at least about 900%, or at least about 1,000%, relative to a similar or otherwise identical cell or tissue that has not been contacted with the sialidase or sialidase conjugated to a half-life extender. Gene expression may be measured by any suitable method known in the art, for example, by ELISA, by Luminex multiplex assays, or by flow cytometry as described in the examples herein.

The invention also provides a method of removing sialic acid from a cell or tissue. The method comprises contacting the cell or tissue with an effective amount of a sialidase or sialidase conjugated to a half-life extender. The invention also provides a method of removing sialic acid from a cell in a subject, the method comprising administering to the subject an effective amount of a pharmaceutical composition comprising a sialidase or sialidase conjugated to a half-life extender thereby to remove sialic acid from the cell.

In certain embodiments, the cell is tumor cell, dendritic cell (DC) or monocyte. In certain embodiments, the cell is a monocyte, and the method results in increased expression of an MHC-II molecule (e.g., HLA-DR) on the monocyte. In certain embodiments, expression of an MHC-II molecule in the cell or tissue is increased by at least about 10%, at least about 20%, at least about 50%, at least about 75%, at least about 100%, at least about 150%, at least about 200%, at least about 250%, at least about 300%, at least about 400%, at least about 500%, at least about 600%, at least about 700%, at least about 800%, at least about 900%, or at least about 1,000%, relative to a similar or otherwise identical cell or tissue that has not been contacted with the sialidase or sialidase conjugated to a half-life extender. Gene expression may be measured by any suitable method known in the art, for example, by ELISA, by Luminex multiplex assays, or by flow cytometry as described in the examples herein.

The invention also provides a method of increasing phagocytosis of a tumor cell. The method comprises contacting the tumor cell with a sialidase or sialidase conjugated to a half-life extender in an amount effective to remove sialic acid from the tumor cell, thereby increasing phagocytosis of the tumor cell. In certain embodiments, the disclosure relates to a method of increasing phagocytosis of a tumor cell in a subject, the method comprising administering to the subject an effective amount of a pharmaceutical composition comprising a sialidase or sialidase conjugated to a half-life extender in an amount effective to remove sialic acid from the tumor cell, thereby increasing phagocytosis of the tumor cell.

In certain embodiments, phagocytosis is increased by at least about 10%, at least about 20%, at least about 50%, at least about 75%, at least about 100%, at least about 150%, at least about 200%, at least about 250%, at least about 300%, at least about 400%, at least about 500%, at least about 600%, at least about 700%, at least about 800%, at least about 900%, or at least about 1,000%, relative to a similar or otherwise identical tumor cell or population of tumor cells that has not or have not been contacted with the sialidase or sialidase conjugated to a half-life extender. Phagocytosis may be measured as described in Example 9 herein.

The invention also provides a method of activating a dendritic cell (DC) or a population of DCs. The method comprises contacting the DC or population of DCs with a tumor cell that has been treated with a sialidase or sialidase conjugated to a half-life extender. In certain embodiments, the disclosure relates to a method of activating a dendritic cell (DC) or a population of DCs in a subject, the method comprising administering to the subject an amount of a pharmaceutical composition comprising a sialidase or sialidase conjugated to a half-life extender effective to remove sialic acid from a tumor cell in the subject, thereby to activate the DC or the population of DCs in the subject.

In certain embodiments, activation of the DC or a population of DCs is increased by at least about 10%, at least about 20%, at least about 50%, at least about 75%, at least about 100%, at least about 150%, at least about 200%, at least about 250%, at least about 300%, at least about 400%, at least about 500%, at least about 600%, at least about 700%, at least about 800%, at least about 900%, or at least about 1,000%, relative to a similar or otherwise identical DC or population of DCs that has not or have not been contacted with a tumor cell that has been treated with the sialidase or sialidase conjugated to a half-life extender. Activation may be measured as described in Example 8 herein.

The invention also provides a method of reducing Siglec-15 binding activity, thereby increasing anti-tumor activity in a tumor microenvironment, the method comprising contacting a T cell with a sialidase or sialidase conjugated to a half-life extender. In certain embodiments, the disclosure relates to a method of reducing Siglec-15 binding activity, thereby increasing anti-tumor activity in a tumor microenvironment of a patient, the method comprising administering to the subject an effective amount of a pharmaceutical composition comprising a sialidase or sialidase conjugated to a half-life extender, thereby increasing anti-tumor activity (e.g., T cell activity) in the subject.

In certain embodiments, Siglec-15 binding activity is reduced by at least about 10%, at least about 20%, at least about 50%, at least about 75%, or about 100%, relative to Siglec-15 that has not been contacted with the sialidase or sialidase conjugated to a half-life extender. Binding may be measured as described in Example 16 herein.

The invention also provides a method of promoting infiltration of immune cells into a tumor in a subject in need thereof. The method comprises administering to the subject an effective amount of a sialidase or sialidase conjugated to a half-life extender, e.g., a sialidase or sialidase conjugated to a half-life extender disclosed herein. In certain embodiments, the immune cells are T-cells, e.g., CD4+ and/or CD8+ T-cells, e.g., CD69⁺CD8⁺ and/or GzmB⁺CD8⁺ T-cells. In certain embodiments, the immune cells are natural killer (NK) cells.

In certain embodiments, the infiltration of immune cells into the tumor in the subject is increased by at least about 10%, at least about 20%, at least about 50%, at least about 75%, at least about 100%, at least about 150%, at least about 200%, at least about 250%, at least about 300%, at least about 400%, at least about 500%, at least about 600%, at least about 700%, at least about 800%, at least about 900%, or at least about 1,000%, relative to a similar or otherwise identical tumor and/or subject that has not been administered the sialidase or sialidase conjugated to a half-life extender. Infiltration of immune cells into a tumor may be measured by any suitable method known in the art, for example, antibody staining.

The invention also provides a method of increasing the number of circulating natural killer (NK) cells in a subject in need thereof. The method comprises administering to the subject an effective amount of a sialidase or sialidase conjugated to a half-life extender, e.g., a sialidase or sialidase conjugated to a half-life extender disclosed herein, so as to increase the number of circulating NK cells relative to prior to administration of the sialidase or sialidase conjugated to a half-life extender or pharmaceutical composition.

In certain embodiments, the number of circulating NK cells in the subject is increased by at least about 10%, at least about 20%, at least about 50%, at least about 75%, at least about 100%, at least about 150%, at least about 200%, at least about 250%, at least about 300%, at least about 400%, at least about 500%, at least about 600%, at least about 700%, at least about 800%, at least about 900%, or at least about 1,000%, relative to a similar or otherwise identical subject that has not been administered the sialidase or sialidase conjugated to a half-life extender. Circulating NK cells in a subject may be measured by any suitable method known in the art, for example, antibody staining.

The invention also provides a method of increasing the number of T-cells in the draining lymph node in a subject in need thereof. The method comprises administering to the subject an effective amount of a sialidase or sialidase conjugated to a half-life extender, e.g., a sialidase or sialidase conjugated to a half-life extender disclosed herein, so as to increase the number of T-cells in the draining lymph node relative to prior to administration of the sialidase or sialidase conjugated to a half-life extender or pharmaceutical composition. In certain embodiments, the immune cells are T-cells, e.g., CD4+ and/or CD8+ T-cells.

In certain embodiments, the number of T-cells in the draining lymph node in the subject is increased by at least about 10%, at least about 20%, at least about 50%, at least about 75%, at least about 100%, at least about 150%, at least about 200%, at least about 250%, at least about 300%, at least about 400%, at least about 500%, at least about 600%, at least about 700%, at least about 800%, at least about 900%, or at least about 1,000%, relative to a similar or otherwise identical subject that has not been administered the sialidase or sialidase conjugated to a half-life extender. T-cells in the draining lymph node in a subject may be measured by any suitable method known in the art, for example, antibody.

The invention also provides a method of increasing expression of Cd3, Cd4, Cd8, Cd274, Ctla4, Icos, Pdcd1, Lag3, Il6, Il1b, Il2, Ifng, Ifna1, Mx1, Gzmb, Cxcl9, Cxcl12, and/or Ccl5 in a cell, tissue, or subject. The method comprises contacting the cell, tissue, or subject with an effective amount of a sialidase or sialidase conjugated to a half-life extender, e.g., a sialidase or sialidase conjugated to a half-life extender disclosed herein, so as to increase the expression of Cd3, Cd4, Cd8, Cd274, Ctla4, Icos, Pdcd1, Lag3, Il6, Il1b, Il2, Ifng, Ifna1, Mx1, Gzmb, Cxcl9, Cxcl12, and/or Ccl5 relative to the cell, tissue or subject prior to contact with the sialidase or sialidase conjugated to a half-life extender or pharmaceutical composition.

In certain embodiments, expression of Cd3, Cd4, Cd8, Cd274, Ctla4, Icos, Pdcd1, Lag3, Il6, Il1b, Il2, Ifng, Ifna1, Mx1, Gzmb, Cxcl9, Cxcl12, and/or Ccl5 in the cell, tissue, or subject is increased by at least about 10%, at least about 20%, at least about 50%, at least about 75%, at least about 100%, at least about 150%, at least about 200%, at least about 250%, at least about 300%, at least about 400%, at least about 500%, at least about 600%, at least about 700%, at least about 800%, at least about 900%, or at least about 1,000%, relative to a similar or otherwise identical cell, tissue, or subject that has not been contacted with the sialidase or sialidase conjugated to a half-life extender. Gene expression may be measured by any suitable method known in the art, for example, by ELISA, Luminex multiplex assays, or Nanostring technology.

Throughout the description, where compositions are described as having, including, or comprising specific components, or where processes and methods are described as having, including, or comprising specific steps, it is contemplated that, additionally, there are compositions of the present invention that consist essentially of, or consist of, the recited components, and that there are processes and methods according to the present invention that consist essentially of, or consist of, the recited processing steps.

In the application, where an element or component is said to be included in and/or selected from a list of recited elements or components, it should be understood that the element or component can be any one of the recited elements or components, or the element or component can be selected from a group consisting of two or more of the recited elements or components.

Further, it should be understood that elements and/or features of a composition or a method described herein can be combined in a variety of ways without departing from the spirit and scope of the present invention, whether explicit or implicit herein. For example, where reference is made to a particular compound, that compound can be used in various embodiments of compositions of the present invention and/or in methods of the present invention, unless otherwise understood from the context. In other words, within this application, embodiments have been described and depicted in a way that enables a clear and concise application to be written and drawn, but it is intended and will be appreciated that embodiments may be variously combined or separated without parting from the present teachings and invention(s). For example, it will be appreciated that all features described and depicted herein can be applicable to all aspects of the invention(s) described and depicted herein.

It should be understood that the expression “at least one of” includes individually each of the recited objects after the expression and the various combinations of two or more of the recited objects unless otherwise understood from the context and use. The expression “and/or” in connection with three or more recited objects should be understood to have the same meaning unless otherwise understood from the context.

The use of the term “include,” “includes,” “including,” “have,” “has,” “having,” “contain,” “contains,” or “containing,” including grammatical equivalents thereof, should be understood generally as open-ended and non-limiting, for example, not excluding additional unrecited elements or steps, unless otherwise specifically stated or understood from the context.

Where the use of the term “about” is before a quantitative value, the present invention also includes the specific quantitative value itself, unless specifically stated otherwise. As used herein, the term “about” refers to a ±10% variation from the nominal value unless otherwise indicated or inferred.

It should be understood that the order of steps or order for performing certain actions is immaterial so long as the present invention remain operable. Moreover, two or more steps or actions may be conducted simultaneously.

The use of any and all examples, or exemplary language herein, for example, “such as” or “including,” is intended merely to illustrate better the present invention and does not pose a limitation on the scope of the invention unless claimed. No language in the specification should be construed as indicating any non-claimed element as essential to the practice of the present invention.

### EXAMPLES

### Example 1: Construction and Expression of Recombinant Sialidases

This example describes the construction of recombinant human sialidases (Neu1, Neu2, Neu3, and Neu4). The human sialidases Neu1, Neu2, Neu3 (isoform 1), and Neu4 (isoform 1) were expressed as secreted proteins with a 10×His tag.

To express Neu1 as a secreted protein, the native N terminal signal peptide (MTGERPSTALPDRRWGPRILGFWGGCRVWVFAAIFLLLSLAASWSKA; SEQ ID NO: 27) was replaced by MDMRVPAQLLGLLLLWLPGARC (SEQ ID NO: 28), and the C terminal lysosomal signal motif (YGTL; SEQ ID NO: 29) was removed. To express Neu2, Neu3, and Neu4 as secreted proteins, the N terminal signal peptide MDMRVPAQLLGLLLLWLPGARC (SEQ ID NO: 28) was added to each.

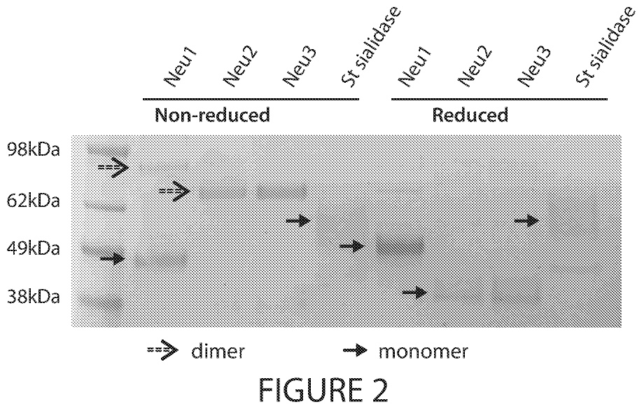

Sialidases were expressed in a 200 mL transfection of HEK293F human cells in 24-well plates using the pCEP4 mammalian expression vector. Sialidases were purified using Ni-NTA columns, quantified with a UV-Vis spectrophotometer (NanoDrop), and examined by SDS-PAGE as shown in FIG. 2. Neu1 expressed well, with a yield of ˜3 μg/ml, and was present primarily in a monomeric form. Neu2 and Neu3 expression each gave yields of ˜0.15 μg/mL and each were present primarily in a dimeric form. Neu4 had no detectable expression yield as measured by NanoDrop. Bacterial sialidase from Salmonella typhimurium (bacterial-sialidase; SEQ ID NO: 30) was expressed in the same manner as Neu1-4 (above) and gave a comparable yield to Neu1, and was present primarily in a monomeric form.

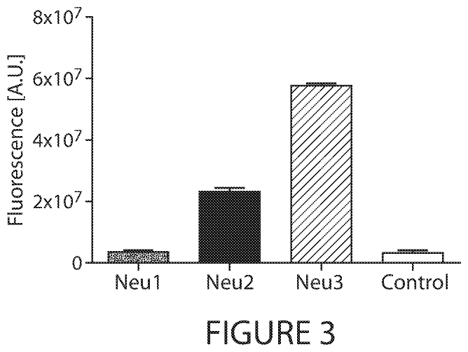

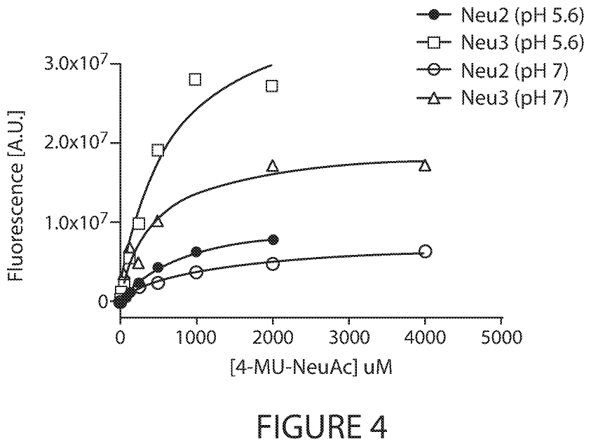

The activity of the recombinantly expressed sialidases was assayed by measuring the release of sialic acid from the fluorogenic substrate 4-methylumbelliferyl-N-acetylneuraminic acid (4MU-NeuAc). As shown in FIG. 3, Neu1 has no detectable activity beyond a no-enzyme control, which is consistent with previous reports indicating that Neu1 is inactive unless it is in complex with beta-galactosidase and protective protein/cathepsin A (PPCA). Neu2 and Neu3 were active, as was the bacterial sialidase. An enzyme kinetics assay was performed with Neu2 and Neu3. A fixed concentration of enzyme at 1 nM was incubated with fluorogenic substrate 4MU-NeuAc at concentrations ranging from 4000 μM to 7.8 μM. Assays were conducted at both acidic (pH 5.6) and neutral (pH 7) conditions. As shown in FIG. 4, both Neu2 and Neu3 were active at acidic and neutral conditions and showed enzyme kinetics that were comparable to those previously reported.

### Example 2: Construction and Expression of Recombinant Sialidase-Fc Fusion Proteins

This example describes the construction of recombinant Fc sialidase genetic fusions. In particular, Neu2-Fc, Neu3-Fc and ST sialidase-Fc.

Fc-sialidases utilizing wild type Neu2 (Neu2-Fc; SEQ ID NO:113, encoded by SEQ ID NO: 114) and a variant Fc-Sialidase designated M106 (SEQ ID NO: 115, encoded by SEQ ID NO: 116) (M1D, V6Y, P62G, A93E, I187K, C332A, and human IgG1 Fc with hole (Y407T) mutation) were expressed, purified and characterized. The Neu2-Fc molecules were expressed in a 1L transfection of Expi293 human cells in using the pCEP4 mammalian expression vector. Neu2-Fc was purified using Protein A, followed by cation exchange chromatography (Hitrap SP-HP, GE Lifesciences). Neu2-Fc had a yield of 0.3 mg/liter, and M106 had a yield of 20 mg/liter.

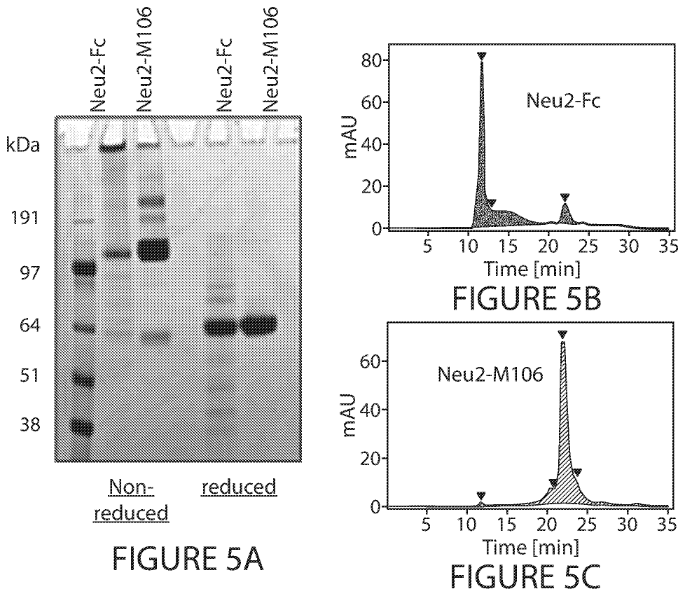

FIG. 5A depicts an SDS-PAGE gel showing recombinant wildtype human Neu2-Fc and M106 under non-reducing and reducing conditions. FIGS. 5B-C shows SEC-HPLC traces comparing wildtype Neu2-Fc versus M106. Monomer species have a retention time of 21 mins. Neu2-Fc (FIG. 5B) had an SEC monomer purity of 7% and M106 (FIG. 5C) had an SEC monomer purity of 85%.

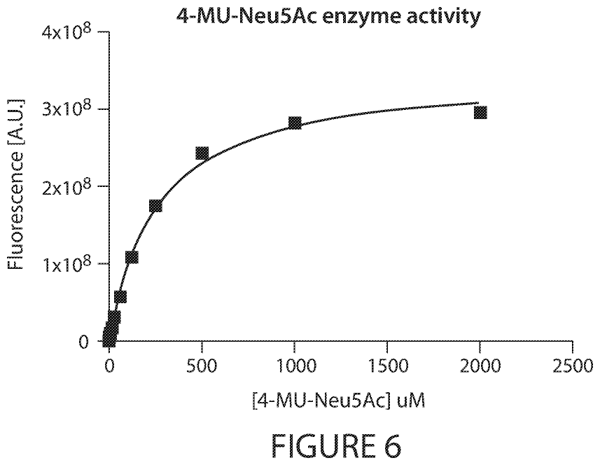

The activity of M106 was assayed by measuring the release of sialic acid from the fluorogenic substrate 4-methylumbelliferyl-N-acetylneuraminic acid (4MU-NeuAc). An enzyme kinetics assay was performed using a fixed concentration of enzyme at 2 μg/well was incubated with fluorogenic substrate 4MU-NeuAc at concentrations ranging from 4 mM to 0.03 μM. FIG. 6 depicts the enzyme activity of M106.

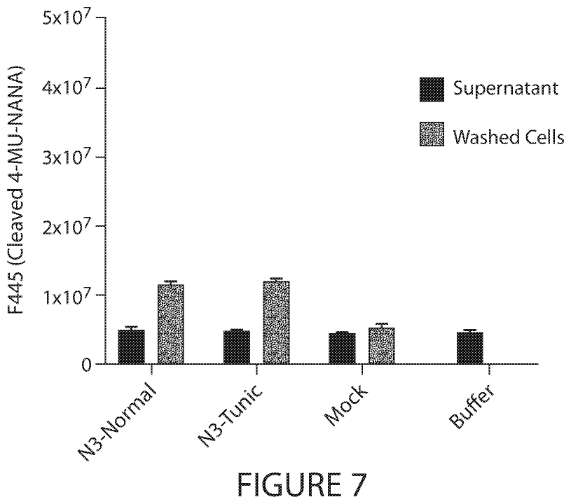

An FC Sialidase utilizing wild type Neu3 (Neu3-Fc; SEQ ID NO: 117, encoded by SEQ ID NO: 118) was expressed in a 100 ml transfection of Expi293 human cells using the pCEP4 mammalian expression vector. Activity was determined using Neu3-Fc expressing cells (N3-Normal), Neu3-Fc expressing cells treated with tunicamycin (N3-Tunic), and mock transfected cells in both the cell conditioned media (Supernatant) and washed cell pellets. FIG. 7 shows that Neu3-Fc activity was detected in the cell pellet, representing surface bound activity, and that low levels of activity detected in the supernatant, representing secreted Neu3-Fc. Treatment with tunicamycin, an inhibitor of S-acylation and N-glycosylation, did not change the surface associated activity or activity in the supernatant.

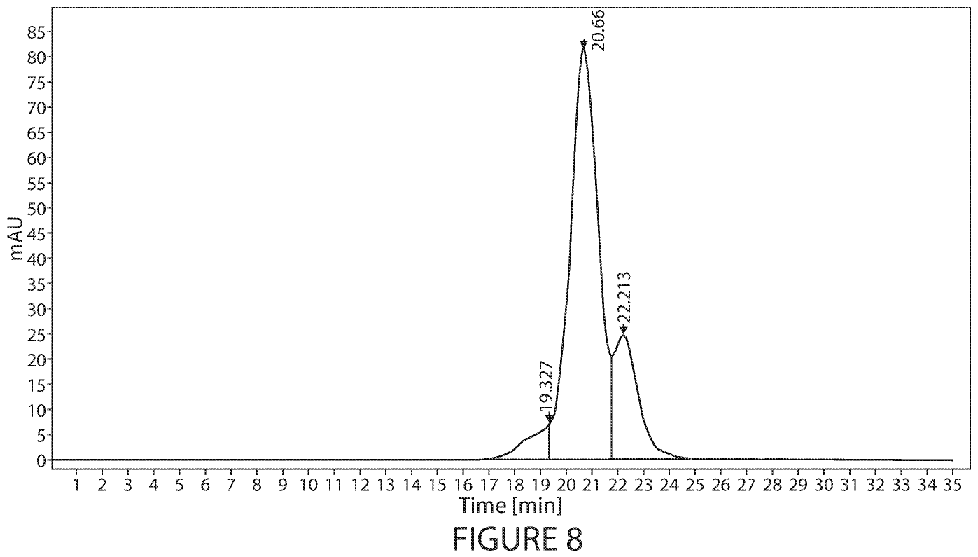

An Fc bacterial sialidase using Salmonella typhimurium (Fc-ST Sialidase) was constructed using a knob in hole-based Fc design. The Fc-ST Sialidase comprised a dimer of two polypeptides: SEQ ID NOs: 119 (pCEP-StSia-G452-hIgG1Fc-Hole, encoded by SEQ ID NO: 121) and SEQ ID NO: 120 (pCEP-StSia-G452-hIgG1Fc-Knob, encoded by SEQ ID NO: 122). Fc-ST Sialidase was expressed in a 1L transfection of Expi293 human cells in using the pCEP4 mammalian expression vector. Fc-ST Sialidase was purified using Protein A, followed by cation exchange chromatography (Hitrap SP-HP, GE Lifesciences). FIG. 8 depicts an SEC-HPLC trace showing that the expressed Fc-ST Sialidase was a monomer species with a retention time of 21 minutes and an SEC monomer purity of 75%.

The activity of Fc-ST Sialidase was assayed by measuring the release of sialic acid from the fluorogenic substrate 4-methylumbelliferyl-N-acetylneuraminic acid (4MU-NeuAc). An enzyme kinetics assay was performed using a fixed concentration of enzyme at 2 μg/well was incubated with fluorogenic substrate 4MU-NeuAc at concentrations ranging from 4 mM to 0.03 μM. FC ST had an activity approaching 3×10⁸ Fluorescence AU.

### Example 3: In Vivo Administration of Fc Sialidases Reduces Tumor Volume

This Example shows that in vivo administration of Fc sialidases of the current invention reduce tumor volume in syngeneic mouse tumor models.

The Fc Salmonella typhimurium sialidase construct (Fc-ST Sialidase) described in Example 2 was compared to Avelumab (anti-PD-L1 antibody) in a mouse syngeneic tumor model injected with a murine lymphoma cancer cell line A20. Female BALB/c mice, 6-8 weeks of age, were inoculated subcutaneously in the right lower flank region with A20 tumor cells (5×10⁵) in 0.1 ml of PBS for tumor development. Mice were randomly allocated to 4 groups of 8 animals each when tumors reached 50-100 mm³, mean ˜75-100 mm³.

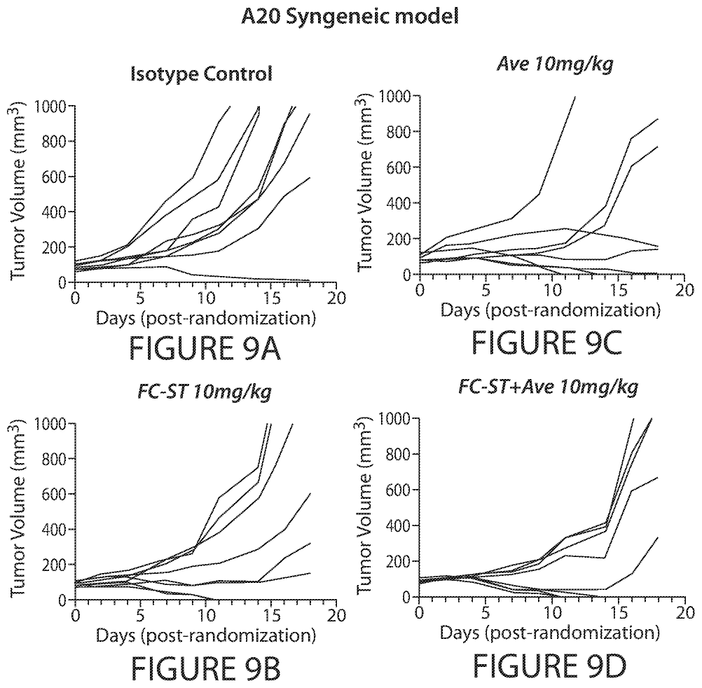

Mice were administered a negative control (“Isotype Control,” FIG. 9A), Fc-ST Sialidase (FIG. 9B), Avelumab (anti-mouse PD-L1 antibody, FIG. 9C), or the combination of Fc-ST Sialidase and Avelumab (FIG. 9D) via intraperitoneal injection of 10 mg/kg twice a week for 15 days and tumor volume (mm³) was measured over time. This example demonstrates that Fc sialidases of the present invention can reduce tumor volume in vivo.

The Fc-ST Sialidase was evaluated in a second model using a mouse tumor cell line engineered to express human Her2 (EMT6-Her2 cells). Fc-ST Sialidase and the human Neu2 Fc construct M106 (described in Example 2) was compared to trastuzumab (anti-HER2 antibody) in a mouse syngeneic tumor model injected with EMT6-Her2 cells. Female BALB/c mice, 6-8 weeks of age, were inoculated subcutaneously in the right lower flank region with EMT6-Her2 tumor cells (5×10⁵) in 0.1 ml of PBS for tumor development. Mice were randomly allocated to 4 groups of 8 animals each when tumors reached 50-100 mm³, mean ˜75-100 mm³.

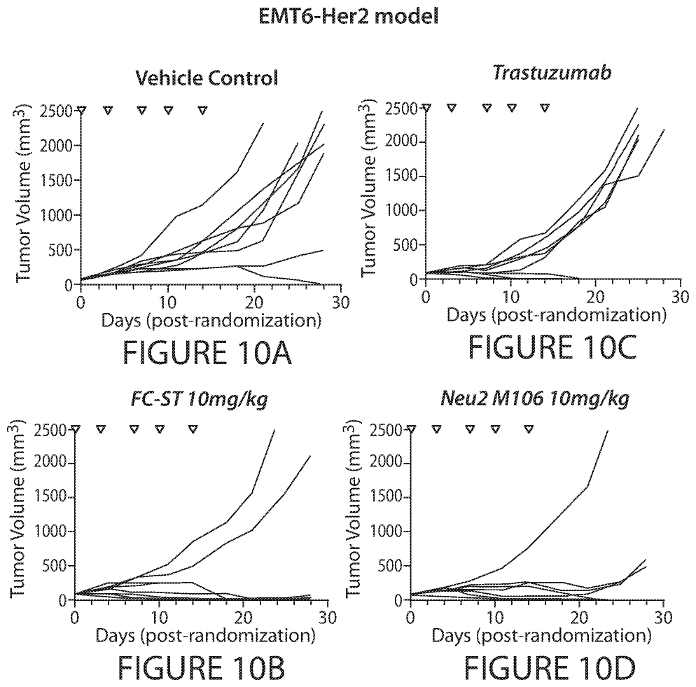

Mice were administered isotype control (Vehicle Control, FIG. 10A), Fc-ST Sialidase (FC-ST, FIG. 10B), trastuzumab (anti human Her2 antibody, FIG. 10C), or Fc human Sialidase (M106, FIG. 10D) via intraperitoneal injection at 10 mg/kg twice a week for 15 days, as indicated by the triangles, and tumor volume was measured over time. This example demonstrates that Fc sialidases of the present invention can reduce tumor volume in vivo.

### Example 4: Divalent Cations can Stabilize the Activity of Sialidases

This example describes the ability of divalent cations, and in particular calcium, to stabilize the activity of sialidases of the present invention. In particular, an Fc Neu2 Sialidase (SEQ ID NO:123) (M1D, V6Y, I187K, C332A) was expressed along with the heavy and light chain of trastuzumab (including a first polypeptide chain with amino acid sequence SEQ ID NO: 124, encoded by nucleotide sequence SEQ ID NO: 125, a second polypeptide chain with amino acid sequence SEQ ID NO: 126, encoded by nucleotide sequence SEQ ID NO: 127, and a third polypeptide chain with amino acid sequence SEQ ID NO: 123, encoded by nucleotide sequence SEQ ID NO: 128).

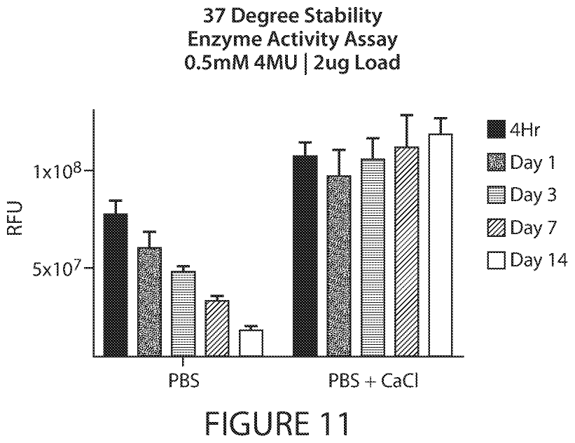

Purified protein in PBS or PBS with 4 mM CaCl₂ was incubated at 37° C. for up to 2 weeks. Samples containing about 2 μg of protein were assayed by measuring the release of sialic acid from the fluorogenic substrate 4-methylumbelliferyl-N-acetylneuraminic acid (4MU-NeuAc). Assays were done following 4 hours and days 1, 3, 7 and 14 at 37° C. The results are shown in FIG. 11. As can be seen, the addition of CaCl₂ to the enzyme preparation greatly stabilized the enzyme activity.

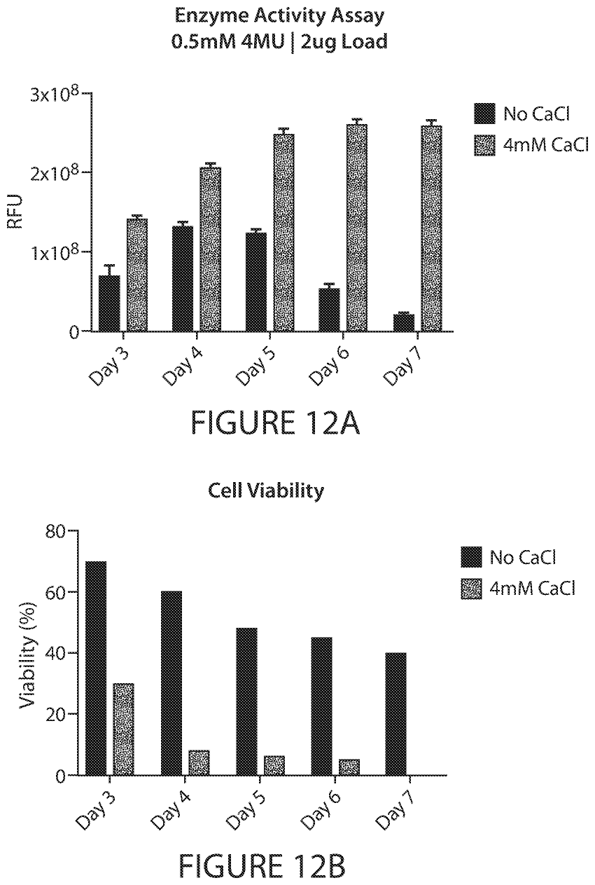

To see if CaCl₂ could stabilize enzyme activity during expression in mammalian cells, 4 mM CaCl₂ was added to transiently transfected Expi293 cell expression media starting 24 hours after transfection. As shown in FIG. 12A, the addition of CaCl₂ greatly increased the amount of secreted enzyme activity through day 7. However, as shown in FIG. 12B, 4 mM CaCl₂) led to a decrease in cell viability.

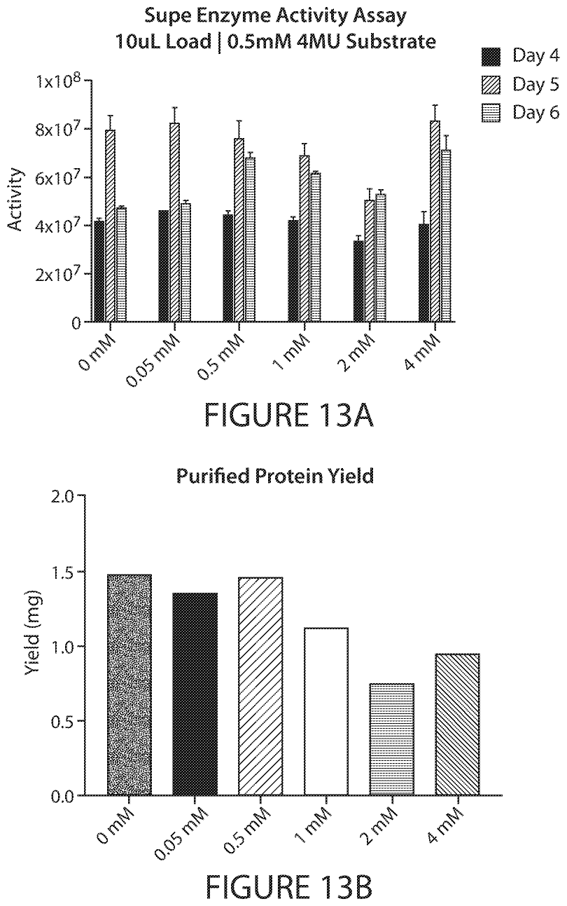

To optimize a CaCl₂ concentration that can stabilize enzyme activity but maintain cell viability, five concentrations of CaCl₂ ranging from 0.05 mM, 0.5 mM, 1 mM, 2 mM and 4 mM were added at day 1 following transfection. Conditioned media was collected over a three day time course on days 4-6 and enzymatic activity (and thus viability) determined as shown in FIG. 13A. Protein yield was also measured (FIG. 13B). It was found that 4 mM CaCl₂ stabilized activity and gave a moderate yield, but gave poor viability. It was found that, under the conditions tested, the use of 0.5 mM CaCl₂ maintained the activity of the sialidase, provided a higher protein yield and was less toxic to cells.

### Example 5: Sialoglycan Profiles of Subsets of Human PBMCs

This example describes the sialoglycan profile on different subsets of human peripheral blood mononuclear cells (PBMCs) using flow cytometry. Sialoglycans present on immune cell surfaces play important roles in maintaining homeostasis. Imbalance in sialoglycan profile on immune cells is documented in autoimmunity, mechanisms of immune surveillance escape by tumor cells and so forth.

Following isolation of PBMCs using a Ficoll method, cells were washed twice with ice cold PBS using a tabletop centrifuge at 350×g for 5 minutes, cells were counted using Countess™ II Automated Cell Counter (Thermo Fisher Scientific, Waltham, Mass.) and 250K cells were aliquoted into each well of a 96-well plate. Fc blocking solution containing Human Trustain FcX (1/20 dilution) and LIVE/DEAD™ Fixable Near-IR Dead Cell Stain (1/2000 dilution) in PBS was prepared and cells were incubated for 10 minutes on ice. Cells were washed using ice-cold PBS (1% BSA) at 350×g for 5 min. Cell surface sialoglycan staining was performed using Hydra and lectin reagents as shown in TABLE 10. Hydra-3, Hydra-7 and Hydra-9 are hexameric versions of the extracellular domain of human Siglec 3, Siglec 7, and Siglec 9, respectively, (as described in International (PCT) Application Publication No. WO2019/237070). Lectins used included Biotinylated Sambucus Nigra (SNA, Vector Laboratories, B-1305-2), Biotinylated Machia Amurensis (MAL-II, Vector Laboratories, B-1265-1) and Biotinylated Peanut Agglutinin (PNA, Vector Laboratories, B-1075-5). SNA is a lectin that preferentially binds to sialic acid attached to terminal galactose in α-2,6 and to a lesser degree, α-2,3 linkage. MAL-II is a lectin that binds to sialic acid in an α-2,3 linkage. PNA is a lectin that binds to terminal galactose residues. An increase in PNA staining can be indicative of the removal of terminal sialic acids by a sialidase and exposure of the underlying galactose.

TABLE 10

| | | Working | | |
|---|---|---|---|---|
| Reagent | Stock | conc. | | Buffer |
| Hydra-3 | Varies | 250 | nM | FACS staining |
| | | | | buffer |
| Hydra-7 | Varies | 25 | nM | FACS staining |
| | | | | buffer |
| Hydra-9 | Varies | 75 | nM | FACS staining |
| | | | | buffer |
| MAL-II | 1 mg/mL | 2 | μg/mL | PBS |
| PNA | 5 mg/mL | 1 | μg/mL | FACS staining |
| | | | | buffer |
| SNA | 2 mg/mL | 0.5 | μg/mL | FACS staining |

buffer

PBMCs were incubated with the various Hydra and lectin reagents on ice for 30 minutes. Cells were washed using 150 μL of PBS (1% BSA) into each well and centrifuged at 350×g for 5 minutes. Plate solution was quickly decanted. AF-647 goat anti-mouse IgG was used with a 1/2000 dilution in PBS as a secondary stain for Hydra reagents (Hydra-7 and Hydra-9). Streptavidin conjugated Alexa Fluor 647 was used at a dilution of 1/2000 in PBS as a secondary stain for lectin reagents (PNA, MAL-II and SNA). Cells were incubated for 15 minutes on ice. Cell lineage-specific staining was performed as shown in TABLE 11 with indicated antibodies. All antibodies were purchased from Biolegend® (San Diego, Calif.) with the exception of Live Dead stain, which was purchased from Thermo Fisher Scientific (Waltham, Mass.).

TABLE 11

| Marker | Fluorochrome | Clone | Cat no. | Marker for |
|---|---|---|---|---|
| CD3 | PE | UCHTI | 300441 | Pan-T cells |
| PNA/Hydra/ | APC/AF647 | N/A | N/A | |
| MAL-II | | | | |
| CD14 | BV421 | MSE2 | 301830 | Myeloid in |
| | | | | PBLs |
| HLA-DR | PerCPcy5.5 | L243 | 307628 | |
| CD 163 | BV510 | GH1/61 | 333628 | |
| CD4 | BV605 | SK3 | 344646 | CD4+T |
| CD56 | BV650 | 5.1H11 | 362532 | NK |
| CD8 | BV786 | SKI | 344740 | CD8+T |
| CD20 | PE-Cy-7 | 2H7 | 302312 | B cell |
| Live dead | APC-Cy-7 | N/A | L34975 | |
| CD11c | APC-R-700 | Bul5 | 337220 | |

A master mix was prepared using the reagents in TABLE 11 in FACS staining buffer (“stain mix”) and 30 μg of stain mix was aliquoted into each well/tube for a final active antibody concentration ˜1 μg/ml. Cells were incubated on ice for 15 minutes. In addition, individual cellular compensation controls were prepared. Cells were washed with PBS (1% BSA) and resuspended in 4% paraformaldehyde at room temperature for 10 min. Cells were washed twice using PBS, and pellets were resuspended in 150 μl PBS. Samples were run using a flow cytometer (BD FACSCelesta™ (BD Biosciences)).

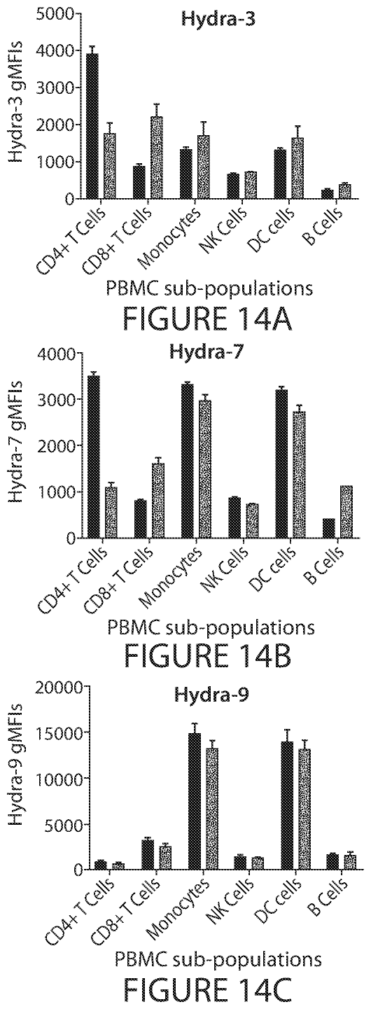

Human PBMCs from two different healthy donors were stained with Hydra-3, Hydra-7 and Hydra-9, as depicted in FIG. 14 (black and grey bars represent the two donors). As shown, monocytes and DC cell populations exhibit increased Hydra-9 staining in comparison with other cell populations (FIG. 14A). Monocytes and DC cell populations exhibit increased Hydra-7 staining in comparison with other cell populations (FIG. 14B). One donor demonstrated increased Hydra-7 staining on CD4+ T cells. Monocytes and DC cell populations exhibited increased Hydra-3 staining in comparison with other cell populations (FIG. 14C). One donor demonstrated increased Hydra-3 staining on CD4+ T cells.

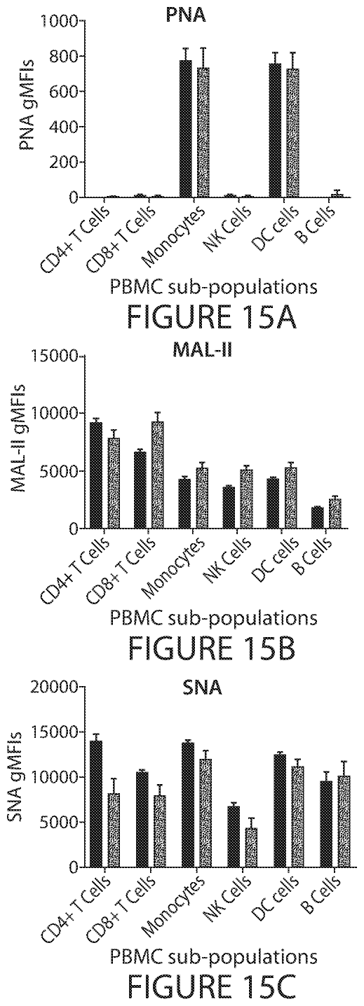

Lectin staining (MAL-II, PNA and SNA) of human PBMCs from healthy donors is depicted in FIG. 15 (black and grey bars represent two independent donors). As shown, PNA staining is relatively low in comparison with Hydra-9 staining (see scale of Y-axis in comparison to FIG. 14) but specific for monocytes and DCs (FIG. 15A). MAL-II stains most of the immune cell populations (FIG. 15B). T cells (CD4+ and CD8+) exhibit increased MAL-II staining as compared to other cell populations. SNA stains most of the immune cell populations (FIG. 15C), with NK cells exhibiting less SNA staining in comparison with other cell populations.

### Example 6: Sialidases Efficiently Desialylate Dendritic Cells (DCs)

This example demonstrates the desialylation efficiency of sialidase molecules of the current invention on human monocyte-derived dendritic cells (DCs).

DCs are known to express high levels of Siglecs (sialic acid-binding immunoglobulin-like lectins, e.g., Siglec-3, -7, and -9), which inhibit the NK cell-mediated killing of tumor cells. Additionally, DCs express numerous sialoglycans that are ligands for Siglec molecules, as demonstrated in the previous example. Interactions of the Siglecs on DCs with sialoglycans, either on the same cell or on another interacting cell (e.g., a cancer cell), regulate DC activation.

PBMCs were isolated from leukopaks (blood samples enriched in PBMCs) using a standard Ficoll density gradient method. After PBMC isolation, the cells were washed twice with cold autoMACS® rinsing solution (containing 5% BSA; Miltenyi Biotec) by centrifuging at 350×g for 5 minutes. CD14+ monocytes were magnetically purified using CD14 microbeads (Miltenyi Biotec) and differentiated into dendritic cells. Specifically, CD14+ cells were resuspended in complete medium (10% FBS-containing RPMI media) containing 50 ng/ml of recombinant human GM-CSF and 50 ng/mL of recombinant human IL-4 at a concentration of 0.8 cells×10⁶/mL. On Day 0, the cells were plated in 6-well plates with 3 ml of cell suspension per well (2.4×10⁶ cells/well). At Day 3 and Day 6, half of the medium from each well was removed, taking care not to disturb the loosely attached cells. Each well was replenished with 1.5 mL of fresh medium containing 100 ng/mL each of rhGM-CSF and rhIL-4. At Day 7, the differentiated DCs were harvested by gentle flushing with medium, washed once with complete medium and resuspended at 2×10⁶/mL.

For the desialylation assay, M106 (M1D, V6Y, P62G, A93E, I187K, C332A, and human IgG1 Fc with hole (Y407T) mutation and an EPKSS (SEQ ID NO: 163) linker) (SEQ ID NO: 152, encoded by SEQ ID NO: 193) was used. This is construct is as described in Example 2, but with an EPKSS (SEQ ID NO: 163) linker instead of a GGGGSGGGGS (SEQ ID NO: 162) linker. The term “M106: used in the Examples going forward refers to this construct. In addition, a Neu2-FC variant termed LOF (M1D, V6Y, K9D, I187K, C332A, A93E, V363R, L365R, E218A, C219N, and human IgG1 Fc with hole (Y407T) mutation (SEQ ID NO: 175, encoded by SEQ ID NO: 176)) was used as a negative control. 100,000 DCs per well were plated out in a 96-well U bottom format, with 200 μl dispensed per well. LPS was used where indicated at 0.3 ng/mL, and M106 and LOF constructs were used at the following concentrations (in μg/mL): 0, 6.25, 12.5, 25, 50 and 100. DCs were incubated overnight (16 hours) followed by flow analysis of CD83, CD86 and MHCII (HLA-DR). Desialylation was measured by PNA staining as described in Example 5.

After incubation, the plates were centrifuged at 350×g for 4 minutes and the medium was removed. Cells were washed once with FACS staining buffer. Cells were blocked and stained for dead cells simultaneously by adding 100 μl of solution containing Human Trustain FcX (1/20 dilution) and LIVE/DEAD™ Fixable Near-IR Dead Cell Stain (1/2000 dilution) in PBS and incubating on ice for 10 minutes. Cells were centrifuged and washed once with FACS buffer. 50 μL of PNA-biotin (1 μg/mL in FACS staining buffer) was added to each well and incubated on ice for 10 minutes. Cells were centrifuged and washed twice with FACS buffer. 50 μL of the antibody cocktail (described in TABLE 12 below) including Streptavidin Alexa Fluor™ 647 was added to each well and incubated on ice for 30 minutes. After incubation, the cells were washed twice with 150 μL of FACS buffer and resuspended in 125 μL of FACS buffer for flow cytometric acquisition. The flow cytometric data was acquired on a flow cytometer (BD FACSCelesta™ (BD Biosciences)) using a HTS (High Throughput Sampler) option. After data acquisition signals were analyzed using FlowJo flow analysis software (BD Biosciences).

TABLE 12

| | | | Dilution/Concentration |
|---|---|---|---|
| Reagent | Vendor | Cat# | used |
| PNA | Vector Labs | NB-1075-5 | 1 μg/mL |
| Streptavidin Alexa | Thermo | S21734 | 1/2000 |
| Fluor 647 | Fisher | | |
| LIVE/DEAD ™ | Thermo | L34976 | 1/2000 |
| Fixable Near-IR | Fisher | | |
| Dead Cell Stain | | | |
| Anti-CD209-FITC | Biolegend ® | 330104 | 1/25 |
| Anti-CD83-PE | Biolegend ® | 305308 | 1/25 |
| Anti-HLA-DR-PE- | Biolegend ® | 307616 | 1/100 |
| Cy7 | | | |
| Anti-CD11c-Alexa | Biolegend ® | 337220 | 1/50 |
| Fluor 700 | | | |
| Anti-CD86-Brilliant | Biolegend ® | 305442 | 1/50 |
| Violet 785 | | | |

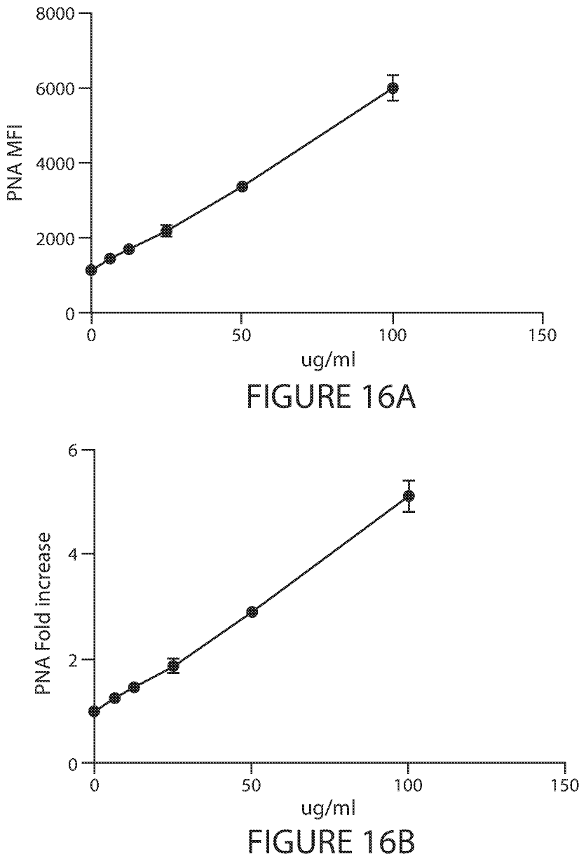

FIG. 16 depicts the degree of desialylation of DCs by M106 based on PNA staining. An increase in PNA staining is indicative of removal of the terminal sialic acid, exposing the underlying galactose residues recognized by the PNA lectin. FIG. 16A shows the increase in fluorescence (MFI), indicative of PNA staining, with increasing M106 concentration. FIG. 16B shows the fold increase in PNA signal as compared to untreated DCs. A clear dose-dependent increase in PNA signal was observed indicating a robust desialylation of the DCs.

Taken together, this example shows that M106 causes a robust desialylation of DCs in a dose-dependent manner.

### Example 7: Desialylation of Tumor Cell Lines by Sialidases

Sialoglycans play role in maintaining tolerance and homeostasis in human physiological conditions. Overexpression of sialoglycans is observed in tumor cell lines. This example demonstrates the ability of M106 to desialylate tumor cell lines BT-20, SKBR-3, HT-29 as determined by Hydra-9 and lectin staining.

BT-20 and HT-29 cells were grown to 70-80% confluence on plates using appropriate media. Cells were dissociated using Accutase® (Innovative Cell Technologies, Inc.), an enzyme mixture containing proteolytic and collagenolytic enzyme activity, by incubating the plates at 37° C. for 15 minutes. When the cells were dissociated, an equal volume of complete media was added to neutralize Accutase®. The cell suspension was transferred and centrifuged at 300×g for 5 min. The supernatant was discarded and cells were washed twice with cold PBS. Cells were counted and resuspended in media at 1×10⁶ cells per ml. M106 and LOF were added at varying dilutions to cells. Cells were incubated for 10 hours at 37° C. After the incubation, cells were washed with PBS and transferred to 96-well round bottom plates for staining. Staining was performed with Hydra-9 and PNA as in Example 5.

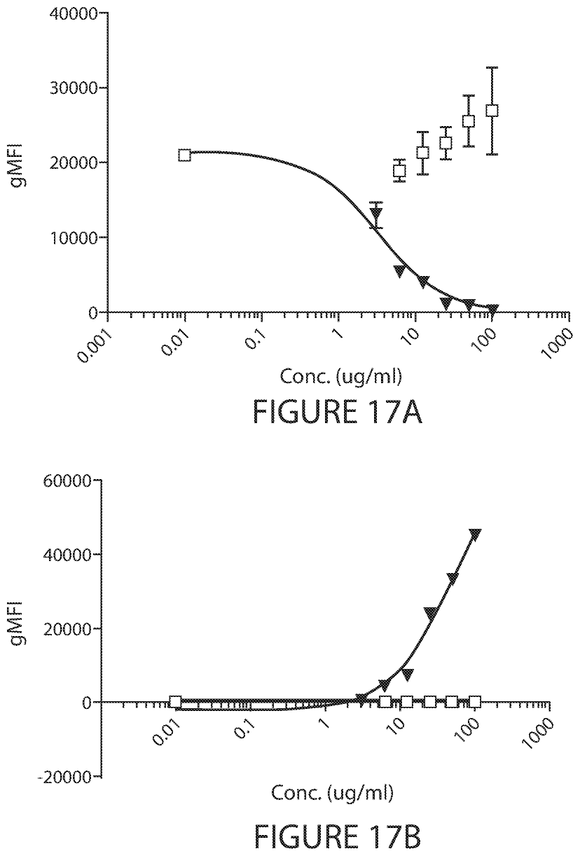

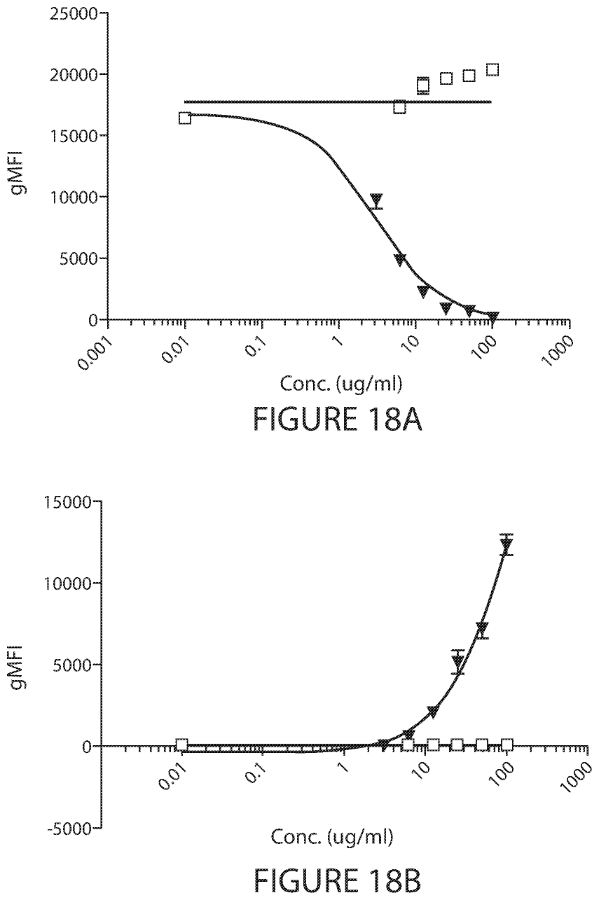

FIG. 17 depicts the degree of desialylation of BT-20 cells following treatment with M106 (triangles) or LOF control (squares) as determined by loss of Hydra 9 binding (FIG. 17A) or increase in PNA staining (FIG. 17B), as measured by fluorescence (gMFI). An IC50 for desialylation by M106 was 3.088 μg/mL for Hydra 9 and 58.75 μg/mL for SNA. FIG. 18 depicts the degree of desialylation of BT-20 cells following treatment with M106 (triangles) or LOF control (squares) as determined by loss of Hydra 9 binding (FIG. 18A) or increase in PNA staining (FIG. 18B), as measured by fluorescence (gMFI). The IC50 for desialylation by Neu2-Fc variant M106 was 2.95 μg/ml for Hydra 9 and 131.5 μg/mL for SNA.

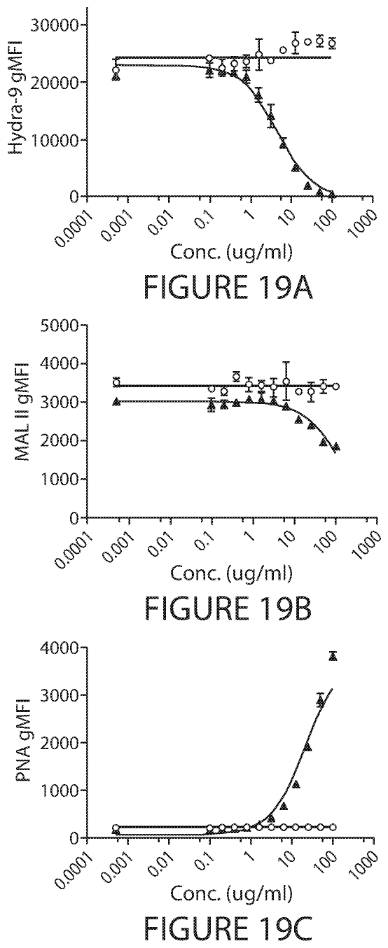

A similar experiment was performed with SKBR-3 cells, in which cells were stained with MAL-II lectin in addition to Hydra 9 and PNA. For MAL-II staining, a final concentration of 2 μg/mL in PBS was used and cells were stained for 10 minutes at room temperature. FIG. 19 depicts the degree of desialylation of SKBR-3 cells following treatment with M106 (triangles) or LOF control (circles) as determined by loss of Hydra 9 binding (FIG. 19A), loss of MAL-II staining (FIG. 19B) or increase in PNA staining (FIG. 19C), as measured by fluorescence. An IC50 for desialylation by M106 was 4.4 μg/ml with Hydra 9, approximately 120 μg/mL for MAL-II and 22 μg/ml for SNA.

Taken together, this example shows that M106 demonstrated a dose dependent removal of cell surface sialic acid from tumor cells. Loss of Hydra 9 staining is a more sensitive indicator with EC50s around 3 to 4 ug/mL of M106 as compared to loss of MAL II staining or gain of PNA staining.

### Example 8: Desialylation of Tumor Cell Lines by Sialidases Enhances Human Dendritic Cell Activation

Sialoglycans play role in maintaining tolerance and homeostasis in human physiological conditions. Although overexpression of sialoglycans is observed in tumor cell lines, the resulting sialoglycans can be removed using sialidases of the present invention as shown in the previous examples. This example demonstrates the effect of desialylation of tumor cell lines on dendritic cell activity.

Briefly, dendritic cells (DCs) were generated from CD14+ monocytes isolated from PBMCs of healthy donors. CD14+ cells were magnetically purified using manufacturer's protocol (Miltenyi Cat #130-050-201). The purified cells were then cultured for 7 days in presence of GM-CSF (R&D Systems Cat #7954-GM/CF) and IL-4 (R&D Systems Cat #6507-IL/CF) to generate immature DCs.

On the day of the experiment, SKBR-3 tumor cells were harvested from T-75 flasks using Accutase® and washed twice with 10% FBS McCoy's 5A medium. The cells were then resuspended at 5×10⁶/mL of 10% FBS McCoy's 5A medium. 100 μg/mL of M106 was added to the sample and incubated at 37° C. for 4 hours. The no treatment group was treated identically except for the addition of M106 to the tube. After 4 hours, the cells were washed twice with 10% FBS McCoy's 5A medium and resuspended at 2×10⁶/mL in complete medium (10% FBS RPMI). 50 μl (100,000 DCs) of the suspension was added to the designated wells.

DCs were harvested, washed in complete medium (10% FBS RPMI) and resuspended at 2×10⁶/ml. 50 μL (100,000 DCs) of the suspension was added to the designated wells.

LPS (InvivoGen Cat # tlrl-pb5lps) was added to a final concentration of 0.3 ng/mL. Complete medium (10% FBS RPMI) was added where needed to reach a final volume of 200 μL per well. The assay plate was incubated overnight at 37° C. On the following day, the cells were washed with staining buffer and stained for DC markers (CD11c, CD209, CD1c, CD83, CD86 and HLA-DR). The desialylation of the tumor cells was confirmed by staining with Hydra-9 as described in Example 6.

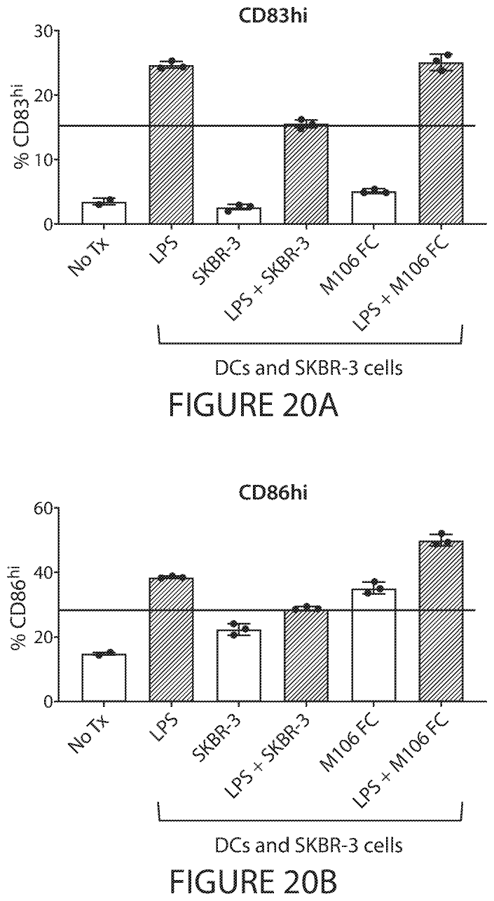

FIG. 20 depicts the effects of dendritic cell activation under various conditions as determined by CD83hi expression (FIG. 20A) or CD86hi expression (FIG. 20B). Untreated DCs (“No Tx”) have a low percentage of CD83hi and CD86hi. Addition of LPS to the DCs strongly induces activation, as shown by an increased percentage of CD83hi and CD86hi (“LPS”). LPS-induced expression of both CD83 and CD86 was inhibited when DCs were co-incubated with untreated SKBR-3 tumor cells (see horizontal line in FIGS. 20A and 20B). The inhibition of DCs by SKBR-3 tumor cells is reversed following desialylation of the SKBR-3 tumor cells by M106 prior to co-incubation with DCs and LPS (“LPS+M106 FC”). In addition, sialidase treatment slightly enhances activation of DCs in the absence of LPS (compare no treatment and untreated SKBR-3 tumor cells to M106-treated SKBR-3 tumor cells (“M106 FC”)).

This example demonstrates that desialylation of tumor cells can reverse the sialoglycan-induced immunosuppression of DCs which suggests that desialylation of tumor cells can lead to a stronger anti-tumor response.

### Example 9: Effect of Sialidases on Phagocytosis of Tumor Cells by Macrophages

Sialoglycans present on immune cell surfaces play important roles in maintaining homeostasis. This example demonstrates the effect of sialidases of the present invention on phagocytosis of HT-29 tumor cells by M2-like human macrophages.

PBMCs from whole blood of human volunteers were isolated by a Ficoll method. CD14+ monocytes were magnetically purified using CD14 microbeads. Monocytes were differentiated into M2-like macrophages by resuspending CD14+ cells in RPMI media (10% FBS) at a concentration of 1×10⁶/mL with 50 ng/mL of recombinant human M-CSF. On Day 0, the cells were plated in 150 mm tissue culture plates in 20 mL volume (˜20×10⁶ cells seeded per plate). At Day 3 and Day 6, half of the medium from each well was removed, taking care not to disturb the attached cells. M-CSF was replenished to a final concentration of 50 ng/mL. On Day 7, the supernatant media was collected in 50 mL tubes and the plate was gently washed with 20 mL PBS. 20 mL Accutase® was added and plates were incubated for 20 minutes to dissociate cells from the plate. Cells were resuspended in complete RPMI media supplemented with 10% FBS and non-essential amino acids (NEAA), sodium pyruvate and HEPES with 10 ng/ml M-CSF, and seeded at 50K cells/well/100 μL in flat bottom 96-well plate.

HT-29 cells were harvested from flask using Accutase®. Cells were washed with PBS. Cells were labelled with Cell Trace™ CFSE labelling dye (FITC) conjugate (Thermo Fisher) at a 1:1000 dilution by volume (final concentration of 10 Cells were incubated at room temperature for 10 minutes and the labelling reaction was quenched by adding an equal volume of chilled FBS. Cells were washed twice and resuspended in media (10% FBS supplemented McCoy's media) at 1.2×10⁶/ml cells. M106 and LOF were added at a top concentration of 100 μg/ml followed by 2-fold dilutions. A no treatment control group was reserved with untreated HT-29 cells. Cells were incubated at 37° C. for ˜20 hours.

Following the incubation, cells were spun down, washed with PBS, and resuspended into complete RPMI (10% FBS) media at a final cell density of 2.5×10⁶ cells/mL. 100 μL HT-29 cell suspension was added to M2-like macrophages in appropriate wells at a macrophage: tumor cell ratio of 1:5 (E:T). Plates with macrophage and tumor cells were incubated for 2 hours to allow for phagocytosis. After 2 hours, the media was gently removed using multi-channel pipette and 200 μL of Accutase® was added to the plates incubated for 45 minutes on ice to detach both HT-29 and macrophages from the plate. The cells were resuspended and collected in a new 96-well bottom plate. The plates were spun down, the supernatant was discarded, and the cell pellets were washed in 200 μL of PBS.

Cell pellets were resuspended and blocked using Human Trustain Fc blocker on ice for 5-7 minutes. After incubation, cells were washed with PBS. Cells were stained for CD45 and CD14 fluorochrome markers as below in TABLE 13. Antibodies were purchased from Biolegend®.

TABLE 13

| Marker | Fluorochrome | Clone | Cat no. | Marker for |
|---|---|---|---|---|
| CD14 | BV421 | MSE2 | 301830 | Macrophages |
| CD45 | APC | 2D1 | 368512 | Macrophages |

A master mix was made in FACS staining buffer with staining antibodies added at 1:30 dilution. 30 μl of master mix was added/well. Appropriate compensation controls (e.g., single color staining controls for compensation as per standard flow cytometry practice for multi-color flow cytometry) were stained in parallel. Cells were incubated on ice for 15 minutes and then washed with PBS at 350 g for 8 minutes. The cells were then fixed with 4% formaldehyde for 10 minutes at room temperature and afterwards washed twice with PBS. Cells were resuspended in 150 μL of PBS and run on a flow cytometer (BD FACSCelesta™ (BD Biosciences)).

The percentage of CFSE-positive, CD14+CD45+ macrophages were determined. CFSE-positive, CD14+CD45+ macrophages are indicative of percentage phagocytosis of tumor cells by macrophages, because CFSE-positive tumor cells that are phagocytosed by CD14+CD45+ macrophages are CFSE positive.

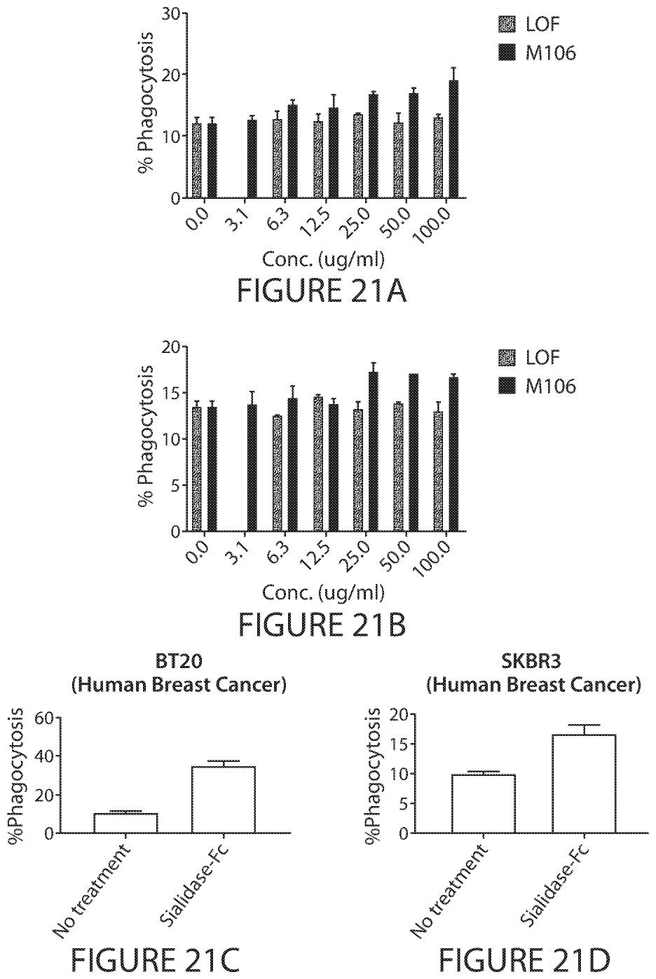

FIG. 21 depicts the dose dependent enhancement of phagocytosis of desialylated HT-29 tumor cells by M2 like macrophages derived from two different healthy donors (FIG. 21A and FIG. 21B). HT-29 pretreated with sialidase at concentrations above 25 μg/mL demonstrated a reproducible increase in phagocytosis by macrophages. A similar increase in phagocytosis of desialylated BT20 and SKBR-3 tumor cells by M2-like macrophages was observed (FIG. 21C and FIG. 21D respectively).

Accordingly, treating tumor cells with a sialidase as described herein resulted in an increase in phagocytosis of the tumor cells by macrophages.

### Example 10: Sialidase Treatment Enhances MHC Class-II Expression on Monocytes

This example demonstrates the effect of sialidases of the current invention on MHC class-II (HLA-DR) expression on monocytes. MHC-II expression represents antigen presentation capacity on the monocytes. Enhanced class-II expression is indicative of enhanced antigen presentation to T cells to generate an effective immune response.

PBMCs were isolated from healthy volunteers using Ficoll method and cells were washed twice with ice cold PBS using a tabletop centrifuge at 350×g for 10 minutes. Cells were resuspended in media and counted using a Countess™ II Automated Cell Counter. The final suspension was adjusted to 2.5×10⁶ cells/L. About 250,000 cells (100 μL) were seeded in 96-well round bottom plates. The cells were incubated with M106 or LOF at a top concentration of 50 μg/mL, with 2-fold dilutions. A no treatment group was included. The cells were incubated for 18 hours at 37° C. The plates were spun at 350×g for 10 minutes. Cell pellets were washed with cold PBS, and subjected to blocking and staining steps using the FACS staining panel described in TABLE 14. All antibodies were purchased from Biolegend® with the exception of Live Dead stain, which was purchased from Thermo Fisher. Sialoglycan staining was performed using PNA lectin as confirmation of desialylation using the methods described in Example 7.

TABLE 14

| Marker | Fluorochrome | Clone | Cat no. | Marker for |
|---|---|---|---|---|
| CD3 | PE | UCHT1 | 300441 | Pan-T cells |
| PNA | APC | N/A | | Glycosylation |
| | | | | specific Lectin |
| CD14 | BV421 | MSE2 | 301830 | Myeloid in |
| | | | | PBLs |
| CD19 | PerCPcy5.5 | HIB19 | 302230 | B cell |
| CD163 | BV510 | GH1/61 | 333628 | |
| CD4 | BV605 | SK3 | 344646 | CD4+ T |
| CD56 | BV650 | 5.1H11 | 362532 | NK |
| CD8 | BV786 | SK1 | 344740 | CD8+ T |
| HLA-DR+ | PE-Cy-7 | L243 | 307628 | |
| Live dead | APC-Cy-7 | N/A | L34975 | |
| CD11c | APC-R-700 | Bu15 | 337220 | |

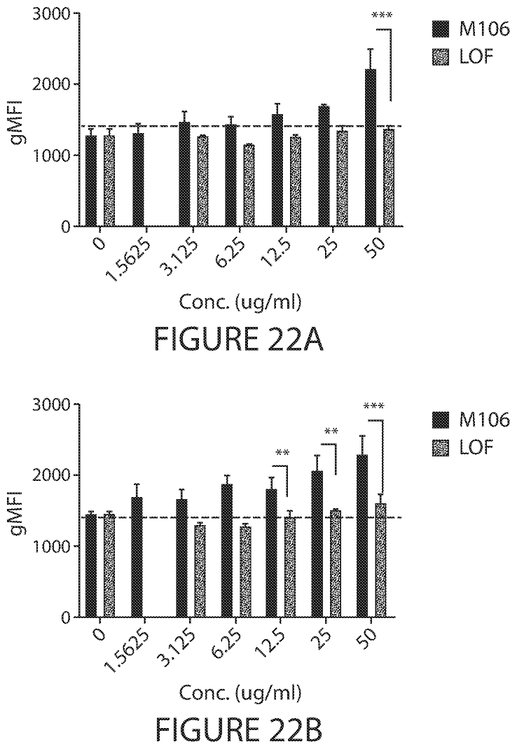

FIG. 22 depicts the dose dependent enhancement of HLA-DR expression following M106 desialylation compared to LOF in monocytes from two different healthy donor (FIG. 22A and FIG. 22B).

Accordingly, this example shows that desialylation of monocytes by a sialidase described herein leads to an increased MHC class-II (HLA-DR) expression on monocytes. MHC-II expression represents antigen presentation capacity on the monocytes. Thus, enhanced class-II expression is indicative of enhanced antigen presentation to T cells, which can enhance the ability of T cells to generate an effective immune response.

### Example 11: Sialidase Treatment does not Result in Adverse Cytokine Release

Conditioned media from PBMCs incubated with M106 or LOF was assayed for stimulation of cytokine release. LPS (1 ng/mL) was used as a positive control. M106 (as well as LOF) treatment of PBMCs demonstrated no increase across all treatment doses of TNF-alpha, IL-6, IL-1beta, IL-1RA or IL-10 in two independent donors as measured by LEGENDplex™ Human M1/M2 Macrophage Panel (10-plex; BioLegend®). In contrast, LPS demonstrated a clear cytokine induction. These results demonstrate that sialidase treatment of PBMCs does not result in adverse cytokine release.

### Example 12: Sialidase Treatment Leads to Complete and Partial Remission of Tumor Growth Alone and in Combination with an Anti-PD-1 Antibody

This example shows that in vivo administration of sialidases of the present invention can result in complete and partial remission of tumor growth in various mouse syngeneic tumor models.

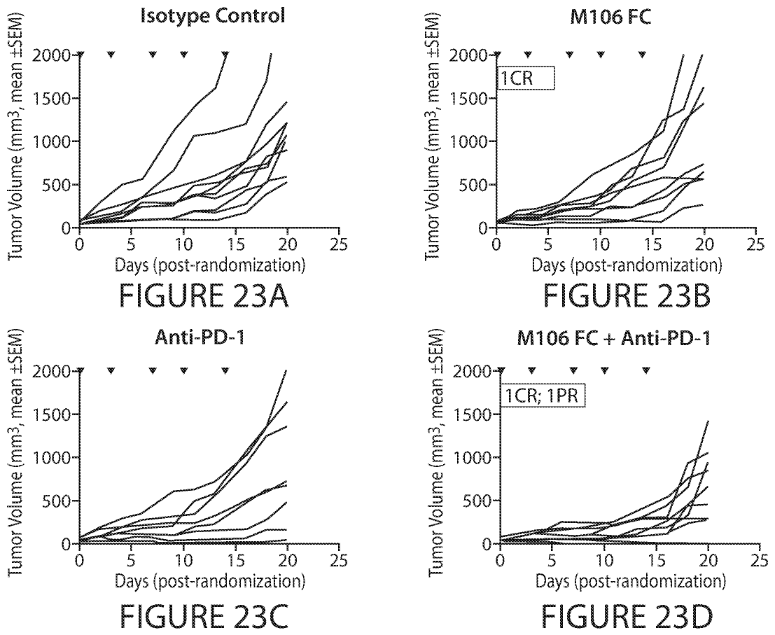

Sialidase treatment alone and in combination with other cancer treatments were tested using the MC38 colon cancer cell model. Each mouse was inoculated subcutaneously in the right lower flank region with 5×10⁵ tumor cells in 0.1 mL of PBS to induce tumor development. Mice were randomized when the mean tumor size reached approximately 50 mm³. 32 mice were randomly allocated to 4 study groups. The mice were dosed with either M106, anti-mouse PD-1, a combination of Neu2-Fc variant M106 and anti-PD-1 or isotype control at 10 mg/kg of each agent twice per week for 5 doses. FIG. 23 depicts the tumor growth for each mouse in either the isotype control group (FIG. 23A), M106 group (FIG. 23B), anti-PD-1 group (FIG. 23C) or a combination of M106 and anti-PD-1 (FIG. 23D). M106-treated mice demonstrated complete remission (CR) of tumor growth in one animal compared to no mice responding in the isotype treated group. The combination of M106 and anti-PD-1 demonstrated 1 CR and 1 partial response (PR) as well as an overall reduction in tumor growth in all mice compared to isotype control.

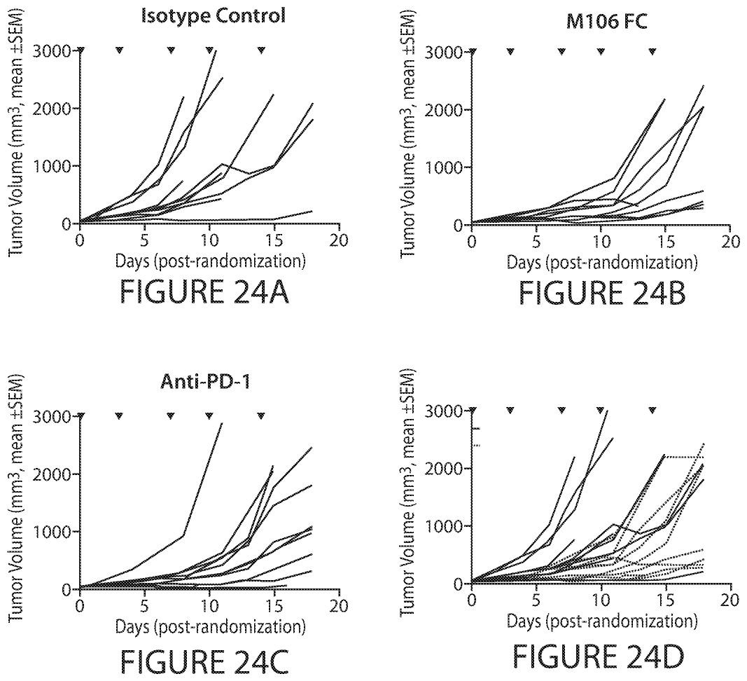

Next, sialidase treatment alone and in combination with other cancer treatments were tested using the B16F10 melanoma cancer cell model. Each mouse was inoculated subcutaneously in the right lower flank region with 5×10⁵ tumor cells in 0.1 mL of PBS for tumor development. Mice were randomized when the mean tumor size reached approximately 50 mm³. 24 mice were randomly allocated to 3 study groups. The mice were dosed with either M106, anti-mouse PD-1 or isotype control at 10 mg/kg twice per week for 5 doses. FIG. 24 depicts the tumor growth for each mouse in either the isotype control group (FIG. 24A), M106 group (FIG. 24B) or anti-PD-1 group (FIG. 24C). FIG. 24D is an overlay of the isotype control group on the M106 group demonstrating a clear benefit of M106 in reducing tumor growth in what is considered a difficult-to-treat tumor model.

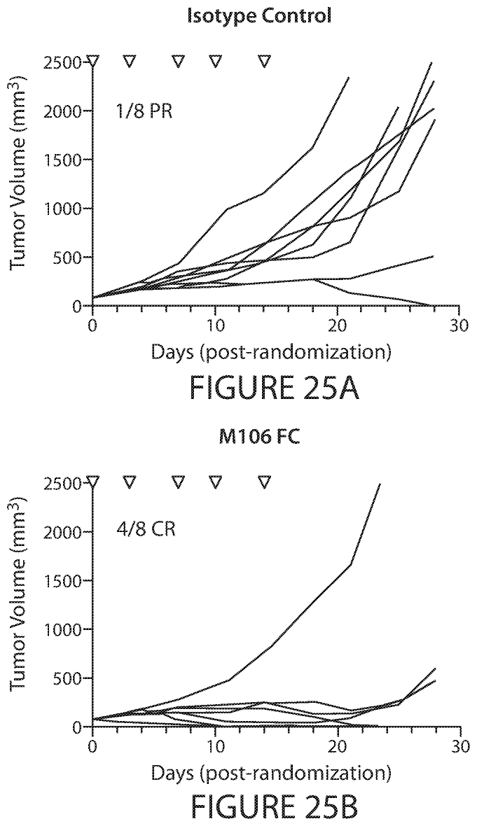

Next, sialidase treatment alone and in combination with other cancer treatments were tested using the cell line EMT6 expressing human Her2 as a polyclonal cell line. Each mouse was inoculated subcutaneously in the right lower flank region with 5×10⁵ tumor cells in 0.1 mL of PBS for tumor development. The mice were randomized when the mean tumor size reached approximately 100 mm³. 16 mice were randomly allocated to 2 study groups. The mice were dosed with either M106 or isotype control at 10 mg/kg twice per week for 5 doses. FIG. 25 depicts the tumor growth for each mouse in either the isotype control group (FIG. 25A) or M106 FC group (FIG. 25B). 4 out of 8 M106-treated mice demonstrated complete remissions (CR) of tumor growth compared to only 1 out of 8 mice in the isotype treated group.

Accordingly, as demonstrated in this example, treatment with the sialidases disclosed herein leads to the reduction in cancer growth and, in some instances, complete remission, in a variety of cancer types.

### Example 13: Sialidase Treatment Leads to Complete and Partial Remission of Tumor Growth Alone and in Combination with an Anti-PD-L1 Antibody

This example describes in vivo testing of M106 and/or avelumab (anti-PD-L1) in an A20 syngeneic mouse model. Mouse A20 cells express endogenous mouse PD-L1 which is bound by avelumab. Female Balb/c mice, 5-6 weeks of age, were inoculated subcutaneously in the right lower flank region with murine A20 B cell lymphoma cells in matrigel (1:1 by volume). Mice were randomly allocated into groups of 8 mice when tumors reached approximately 100 mm³ (the average tumor volume of each group ranged from 86 to 90 mm³). TABLE 15 describes the various arms of the study. The mice were treated intraperitoneally with 5 or 10 mg/kg M106, avelumab, and/or antibody isotype control (as indicated) twice a week for a total of 5 doses. Tumor volumes and body weights were recorded three times a week.

TABLE 15

| Group | No. Mice | Treatment | Dose | Schedule |
|---|---|---|---|---|
| 1 | 8 | Isotype control | 10 mg/kg | Twice weekly for 5 |
| | | | | doses |
| 2 | 8 | Avelumab | 5 mg/kg | Twice weekly for 5 |
| | | | | doses |
| 3 | 8 | Avelumab | 10 mg/kg | Twice weekly for 5 |
| | | | | doses |
| 4 | 8 | M106-Fc | 10 mg/kg | Twice weekly for 5 |
| | | | | doses |
| 5 | 8 | Avelumab in | 10 mg/kg | Twice weekly for 5 |
| | | combination | each | doses |
| | | with M106-FC | | |

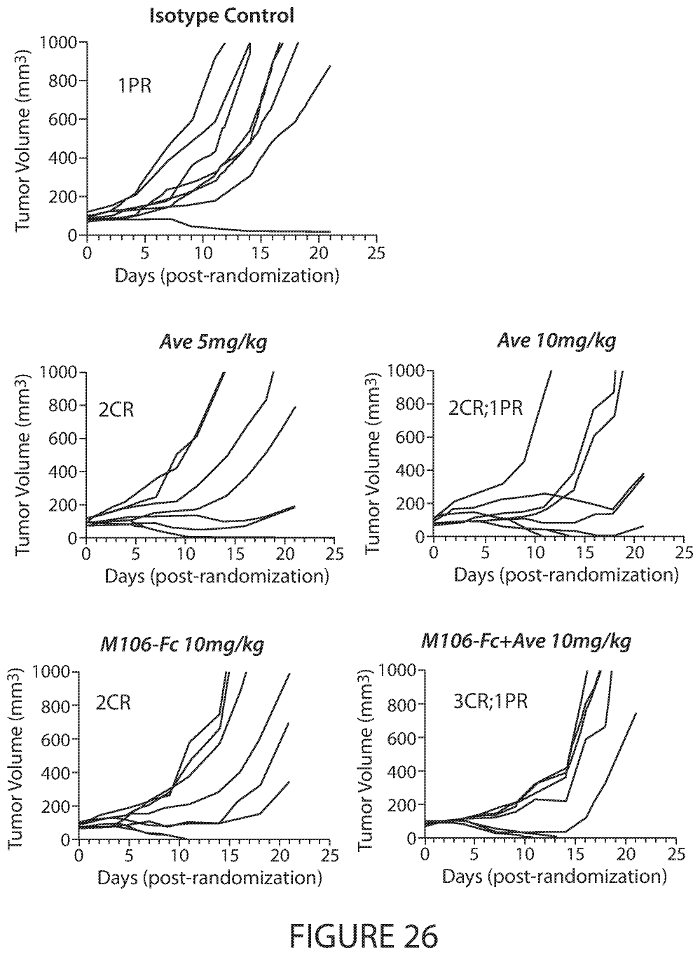

FIG. 26 depicts the tumor growth in each mouse in each of the groups. Complete responders (CR) and partial responders (PR) for each group are shown. As can be seen, M106 demonstrated anti-tumor activity alone and in combination with avelumab (“Ave”).

Mice with tumors that demonstrated CR from M106 treatment groups (alone or in combination with avelumab) were grouped and rechallenged with murine A20 cells (all approximately 12 weeks of age) and compared to naïve control mice injected with A20 cells of either 6 or 12 weeks of age. Tumor volumes and body weights were recorded three times a week. Tumors grew as expected in both the 6 week and 12 week naïve mice, no tumor growth was observed in the rechallenged mice (data not shown.)

Accordingly, as demonstrated in this example, treatment with a sialidase disclosed herein leads to the reduction in cancer growth and, in some instances, complete remission, in a B cell lymphoma model.

### Example 14: Sialidase Treatment Leads to Complete and Partial Remission of Tumor Growth Alone and in Combination with an Anti-PD-L1 Antibody

This example describes in vivo testing of M106 and/or avelumab (anti-PD-L1) in an A20 syngeneic mouse model. The experiment was performed as in Example 13, except that 6 doses were given (twice weekly for 3 weeks). TABLE 16 describes the various arms of the study. Mice were treated intraperitoneally with 10 mg/kg M106, avelumab and/or antibody isotype control twice a week for a total of 6 doses. Tumor volumes and body weights were recorded three times a week.

TABLE 16

| Group | Treatment | Route/dose | Schedule |
|---|---|---|---|
| 1 | Isotype | 10 mg/kg IP | Twice weekly |
| 2 | Avelumab | 10 mg/kg IP | for 3 weeks |
| 3 | Avelumab + M106 FC | 10 mg/kg IP + 10 mg/kg IP | |
| 4 | M106 FC | 10 mg/kg IP | |

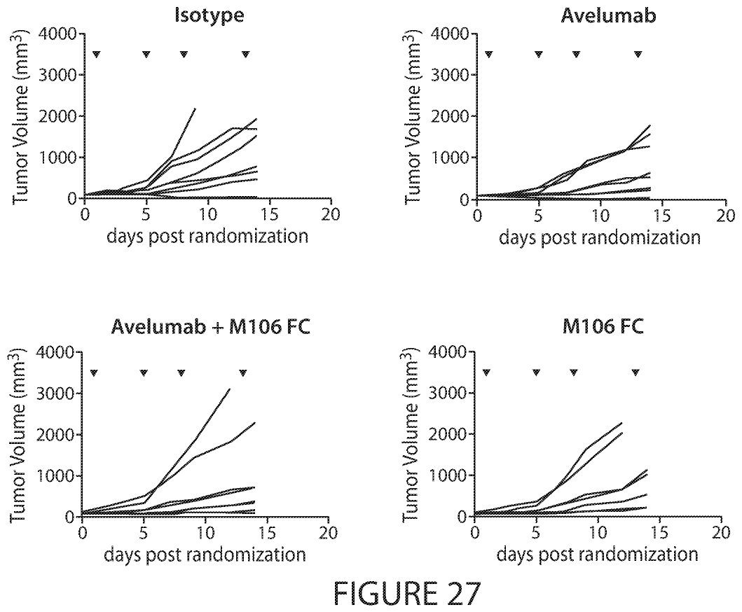

FIG. 27 depicts the results of tumor growth in each mouse in each of the groups. The avelumab-based ASCs demonstrated varying degrees of efficacy. As in Example 13, M106 demonstrated activity as does M106 combined with avelumab.

Accordingly, as demonstrated in this example, treatment with a sialidase disclosed herein, alone or in combination with an anti-PD-L1 antibody, leads to the reduction in cancer growth and, in some instances, complete remission, in a B cell lymphoma model.

### Example 15: Sialidase Treatment Leads to Improved Survival in Combination with an Anti-CD20 Antibody in Tumor-Bearing Mice

This Example describes the in vivo administration of M106 in combination with an anti-CD20 antibody (ofatumumab) in a mouse syngeneic intravenous dissemination model using a murine breast cancer cell line expressing human CD20 (EL4 CD20 cells). Female C57/BL6 mice, 6-8 weeks of age, were IV injected with 500,000 cells per mouse. Mice were subsequently dosed as described in TABLE 17 with isotype control, ofatumumab, an or a combination of ofatumumab and M106. Body weights and clinical observations were recorded daily.

TABLE 17

| | # of | | Dose | Tumor | | Dose volume | |
|---|---|---|---|---|---|---|---|
| Group | mice | Treatment | (mg/kg) | route | Route | mL/kg | Schedule |
| 1 | 10 | Isotype control | 10 | IV | IP | 5 | Twice weekly |
| | | | | | | | for 4 doses |
| 2 | 20 | Ofatumumab | 10 | IV | IP | 10 | Twice weekly |
| | | | | | | | for 4 doses |
| 5 | 20 | Ofatumumab | 10 each | IV | IP | 10 and 5 | Twice weekly |
| | | and M106 | | | | | for 4 doses |

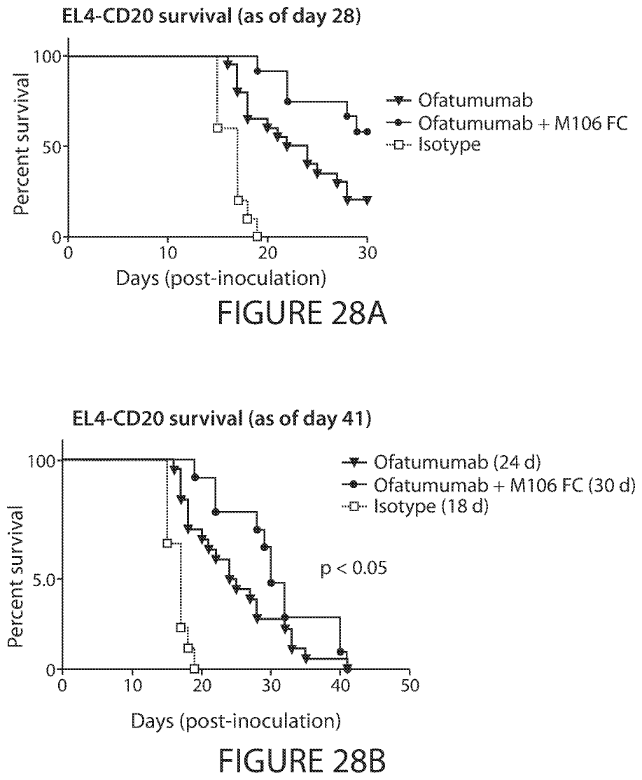

FIG. 28 depicts survival curves for mice in each group. FIG. 28A depicts the survival as of day 28 and FIG. 28B depicts the overall survival (as of day 41). Compared to isotype control, mice treated with ofatumumab demonstrated a shift in survival, with the 50% survival point shifting from 17 days to 24 days. Mice treated with a combination of ofatumumab and M106 demonstrated an even greater shift in survival to 30 days.

Accordingly, this example showed that treatment with a sialidase of the invention led to increased survival in mice treated with an anti-CD20 antibody.

### Example 16: Sialidase Treatment Disrupts Siglec-15 Activity on T Cells

Siglec-15 is an important immune suppressor. Siglec-15 is only expressed on certain myeloid cells under normal conditions, but it is broadly upregulated on human cancer cells and tumor-infiltrating myeloid cells. Siglec-15 acts as a ligand and suppresses antigen-specific T cell responses in vitro and in vivo. Genetic ablation or antibody blockade of Siglec-15 increases anti-tumor immunity in the tumor microenvironment (TME) and inhibits tumor growth in some mouse models.

This example demonstrates that neuraminidase treatment removes the Siglec-15 ligand, thereby disrupting Siglec-15 binding activity. It is believed that the disruption of Siglec-15 binding activity in vivo would cause increased anti-tumor immunity in the TME and inhibit tumor growth.

Human PBMCs were thawed and stimulated with anti-CD3 (OKT3 clone) and anti-CD28 (clone CD28.2) antibodies (both from eBiosciences, Thermo Fisher Scientific) at final concentration of 1 μg/mL in complete RPMI media (supplied with 10% heat inactivated FBS, non-essential amino acids and sodium pyruvate). On day 2, the floating cells were collected and re-plated in fresh complete RPMI media, anti-CD3 and anti-CD28 antibodies were replenished at 1 μg/mL to stimulate cells continuously. After 3 more days, cells were re-seeded in 15 mL conical tube at 10⁶/ml density and treated with different groups as follows: (1) no treatment; (2) a loss of function sialidase (“LOF FC,” as described in previous examples) at a 50 μg/mL final concentration; (3) M106 at a 50 μg/mL final concentration, and (4) BiNanH2—2 μg/mL final concentration. BiNanH2 is a strong sialidase from Bifidobacterium infantis that was used as a positive control.

Cells were supplied with anti-CD3 anti-CD28 antibodies and incubated in the 37° C. incubator overnight. The next day (˜14 hours later), cells were spun down, media was removed and then cells were blocked with Human TruStain FcX Fc receptor blocker (Biolegend®) along with LIVE/DEAD™ Fixable Near-IR Dead Cell Stain in PBS. The cells were then blocked with heat inactivated human serum (5% in PBS).

Cells were stained with Human Siglec-15-Fc (prepared by Palleon Pharmaceuticals; MW: ˜100 KDa) at final concentration of 1 μM/100 μg/mL. Cells were incubated on ice for 15 minutes and then washed with PBS.

Next, cells were stained with anti-human Fc-AF647 antibody in FACS staining buffer. Cells were incubated on ice for 5 minutes and then washed.

Then, cells were stained for CD4 and CD8 markers in FACS staining buffer as described in earlier examples. Cells were incubated on ice for 15 minutes and then washed. Cells were fixed and run on a flow cytometer (BD FACSCelesta™ (BD Biosciences)) and data was analyzed.

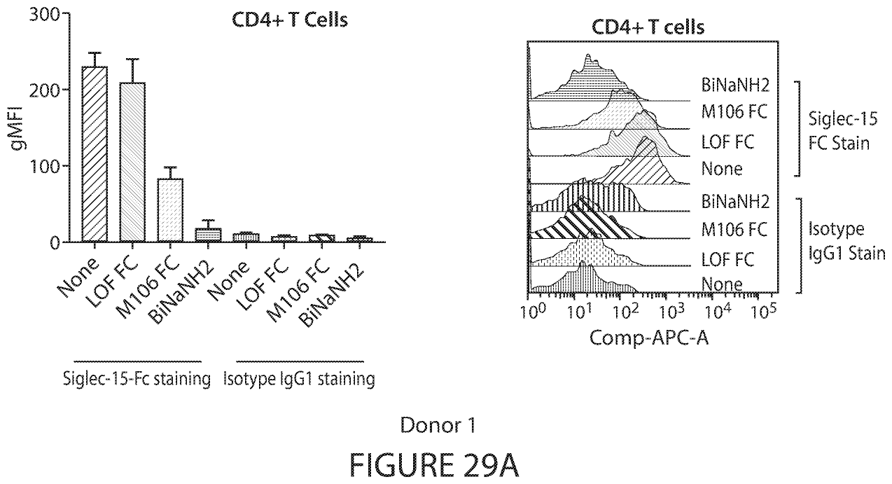

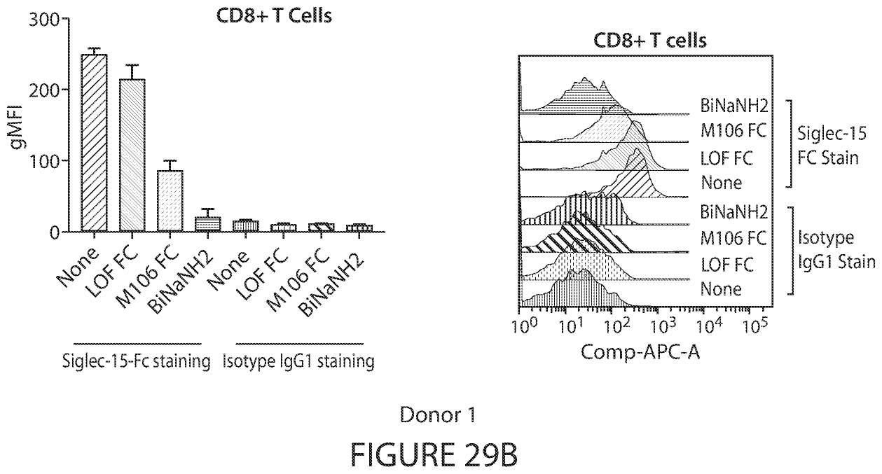

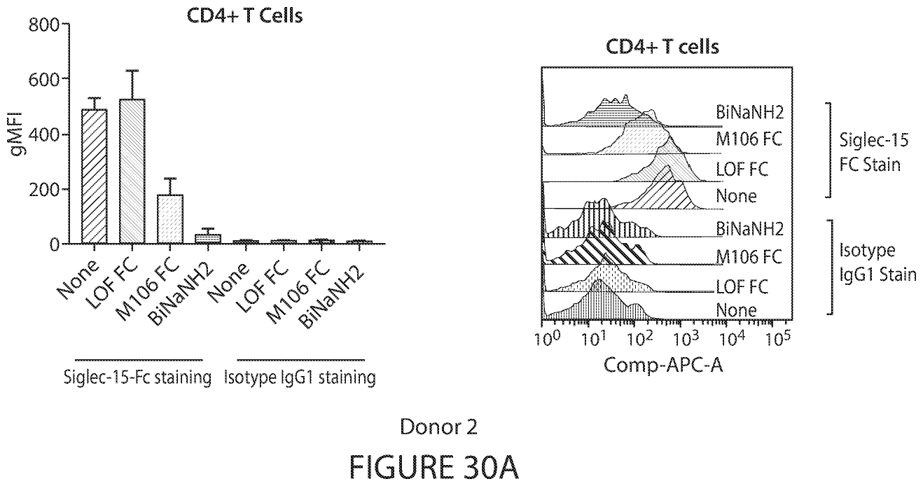

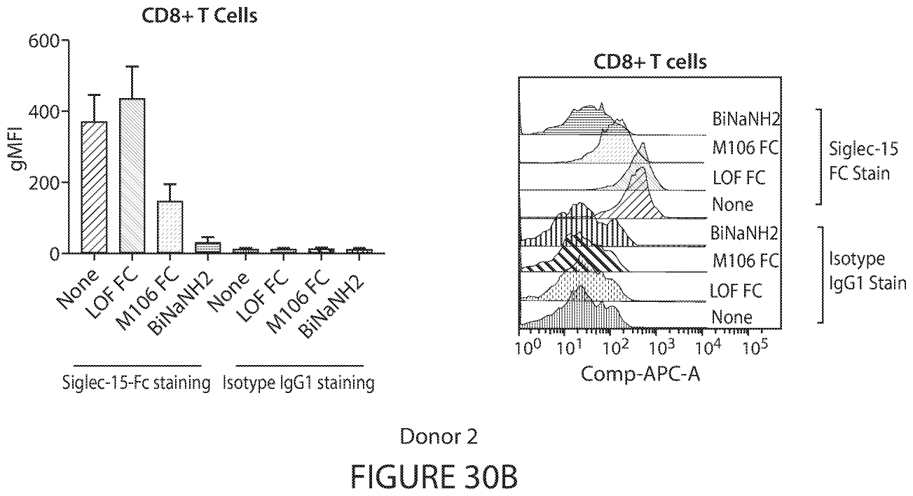

FIG. 29 depicts the results of Siglec-15-Fc staining of CD4+ cells (FIG. 29A) and CD8+ cells (FIG. 29B) following the various treatments. As a control, Isotype IgG1 staining is also shown. As shown, treatment of activated CD4 and CD8 cells with M106 FC or BiNaNH2 (positive control) decreased Siglec-15-Fc staining as compared to no treatment or treatment with LOF FC. FIG. 30 depicts the results of Siglec-15-Fc staining of CD4+ cells (FIG. 30A) and CD8+ cells (FIG. 30B) using PBMCs from a second healthy donor. These results demonstrate that Siglec-15 binding to activated T cells is sialic acid-dependent and removal of sialic acids by neuraminidase disrupts this interaction.

Accordingly, this example demonstrates that neuraminidase treatment with a sialidase of the invention removes the Siglec-15 ligand, thereby disrupting Siglec-15 binding activity. It is believed that the disruption of Siglec-15 binding activity in vivo would cause increased anti-tumor immunity in the TME and inhibit tumor growth.

### INCORPORATION BY REFERENCE

The entire disclosure of each of the patent and scientific documents referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes that come within the meaning and range of equivalency of the claims are intended to be embraced therein.

SEQUENCE LISTING

SEQ ID NO: 1:
MASLPVLQKESVFQSGAHAYRIPALLYLPGQQSLLAFAEQRASKKDEHAELIVLRRGDYDAP
THQVQWQAQEVVAQARLDGHRSMNPCPLYDAQTGTLFLFFIAIPGQVTEQQQLQTRANVTRL
CQVTSTDHGRTWSSPRDLTDAAIGPAYREWSTFAVGPGHCLQLHDRARSLVVPAYAYRKLHP
IQRPIPSAFCFLSHDHGRTWARGHFVAQDTLECQVAEVETGEQRVVTLNARSHLRARVQAQS
TNDGLDFQESQLVKKLVEPPPQGCQGSVISFPSPRSGPGSPAQWLLYTHPTHSWQRADLGAY
LNPRPPAPEAWSEPVLLAKGSCAYSDLQSMGTGPDGSPLFGCLYEANDYEEIVFLMFTLKQA
FPAEYLPQ
SEQ ID NO: 2:
MEDLRPMATCPVLQKETLFRTGVHAYRIPALLYLKKQKTLLAFAEKRASKTDEHAELIVLRR
GSYNEATNRVKWQPEEVVTQAQLEGHRSMNPCPLYDKQTKTLFLFFIAVPGRVSEHHQLHTK
VNVTRLCCVSSTDHGRTWSPIQDLTETTIGSTHQEWATFAVGPGHCLQLRNPAGSLLVPAYA
YRKLHPAQKPTPFAFCFISLDHGHTWKLGNFVAENSLECQVAEVGTGAQRMVYLNARSFLGA
RVQAQSPNDGLDFQDNRVVSKLVEPPHGCHGSVVAFHNPISKPHALDTWLLYTHPTDSRNRT
NLGVYLNQMPLDPTAWSEPTLLAMGICAYSDLQNMGQGPDGSPQFGCLYESGNYEEIIFLIF
TLKQAFPTVFDAQ
SEQ ID NO: 3:
EDLRP
SEQ ID NO: 4:
MEDLRP
SEQ ID NO: 5:
DKTHTCPPCPAPELLGGPSVFLFPPKPKDTLMISRTPEVTCVVVDVSHEDPEVKFNWYVDGV
EVHNAKTKPREEQYNSTYRVVSVLTVLHQDWLNGKEYKCKVSNKALPAPIEKTISKAKGQPR
EPQVYTLPPSREEMTKNQVSLTCLVKGFYPSDIAVEWESNGQPENNYKTTPPVLDSDGSFFL
YSKLTVDKSRWQQGNVFSCSVMHEALHNHYTQKSLSLSPGK
SEQ ID NO: 6:
ACAGTGGAAAAGTCCGTGGTGTTCAAGGCCGAGGGCGAGCACTTCACCGACCAGAAAGGCAA
TACCATCGTCGGCTCTGGCAGCGGCGGCACCACCAAGTACTTTAGAATCCCCGCCATGTGCA
CCACCAGCAAGGGCACCATTGTGGTGTTCGCCGACGCCAGACACAACACCGCCAGCGATCAG
AGCTTCATCGATACCGCTGCCGCCAGATCTACCGATGGCGGCAAGACCTGGAACAAGAAGAT
CGCCATCTACAACGACCGCGTGAACAGCAAGCTGAGCAGAGTGATGGACCCTACCTGCATCG
TGGCCAACATCCAGGGCAGAGAAACCATCCTGGTCATGGTCGGAAAGTGGAACAACAACGAT
AAGACCTGGGGCGCCTACAGAGACAAGGCCCCTGATACCGATTGGGACCTCGTGCTGTACAA
GAGCACCGATGACGGCGTGACCTTCAGCAAGGTGGAAACAAACATCCACGACATCGTGACCA
AGAACGGCACCATCTCTGCCATGCTCGGCGGCGTTGGATCTGGCCTGCAACTGAATGATGGC
AAGCTGGTGTTCCCCGTGCAGATGGTCCGAACAAAGAATATCACCACCGTGCTGAATACCAG
CTTCATCTACAGCACCGACGGCATCACATGGTCCCTGCCTAGCGGCTACTGTGAAGGCTTTG
GCAGCGAGAACAACATCATCGAGTTCAACGCCAGCCTGGTCAACAACATCCGGAACAGCGGC
CTGCGGAGAAGCTTCGAGACAAAGGACTTCGGAAAGACGTGGACCGAGTTTCCTCCAATGGA
CAAGAAGGTGGACAACCGGAACCACGGCGTGCAGGGCAGCACAATCACAATCCCTAGCGGCA
ACAAACTGGTGGCCGCTCACTCTAGCGCCCAGAACAAGAACAACGACTACACCAGAAGCGAC
ATCAGCCTGTACGCCCACAACCTGTACAGCGGCGAAGTGAAGCTGATCGACGACTTCTACCC
CAAAGTGGGCAATGCCAGCGGAGCCGGCTACAGCTGTCTGAGCTACCGGAAAAATGTGGACA
AAGAAACCCTGTACGTGGTGTACGAGGCCAACGGCAGCATCGAGTTTCAGGACCTGAGCAGA
CATCTGCCCGTGATCAAGAGCTACAAC
SEQ ID NO: 7:
ENDFGLVQPLVTMEQLLWVSGRQIGSVDTFRIPLITATPRGTLLAFAEARKMSSSDEGAKFI
ALRRSMDQGSTWSPTAFIVNDGDVPDGLNLGAVVSDVETGVVFLFYSLCAHKAGCQVASTML
VWSKDDGVSWSTPRNLSLDIGTEVFAPGPGSGIQKQREPRKGRLIVCGHGTLERDGVFCLLS
DDHGASWRYGSGVSGIPYGQPKQENDFNPDECQPYELPDGSVVINARNQNNYHCHCRIVLRS
YDACDTLRPRDVTFDPELVDPVVAAGAVVTSSGIVFFSNPAHPEFRVNLTLRWSFSNGTSWR
KETVQLWPGPSGYSSLATLEGSMDGEEQAPQLYVLYEKGRNHYTESISVAKISV
SEQ ID NO: 8:
MEEVTTCSFNSPLFRQEDDRGITYRIPALLYIPPTHTFLAFAEKRSTRRDEDALHLVLRRGL
RIGQLVQWGPLKPLMEATLPGHRTMNPCPVWEQKSGCVFLFFICVRGHVTERQQIVSGRNAA
RLCFIYSQDAGCSWSEVRDLTEEVIGSELKHWATFAVGPGHGIQLQSGRLVIPAYTYYIPSW
FFCFQLPCKTRPHSLMIYSDDLGVTWHHGRLIRPMVTVECEVAEVTGRAGHPVLYCSARTPN
RCRAEALSTDHGEGFQRLALSRQLCEPPHGCQGSVVSFRPLEIPHRCQDSSSKDAPTIQQSS
PGSSLRLEEEAGTPSESWLLYSHPTSRKQRVDLGIYLNQTPLEAACWSRPWILHCGPCGYSD
LAALEEEGLFGCLFECGTKQECEQIAFRLFTHREILSHLQGDCTSPGRNPSQFKSN
SEQ ID NO: 9:
MRPADLPPRPMEESPASSSAPTETEEPGSSAEVMEEVTTCSFNSPLFRQEDDRGITYRIPAL
LYIPPTHTFLAFAEKRSTRRDEDALHLVLRRGLRIGQLVQWGPLKPLMEATLPGHRTMNPCP
VWEQKSGCVFLFFICVRGHVTERQQIVSGRNAARLCFIYSQDAGCSWSEVRDLTEEVIGSEL
KHWATFAVGPGHGIQLQSGRLVIPAYTYYIPSWFFCFQLPCKTRPHSLMIYSDDLGVTWHHG
RLIRPMVTVECEVAEVTGRAGHPVLYCSARTPNRCRAEALSTDHGEGFQRLALSRQLCEPPH
GCQGSVVSFRPLEIPHRCQDSSSKDAPTIQQSSPGSSLRLEEEAGTPSESWLLYSHPTSRKQ
RVDLGIYLNQTPLEAACWSRPWILHCGPCGYSDLAALEEEGLFGCLFECGTKQECEQIAFRL
FTHREILSHLQGDCTSPGRNPSQFKSN
SEQ ID NO: 10:
MGVPRTPSRTVLFERERTGLTYRVPSLLPVPPGPTLLAFVEQRLSPDDSHAHRLVLRRGTLA
GGSVRWGALHVLGTAALAEHRSMNPCPVHDAGTGTVFLFFIAVLGHTPEAVQIATGRNAARL
CCVASRDAGLSWGSARDLTEEAIGGAVQDWATFAVGPGHGVQLPSGRLLVPAYTYRVDRREC
FGKICRTSPHSFAFYSDDHGRTWRCGGLVPNLRSGECQLAAVDGGQAGSFLYCNARSPLGSR
VQALSTDEGTSFLPAERVASLPETAWGCQGSIVGFPAPAPNRPRDDSWSVGPGSPLQPPLLG
PGVHEPPEEAAVDPRGGQVPGGPFSRLQPRGDGPRQPGPRPGVSGDVGSWTLALPMPFAAPP
QSPTWLLYSHPVGRRARLHMGIRLSQSPLDPRSWTEPWVIYEGPSGYSDLASIGPAPEGGLV
FACLYESGARTSYDEISFCTFSLREVLENVPASPKPPNLGDKPRGCCWPS
SEQ ID NO: 11:
MMSSAAFPRWLSMGVPRTPSRTVLFERERTGLTYRVPSLLPVPPGPTLLAFVEORLSPDDSH
AHRLVLRRGTLAGGSVRWGALHVLGTAALAEHRSMNPCPVHDAGTGTVFLFFIAVLGHTPEA
VQIATGRNAARLCCVASRDAGLSWGSARDLTEEAIGGAVQDWATFAVGPGHGVQLPSGRLLV
PAYTYRVDRRECFGKICRTSPHSFAFYSDDHGRTWRCGGLVPNLRSGECQLAAVDGGQAGSF
LYCNARSPLGSRVQALSTDEGTSFLPAERVASLPETAWGCQGSIVGFPAPAPNRPRDDSWSV
GPGSPLQPPLLGPGVHEPPEEAAVDPRGGQVPGGPFSRLQPRGDGPRQPGPRPGVSGDVGSW
TLALPMPFAAPPQSPTWLLYSHPVGRRARLHMGIRLSQSPLDPRSWTEPWVIYEGPSGYSDL
ASIGPAPEGGLVFACLYESGARTSYDEISFCTFSLREVLENVPASPKPPNLGDKPRGCCWPS
SEQ ID NO: 12:
MASLP
SEQ ID NO: 13:
ASLP
SEQ ID NO: 14:
TVEKSWF
SEQ ID NO: 15:
GDYDAPTHQVQW
SEQ ID NO: 16:
SMDQGSTW
SEQ ID NO: 17:
STDGGKTW
SEQ ID NO: 18:
PRPPAPEA
SEQ ID NO: 19:
QTPLEAAC
SEQ ID NO: 20:
NPRPPAPEA
SEQ ID NO: 21:
SQNDGES
SEQ ID NO: 22:
LSHSLST
SEQ ID NO: 23:
GAGAACGACTTTGGACTGGTGCAGCCTCTGGTCACCATGGAACAGCTGCTGTGGGTTTCCGG
CAGACAGATCGGCAGCGTGGACACCTTCAGAATCCCTCTGATCACCGCCACACCTAGAGGCA
CCCTGCTGGCCTTTGCCGAGGCCAGAAAGATGAGCAGCTCTGACGAGGGCGCCAAGTTTATT
GCCCTGAGGCGGTCTATGGACCAGGGCTCTACATGGTCCCCTACCGCCTTCATCGTGAACGA
TGGCGACGTGCCCGATGGCCTGAATCTGGGAGCTGTGGTGTCCGATGTGGAAACCGGCGTGG
TGTTCCTGTTCTACAGCCTGTGTGCCCACAAGGCCGGTTGTCAGGTGGCCAGCACAATGCTC
GTGTGGTCCAAGGACGACGGCGTGTCCTGGTCTACCCCTAGAAACCTGAGCCTGGACATCGG
CACCGAAGTGTTTGCTCCAGGACCTGGCTCTGGCATCCAGAAGCAGAGAGAGCCCAGAAAGG
GCAGACTGATCGTGTGTGGCCACGGCACCCTTGAGAGAGATGGCGTTTTCTGCCTGCTGAGC
GACGATCATGGCGCCTCTTGGAGATACGGCAGCGGAGTGTCTGGAATCCCTTACGGCCAGCC
TAAGCAAGAGAACGATTTCAACCCCGACGAGTGCCAGCCTTACGAGCTGCCTGATGGCAGCG
TCGTGATCAACGCCCGGAACCAGAACAACTACCACTGCCACTGCCGGATCGTGCTGAGAAGC
TACGACGCCTGCGATACCCTGCGGCCTAGAGATGTGACCTTCGATCCTGAGCTGGTGGACCC
TGTTGTTGCCGCTGGTGCCGTCGTGACATCTAGCGGCATCGTGTTCTTCAGCAACCCTGCTC
ACCCCGAGTTCAGAGTGAATCTGACCCTGCGGTGGTCCTTCAGCAATGGCACAAGCTGGCGG
AAAGAAACCGTGCAGCTTTGGCCTGGACCTAGCGGCTACTCTTCTCTGGCTACACTGGAAGG
CAGCATGGACGGCGAAGAACAGGCCCCTCAGCTGTACGTGCTGTACGAGAAGGGCAGAAACC
ACTAGACCGAGAGCATCAGCGTGGCCAAGATCAGCGTT
SEQ ID NO: 24:
ATGGCCAGCCTGCCTGTGCTGCAGAAAGAAAGCGTGTTCCAGTCTGGCGCCCACGCCTACAG
AATTCCCGCTCTGCTGTATCTGCCAGGCCAGCAGTCTCTGCTGGCTTTCGCTGAACAGCGGG
CCAGCAAGAAGGATGAGCACGCCGAACTGATCGTGCTGCGGAGAGGCGATTACGACGCCCCT
ACACATCAGGTGCAGTGGCAGGCTCAAGAGGTGGTGGCTCAGGCTAGACTGGACGGCCACAG
ATCTATGAACCCCTGTCCTCTGTACGATGCCCAGACCGGCACACTGTTTCTGTTCTTTATCG
CTATCCCCGGCCAAGTGACCGAGCAGCAGCAGCTGCAGACAAGAGCCAACGTGACCAGACTG
TGTCAAGTGACCTCCACCGACCACGGCAGAACCTGGTCTAGCCCTAGAGATCTGACCGACGC
CGCCATCGGACCTGCCTATAGAGAGTGGTCCACCTTCGCCGTTGGACCTGGACACTGTCTCC
AGCTGCACGACAGGGCTAGATCTCTGGTGGTGCCTGCCTACGCCTATAGAAAGCTGCACCCC
ATCCAGCGGCCTATTCCTAGCGCCTTCTGCTTTCTGAGCCACGATCACGGCAGGACATGGGC
CAGAGGACATTTCGTGGCCCAGGACACACTGGAATGCCAGGTGGCCGAAGTGGAAACCGGCG
AGCAGAGAGTCGTGACCCTGAACGCCAGATCTCACCTGAGAGCCAGAGTGCAGGCCCAGAGC
ACAAACGACGGCCTGGATTTCCAAGAGAGCCAGCTGGTCAAGAAACTGGTGGAACCTCCTCC
ACAGGGCTGTCAGGGAAGCGTGATCAGCTTTCCATCTCCTAGAAGCGGCCCTGGCTCTCCTG
CTCAGTGGCTGCTGTATACACACCCCACACACAGCTGGCAGAGAGCCGATCTGGGCGCCTAC
CTGAATCCTAGACCTCCTGCTCCTGAGGCTTGGAGCGAACCTGTTCTGCTGGCCAAGGGCAG
CTGTGCCTACAGCGATCTGCAGTCTATGGGCACAGGCCCTGATGGCAGCCCTCTGTTTGGCT
GTCTGTACGAGGCCAACGACTACGAAGAGATCGTGTTCCTGATGTTCACCCTGAAGCAGGCC
TTTCCAGCCGAGTACCTGCCTCAA
SEQ ID NO: 25:
ATGGAGGAAGTGACCACCTGTAGCTTCAACAGCCCTCTGTTCCGGCAAGAGGACGACCGGGG
CATCACCTACAGAATCCCTGCTCTGCTGTACATCCCTCCTACACACACCTTTCTGGCCTTCG
CCGAGAAGCGGAGCACCAGACGAGATGAAGATGCCCTGCACCTGGTGCTGAGAAGAGGCCTG
AGAATCGGACAGCTGGTGCAGTGGGGACCTCTGAAGCCTCTGATGGAAGCCACACTGCCCGG
CCACAGAACCATGAATCCTTGTCCTGTGTGGGAGCAGAAAAGCGGCTGCGTGTTCCTGTTCT
TCATCTGCGTGCGGGGCCACGTGACCGAGAGACAGCAAATCGTGTCCGGCAGAAACGCCGCC
AGACTGTGCTTCATCTACAGCCAGGATGCCGGCTGCTCTTGGAGCGAAGTTCGGGATCTGAC
CGAAGAAGTGATCGGCAGCGAGCTGAAGCACTGGGCCACATTTGCTGTTGGCCCTGGCCACG
GAATCCAGCTGCAATCTGGCAGACTGGTCATCCCCGCCTACACCTACTATATCCCCAGCTGG
TTCTTCTGCTTCCAACTGCCTTGCAAGACCCGGCCTCACAGCCTGATGATCTACAGCGACGA
TCTGGGCGTGACATGGCACCACGGCAGACTGATCAGACCCATGGTCACCGTGGAATGCGAGG
TGGCCGAAGTGACAGGCAGAGCTGGACACCCTGTGCTGTACTGCTCTGCCAGAACACCCAAC
CGGTGTAGAGCCGAGGCTCTGTCTACAGATCACGGCGAGGGCTTTCAGAGACTGGCCCTCTC
TAGACAGCTGTGCGAACCTCCTCATGGCTGTCAGGGCAGCGTGGTGTCCTTCAGACCTCTGG
AAATCCCTCACCGGTGCCAGGACAGCAGCTCTAAGGATGCCCCTACCATCCAGCAGTCTAGC
CCTGGCAGCAGCCTGAGACTGGAAGAGGAAGCCGGAACACCTAGCGAGAGCTGGCTGCTGTA
CTCTCACCCCACCAGCAGAAAGCAGAGAGTGGACCTGGGCATCTACCTGAATCAGACCCCTC
TGGAAGCCGCCTGTTGGAGCAGACCTTGGATTCTGCACTGTGGCCCTTGCGGCTACTCTGAT
CTGGCCGCTCTGGAAGAAGAGGGCCTGTTCGGCTGCCTGTTTGAGTGCGGCACAAAGCAAGA
GTGCGAGCAGATCGCCTTCCGGCTGTTCACCCACAGAGAGATCCTGAGCCATCTGCAGGGCG
ACTGCACAAGCCCAGGCAGAAATCCCAGCCAGTTCAAGAGCAAC
SEQ ID NO: 26:
ATGGGCGTGCCCAGAACACCCAGCAGAACCGTGCTGTTCGAGAGAGAGAGGACCGGCCTGAC
CTACAGAGTGCCTTCTCTGCTGCCTGTGCCTCCTGGACCTACACTGCTGGCCTTCGTGGAAC
AGAGACTGAGCCCCGATGATTCTCACGCCCACAGACTGGTGCTGAGAAGAGGAACACTGGCT
GGCGGCTCTGTTAGATGGGGAGCACTGCATGTGCTGGGCACAGCTGCTCTTGCCGAGCACAG
ATCCATGAATCCCTGTCCTGTGCACGACGCCGGAACCGGCACAGTGTTTCTGTTCTTTATCG
CCGTGCTGGGCCACACACCTGAGGCCGTTCAAATTGCCACCGGCAGAAATGCCGCCAGACTG
TGTTGTGTGGCCTCCAGAGATGCCGGCCTGTCTTGGGGATCTGCCAGAGATCTGACCGAGGA
AGCCATTGGCGGAGCCGTTCAGGATTGGGCCACATTTGCTGTTGGACCTGGACACGGCGTGC
AGCTGCCAAGTGGTAGACTGCTGGTGCCTGCCTACACATACAGAGTGGATCGGAGAGAGTGC
TTCGGAAAGATCTGCCGGACAAGCCCTCACAGCTTCGCCTTCTACTCCGACGATCACGGCCG
GACTTGGAGATGTGGTGGCCTGGTGCCTAATCTGAGAAGCGGCGAATGTCAACTGGCCGCCG
TTGATGGTGGACAGGCTGGCAGCTTCCTGTACTGCAACGCCAGATCTCCTCTGGGCTCTAGA
GTGCAGGCCCTGTCTACCGATGAGGGCACCAGTTTTCTGCCCGCCGAAAGAGTTGCCTCTCT
GCCTGAAACAGCCTGGGGCTGTCAGGGCTCTATCGTGGGATTTCCTGCTCCTGCTCCAAACA
GACCCCGGGACGATTCTTGGAGTGTCGGCCCTGGATCTCCACTGCAGCCTCCATTGCTTGGA
CCAGGCGTTCACGAGCCACCTGAAGAGGCTGCCGTTGATCCTAGAGGCGGACAAGTTCCTGG
CGGCCCTTTTAGCAGACTGCAGCCAAGAGGCGACGGCCCTAGACAACCTGGACCAAGACCTG
GCGTCAGCGGAGATGTTGGCTCTTGGACACTGGCCCTGCCTATGCCTTTTGCCGCTCCTCCT
CAGTCTCCTACCTGGCTGCTGTACTCTCACCCTGTTGGCAGACGGGCCAGACTGCACATGGG
CATCAGACTGTCTCAGAGCCCTCTGGACCCCAGAAGCTGGACAGAGCCTTGGGTCATCTATG
AGGGCCCTAGCGGCTACAGCGATCTGGCCTCTATTGGCCCAGCTCCTGAAGGCGGACTGGTG
TTCGCTTGTCTGTATGAGAGCGGCGCCAGAACCAGCTACGACGAGATCAGCTTCTGCACCTT
CAGCCTGCGCGAGGTGCTGGAAAATGTGCCCGCCTCTCCTAAGCCTCCTAACCTGGGCGATA
AGCCTAGAGGCTGTTGCTGGCCATCT
SEQ ID NO: 27:
MTGERPSTALPDRRWGPRILGFWGGCRVWVFAAIFLLLSLAASWSKA
SEQ ID NO: 28:
MDMRVPAQLLGLLLLWLPGARC
SEQ ID NO: 29:
YGTL
SEQ ID NO: 30:
MTVEKSVVFKAEGEHFTDQKGNTIVGSGSGGTTKYFRIPAMCTTSKGTIVVFADARHNTASD
QSFIDTAAARSTDGGKTWNKKIAIYNDRVNSKLSRVMDPTCIVANIQGRETILVMVGKWNNN
DKTWGAYRDKAPDTDWDLVLYKSTDDGVTFSKVETNIHDIVTKNGTISAMLGGVGSGLQLND
GKLVFPVQMVRTKNITTVLNTSFIYSTDGITWSLPSGYCEGFGSENNIIEFNASLVNNIRNS
GLRRSFETKDFGKTWTEFPPMDKKVDNRNHGVQGSTITIPSGNKLVAAHSSAQNKNNDYTRS
DISLYAHNLYSGEVKLIDDFYPKVGNASGAGYSCLSYRKNVDKETLYVVYEANGSIEFQDLS
RHLPVIKSYN
SEQ ID NO: 31:
EPKSCDKTHTCPPCPAPELLGGPSVFLFPPKPKDTLMISRTPEVTCVVVDVSHEDPEVKFNW
YVDGVEVHNAKTKPREEQYNSTYRVVSVLTVLHQDWLNGKEYKCKVSNKALPAPIEKTISKA
KGQPREPQVYTLPPSREEMTKNQVSLTCLVKGFYPSDIAVEWESNGQPENNYKTTPPVLDSD
GSFFLYSKLTVDKSRWQQGNVFSCSVMHEALHNHYTQKSLSLSPGK
SEQ ID NO: 32:
DKTHTCPPCPAPELLGGPSVFLFPPKPKDTLMISRTPEVTCVVVDVSHEDPEVKFNWYVDGV
EVHNAKTKPREEQYNSTYRVVSVLTVLHQDWLNGKEYKCKVSNKALPAPIEKTISKAKGQPR
EPQVYTLPPSREEMTKNQVSLTCLVKGFYPSDIAVEWESNGQPENNYKTTPPVLDSDGSFFL
TSKLTVDKSRWQQGNVFSCSVMHEALHNHYTQKSLSLSPGK
SEQ ID NO: 33:
EPKSCDKTHTCPPCPAPELLGGPSVFLFPPKPKDTLMISRTPEVTCVVVDVSHEDPEVKFNW
YVDGVEVHNAKTKPREEQYNSTYRVVSVLTVLHQDWLNGKEYKCKVSNKALPAPIEKTISKA
KGQPREPQVYTLPPSREEMTKNQVSLYCLVKGFYPSDIAVEWESNGQPENNYKTTPPVLDSD
GSFFLYSKLTVDKSRWQQGNVFSCSVMHEALHNHYTQKSLSLSPGK
SEQ ID NO: 34:
ATGAGACCTGCGGACCTGCCCCCGCGCCCCATGGAAGAATCCCCGGCGTCCAGCTCTGCCCC
GACAGAGACGGAGGAGCCGGGGTCCAGTGCAGAGGTCATGGAAGAAGTGACAACATGCTCCT
TCAACAGCCCTCTGTTCCGGCAGGAAGATGACAGAGGGATTACCTACCGGATCCCAGCCCTG
CTCTACATACCCCCCACCCACACCTTCCTGGCCTTTGCAGAGAAGCGTTCTACGAGGAGAGA
TGAGGATGCTCTCCACCTGGTGCTGAGGCGAGGGTTGAGGATTGGGCAGTTGGTACAGTGGG
GGCCCCTGAAGCCACTGATGGAAGCCACACTACCGGGGCATCGGACCATGAACCCCTGTCCT
GTATGGGAGCAGAAGAGTGGTTGTGTGTTCCTGTTCTTCATCTGTGTGCGGGGCCATGTCAC
AGAGCGTCAACAGATTGTGTCAGGCAGGAATGCTGCCCGCCTTTGCTTCATCTACAGTCAGG
ATGCTGGATGTTCATGGAGTGAGGTGAGGGACTTGACTGAGGAGGTCATTGGCTCAGAGCTG
AAGCACTGGGCCACATTTGCTGTGGGCCCAGGTCATGGCATCCAGCTGCAGTCAGGGAGACT
GGTCATCCCTGCGTATACCTACTACATCCCTTCCTGGTTCTTTTGCTTCCAGCTACCATGTA
AAACCAGGCCTCATTCTCTGATGATCTACAGTGATGACCTAGGGGTCACATGGCACCATGGT
AGACTCATTAGGCCCATGGTTACAGTAGAATGTGAAGTGGCAGAGGTGACTGGGAGGGCTGG
CCACCCTGTGCTATATTGCAGTGCCCGGACACCAAACAGGTGCCGGGCAGAGGCGCTCAGCA
CTGACCATGGTGAAGGCTTTCAGAGACTGGCCCTGAGTCGACAGCTCTGTGAGCCCCCACAT
GGTTGCCAAGGGAGTGTGGTAAGTTTCCGGCCCCTGGAGATCCCACATAGGTGCCAGGACTC
TAGCAGCAAAGATGCACCCACCATTCAGCAGAGCTCTCCAGGCAGTTCACTGAGGCTGGAGG
AGGAAGCTGGAACACCGTCAGAATCATGGCTCTTGTACTCACACCCAACCAGTAGGAAACAG
AGGGTTGACCTAGGTATCTATCTCAACCAGACCCCCTTGGAGGCTGCCTGCTGGTCCCGCCC
CTGGATCTTGCACTGTGGGCCCTGTGGCTACTCTGATCTGGCTGCTCTGGAGGAGGAGGGCT
TGTTTGGGTGTTTGTTTGAATGTGGGACCAAGCAAGAGTGTGAGCAGATTGCCTTCCGCCTG
TTTACACACCGGGAGATCCTGAGTCACCTGCAGGGGGACTGCACCAGCCCTGGTAGGAACCC
AAGCCAATTCAAAAGCAAT
SEQ ID NO: 35:
ATGATGAGCTCTGCAGCCTTCCCAAGGTGGCTGAGCATGGGGGTCCCTCGTACCCCTTCACG
GACAGTGCTCTTCGAGCGGGAGAGGACGGGCCTGACCTACCGCGTGCCCTCGCTGCTCCCCG
TGCCCCCCGGGCCCACCCTGCTGGCCTTTGTGGAGCAGCGGCTCAGCCCTGACGACTCCCAC
GCCCACCGCCTGGTGCTGAGGAGGGGCACGCTGGCCGGGGGCTCCGTGCGGTGGGGTGCCCT
GCACGTGCTGGGGACAGCAGCCCTGGCGGAGCACCGGTCCATGAACCCCTGCCCTGTGCACG
ATGCTGGCACGGGCACCGTCTTCCTCTTCTTCATCGCGGTGCTGGGCCACACGCCTGAGGCC
GTGCAGATCGCCACGGGAAGGAACGCCGCGCGCCTCTGCTGTGTGGCCAGCCGTGACGCCGG
CCTCTCGTGGGGCAGCGCCCGGGACCTCACCGAGGAGGCCATCGGTGGTGCCGTGCAGGACT
GGGCCACATTCGCTGTGGGTCCCGGCCACGGTGTGCAGCTGCCCTCAGGCCGCCTGCTGGTA
CCCGCCTACACCTACCGCGTGGACCGCCGAGAGTGTTTTGGCAAGATCTGCCGGACCAGCCC
TCACTCCTTCGCCTTCTACAGCGATGACCACGGCCGCACCTGGCGCTGTGGAGGCCTCGTGC
CCAACCTGCGCTCAGGCGAGTGCCAGCTGGCAGCGGTGGACGGTGGGCAGGCCGGCAGCTTC
CTCTACTGCAATGCCCGGAGCCCACTGGGCAGCCGTGTGCAGGCGCTCAGCACTGACGAGGG
CACCTCCTTCCTGCCCGCAGAGCGCGTGGCTTCCCTGCCCGAGACTGCCTGGGGCTGCCAGG
GCAGCATCGTGGGCTTCCCAGCCCCCGCCCCCAACAGGCCACGGGATGACAGTTGGTCAGTG
GGCCCCGGGAGTCCCCTCCAGCCTCCACTCCTCGGTCCTGGAGTCCACGAACCCCCAGAGGA
GGCTGCTGTAGACCCCCGTGGAGGCCAGGTGCCTGGTGGGCCCTTCAGCCGTCTGCAGCCTC
GGGGGGATGGCCCCAGGCAGCCTGGCCCCAGGCCTGGGGTCAGTGGGGATGTGGGGTCCTGG
ACCCTGGCACTCCCCATGCCCTTTGCTGCCCCGCCCCAGAGCCCCACGTGGCTGCTGTACTC
CCACCCAGTGGGGCGCAGGGCTCGGCTACACATGGGTATCCGCCTGAGCCAGTCCCCGCTGG
ACCCGCGCAGCTGGACAGAGCCCTGGGTGATCTACGAGGGCCCCAGCGGCTACTCCGACCTG
GCGTCCATCGGGCCGGCCCCTGAGGGGGGCCTGGTTTTTGCCTGCCTGTACGAGAGCGGGGC
CAGGACCTCCTATGATGAGATTTCCTTTTGTACATTCTCCCTGCGTGAGGTCCTGGAGAACG
TGCCCGCCAGCCCCAAACCGCCCAACCTTGGGGACAAGCCTCGGGGGTGCTGCTGGCCCTCC
SEQ ID NO: 36:
MRFKNVKKTALMLAMFGMATSSNAALFDYNATGDTEFDSPAKQGWMQDNTNNGSGVLTNADG
MPAWLVQGIGGRAQWTYSLSTNQHAQASSFGWRMTTEMKVLSGGMITNYYANGTQRVLPIIS
LDSSGNLVVEFEGQTGRTVLATGTAATEYHKFELVELPGSNPSASFYFDGKLIRDNIQPTAS
KQNMIVWGNGSSNTDGVAAYRDIKFEIQGDVIFRGPDRIPSIVASSVTPGVVTAFAEKRVGG
GDPGALSNTNDIITRTSRDGGITWDTELNLTEQINVSDEFDFSDPRPIYDPSSNTVLVSYAR
WPTDAAQNGDRIKPWMPNGIFYSVYDVASGNWQAPIDVTDQVKERSFQIAGWGGSELYRRNT
SLNSQQDWQSNAKIRIVDGAANQIQVADGSRKYVVTLSIDESGGLVANLNGVSAPIILQSEH
AKVHSFHDYELQYSALNHTTTLFVDGQQITTWAGEVSQENNIQFGNADAQIDGRLHVQKIVL
TQQGHNLVEFDAFYLAQQTPEVEKDLEKLGWTKIKTGNTMSLYGNASVNPGPGHGITLTRQQ
NISGSQNGRLIYPAIVLDRFFLNVMSIYSDDGGSNWQTGSTLPIPFRWKSSSILETLEPSEA
DMVELQNGDLLLTARLDFNQIVNGVNYSPRQQFLSKDGGITWSLLEANNANVFSNISTGTVD
ASITRFEQSDGSHFLLFTNPQGNPAGTNGRQNLGLWFSFDEGVTWKGPIQLVNGASAYSDIY
QLDSENAIVIVETDNSNMRILRMPITLLKQKLTLSQN
SEQ ID NO: 37:
TTGTCAATCAAGATGAGTTCACAACGAAGAAGAGCATCGATTCACAAGGAAACAGATTCTAA
TATAAAGGGAGTAGATATGCGTTTCAAAAACGTAAAGAAAACCGCTTTAATGCTTGCAATGT
TCGGTATGGCGACAAGCTCAAACGCCGCACTTTTTGACTATAACGCAACGGGTGACACTGAG
TTTGACAGTCCAGCCAAACAGGGATGGATGCAAGACAACACGAATAATGGCAGCGGCGTTTT
AACCAATGCAGATGGAATGCCCGCTTGGTTGGTGCAAGGTATTGGAGGGAGAGCTCAATGGA
CATATTCTCTCTCTACTAATCAACATGCCCAAGCATCAAGTTTCGGTTGGCGAATGACGACA
GAAATGAAAGTGCTCAGTGGTGGAATGATCACAAACTACTACGCCAACGGCACTCAGCGTGT
CTTACCCATCATTTCATTAGATAGCAGTGGTAACTTAGTTGTTGAGTTTGAAGGGCAAACTG
GACGCACCGTTTTGGCAACCGGCACAGCAGCAACGGAATATCATAAATTTGAATTGGTATTC
CTTCCTGGAAGTAACCCATCCGCTAGCTTTTACTTCGATGGCAAACTCATTCGTGACAACAT
CCAGCCGACTGCATCAAAACAAAATATGATCGTATGGGGGAATGGCTCATCAAATACGGATG
GTGTCGCCGCTTATCGTGATATTAAGTTTGAAATTCAAGGCGACGTCATCTTCAGAGGCCCA
GACCGTATACCGTCCATTGTAGCAAGTAGCGTAACACCAGGGGTGGTAACCGCATTTGCAGA
GAAACGTGTGGGGGGAGGAGATCCCGGTGCTCTGAGTAATACCAATGACATAATCACTCGTA
CCTCACGAGATGGCGGTATAACTTGGGATACCGAGCTCAACCTCACTGAGCAAATCAATGTC
AGTGATGAGTTTGATTTCTCCGATCCTCGGCCTATCTATGATCCTTCCTCCAATACGGTTCT
TGTCTCTTATGCTCGATGGCCGACCGATGCCGCTCAAAACGGAGATCGAATAAAACCATGGA
TGCCAAACGGTATTTTTTACAGCGTCTATGATGTTGCATCAGGGAACTGGCAAGCGCCTATC
GATGTTACCGATCAGGTGAAAGAACGCAGTTTCCAAATCGCTGGTTGGGGTGGTTCAGAGCT
GTATCGCCGAAATACCAGCCTAAATAGCCAGCAAGACTGGCAATCAAACGCTAAGATCCGAA
TTGTTGATGGTGCAGCGAACCAGATACAAGTTGCCGATGGTAGCCGAAAATATGTTGTCACA
CTGAGTATTGATGAATCAGGTGGTCTAGTCGCTAATCTAAACGGTGTTAGTGCTCCGATTAT
CCTGCAATCTGAACACGCAAAGGTACACTCTTTCCATGACTACGAACTTCAATATTCGGCGT
TAAACCACACCACAACGTTATTCGTGGATGGTCAGCAAATCACAACTTGGGCTGGCGAAGTA
TCGCAGGAGAACAACATTCAGTTTGGTAATGCGGATGCCCAAATTGACGGCAGACTGCATGT
GCAAAAAATTGTTCTCACACAGCAAGGCCATAACCTCGTGGAGTTTGATGCTTTCTATTTAG
CACAGCAAACCCCTGAAGTAGAGAAAGACCTTGAAAAGCTTGGTTGGACAAAAATTAAAACG
GGCAACACCATGAGTTTGTATGGAAATGCCAGTGTCAACCCAGGACCGGGTCATGGCATCAC
CCTTACTCGACAACAAAATATCAGTGGCAGCCAAAACGGCCGCTTGATCTACCCAGCGATTG
TGCTTGATCGTTTCTTCTTGAACGTCATGTCTATTTACAGTGATGATGGCGGTTCAAACTGG
CAAACCGGTTCAACACTCCCTATCCCCTTTCGCTGGAAGAGTTCGAGTATCCTAGAAACTCT
CGAACCTAGTGAAGCTGATATGGTTGAACTCCAAAACGGTGATCTACTCCTTACTGCACGCC
TTGATTTTAACCAAATCGTTAATGGTGTGAACTATAGCCCACGCCAGCAATTTTTGAGTAAA
GATGGTGGAATCACGTGGAGCCTACTTGAGGCTAACAACGCTAACGTCTTTAGCAATATCAG
TACTGGTACCGTTGATGCTTCTATTACTCGGTTCGAGCAAAGTGACGGTAGCCATTTCTTAC
TCTTTACTAACCCACAAGGAAACCCTGCGGGGACAAATGGCAGGCAAAATCTAGGCTTATGG
TTTAGCTTCGATGAAGGGGTGACATGGAAAGGACCAATTCAACTTGTTAATGGTGCATCGGC
ATATTCTGATATTTATCAATTGGATTCGGAAAATGCGATTGTCATTGTTGAAACGGATAATT
CAAATATGCGAATTCTTCGTATGCCTATCACATTGCTAAAACAGAAGCTGACCTTATCGCAA
AACTAA
SEQ ID NO: 38:
MVGADPTRPRGPLSYWAGRRGQGLAAIFLLLVSAAESEARAEDDFSLVQPLVTMEQLLWVSG
KQIGSVDTFRIPLITATPRGTLLAFAEARKKSASDEGAKFIAMRRSTDQGSTWSSTAFIVDD
GEASDGLNLGAVVNDVDTGIVFLIYTLCAHKVNCQVASTMLVWSKDDGISWSPPRNLSVDIG
TEMFAPGPGSGIQKQREPGKGRLIVCGHGTLERDGVFCLLSDDHGASWHYGTGVSGIPFGQP
KHDHDFNPDECQPYELPDGSVIINARNQNNYHCRCRIVLRSYDACDTLRPRDVTFDPELVDP
VVAAGALATSSGIVFFSNPAHPEFRVNLTLRWSFSNGTSWLKERVQVWPGPSGYSSLTALEN
STDGKKQPPQLFVLYEKGLNRYTESISMVKISVYGTL
SEQ ID NO: 39:
MTVQPSPWFSDLRPMATCPVLQKETLFRTGVHAYRIPALLYLKKQKTLLAFAEKRASKTDEH
AELIVLRRGSYNEATNRVKWQPEEVVTQAQLEGHRSMNPCPLYDKQTKTLFLFFIAVPGRVS
EHHQLHTKVNVTRLCCVSSTDHGRTWSPIQDLTETTIGSTHQEWATFAVGPGHCLQLRNPAG
SLLVPAYAYRKLHPAQKPTPFAFCFISLDHGHTWKLGNFVAENSLECQVAEVGTGAQRMVYL
NARSFLGARVQAQSPNDGLDFQDNRVVSKLVEPPHGCHGSVVAFHNPISKPHALDTWLLYTH
PTDSRNRTNLGVYLNQMPLDPTAWSEPTLLAMGICAYSDLQNMGQGPDGSPQFGCLYESGNY
EEIIELIFTLKQAFPTVFDAQ
SEQ ID NO: 40:
MEEVPPYSLSSTLFQQEEQSGVTYRIPALLYLPPTHTFLAFAEKRTSVRDEDAACLVLRRGL
MKGRSVQWGPQRLLMEATLPGHRTMNPCPVWEKNTGRVYLFFICVRGHVTERCQIVWGKNAA
RLCFLCSEDAGCSWGEVKDLTEEVIGSEVKRWATFAVGPGHGIQLHSGRLIIPAYAYYVSRW
FLCFACSVKPHSLMIYSDDFGVTWHHGKFIEPQVTGECQVAEVAGTAGNPVLYCSARTPSRF
RAEAFSTDSGGCFQKPTLNPQLHEPRTGCQGSVVSFRPLKMPNTYQDSIGKGAPATQKCPLL
DSPLEVEKGAETPSATWLLYSHPTSKRKRINLGIYYNRNPLEVNCWSRPWILNRGPSGYSDL
AVVEEQDLVACLFECGEKNEYERIDFCLFSDHEVLSCEDCTSPSSD
SEQ ID NO: 41:
METAGAPFCFHVDSLVPCSYWKVMGPTRVPRRTVLFQRERTGLTYRVPALLCVPPRPTLLAF
AEQRLSPDDSHAHRLVLRRGTLTRGSVRWGTLSVLETAVLEEHRSMNPCPVLDEHSGTIFLF
FIAVLGHTPEAVQIATGKNAARLCCVTSCDAGLTWGSVRDLTEEAIGAALQDWATFAVGPGH
GVQLRSGRLLVPAYTYHVDRRECFGKICWTSPHSLAFYSDDHGISWHCGGLVPNLRSGECQL
AAVDGDFLYCNARSPLGNRVQALSADEGTSFLPGELVPTLAETARGCQGSIVGFLAPPSIEP
QDDRWTGSPRNTPHSPCFNLRVQESSGEGARGLLERWMPRLPLCYPQSRSPENHGLEPGSDG
DKTSWTPECPMSSDSMLQSPTWLLYSHPAGRRARLHMGIYLSRSPLDPHSWTEPWVIYEGPS
GYSDLAFLGPMPGASLVFACLFESGTRTSYEDISFCLFSLADVLENVPTGLEMLSLRDKAQG
HCWPS
SEQ ID NO: 42:
GGGTCACATGCTGATGGACTAATTGGAGTCGCGGCAGCGCGGGCTGCGGCCCCCAAGGGGAG
GGGTCGGAGTGACGTGCGCGCTTTTAAAGGGCCGAGGTCAGCTGACGGCTTGCCACCGGTGA
CCAGTTCCTGGACAGGGATCGCCGGGAGCTATGGTGGGGGCAGACCCGACCAGACCCCGGGG
ACCGCTGAGCTATTGGGCGGGCCGTCGGGGTCAGGGGCTCGCAGCGATCTTCCTGCTCCTGG
TGTCCGCGGCGGAATCCGAGGCCAGGGCAGAGGATGACTTCAGCCTGGTGCAGCCGCTGGTG
ACCATGGAGCAGCTGCTGTGGGTGAGCGGGAAGCAGATCGGCTCTGTAGACACTTTCCGCAT
CCCGCTCATCACAGCCACCCCTCGGGGCACGCTCCTGGCCTTCGCTGAGGCCAGGAAAAAAT
CTGCATCCGATGAGGGGGCCAAGTTCATCGCCATGAGGAGGTCCACGGACCAGGGTAGCACG
TGGTCCTCTACAGCCTTCATCGTAGACGATGGGGAGGCCTCCGATGGCCTGAACCTGGGCGC
TGTGGTGAACGATGTAGACACAGGGATAGTGTTCCTTATCTATACCCTCTGTGCTCACAAGG
TCAACTGCCAGGTGGCCTCTACCATGTTGGTTTGGAGTAAGGACGACGGCATTTCCTGGAGC
CCACCCCGGAATCTCTCTGTGGATATTGGCACAGAGATGTTTGCCCCTGGACCTGGCTCAGG
CATTCAGAAACAGCGGGAGCCTGGGAAGGGCCGGCTCATTGTGTGTGGACACGGGACGCTGG
AGCGAGATGGGGTCTTCTGTCTCCTCAGTGATGACCACGGTGCCTCCTGGCACTACGGCACT
GGAGTGAGCGGCATTCCCTTTGGCCAGCCCAAACACGATCACGATTTCAACCCCGACGAGTG
CCAGCCCTACGAGCTTCCAGATGGCTCGGTCATCATCAACGCCCGGAACCAGAATAACTACC
ATTGCCGCTGCAGGATCGTCCTCCGCAGCTATGACGCCTGTGACACCCTCAGGCCCCGGGAT
GTGACCTTCGACCCTGAGCTCGTGGACCCTGTGGTAGCTGCAGGAGCACTAGCCACCAGCTC
CGGCATTGTCTTCTTCTCCAATCCAGCCCACCCTGAGTTCCGAGTGAACCTGACCCTGCGCT
GGAGTTTCAGCAATGGTACATCCTGGCAGAAGGAGAGGGTCCAGGTGTGGCCGGGACCCAGC
GGCTACTCGTCCCTGACAGCCCTGGAAAACAGCACGGATGGAAAGAAGCAGCCCCCGCAGCT
GTTCGTTCTGTACGAGAAAGGCCTGAACCGGTACACCGAGAGCATCTCCATGGTCAAAATCA
GCGTCTACGGCACGCTCTGAGCCCCGTGCCCAAAGGACACCAAGTCCTGGTCGCTGACTTCA
CAGCTCTCTGGACCATCTGCAGAGGGTGCCTGAAACACAGCTCTTCCTCTGAACTCTGACCT
TTTGCAACTTCTCATCAACAGGGAAGTCTCTTCGTTATGACTTAACACCCAGCTTCCTCTCG
GGGCAGGAAGTCCCTCCGTCACCAAGAGCACTTTTTTCCAGTATGCTGGGGATGGCCCCTGT
CCATTCTCTTCCAGGACAACGGAGCTGTGCCTTTCTGGGACAGGATGGGGGAGGGGCTCCCC
CTGGAGAGATGAACAGATACGAACTCAGGGAACTGAGAAGGCCCGGTGTCCTAGGGTACAAA
GGCAGGTACTAGATGTGATTGCTGAAAGTCCCCAGGGCAGAGTGTCCTTTCAGAGCAAGGAT
AAGCACACCTACGTGTGCACCTTTGATTATTTATGAATCGAAATATTTGTAACTTAAAATTT
TTGATGCAGAAAAAGCGTTTGTGGAGTCTGTGGTTCTGTCTGCTCACGCCTTCCCAATTGCC
TCCTGGAGAGACAGGAAGGCAGCTGGAAGAGGAGCCGATGTACTTACTGGGAAGCAGAAACC
CCTAGATTCCATCCTGGCTGCTGCTGTTTGCAAGTGTCAAAGATGGGGGGGCGTGTTTATAT
TTTATATTTCTAAGATGGGGTGGCATAGGAAATAGGGAACAGATGTGTAAAACCAGATGGGA
AGGACAGTCTGTGAGAAAGGAGCAAGCAGTTGCTGCAGGTGTGGGAGAGCAAAGCCCTTCTC
CACGTGGAAAGAGCCCAGATGGACGCTAAGCATGTTGGGCACCTGTAACCCCGCACTCGCTG
GACTGACGGTGTAGCTCAGTGGTGGAGCTAGTACTTGGAACGCCTAAGACTCTGGGTTCAGT
CCTTGGGGGGGGGGGTATGTGTTTATTGAGAGGAAGGTGTACGTACTGTAGGTCAGAGGACA
GCTTACTGGAGTTGTCTCTCTCCTTCACGCTGTGAGTCCTGTGGAATGACCTCAGGTGTCAG
AGTTGGGGGCAGGTGCCTTTGCCAGCTGAGCCATCTTGCTGTCTCTGCTTTAATTTAAAAAA
AAAAAAAAAAAAGAATATTAAGGTCTGAGGGATTCGGGCTGCGTTCATTTCAATTAGAGGGT
CATATTTCTTTTGACATTTCTTCTCTAAGAAATGTTAAGATCATTTGTTCTGTGTGATAGAG
GTATAGCTCCATTGTATGTCAGCAGTGAGGGATCCTGTGCATTTTATCCAGAGTTTGTACGG
TGTTCTAGGGGCTGCTAGTGCAGCCCAGTGCTAAACACTTCAGCATGCACAAGGCCTCAATC
AGTGCATGCATGTGCACACACACACAGACACACACGTAGACACTGACACAGGTACACAAATA
CACACTGGCCCACATGTACACATCGACTCACAGGTAGACAGACCCACTTTGACACACATATA
CACAGACACAAACGCACTGGCACACACATATACACAGGCACACATGGATAGATGGACACACG
TGTAGACATACACACACACACAGAAATACAAATGTTCAGGTTTTCTAAAAAAAAAAAAATTA
GAGACGTGTTGACTTCATTTTTAGCAAAAATCCTGTCATGTATCTTAAAGTGGATTGAACCC
ACTATGTAGCCCAGGCTGGCCTCCAAATGGGCATCCTTCTGCCTCAGTCTCCCGAGGGCTAG
GATAACAGGAGTATGCCATCACACCTGGCTAATAGAAATTTTCAAAATTGTTTGTTTGAAGG
TGACTCTTACTATATTGCCTAACTGATCTCCAGTTCGTGAAATCCTCCTGCCTCAGAACCAG
GACTGTCAATATAACCCACCAAGACAGGCCAACATTCACAATTGATTGTTAGTTTGTGGTCT
GAATCAAGGTCTTATACTGTAGCCCAGGCTAGCCCGGAATACACGATATCTCCAGTGCTTCA
GATCCTCAGTTCTAACTAAGCATGGCCACATCCATGTTTAACTGCAAATTTGATGTTACCAT
GGTTTGGTTTGGTTTGGTTTGGTTTGGTTTGGTTTGGTTTGGTTTTTTGGCCATTTTTTTTT
TCTCATGCTGAGGCCTTGTGCTCTCAAGTTGGGGAGACAGCATGGAGGGTAGCTGCAACTGT
AACCCCAGTTCCAGGGGACCTGACACCCTCTGGCCTCCACAAGTATTAGGCACATCTGTGGT
GCACAGACATACAATCAGGCAAAATATTCATACACATAAAATAAAATAATTTAAAACAAAAG
CAAAAATCAGGACCTAAGAAAAAAATCTATTCCTGATTCTTTTATGTTTTGTTTGTATTTTA
TCAAGACAGGGTTGTTTCTCTGTATAGCCCTGGCTGTCTTGGAATTCACTCTGTAGACCAGG
CTGGCCTCAAACTCAGAAATCCTCCTGCCTTTGCCTTCCAAGTGCTGGAATTAAAGGCATGC
GCCACC
SEQ ID NO: 43:
GACATGACCCAAACGGCCCCTGGCTGCAAGGTAATATCGGAAGTTGACTAAGAATGGACGCC
CCACCACTGACTGACCCGCCCCCTGAGTCTGAGATTGGACTTGTCTCTGGATACAGTCATAC
TTTGAGGTACTAGAAGTTAGAAACTGTTAGGTTACTCAGTTCAGTCCATGACAGTCCAACCT
TCTCCATGGTTTTCCGATCTCAGGCCCATGGCGACCTGCCCTGTCCTGCAGAAGGAGACACT
GTTCCGCACAGGCGTCCATGCTTACAGAATCCCTGCTCTGCTCTACCTGAAGAAGCAGAAGA
CCCTGCTGGCCTTTGCGGAAAAGCGAGCCAGCAAGACGGATGAGCACGCAGAGTTGATTGTC
CTGAGAAGAGGAAGCTACAACGAAGCCACCAACCGTGTCAAGTGGCAGCCTGAGGAAGTGGT
GACCCAAGCCCAGCTGGAAGGCCACCGCTCCATGAATCCATGTCCCTTGTATGACAAGCAAA
CAAAGACCCTCTTCCTTTTCTTCATCGCTGTCCCTGGGCGTGTATCAGAACATCATCAGCTC
CACACTAAGGTTAATGTCACACGGCTGTGCTGTGTCAGCAGCACTGACCATGGGAGGACCTG
GAGCCCCATCCAGGACCTCACAGAGACCACCATTGGCAGCACTCATCAGGAATGGGCCACAT
TTGCTGTGGGTCCTGGGCATTGTCTGCAGCTGCGGAACCCAGCTGGGAGCCTGCTGGTACCT
GCTTATGCCTACCGGAAACTGCACCCTGCTCAGAAGCCTACCCCCTTTGCCTTCTGCTTCAT
CAGCCTTGACCATGGGCACACATGGAAACTAGGCAACTTTGTGGCTGAAAACTCACTGGAGT
GCCAGGTGGCTGAGGTTGGCACTGGAGCTCAGAGGATGGTATATCTCAATGCTAGGAGCTTC
CTGGGAGCCAGGGTCCAGGCACAAAGTCCTAATGATGGTCTGGATTTCCAGGACAACCGGGT
AGTGAGTAAGCTTGTAGAGCCCCCCCACGGGTGTCATGGAAGTGTGGTTGCCTTCCACAACC
CCATCTCTAAGCCACATGCCTTAGACACATGGCTTCTTTATACACACCCTACAGACTCCAGG
AATAGAACCAACCTGGGTGTGTACCTAAACCAGATGCCACTAGATCCCACAGCCTGGTCAGA
GCCCACCCTGCTGGCCATGGGCATCTGTGCCTACTCAGACTTACAGAACATGGGGCAAGGCC
CTGATGGCTCCCCACAGTTTGGGTGTCTGTATGAATCAGGTAACTATGAAGAGATCATTTTC
CTCATATTCACCCTGAAGCAAGCTTTCCCCACTGTATTTGATGCCCAGTGATCTCAGTGCAC
GTGGCCCAAAGGGCTTCCTTGTGCTTCAAAACACCCATCTCTCTTTGCTTCCAGCATCCTCT
GGACTCTTGAGTCCAGCTCTTGGGTAACTTCCTCAGGAGGATGCAGAGAATTTGGTCTCTTG
ACTCTCTGCAGGCCTTATTGTTTCAGCCTCTGGTTCTCTTTTCAGCCCAGAAATCAAAGGAG
CCTGGCTTTCCTCAGCCTGTTGGCAGGGCAGGTGGGGACAGTATATATAGAGGCTGCCATTC
TGCATGTCGGTTGTCACTATGCTAGTTTAACCTGCCTGTTTCCCCATGCCTAGTGTTTGAAT
GAGTATTAATAAAATATCCAACCCAGCCCATTTCTTCCTGGAAAAAAA
SEQ ID NO: 44:
ACTGCGCGGTGAAGGGGCGTGGCCTGGCCGGGGAGGTTGACACCCAGACGCTGCTCTCAGTC
CTCTGGCGCCTGCTCCCCAGCGCATTCCTTCTGCTCCTGGGATATTTGTCTCATTACTGCCA
GTTCTTGCGCAGCGGTCACTGGGTTCGTTTCAGCGTCTGTGGTTTCTGTCGCTGTTATCCAG
TCTCCATCGCCCCAGCTCAGCTTCAGGCCTTCTTCCGAGACTCCACGGGAGAGCCCAGAGAG
CCTCCGGAGCCGAAGCCATGGAGGAAGTCCCACCCTACTCCCTCAGCAGCACCCTGTTCCAG
CAGGAAGAACAGAGTGGGGTGACCTACCGGATCCCAGCCCTGCTGTACCTTCCTCCCACCCA
CACCTTCCTGGCCTTTGCAGAGAAGCGGACCTCAGTCAGAGATGAGGATGCTGCCTGCCTGG
TGCTCAGACGAGGGCTGATGAAGGGGCGCTCTGTACAGTGGGGCCCCCAACGGCTACTGATG
GAGGCCACATTACCTGGGCATCGCACCATGAACCCCTGCCCTGTGTGGGAGAAAAATACTGG
CCGTGTGTACCTGTTTTTCATCTGTGTGCGGGGCCATGTTACTGAGAGGTGCCAGATTGTGT
GGGGCAAAAATGCCGCCCGTCTCTGCTTCCTTTGCAGTGAAGATGCCGGCTGCTCTTGGGGT
GAAGTGAAAGACTTGACCGAGGAGGTCATTGGCTCAGAGGTGAAGCGCTGGGCCACATTTGC
TGTGGGCCCAGGTCATGGCATCCAGCTACACTCGGGAAGGCTGATCATCCCCGCCTATGCCT
ACTATGTCTCACGTTGGTTTCTCTGCTTTGCGTGTTCAGTCAAGCCCCATTCCCTGATGATC
TACAGTGATGACTTTGGAGTCACATGGCACCATGGCAAGTTCATTGAGCCCCAGGTGACAGG
GGAGTGCCAAGTGGCCGAAGTGGCTGGGACGGCTGGTAACCCTGTGCTCACTGCAGTGCCCG
AACACCAAGCCGATTTCGAGCAGAGGCTTTTAGTACTGATAGTGGTGGCTGCTTTCAGAAGC
CAACCCTGAACCCACAACTCCATGAGCCTCGAACCGGCTGCCAAGGTAGTGTAGTGAGCTTC
CGGCCTTTGAAGATGCCAAATACCTATCAAGACTCAATTGGCAAAGGTGCTCCCGCTACTCA
GAAGTGCCCTCTGCTGGACAGTCCTCTGGAGGTGGAGAAAGGAGCTGAAACACCATCAGCAA
CATGGCTCTTGTACTCACATCCAACTAGCAAGAGGAAGAGGATTAACCTAGGCATCTACTAC
AACCGGAACCCCTTGGAGGTGAACTGCTGGTCCCGCCCGTGGATCTTGAACCGTGGGCCCAG
TGGCTACTCTGATCTGGCTGTTGTGGAAGAACAGGACTTGGTGGCGTGTTTGTTTGAGTGTG
GGGAGAAGAATGAGTATGAGCGGATTGACTTCTGTCTGTTTTCAGACCATGAGGTCCTGAGC
TGTGAAGACTGTACCAGCCCTAGTAGCGACTAAAGCCAAATCAAGACGGATGAGTGAGGCCC
AGCTTCCCACAGAAAGGAATGGCAGCTACAGCCAGGGTAACAGAGGTCTCTGATGTCTAGAG
AAAACTCTAAAAACTAATAATCTGCTCCTTGAATTTTTTCACTTTTCCCTTCAATGAGCATG
GTGAAAATTGTGCCATATCTTACATAACGAGGCTCTTGAACTGGGAGTTTGAATCTCTTCTC
TTCCCATTAAAAGGAGAGGCCATGTGCTCGCTTCGCGTTCGACAAAGCCTGGATTCTGATCT
TGAGTGGAAGCCACAGGCTTGTCTTTTCCAATGGTTCACTGCTCACCTGAGTATTAGGTGAT
GTGTAGGTGCCTTGGCCAGAAGAAAGATCTGTGTTGTTGTATTTTTTTAAATTTATTTATTT
ACTATATGTAAGTAGACTGCAGCTGTCTTCAGACACACCAGAAGAGGGCGTCAGATCTCATT
AGAGATGGTTGTGAGCCACCATGTGGTTGCTGGGATTTGAACTCAGGACCTTCAGAAGAGCA
GTCAGTGCTCTTAACTACTGAGCCATCTCTCAAGCCCCGCATTGCTGTATTTTTAATAAGAA
AAATGCCCTTATCCTTCCAATAATGCCTGGAGCTGTACAAATTCTCTGTCTTAGAAGACTTG
AGAAAGCAGAACTGTAAGGTCAGATGCTTTCTCCAGCCTTGATGCTGTGTTCCACCTTCCCT
TCCTCATCCAGAAAACAGTTACTAGGGAGAAAATGAGAAACCCATGCCAGCTGCCCTTGATG
ATGGTTGATAACGGTGCTTATTGCTTTTGATGTCATTACCTCTGTTAGAGATGAATCAGAGT
CAGAGGTCCTTAGCTGCATCCACCCATTTCCAGGGGGACATTCTAACACTGCTGAACAGTCA
GCTAAAATGAGAGCTGTGTGTCCTAGCCTGATTCCAGGTTAGTCATGATGCTTCCTGGAGCT
GGGCTTTTATCTAATCCCAGGAGCCATCTAGGGGAGGCTCAGAGCTAGCAGGTGATCTTCCT
GAGATGGTTTCACCGTGACAGGTGAACCATGAGCCCTTCCAAGCAAGGCCAAAGGACAACAT
TATAGGAAAGATTTCTAGTATTAATATGCCTTTTCTCTGTGTGTGTACTGTCTTGTAGTGAT
GCTATATAGACAAATAGATGATTTCTTATTTTTTGTTTGTTTGTTTGTTTTTTTGTTTTTCT
GTAGCCCTAGCTGTCCTGGAACTCACTTTGTAAACCAGGCTGGCCTCGATCTCAGAAATCCG
CCTGCCTCTGCCTCCCGAGTGCTGGGATTAAAGGTGTGCACCACCACACCTTAATGATGATC
CTATAAGTATTCCTAAAATTATACTAGTAATTATTAACTCCTTTATAATAGGACTGCTATTA
AAGCCCTCGCTGATATGAAAACTAGAGTGAGAACTCTGCCAGTCTTCACATGTCATAATTAC
TTCTGAGATAGAAAGCAGGCATTTACAACTTAGAACACATTTCTTAGAGCTGTAAAACAATT
AACTAGAGGTCATAAAAGGGAATGAAAGATTTATTGTAGGTGCTAGGACAGAACATAAAATA
TTGACTGGGCTTATCTATATGAAACTTCATTGTTAACTTTTACACAAGAATTATGGTTTTTA
ACTTTCAGTGAACCTGCGGAGCTAGTGACAGAAGAGAAATGTCTAGTTAGATAACTACTCTT
AATGGAAATTCACATAAACATCTGTTGCCATCTTCTTTTTGAATTTATGTTTAAACTTGTGA
ATGTTTGAATTAGACACTACGCGAGCACATAGAAAATAAAGAACTAAGCGTGAA
SEQ ID NO: 45:
GGACAGTGTGCATCACGGAGCTTGTGGCCCAGACTGTGCCTGGCAGACCCAGAGGACCTAAG
GCTTGGCTCTAGTGGTGGTCAGCACAGCCCTCGGTGGTCTGCGGAGCCTGATATTGCTTTAC
GTAAGGGCTGTTCTGCTGTGCATCTCCTGTGTCTGAAGCTATTCGCCATGGAGACTGCTGGA
GCTCCCTTCTGCTTCCATGTGGACTCCCTGGTACCTTGCTCCTACTGGAAGGTTATGGGGCC
CACGCGTGTTCCCAGGAGAACGGTGCTCTTCCAGAGGGAAAGGACGGGCCTGACCTACCGTG
TGCCTGCGTTACTCTGTGTGCCTCCCAGGCCTACTCTGCTGGCCTTCGCGGAACAGCGACTT
AGCCCTGATGACTCCCATGCCCACCGCCTGGTGCTACGGAGGGGCACGCTGACCAGGGGCTC
AGTGCGGTGGGGCACTCTGAGTGTACTGGAGACTGCAGTACTGGAGGAGCACAGGTCTATGA
ACCCTTGCCCGGTGCTGGATGAGCACTCTGGTACCATCTTCCTCTTCTTCATTGCCGTGCTG
GGCCACACACCGGAGGCCGTGCAAATCGCCACTGGCAAGAACGCTGCTCGCCTCTGCTGTGT
GACCAGCTGTGACGCTGGCCTCACCTGGGGCAGTGTTCGAGATCTCACTGAGGAAGCCATTG
GTGCTGCATTGCAGGACTGGGCCACCTTTGCTGTGGGTCCGGGCCATGGAGTTCAGCTGCGC
TCGGGTCGCCTGCTTGTTCCTGCTTACACCTATCATGTGGACCGACGGGAATGTTTTGGCAA
GATCTGCTGGACCAGTCCCCACTCCTTGGCATTCTACAGTGATGATCATGGGATCTCCTGGC
ATTGTGGAGGCCTTGTGCCCAACCTACGCTCTGGAGAGTGCCAACTGGCTGCGGTAGATGGA
GACTTTCTCTACTGTAATGCTCGAAGCCCTCTGGGTAACCGTGTGCAGGCACTGAGTGCTGA
TGAAGGCACGTCCTTCCTACCAGGGGAGCTGGTGCCTACATTGGCAGAGACGGCTCGTGGTT
GCCAGGGTAGCATTGTGGGCTTCCTAGCTCCACCCTCAATCGAGCCTCAGGATGACCGGTGG
ACAGGGAGTCCTAGGAACACCCCACATTCCCCATGCTTCAATCTCAGAGTACAGGAGTCTTC
GGGGGAAGGTGCCAGAGGTCTTCTTGAACGTTGGATGCCCAGGTTGCCTCTCTGCTACCCAC
AGTCCCGGAGCCCAGAGAATCATGGCCTAGAGCCTGGGTCAGATGGAGATAAGACATCCTGG
ACTCCGGAATGTCCTATGTCCTCTGATTCCATGCTTCAGAGCCCCACATGGCTACTATATTC
CCACCCAGCAGGGCGTAGAGCTCGGCTCCACATGGGAATCTACCTGAGCCGATCCCCCTTGG
ATCCCCACAGCTGGACAGAGCCCTGGGTGATCTATGAGGGCCCCAGTGGCTACTCTGACCTT
GCCTTTCTTGGGCCTATGCCTGGGGCATCCCTGGTTTTTGCCTGTCTGTTTGAGAGCGGGAC
CAGGACTTCCTATGAAGACATTTCTTTTTGCTTGTTCTCACTGGCGGATGTCCTGGAGAATG
TGCCCACTGGCTTAGAGATGCTAAGTCTCAGGGATAAGGCTCAGGGGCATTGCTGGCCCTCT
TGATGGCCTCACCCTCTCGTAGCCGCCTGGAGAGGAAGGGTAGACTATATAGAGGAGGTTAG
GGGTAGGTCAGCATGATGCTAGGATGGAGAGAGCTCTGTCCCCTCGTGGATGGTGGTGGTGA
CTCACCCGGGGGGCCAGCTGCTTTCTGAGTGCAAATGAGAAAAATAAAGAGCTGCGCTGTGA
CTTTTCTTTCCACATCAAAGCTTGGGTGTCAGTGCTTTAGCTTGATGCTCTGATCACCATGC
AAATCTTCCACCGGCGCCTTGCTCAGCTTTCATATCCCAAGGGTGCCTGGGAGGAAGGCAAC
AGGGACAGTGGACATCACTGCACCACTTTCCACGACCCTGTGTGCCAACCTCAGCCACTTTG
AAACATGCTGATGACTGAGGTCTGTTCACTTTCTTAATTTCAAGCAGGAGAAGCAGGTTGGG
GAGCCAGCCTCCCCAGCTAGAGGGGACAGAACTTGACTTGAGCAGGGGGGTACCTCCTAGGA
CCTGCTCCATGTGCCTACTTCTTTACCCTTCTCTAGAGAGGGCTCTTGTCCTGTCAGAGCTG
TTTTCTCCCTTCTCTTGTTTTTTCTTTTTCAAGACTGTTTCTCTGTGTTAGCCCTGGCTGTC
CTGGATCTCACTCTGTAGATCAGGCTGACCTTGAGTTCAAAGCTCCATCTGCCTCTACTTCT
CACATTACTGTGATTAAAGGCATATACTACCACTGCCTGGTGCCCTTTTGTATTTCTTATTA
AAGTCCTAATGTCTGATTATAAAAACAGTCTGTGTGGGCTGGAGTGATGGCTTACTCAGTAA
AGCACTTGCCATGGAATCTGGGCAATCTGAGTTTCATTTTTAGCATCCTGTAAAAATCCCAA
TTTGATGGTGTACTTGTAATGTCAGCATGGAGAGGCAGAGATAGGTAAGTTCCCCAAGACTC
TTTGAACCGACAGCTTGGCCTCACTGGCACATTCCAGGTCTCAGTGAGAGACCCTGCCTCAA
AATACAAAGAAAGAGCTGCTGAAGAGTGGGTCAGAGTTGACCTCTGATCTCCGGAAGTATAT
GATACACACCCGTGCATGCACTCTTCCTTACAAAATAAAAAGCAAAACAAAACCCCAACAGG
TATATGGCCATTTTAGAAAAATTAGAAGATTTAGAAAGCTATACATAAAAAAAAATGACCTA
AAGAAAAATCTTTACTGTTCTGGGCACTATCCCTATCAAACCACTGTGTTCTTTGGCCAAGC
CTTGGGGTGGACACTGTTTTGAGGTGGGTCCTGTTATCTCCACTAGGTAGTGGAGTTTTGTG
TCAGACTAACTGGGTCTTAAAGCTGTCTTTAAGGCCATCAGGAGCTACTGACTTGCCTGCCT
CAGCAGAGCATATCCTGAAGGTCGGGGTTAAGTCTCCTTCCCGAGCGAGTTGCCTTCCAGTG
GGCCCCTGGACTCCTAGGTCCTCAGCGCTCATCAGCTGCCAAGGACTCTGAGGGAATGTCCT
CTGACTGTGGCCCCGAAAGGTAGGGGAGGGGGATGTGCTTAGGCTTAGGACAGGGTCCTGTT
TCAGTCTGCCTTCACTGTTAGTAGCACTGTGCCACATGGCACAGACTGGGCGAGCTTTAAAG
GAAGGAGGTTGATATTGGTTCCCACTTCTGGGGATCATGGTTGAGCAGCCTTGTCTGATGAT
GGTTGTCTTGATGGTAGATCGTGAGGTAGTTGATGAAGGTATGACATGGTGAGAAACTCTGT
GTGTGTGTGTTATTTTCTCTGTGTTCTACCTATACATCTATCTATGTATATATGTATCTATC
TATCTACCTGGAGGCTGGAGAGATAGCTTAGTGGTTAAGAACATTTGTTGTTCTTGCATAGT
CCTGGATTTAAATTTTCAGCACCCACATGGGAGCTCACAACAACCCATAAATCCAGTTTCAG
AGGATCCAACCTCTGATATACCATGTCAGCCAGAGCAGACACGGCTGAAGGTGGTTTGATCC
CCGTATGGAGAGGTGACAATTGGGAAGAGAGAAAGATCAACTTAACCATGCAAGGAACAGGA
AGTTAAATACTGAACAGGGAAGGTAAAGGCAGGAAGTAGATGTAGAGGGCAAATCAATGAAA
CCCAAACATACCCAAATTACGCTAAACACACACTGACATGCCAATTAAAAGGACAAATTGGC
TCCACTGGCAAAACCAAAACAGACACTGAAGATCCAAACAGTCACATGCCAACTACCGCGGA
GGGAGACAGACACAGAGAAGACCGTGACAGACACTTGGACACTCTTGAGAGTGGATGTGCAG
GAAGAGAGCTCTGCCAGTGGAGAAGAAAGCACTCAGAAGAAAGTGACAGCAGCTGTAAATTT
GTATTCTGCTAATGTTATGTTCCAAAGTTGAAAGCAAAATTGTACCAATTCATAAGAACAAA
CAGGCTGACTCTCAGTTGTGACTGAACGTCTCTCAGTAACTGACGGGGCGAGCAGGCCAAAG
GAGAGTCGGCTCAGAAGGGTGCATAGCCACGCCAAATCAAATAAGCAAGTACAACCGGCAGG
CTCTATTTCTAGCACAAAGGGGTCTGTGCCTCATTCTGTGCTTGGGTCAGAGCTTGGGTCTC
TCATTTGGATGTAAGTGGTGTAGTGGAGAAGCAGGAAATAATCCGGAGCGCATATTTTGATT
TTAACATAAGTGCTGATTTGGGAGGGAGTTTTGTCAAATTGTGTTTTTACAATGTTTTTTTT
TTTTTAAATGATGCTTTTTTGTAAAGTGTACAAATGTGATATAAGATTGGTTCTGCTACATT
CAGTTTCTATAAAAGTGGTTCTAAAATATTGTACTGTCAATCATCTCATGATTATTCTACTG
TAGACATTACTGACTTTGTATGTAATAATTAATATTAGAAGAAAATATAATTTATTTGAATA
TAAAAAAAAAAAAAAAAAAA
SEQ ID NO: 46:
X₁ASLPX₂LQX₃ESVFQSGAHAYRIPALLYLPGQQSLLAFAEQRASKKDEHAELIVLRRGDYD
AX₄THQVQWQAQEVVAQARLDGHRSMNPCPLYDX₅QTGTLFLFFIAIPGQVTEQQQLQTRANV
TRLCQVTSTDHGRTWSSPRDLTDAAIGPAYREWSTFAVGPGHCLQLHDRARSLVVPAYAYRK
LHPX₆QRPIPSAFCFLSHDHGRTWARGHFVAQDTLECQVAEVETGEQRWTLNARSHLRARVQ
AQSTNDGLDFQESQLVKKLVEPPPX₇GCQGSVISFPSPRSGPGSPAQWLLYTHPTHX₈X₉QRA
DLGAYLNPRPPAPEAWSEPVLLAKGSX₁₀AYSDLQSMGTGPDGSPLFGCLYEANDYEEIX₁₁FX₁₂
MFTLKQAFPAEYLPQ
SEQ ID NO: 47:
X₁X₂SX₃X₄X₅LQX₆ESVFQSGAHAYRIPALLYLPGQQSLLAFAEQRASX₇X₈DEHAELIVX₉RR
GDYDAX₁₀THQVQWX₁₁AQEVVAQAX₁₂LDGHRSMNPCPLYDX₁₃QTGTLFLFFIAIPX₁₄X₁₅VT
EX₁₆QQLQTRANVTRLX₁₇X₁₈VTSTDHGRTWSSPRDLTDAAIGPX₁₉YREWSTFAVGPGHX₂₀LQ
LHDRX₂₁RSLVVPAYAYRKLHPX₂₂QRPIPSAFX₂₃FLSHDHGRTWARGHFVAQDTX₂₄ECQVAE
VETGEQRVVTLNARSHLRARVQAQSX₂₅NX₂₆GLDFQX₂₇SQLVKKLVEPPPX₂₈GX₂₉QGSVISF
PSPRSGPGSPAQX₃₀LLYTHPTHX₃₁X₃₂QRADLGAYLNPRPPAPEAWSEPX₃₃LLAKGSX₃₄AYS
DLQSMGTGPDGSPLFGX₃₅LYEANDYEEIX₃₆FX₃₇MFTLKQAFPAEYLPQ
SEQ ID NO: 48:
DASLPYLQKESVFQSGAHAYRIPALLYLPGQQSLLAFAEQRASKKDEHAELIVLRRGDYDAG
THQVQWQAQEVVAQARLDGHRSMNPCPLYDEQTGTLFLFFIAIPGQVTEQQQLQTRANVTRL
CQVTSTDHGRTWSSPRDLTDAAIGPAYREWSTFAVGPGHCLQLHDRARSLVVPAYAYRKLHP
KQRPIPSAFCFLSHDHGRTWARGHFVAQDTLECQVAEVETGEQRVVTLNARSHLRARVQAQS
TNDGLDFQESQLVKKLVEPPPQGCQGSVISFPSPRSGPGSPAQWLLYTHPTHSWQRADLGAY
LNPRPPAPEAWSEPVLLAKGSAAYSDLQSMGTGPDGSPLFGCLYEANDYEEIVFLMETLKQA
FPAEYLPQ
SEQ ID NO: 49:
DASLPYLQDESVFQSGAHAYRIPALLYLPGQQSLLAFAEQRASKKDEHAELIVLRRGDYDAP
THQVQWQAQEVVAQARLDGHRSMNPCPLYDEQTGTLFLFFIAIPGQVTEQQQLQTRANVTRL
CQVTSTDHGRTWSSPRDLTDAAIGPAYREWSTFAVGPGHCLQLHDRARSLVVPAYAYRKLHP
KQRPIPSAFCFLSHDHGRTWARGHFVAQDTLECQVAEVETGEQRVVTLNARSHLRARVQAQS
TNDGLDFQESQLVKKLVEPPPQGCQGSVISFPSPRSGPGSPAQWLLYTHPTHSWQRADLGAY
LNPRPPAPEAWSEPVLLAKGSAAYSDLQSMGTGPDGSPLFGCLYEANDYEEIRFIMFTLKQA
FPAEYLPQ
SEQ ID NO: 50:
DASLPYLQKESVFQSGAHAYRIPALLYLPGQQSLLAFAEQRASKKDEHAELIVLRRGDYDAN
THQVQWQAQEVVAQARLDGHRSMNPCPLYDAQTGTLFLFFIAIPGQVTEQQQLQTRANVTRL
CQVTSTDHGRTWSSPRDLTDAAIGPAYREWSTFAVGPGHCLQLHDRARSLVVPAYAYRKLHP
KQRPIPSAFCFLSHDHGRTWARGHFVAQDTLECQVAEVETGEQRVVTLNARSHLRARVQAQS
TNDGLDFQESQLVKKLVEPPPQGCQGSVISFPSPRSGPGSPAQWLLYTHPTHSWQRADLGAY
LNPRPPAPEAWSEPVLLAKGSAAYSDLQSMGTGPDGSPLFGCLYEANDYEEIVFLMFTLKQA
FPAEYLPQ
SEQ ID NO: 51:
DASLPYLQKESVFQSGAHAYRIPALLYLPGQQSLLAFAEQRASKKDEHAELIVLRRGDYDAP
THQVQWQAQEVVAQARLDGHRSMNPCPLYDAQTGTLFLFFIAIPGQVTEQQQLQTRANVTRL
CQVTSTDHGRTWSSPRDLTDAAIGPAYREWSTFAVGPGHCLQLHDRARSLVVPAYAYRKLHP
KQRPIPSAFCFLSHDHGRTWARGHFVAQDTLECQVAEVETGEQRVVTLNARSHLRARVQAQS
TNDGLDFQESQLVKKLVEPPPAGCQGSVISFPSPRSGPGSPAQWLLYTHPTHRKQRADLGAY
LNPRPPAPEAWSEPVLLAKGSAAYSDLQSMGTGPDGSPLFGCLYEANDYEEIVFLMFTLKQA
FPAEYLPQ
SEQ ID NO: 52:
DASLPYLQKESVFQSGAHAYRIPALLYLPGQQSLLAFAEQRASKKDEHAELIVLRRGDYDAS
THQVQWQAQEVVAQARLDGHRSMNPCPLYDAQTGTLFLFFIAIPGQVTEQQQLQTRANVTRL
CQVTSTDHGRTWSSPRDLTDAAIGPAYREWSTFAVGPGHCLQLHDRARSLVVPAYAYRKLHP
KQRPIPSAFCFLSHDHGRTWARGHFVAQDTLECQVAEVETGEQRVVTLNARSHLRARVQAQS
TNDGLDFQESQLVKKLVEPPPAGCQGSVISFPSPRSGPGSPAQWLLYTHPTHRKQRADLGAY
LNPRPPAPEAWSEPVLLAKGSAAYSDLQSMGTGPDGSPLFGCLYEANDYEEIVFLMFTLKQA
FPAEYLPQ
SEQ ID NO: 53:
DASLPYLQKESVFQSGAHAYRIPALLYLPGQQSLLAFAEQRASKKDEHAELIVLRRGDYDAT
THQVQWQAQEVVAQARLDGHRSMNPCPLYDAQTGTLFLFFIAIPGQVTEQQQLQTRANVTRL
CQVTSTDHGRTWSSPRDLTDAAIGPAYREWSTFAVGPGHCLQLHDRARSLVVPAYAYRKLHP
KQRPIPSAFCFLSHDHGRTWARGHFVAQDTLECQVAEVETGEQRVVTLNARSHLRARVQAQS
TNDGLDFQESQLVKKLVEPPPAGCQGSVISFPSPRSGPGSPAQWLLYTHPTHRKQRADLGAY
LNPRPPAPEAWSEPVLLAKGSAAYSDLQSMGTGPDGSPLFGCLYEANDYEEIVFLMFTLKQA
FPAEYLPQ
SEQ ID NO: 54:
DASLPYLQKESVFQSGAHAYRIPALLYLPGQQSLLAFAEQRASKKDEHAELIVLRRGDYDAN
THQVQWQAQEVVAQARLDGHRSMNPCPLYDAQTGTLFLFFIAIPGQVTEQQQLQTRANVTRL
CQVTSTDHGRTWSSPRDLTDAAIGPAYREWSTFAVGPGHCLQLHDRARSLVVPAYAYRKLHP
KQRPIPSAFCFLSHDHGRTWARGHFVAQDTLECQVAEVETGEQRVVTLNARSHLRARVQAQS
TNDGLDFQESQLVKKLVEPPPAGCQGSVISFPSPRSGPGSPAQWLLYTHPTHRKQRADLGAY
LNPRPPAPEAWSEPVLLAKGSAAYSDLQSMGTGPDGSPLFGCLYEANDYEEIVFLMFTLKQA
FPAEYLPQ
SEQ ID NO: 55:
ASLPYLQKESVFQSGAHAYRIPALLYLPGQQSLLAFAEQRASKKDEHAELIVLRRGDYDAPT
HQVQWQAQEVVAQARLDGHRSMNPCPLYDAQTGTLFLFFIAIPGQVTEQQQLQTRANVTRLC
QVTSTDHGRTWSSPRDLTDAAIGPAYREWSTFAVGPGHCLQLHDRARSLVVPAYAYRKLHPK
QRPIPSAFCFLSHDHGRTWARGHFVAQDTLECQVAEVETGEQRVVTLNARSHLRARVQAQST
NDGLDFQESQLVKKLVEPPPQGCQGSVISFPSPRSGPGSPAQWLLYTHPTHSWQRADLGAYL
NPRPPAPEAWSEPVLLAKGSCAYSDLQSMGTGPDGSPLFGCLYEANDYEEIVFLMFTLKQAF
PAEYLPQ
SEQ ID NO: 56:
MASLPYLQKESVFQSGAHAYRIPALLYLPGQQSLLAFAEQRASKKDEHAELIVLRRGDYDAP
THQVQWQAQEVVAQARLDGHRSMNPCPLYDAQTGTLFLFFIAIPGQVTEQQQLQTRANVTRL
CQVTSTDHGRTWSSPRDLTDAAIGPAYREWSTFAVGPGHCLQLHDRARSLVVPAYAYRKLHP
KQRPIPSAFCFLSHDHGRTWARGHFVAQDTLECQVAEVETGEQRVVTLNARSHLRARVQAQS
TNDGLDFQESQLVKKLVEPPPQGCQGSVISFPSPRSGPGSPAQWLLYTHPTHSWQRADLGAY
LNPRPPAPEAWSEPVLLAKGSCAYSDLQSMGTGPDGSPLFGCLYEANDYEEIVFLMFTLKQA
FPAEYLPQ
SEQ ID NO: 57:
ASLPYLQKESVFQSGAHAYRIPALLYLPGQQSLLAFAEQRASKKDEHAELIVLRRGDYDAPT
HQVQWQAQEVVAQARLDGHRSMNPCPLYDAQTGTLFLFFIAIPGQVTEQQQLQTRANVTRLC
QVTSTDHGRTWSSPRDLTDAAIGPAYREWSTFAVGPGHCLQLHDRARSLVVPAYAYRKLHPK
QRPIPSAFCFLSHDHGRTWARGHFVAQDTLECQVAEVETGEQRVVTLNARSHLRARVQAQST
NDGLDFQESQLVKKLVEPPPQGCQGSVISFPSPRSGPGSPAQWLLYTHPTHSWQRADLGAYL
NPRPPAPEAWSEPVLLAKGSAAYSDLQSMGTGPDGSPLFGCLYEANDYEEIVFLMFTLKQAF
PAEYLPQ
SEQ ID NO: 58:
DASLPYLQKESVFQSGAHAYRIPALLYLPGQQSLLAFAEQRASKKDEHAELIVLRRGDYDAP
THQVQWQAQEVVAQARLDGHRSMNPCPLYDAQTGTLFLFFIAIPGQVTEQQQLQTRANVTRL
CQVTSTDHGRTWSSPRDLTDAAIGPAYREWSTFAVGPGHCLQLHDRARSLVVPAYAYRKLHP
KQRPIPSAFCFLSHDHGRTWARGHFVAQDTLECQVAEVETGEQRVVTLNARSHLRARVQAQS
TNDGLDFQESQLVKKLVEPPPQGCQGSVISFPSPRSGPGSPAQWLLYTHPTHSWQRADLGAY
LNPRPPAPEAWSEPVLLAKGSAAYSDLQSMGTGPDGSPLFGCLYEANDYEEIVFLMFTLKQA
FPAEYLPQ
SEQ ID NO: 59:
DASLPYLQKESVFQSGAHAYRIPALLYLPGQQSLLAFAEQRASKKDEHAELIVLRRGDYDAP
THQVQWQAQEVVAQARLDGHRSMNPCPLYDAQTGTLFLFFIAIPGQVTEQQQLQTRANVTRL
CQVTSTDHGRTWSSPRDLTDAAIGPAYREWSTFAVGPGHCLQLHDRARSLVVPAYAYRKLHP
KQRPIPSAFCFLSHDHGRTWARGHFVAQDTLECQVAEVETGEQRVVTLNARSHLRARVQAQS
TNDGLDFQESQLVKKLVEPPPQGCQGSVISFPSPRSGPGSPAQWLLYTHPTHSWQRADLGAY
LNPRPPAPEAWSEPVLLAKGSCAYSDLQSMGTGPDGSPLFGCLYEANDYEEIVFLMFTLKQA
FPAEYLPQ
SEQ ID NO: 60:
AASLPYLQKESVFQSGAHAYRIPALLYLPGQQSLLAFAEQRASKKDEHAELIVLRRGDYDAP
THQVQWQAQEVVAQARLDGHRSMNPCPLYDAQTGTLFLFFIAIPGQVTEQQQLQTRANVTRL
CQVTSTDHGRTWSSPRDLTDAAIGPAYREWSTFAVGPGHCLQLHDRARSLVVPAYAYRKLHP
KQRPIPSAFCFLSHDHGRTWARGHFVAQDTLECQVAEVETGEQRVVTLNARSHLRARVQAQS
TNDGLDFQESQLVKKLVEPPPQGCQGSVISFPSPRSGPGSPAQWLLYTHPTHSWQRADLGAY
LNPRPPAPEAWSEPVLLAKGSAAYSDLQSMGTGPDGSPLFGCLYEANDYEEIVFLMFTLKQA
FPAEYLPQ
SEQ ID NO: 61:
MASLPYLQKESVFQSGAHAYRIPALLYLPGQQSLLAFAEQRASKKDEHAELIVLRRGDYDAP
THQVQWQAQEVVAQARLDGHRSMNPCPLYDAQTGTLFLFFIAIPGQVTEQQQLQTRANVTRL
CQVTSTDHGRTWSSPRDLTDAAIGPAYREWSTFAVGPGHCLQLHDRARSLVVPAYAYRKLHP
KQRPIPSAFCFLSHDHGRTWARGHFVAQDTLECQVAEVETGEQRVVTLNARSHLRARVQAQS
TNDGLDFQESQLVKKLVEPPPQGCQGSVISFPSPRSGPGSPAQWLLYTHPTHSWQRADLGAY
LNPRPPAPEAWSEPVLLAKGSAAYSDLQSMGTGPDGSPLFGCLYEANDYEEIVFLMFTLKQA
FPAEYLPQ
SEQ ID NO: 62:
AASLPYLQKESVFQSGAHAYRIPALLYLPGQQSLLAFAEQRASKKDEHAELIVLRRGDYDAP
THQVQWQAQEVVAQARLDGHRSMNPCPLYDAQTGTLFLFFIAIPGQVTEQQQLQTRANVTRL
CQVTSTDHGRTWSSPRDLTDAAIGPAYREWSTFAVGPGHCLQLHDRARSLVVPAYAYRKLHP
KQRPIPSAFCFLSHDHGRTWARGHFVAQDTLECQVAEVETGEQRVVTLNARSHLRARVQAQS
TNDGLDFQESQLVKKLVEPPPQGCQGSVISFPSPRSGPGSPAQWLLYTHPTHSWQRADLGAY
LNPRPPAPEAWSEPVLLAKGSCAYSDLQSMGTGPDGSPLFGCLYEANDYEEIVFLMFTLKQA
FPAEYLPQ
SEQ ID NO: 63:
MNPNQKIIALGSVSLTIAAICLLMQIAILATTMTLHLQQDGCTNPPNNQAVPHEPIIIERNR
TEIVYVNNTTIEKENCPKVAEYKNWSKPQCQITGFAPFSKDNSIRLSAGGDIWVTREPYVSC
GLGKCYQFALGQGTTLNNRHSNGTTHDRSPHRTLLMNELGVPFHLGTKQVCIAWSSSSCHDG
KAWLHVCITGDDRNATASIIYDGLLTDSIGTWSKNILRTQESECICINGTCTVVMTDGSASG
WADTRILFIREGKIVHISPLSGSAQHVEECSCYPRYPEVRCVCRDNWKGSNR
PVLYINVEDYSIDSSYLCSGLVGDTPRNEDSSSSSNCRDPNNERGGPGVKGWAFDSGNDVWM
GRTIRKDSREGYETFRVVGGWTTANSKSQINRQVIVDSDNLSGYSGMFSVEGKSCINRCFYV
ELIRGRPQETRVWWTSNSIIVFCGTSGTYGTGSWPDGANINFMSI
SEQ ID NO: 64:
MLPSTIQTLTLFLTSGGVLLSLYVSASLSYLLYSGILLKFSPTEITAPTMPLNCANASNVQA
VNRSATKGVTLLLPEPEWTYPRLSCPGSTFQKALLISPHRFGETKGNSAPLIIREPFIACGP
KECKHFALTHYAAQPGGYYNGTREDRNKLRHLISVKLGKIPTVENSIFHMAAWSGSACHDGR
EWTYIGVDGPDSNALLKIKYGEAYTDTYHSYANNILRTQESACNCIGGDCYLMITDGSASGI
SECRFLKIREGRIIKEIFPTGRVEHTEECTCGFASNKTIECACRDNSYTAKRPFVKLNVETD
TAEIRLMCTETYLDTPRPDDGSITGPCESHGDKGSGGIKGGFVHQRMASKIGRWYSRTMSKT
KRMGMGLYVKYDGDPWIDSGALTLSGVMVSMEEPGWYSFGFEIKDKKCDVPCIGIEMVHDGG
KKTWHSAATAIYCLMGSGQLLWDTVTGVDMAL
SEQ ID NO: 65:
MEYWKHTNHGKDVGNELETSTATHGNKLTNKITYILWTITLVLLSIVFIIVLTNSIKSEKAR
ESLLQDINNEFMEVTEKIQVASDNTNDLIQSGVNTRLLTIQSHVQNYIPISLTQQISDLRKF
ISEITIRNDNQEVPPQRITHDVGIKPLNPDDFWRCTSGLPSLMKTPKIRLMPGPGLLAMPTT
VDGCVRTPSLVINDLIYAYTSNLITRGCQDIGKSYQVLQIGIITVNSDLVPDLNPRISHTFN
INDNRKSCSLALLNTDVYQLCSTPKVDERSDYASSGIEDLVLDIVNYDGSISTTRFKNNNIS
FDQPYAALYPSVGPGIYYKGKIIFLGYGGLEHPINENAICNTTECPGKTQRDCNQASHSPWF
SDRRMVNSIIVVDKGLNSVPKLKVWSISMRQNYWGSEGRLLLLGNKIYIYTRSTSWHSKLQL
GIIDITDYSDIRIKWTWHNVLSRPGNNECPWGHSCPDGCITGVYTDAYPLNPTGSIVSSVIL
DSQKSRVNPVITYSTATERVNELAIRNETLSAGYTTTSCITHYNKGYCFHIVEINHKSLNTF
QPMLFKTEIPKSCS
SEQ ID NO: 66:
MEYWRHTNNAKNTNTEFQTETTRRNNKVTNVMMSIFGAILSTILLTVFIMILVGLIQEGNHN
KIASQQMRREFAEIERKIQQATDEIGTSIQSGINTRLLTIQSHVQNYIPLSLTQQISDLRKF
INELANKRDQQEVPIQRMTHDSGIEPLNPDKFWRCTSGNPSLASNPKIRLIPGPSLLAASTT
VNGCIRIPSFVINNLIYAYTSNLIVQGCQDIGKSYQVLQIGIITINSDLVPDLNPRVTHTFN
IDDNRKSCSLALLNTDVYQLCSTPKVDERSDYASTGIEDIVLDIITNNGLIITTRFTNDNIT
FDKPYAALYPSVGPGIYYKGKVIFLGYGGLEHAENGDVICNLTGCPGKTQRDCNQASYSPWF
SNRRMVNSIIVVNKGVDTTFNLRVWTIPMRQNYWGSEGRLLLLGNKIYIYTRSTSWHSKLQL
GTIDINNYSDIRINWTWHDALSRPGNDDCPWGHSCPDGCITGVYTDAYPLNPSGSVVSSVIL
DSRKSRENPIITYATDTRRVNELAIYNRTLPAAYTTTNCIMHYDKGYCFHIVEINHRSLNTF
QPMLFKTEIPKNCS
SEQ ID NO: 67:
MDGDRGKRDSYWSTSPSGSTTKPASGWERSSKADTWLLILSFTQWALSIATVIICIIISARQ
GYSMKEYSMTVEALNMSSREVKESLTSLIRQEVIARAVNIQSSVQTGIPVLLNKNSRDVIQM
IDKSCSRQELTQHCESTIAVHHADGIAPLEPHSFWRCPVGEPYLSSDPEISLLPGPSLLSGS
TTISGCVRLPSLSIGEAIYAYSSNLITQGCADIGKSYQVLQLGYISLNSDMFPDLNPVVSHT
YDINDNRKSCSVVATGTRGYQLCSMPTVDERTDYSSDGIEDLVLDVLDLKGRTKSHRYRNSE
VDLDHPFSALYPSVGNGIATEGSLIFLGYGGLTTPLQGDTKCRTQGCQQVSQDTCNEALKIT
WLGGKQVVSVIIQVNDYLSERPKIRVTTIPITQNYLGAEGRLLKLGDRVYIYTRSSGWHSQL
QIGVLDVSHPLTINWTPHEALSRPGNKECNWYNKCPKECISGVYTDAYPLSPDAANVATVTL
YANTSRVNPTIMYSNTTNIINMLRIKDVQLEAAYTTTSCITHFGKGYCFHIIEINQKSLNTL
QPMLFKTSIPKLCKAES
SEQ ID NO: 68:
MTSHSPFSRRRLPALLGSLPLAATGLIAAAPPAHAVPTSDGLADVTITQVNAPADGLYSVGD
VMTFNITLTNTSGEAHSYAPASTNLSGNVSKCRWRNVPAGTTKTDCTGLATHTVTAEDLKAG
GFTPQIAYEVKAVEYAGKALSTPETIKGATSPVKANSLRVESITPSSSQENYKLGDTVSYTV
RVRSVSDKTINVAATESSFDDLGRQCHWGGLKPGKGAVYNCKPLTHTITQADVDAGRWTPSI
TLTATGTDGATLQTLTATGNPINVVGDHPQATPAPAPDASTELPASMSQAQHLAANTATDNY
RIPAITTAPNGDLLISYDERPKDNGNGGSDAPNPNHIVQRRSTDGGKTWSAPTYIHQGTETG
KKVGYSDPSYVVDHQTGTIFNFHVKSYDQGWGGSRGGTDPENRGIIQAEVSTSTDNGWTWTH
RTITADITKDKPWTARFAASGQGIQIQHGPHAGRLVQQYTIRTAGGAVQAVSVYSDDHGKTW
QAGTPIGTGMDENKVVELSDGSLMLNSRASDGSGFRKVAHSTDGGQTWSEPVSDKNLPDSVD
NAQIIRAFPNAAPDDPRAKVLLLSHSPNPRPWSRDRGTISMSCDDGASWTTSKVFHEPFVGY
TTIAVQSDGSIGLLSEDAHNGADYGGIWYRNFTMNWLGEQCGQKPAEPSPAPSPTAAPSAAP
TEKPAPSAAPSAEPTQAPAPSSAFEPSAAPEPSSAPAPEPTTAPSTEPTPAPAPSSAPEQTD
GPTAAPAPETSSAPAAEPTQAPTVAPSVEPTQAPGAQPSSAPKPGATGRAPSVVNPKATGAA
TEPGTPSSSASPAPSRNAAPTPKPGMEPDEIDRPSDGTMAQPTGGASAPSAAPTQAAKAGSR
LSRTGTNALLILGLAGVAVVGGYLLLRARRSKN
SEQ ID NO: 69:
MRSNSTSAPSLVRRAATGVLTCAVSIGLLAGLGLPAQAAPTPPNSPTLPPGSFSETNLAADR
TAANFFYRIPALTYLGNDVVLAAWDGRPGSAADAPNPNSIVQRRSTDGGKTWGPVQVIAAGH
VADASGPRYGYSDPSYIYDAEANKVFAFFVYSKDQGFGGSQFGNDDADRNVISSAVIESSDA
GVTWSQPRLITSVTKPGTSKTNPAAGDVRSNFASSGEGIQLKYGPHKGRLIQQYAGDVRQAD
GSNKIQAYSVYSDDHGVTWHKGANVGDRMDENKTVELSDGRVLLNSRDNANRGYRKVAVSTD
GGATYGPVSQDTELPDPANNGAIARMFPNAAQGSADAKKLIFTNANSKTGRENVSARVSCDD
GETWPGVRTIRSGFSAYSTVTRLADGKFGVLYEGNYTDNMPFATFDDAWLNYVCAPLAVPAV
NIAPSATQEVPVTVTNQEATTLSGATATVYTPSGWSATTVPVPDVAPGASVTVTVALTAPAD
ASGPRSLNAAFTTADGRVSQFTFTATTPVAPQVGLTITGSAPARDVAANPYQAGDVLGYTLN
VKSTANVAANSVPLTGTFDSGFLPPAAPNCRYNNLAAGASYNCTTAKHTITAADMERGYFVP
EATFSITSTTTPSLTKTVQFTGAAVALRDGLISADISGARTDVGRDLATRPYAAGELVPYAF
TVKNTSPFVEQVVPTAGNFSPFLPAGAGNCRYLSLPAGQSYECATPRHAVTAEEVEQGFFVP
DTTWEVSAAGQSTRTYRINGGEVDLLVRDAALSATVVAEWKDADGDRFASAGDPVTFTYTVG
NAGNVALTGLEAPDAGISLPFLAPGDTATATREHVLTAADVAGGSLAASAFEATARNGSKEV
TATAEGQPLELKVQPAQPSKEPELTVQDLEDQTPPFDLGTAFKYRTGQKVSLAGLEYGQWYY
VYLNKTGYRLGWMFPTTGDTVEFILPPEVRNGRDDVVVLDKDGRRVSFDRLQVTPKGEKI
SEQ ID NO: 70:
MCNKNNTFEKNLDISHHPEPLILFNKDANIWNSKYFRIPNVQLLNDVTILTFSDIRYNAPDD
HAYIDIASARSTDFGKTWSYNIAMKNNRIDSTYSRAMDSTTLITNTVRIILIASSWNTNGNW
AMTTSARRSDWSVQMIYSDDNGLTWSNKIDLTKDSSKVKNQPSNTIGWLGGVGSGITMDDGT
IVMPSQISARENNENNYYSLIIYSKDNGETWTMGNKVPNSNTSENMVIELDVALIMSTRYDY
SGYRAAYISHDLGTTWEIYEPLNGKILTGKGSGCQGSFIHATTSNGKRIALISAPKNTHGEY
IRDQIAVYMIDFDDLSKGVQEICIPYPEDGNKLGGGYSCLSFKNNHLGIVYDFNGNIEYQDL
YPYYSLINKQ
SEQ ID NO: 71:
MNYKGITLILTAAMVISGGNYVLVKGSTLDSGKNNSGYEIKVNNSENLSSLGEYKDINLESS
NASNITYDLEKYKNLDEGTIVVRFNSKDSKIQSLLGISNSKTKNGYFNFYVTNSRVGFELRN
QKNEGNTQNGTENLVHMYKDVALNDGDNTVALKIEKNKGYKLELNGKMIKEVKDTNTKELNN
IENLDSAFIGKTNRYGQSNEYNFKGNIGFMNIYNEPLGDDYLLSKTGETKAKEEVLVEGAVK
TEPVDLFHPGFLNSSNYRIPALFKTKEGTLIASIDARRQGGADAPNNDIDTAVRRSEDGGKT
WDEGQIIMDYPDKSSVIDTTLIQDDETGRIFLLVTHFPSKYGFWNAGLGSGFKNIDGKEYLC
LYDSSGKEFTVRENVVYDKDGNKTEYTTNALGDLFRNGTKIDNINSSTAPLKAKGTSYINLV
YSDDDGKTWSEPQNINFQVKKDWMKFLGIAPGRGIQIKNGEHKGRIVVPVYYTNEKGKQSSA
VIYSDDSGKNWTIGESPNDNRKLENGKIINSKTLSDDAPQLTECQVVEMPNGQLKLFMRNLS
GYLNIATSFDGGATWDETVEKDTNVLEPYCQLSVINYSQKIDGKDAVIFSNPNARSRSNGTV
RIGLINQVGTYENGEPKYEFDWKYNKLVKPGYYAYSCLTELSNGNIGLLYEGTPSEEMSYIE
MNLKYLESGANK
SEQ ID NO: 72:
MKSKKIIATLVASLVISNMGGYLVKANPNVNHKAVIIEDRQAIIETAIPQSEMTASATSEEG
QDPASSAIDGNTNTMWHTKWNGSDALPQSLSVNLGSSRKVSSIAITPRTSGNNGFITKYEIH
AINNGVEALVAEGTWEENNLVKTVTFDSPIDAEEIKITAIQGVGGFASIAELNVYEIKGEVD
EIANYGNLKITKEEERVNITGDLEKFSSLEEGTIVTRFNMNDTSIQSLIGLSDGNKANNYFS
LYVSGGKVGYELRRQEGNGDFNVHHSADVTFNRGINTLALKIEKGIGAKIFLNGSLVKTVSD
PNIKFLNAINLNSGFIGKTDRANGYNEYLFRGNIDFMNIYDKPVSDNYLLRKTGETKAPLED
SLLPDDVYKTQPVELFYPGYLESRGYRIPALETTKKGTVLASIDVRNNGDHDAPNNNIDVGI
RRKEVNGEWEEGKVILDYPGKSAAIDTSLMSATIEENGIEKERIFLIVTHFPEGYGFPNTEG
GSGYREIDGKYYFILKDAQNNEYTVREDGIVYNSEGNQTDYVMKNDKTLIQNGEEVGNALLS
NSPLKAVGTAHIEMIYSDDDGKTWSEPEDLNPGLKKEWMKFFGTAPGKGIQIKNGEHKDRLI
EPIYYTNQNNFQSSAVIYSDDFGETWKLGESPIDTASVSSETVSSGTQLTECQVVEMPNGQL
KLFMRNTGSYTRIATSFDGGATWHDEVPEDTSLREPYCQLSVINYSGKINGKDAIIFSNPDA
SSRVNGSVKVGLINENGTYDNGEPRYEFDWIYNKTVKPGSFAYSCLTELPDGNLGLFYEGEG
AGRMAYTEFDLNYLKFNASEDSPSASVQSIEVLDEDLAYNSGEEVSIKVNFNQLVSIIGDRK
ITLDIGGVDVPLNMVNYEGKSSAIFKGTIPEGINQGNYEIKLKENNTLELNTVYNKVSTFNG
LDNTGINVQIGELKTTVGNSTIKVNDEVQVGSAFEAILGIEGLNGDTEVYSAEYLFEYNAEA
FILNEITSFNDSLFVKSKEVEPGKVRILVASLGNEIEKDSDLVKVNLTPKISSELEVLGLTT
ALVGAGDGNTHDLELS SKEVKINEEASGEIWNPVQNFEIPEINKKNVKLTWNAPITTEGLE
GYVIYKDGKKLSEVPAESTEFVVSKLNRHTIYNFKVAAKYSNGELSAKESKTIRTAR
SEQ ID NO: 73:
MYSLIKKSISTIALSTLAITSLPTYSVSSQTTEEYGARKYFINNNIENIKNIENKSFDLIQN
LNTKILEKENIETLSGTVVDFTKEATSNSTIPNGLIIEKSNINITAGKGYDLSSEMGSEYVK
ALEKGTIIVSYKSTSNNSIQSLVSIGNNTSGNRDRHFHIYITNTGEVGMELRNTDSVLKYTL
GRPAAVRSIYKNNLVFNTIAFKADPSNKQYKLFANGELLATLNTDVYKFINDITGVNNVMLG
GTVRDGVIAYPFGGTIGNVKIYNEILTDEALKAETGATTYGKNIFYAGDSTKSNYFRIPSLL
SLRSGTVVSAADARYGGTHDSKSNIDIAFSKSLDGGIIWKNPTIPLQFNDYVARNIDWPRDS
IGKNVQIQGSASFIDPVLLEDKETKRLFIFADAMPAGIGSSNASTGSGYKDIAGKKYMKLRW
HQDGSSTYNYSIRENGVIYNDVTNLPTEYKIDGDYNLYKNGIALLYKQYDYNFSGTTLLETA
TNIDVNMNVFYKDSLFKVFPTTYLDMKYSDDEGETWSNLNIVSSFKPENSKFLVLGPGVGKQ
ISKGQYKGRLIVPLYSSSYAELGFMYSDDHGQTWNYVAADNRNTGTTAEAQIVEMPDGSLKS
YLRTGSGVIAEVTSINGGETWSDRVTVPNMHTTSYGTQLSVINYAGLIDGKEAIILSAPDSS
SARRNGKIWIGLISDTGASGINKYSIEWKYCYSVDSSNMGYSYSCLTELPNGDIGLLYEKYD
SWSRNELHLKNILKYETFSINELKQPISN
SEQ ID NO: 74:
MYSSNRTYSRAILGLSAVLTLSFTSLVAPVNAEEPETVVPATAELEGEVAATLPSAETGLLD
AAPPKPVARGAAGDLQLPAVNEKEVFEEGRVIRAPEPDQSRCYRIPALVTAKNGDLLLAFDN
RYGGGDGAKTWCRDAPYENMKRINRQNMQTDIQLYRSVDNGQSFEDFGYIAQGTADVRELSY
TDPALVTDRTTGKIFAFFVRAYDYRVGQSSAGFNEGDVEAEIQKRDVQDTVVVESLDGGQTW
GNMRLLSALTAKVSSISTGDTIFDGRGRFVTSGAGIQLQYGEHAGRLIVPISVDIDPKDSAK
FINLAIYSDDHGQTWQAGIGTAGGAGFSGDVSKIVELSDGRLMMSSKDNDKPRWVSYSEDQG
ENWSTPKRKIIAPPQHPEKHNTGINVGLIRAYPNAPENSAAARVLLYSAPIDQRYSHKHTED
GRNNGWVMGSCDDGKTWSFGRQIEKNRFQYSSMTVMSDGNIGMVYESGDFSTGMNLKFAKFN
MAWLGADCHSNEALGLTGDIDKEIVEAQEKAAEATKEAQEAAEKVQKLTEELAAARKENDEL
KNQVKGFKEAVGDLANEAEDLADKVFKLETAVTEAKEKATVAEKAASDAVTQLQKAESIAEE
QKAKAESAAAEAQALREKLERLEGSILTVKENPEAEEIADLSSTAKDAADAARRAATDANGA
LSGQKQDEEKPAMGLMGILKVLAGIIPLVAIIATIFQTFRLPFNIPGMR
SEQ ID NO: 75:
MTANPYLRRLPRRRAVSFLLAPALAAATVAGASPAQAIAGAPVPPGGEPLYTEQDLAVNGRE
GFPNYRIPALTVTPDGDLLASYDGRPTGIDAPGPNSILQRRSTDGGRTWGEQQVVSAGQTTA
PIKGFSDPSYLVDRETGTIFNFHVYSQRQGFAGSRPGTDPADPNVLHANVATSTDGGLTWSH
RTITADITPDPGWRSRFAASGEGIQLRYGPHAGRLIQQYTIINAAGAFQAVSVYSDDHGRTW
RAGEAVGVGMDENKTVELSDGRVLLNSRDSARSGYRKVAVSTDGGHSYGPVTIDRDLPDPTN
NASIIRAFPDAPAGSARAKVLLFSNAASQTSRSQGTIRMSCDDGQTWPVSKVFQPGSMSYST
LTALPDGTYGLLYEPGTGIRYANFNLAWLGGICAPFTIPDVALEPGQQVTVPVAVTNQSGIA
VPKPSLQLDASPDWQVQGSVEPLMPGRQAKGQVTITVPAGTTPGRYRVGATLRTSAGNASTT
FTVTVGLLDQARMSIADVDSEETAREDGRASNVIDGNPSTFWHTEWSRADAPGYPHRISLDL
GGTHTISGLQYTRRQNSANEQVADYEIYTSLNGTTWDGPVASGRFTTSLAPQRAVFPARDAR
YIRLVALSEQTGHKYAAVAELEVEGQR
SEQ ID NO: 76:
MKKPVFLLSLLALSTSMAVHGNSFWKADLHENLTNVTKRVGVDGFTVNKEGQPWPGIGPNGE
AGGTVTLPYSRIPAMT1TDDNKMVVMFDLRWKTASDONRIDPGAAISEDGGHSWKRITAWNE
NDSKISLRRAMDPTLLFNSFDGSLYVMHGTWAAGTQNWYRDRLSYFNQNIWAATIYKSTDGG
LSWQKNTEFSNTVNRDVFMKVQKGVGNPTIGFLGGVGTGIVMKDGTLVFPIQTAHREGIATT
IMYSKDNGRTWDMPTINNALAPNPSSLENMVFEIDNKLVMTGREDNGKKTRWAYYTEDLGQT
WHVYEPVNGESATTAAPSQGSSIYVTLPIGKRFLLLSKPNGNGNDRYAKGNLALWMLNAKNP
NHKHQVPIIKPGSGNSAGAGYSPLAYKKGNLFIAFENNGDITVKNLSAHMQAIEKKATEWGL
TDEIATEVEKINSLEHLNKGQKETLSAKMRRANDNAVAESNVLNREMHELKDEATSLEQKSV
AMRKALPSKMKQFKRDLGEVRDLTQLTNETYLNYLGIQGLMAMLNGSFLALNTPLDFSKYIK
QGEKLNSYDTDILYSTYNKVFVEYDSVIKNSQHRPTIALGLNTRLTDQTQAGVFYEYENKKQ
KVDAFGVRAQYTKGDNVLAPFLRYRTVKHDDVIDRNHNVDLYINYAKNVNIDPHLTLSPFVG
AYTSLSSRTLLDEDVAVNKRLVMAGDVGLDIRYRLADISVSIRPNIAFIKDGFTFSQAIYRD
NPF
SEQ ID NO: 77:
MMKKFNPSVLALSISSLLLTSTLTFGQIQQQDKAHFGVKEHQESLLFHQSLVKQGSDNVPIW
RIPSLLRTKDGVLIAAADKRWQHRGDWGDIDTAIRISHDDGKTWGNITTILDLPSQNGEKSP
IRDDAPTFNPWAHRNNSSVATYRNSAFLIDAQMVQDKRNGRIFLAVDMFPESTGLSGPSDNG
VTEFGSGYVNIDGKQYLRLNKKEGYTSKQVVTLRENGIVFNEKNEKTGYRWINGDPKKNFKD
LGDVYDQDNNKLGNIYLKQTERNATVPFIAPNTSYFWLTHSDDNGKTWSSPIDLTSQVKKDW
MRFFGTGPGVGIQTKKGNLLFPIYYINRHGKQSSALIISKDGGKTWDLGQSPNDTRTELYGK
NSETLNSNSSGHELTESQLVELQNGDLKLFMRNTSGRVMMSTSKDGGYSWIETKQVPELNHG
YSQLSVIKYSKKINGKEYIVFSGQSVSGNSGDKLRRDGKLFLGEVQDNGDINWDTTNLVRNI
KSSGLAKQGSEVYPNGYVYSSMAELGDGSIGLAYENTTDYTTIMYLPIEMQEFFWKAGKIFS
DVRQKEPLVFTYDGTETLEKIGDGIAIKRGEGESQSGINVSEGLLVLDQQTKDGKNKAFTQL
TLNNSGVAQVNSTQNIDRFVVNNGATGYLQFTVTDTHSPRLKINQDVTAHGQIVAVQVNLQK
KLKPNDKGYYHAQGEELIAFKDNGQVKWRLVNDELKDGMYVYTLASVAKPSGLRTPSQPHSL
YLTNKLITADGKAVSTVAPLKAPLTVNARPQVNPVLASYLTANLALNKMSEQLQQSFMHETR
LLQEKDRSIFVKYLNGKQKYGSNLSFYDYGYDFNASYSGVMLGGKVWQSERGNHALYTALNK
TSYKVTPKAVDGETKAKYQSWGGSINWHSNLPHNLIVDLSTGYQKHKGDIEHAGHVKGYTFN
IGADLGYRYQWMKNAFITPMVGLHYLYASLSDVNDQANKALLKYNNFNALKTNLGVDVNYRI
GKFEVKGLLSYDMYQQKTRQLYVDDVAYKQGKLADTLHLNTQFVAHLTPRFAFSTEVGFQHA
RNKGQSSFAVGAHYQF
SEQ ID NO: 78:
MNTYFDIPHRLVGKALYESYYDHFGQMDILSDGSLYLIYRRATEHVGGSDGRVVFSKLEGGI
WSAPTIVAQAGGQDFRDVAGGTMPSGRIVAASTVYETGEVKVYVSDDSGVTWVHKFTLARGG
ADYNFAHGKSFQVGARYVIPLYAATGVNYELKWLESSDGGETWGEGSTIYSGNTPYNETSYL
PVGDGVILAVARVGSGAGGALRQFISLDDGGTWTDQGNVTAQNGDSTDILVAPSLSYIYSEG
GTPHVVLLYTNRTTHFCYYRTILLAKAVAGSSGWTERVPVYSAPAASGYTSQVVLGGRRILG
NLFRETSSTTSGAYQFEVYLGGVPDFESDWFSVSSNSLYTLSHGLQRSPRRVVVEFARSSSP
STWNIVMPSYFNDGGHKGSGAQVEVGSLNIRLGTGAAVWGTGYFGGIDNSATTRFATGYYRV
RAWI
SEQ ID NO: 79:
MTRHLLNCRILYMHPPLDMHTHPFIKEGKSMTVEKSVVFKAEGEHFTDQKGNTIVGSGSGGT
TKYFRIPAMCTTSKGTIVVFADARHNTASDQSFIDTAAARSTDGGKTWNKKIAIYNDRVNSK
LSRVMDPTCIVANIQGRETILVMVGKWNNNDKTWGAYRDKAPDTDWDLVLYKSTDDGVTFSK
VETNIHDIVTKNGTISAMLGGVGSGLQLNDGKLVFPVQMVRTKNITTVLNTSFIYSTDGITW
SLPSGYCEGFGSENNIIEFNASLVNNIRNSGLRRSFETKDFGKTWTEFPPMDKKVDNRNHGV
QGSTITIPSGNKLVAAHSSAQNKNNDYTRSDISLYAHNLYSGEVKLIDDFYPKVGNASGAGY
SCLSYRKNVDKETLYVVYEANGSIEFQDLSRHLPVIKSYN
SEQ ID NO: 80:
MNKRGLYSKLGISVVGISLLMGVPTLIHANELNYGQLSISPIFQGGSYQLNNKSIDISSLLL
DKLSGESQTVVMKFKADKPNSLQALFGLSNSKAGFKNNYFSIFMRDSGEIGVEIRDAQEGIN
YLFSRPASLWGKHKGQAVENTLVFVSDSKDKTYTMYVNGIEVFSETVDTFLPISNINGIDKA
TLGAVNREGKEHYLAKGSIGEISLFNKAISDQEVSNIPLSNPFQLIFQSGDSTQANYFRIPT
LYTLSSGRVLSSIDARYGGTHDSKSKINIATSYSDDNGKTWSEPIFAMKFNDYEEQLVYWPR
DNKLKNSQISGSASFIDSSIVEDKKSGKTILLADVMPAGIGNNNANKADSGFKEINGHYYLK
LKKNGDNDFRYTVRENGVVYDETTNKPTNYTINDKYEVLEGGKSLTVEQYSVDFDSGSLRER
HNGKQVPMNVFYKDSLFKVTPTNYIAMTTSQNRGESWEQFKLLPPFLGEKHNGTYLCPGQGL
ALKSSNRLIFATYTSGELTYLISDDSGQTWKKSSASIPFKNATAEAQMVELRDGVIRTFFRT
TTGKIAYMTSRDSGETWSKVSYIDGIQQTSYGTQVSAIKYSQLIDGKEAVILSTPNSRSGRK
GGQLVVGLVNKEDDSIDWRYHYDIDLPSYGYAYSAITELPNHHIGVLFEKYDSWSRNELHLS
NVVQYIDLEINDLTK
SEQ ID NO: 81:
MKKFFWIIGLFISMQMTRAADSVYVQNPQIPILIDRTDNVLFRIRIPDATKGDVLNRLTIRF
GNEDKLSEVKAVRLFYAGTEAATKGRSRFAPVTYVSSHNIRNTRSANPSYSVRQDEVTTAAN
TLTLKTRQPMVKGINYFWVSVEMDRNTSLLSKLTPTVTEAVINDKPAVIAGEQAAVRRMGIG
VRHAGDDGSASFRIPGLVTTNEGTLLGVYDVRYNNSVDLQEHVDVGLSRSTDKGQTWEPMRI
AMSFGETDGLPSGQNGVGDPSILVDERTNTVWVVAAWTHGMGNARAWTNSMPGMTPDETAQL
MMVKSTDDGRTWSEPINITSQVKNPSWCFLLQGPGRGITMRDGTLVFPIQFIDSLRVPHAGI
MYSKDRGETWHIHQPARTNTTEAQVAEVEPGVLMLNMRDNRGGSRAVSITRDLGKSWTEHSS
NRSALPESICMASLISVKAKDNIIGKDLLLFSNPNTTEGRHHITIKASLDGGVTWLPAHQVL
LDEEDGWGYSCLSMIDRETVGIFYESSVAHMTFQAVKIKDLIR
SEQ ID NO: 82:
MSIKMTSQRRRASIHKETDSNIKGVDMRFKNVKKTALMLAMFGMATSSNAALFDYNATGDTE
FDSPAKQGWMQDNTNNGSGVLTNADGMPAWLVQGIGGRAQWTYSLSTNQHAQASSFGWRMTT
EMKVLSGGMITNYYANGTQRVLPIISLDSSGNLVVEFEGQTGRTVLATGTAATEYHKFELVF
LPGSNPSASFYFDGKLIRDNIQPTASKQNMIVWGNGSSNTDGVAAYRDIKFEIQGDVIFRGP
DRIPSIVASSVTPGVVTAFAEKRVGGGDPGALSNTNDIITRTSRDGGITWDTELNLTEQINV
SDEFDFSDPRPIYDPSSNTVLVSYARWPTDAAQNGDRIKPWMPNGIFYSVYDVASGNWQAPI
DVTDQVKERSFQIAGWGGSELYRRNTSLNSQQDWQSNAKIRIVDGAANQIQVADGSRKYVVT
LSIDESGGLVANLNGVSAPIILQSEHAKVHSFHDYELQYSALNHTTTLFVDGQQITTWAGEV
SQENNIQFGNADAQIDGRLHVQKIVLTQQGHNLVEFDAFYLAQQTPEVEKDLEKLGWTKIKT
GNTMSLYGNASVNPGPGHGITLTRQQNISGSQNGRLIYPAIVLDRFFLNVMSIYSDDGGSNW
QTGSTLPIPFRWKSSSILETLEPSEADMVELQNGDLLLTARLDFNQIVNGVNYSPRQQFLSK
DGGITWSLLEANNANVFSNISTGTVDASITRFEQSDGSHFLLFTNPQGNPAGTNGRQNLGLW
FSFDEGVTWKGPIQLVNGASAYSDIYQLDSENAIVIVETDNSNMRILRMPITLLKQKLTLSQ
N
SEQ ID NO: 83
MYSSNRTYSRAILGLSAVLTLSFTSLVAPVNAEEPETVVPATAELEGEVAATLPSAETGLLD
AAPPKPVARGAAGDLQLPAVNEKEVFEEGRVIRAPEPDQSRCYRIPALVTAKNGDLLLAFDN
RYGGGDGAKTWCRDAPYENMKRINRQNMQTDIQLYRSVDNGQSFEDFGYIAQGTADVRELSY
TDPALVTDRTTGKIFAFFVRAYDYRVGQSSAGFNEGDVEAEIQKRDVQDTVVVESLDGGQTW
GNMRLLSALTAKVSSISTGDTIFDGRGRFVTSGAGIQLQYGEHAGRLIVPISVDIDPKDSAK
FINLAIYSDDHGQTWQAGIGTAGGAGFSGDVSKIVELSDGRLMMSSKDNDKPRWVSYSEDQG
ENWSTPKRKIIAPPQHPEKHNTGINVGLIRAYPNAPENSAAARVLLYSAPIDQRYSHKHTED
GRNNGWVMGSCDDGKTWSFGRQIEKNRFQYSSMTVMSDGNIGMVYESGDFSTGMNLKFAKFN
MAWLGADCHSNEALGLTGDIDKEIVEAQEKAAEATKEAQEAAEKVQKLTEELAAARKENDEL
KNQVKGFKEAVGDLANEAEDLADKVFKLETAVTEAKEKATVAEKAASDAVTQLQKAESIAEE
QKAKAESAAAEAQALREKLERLEGSILTVKENPEAEEIADLSSTAKDAADAARRAATDANGA
LSGQKQDEEKPAMGLMGILKVLAGIIPLVAIIATIFQTFRLPFNIPGMR
SEQ ID NO: 84
MSRRKAVFSALGAAALIGAALPTIPTAQAQTPTGYGFDATASIGEEPEFSTQQLADGGTLGF
DCYRIPSLGVAPNGNVLASWDGRPNNCSDAPQPNSIVGKVSTDNGATWGEQHDISAGITAEP
KTGYSDPSIVVDWERGDVFNFHVKSFDAGYFTSQPGTDPDDRNVAHVAYAKSSDNGSTWVAD
TVITDQVVADDTWDSRFATSGNGIQLQYGAYKGRLVQPSVTRMTNGRVAAVAMLSDDHGTTW
YPSAPWGNSMDENKIVELSDGTLMNNSRSSGADTYRKVSYSTDGGVTWTEPTLDTQLPDPRN
NASLIRVFPTAPEGSAQAKVLLFSNTATTSGRTNGTVRMSCDDGQTWPVSKVFEPGAIQYTS
MATLPNGDIGMLWENSGSNIDIFYSQFNLSWLEAGCIGVDADETPVTAGETTTMNVTLTNPF
ANAIFDRAVSLELPEGWQAEDVRVSIPSGESVTIPVQVTAPLVADNGELPVEVSILDGADRY
TGRLNLTVQGGQEPASTSVKVSIPNLKDTYVAGEKISINFAVNNPFDVTVNSVPSLGEGENW
MPANLRGFDPEQGAPNCRYRNLGANQSYNCTTTTYEVSDSDVERGYVDIPTVWTFTNSAGET
VWSKNVDVPRIELNGTQDAVTDAIVTVDPINPVHSNGQSQTVEVQANVTSEGDLPAGSKVAF
YLDSSPIDAAAVDAEGHASISIDVDNIASEQPERTFEVRARLVVPEDAPRSIARDALARFTV
LPEQVQQNSLVIMNHPDVVSDGQTKTIVIAVKATAHDGSPVAIGTLITFRVNGIERDVVPTN
AQGTAKLQLDLKPVNTEDEEYEVTVEAELDELTAQTTFKVLAGEEEEPTSTEEQPSETEQPS
EPEEEPTAPTGSSNGGSFAALLALLAALGGIVGAVLGLLKL
SEQ ID NO: 85
MNYKGITLILTAAMVISGGNYVLVKGSTLDSGKNNSGYEIKVNNSESLSSLGEYKDINLESS
NASNITYDLEKYKNLDEGTIVVRFNSKDSKIQSLLGISNSKTKNGYFNFYVTNSRVGFELRN
QKNEGNTQSGTENLVHMYKDVALNDGDNTVALKIEKNKGYKLFLNGKIIKEVKDTNTKFLNN
IENLDSAFIGKTNRYGQSNEYNFKGNIGFMNIYNEPLGDDYLLSKTGETKAKEEVLVEGAVK
TEPVDLFHPGFLNSSNYRIPALFKTKEGTLIASIDARRHGGADAPNNDIDTAVRRSEDGGKT
WDEGQIIMDYPDKSSVIDTTLIQDDETGRIFLLVTHFPSKYGFWNAGLGSGFKNIDGKEYLC
LYDSSGKEFTVRENVVYDKDGNKTEYTTNALGDLFKNGTKIDNINSSTAPLKAKGTSYINLV
YSDDDGKTWSEPQNINFQVKKDVVMKFLGIAPGRGIQIKNGEHKGRIWPVYYTNEKGKQSSA
VIYSDDSGKNWTIGESPNDNRKLENGKIINSKTLSDDAPQLTECQVVEMPNGQLKLFMRNLS
GYLNIATSFDGGDTWDETVEKDTNVLEPYCQLSVINYSQKVDGKDAVIFSNPNSRSRSNGTV
RIGLINQVGTYENGEPKYEFDWKYNKLVKPGYYAYSCLTELSNGNIGLLYEGTPSEEMSYTE
MNLKYLESGANK
SEQ ID NO: 86:
MKKAVILFSLFCFLCAIPVVQAADTIFVRETRIPILIERQDNVLFYLRLDAKESQTLNDVVL
NLGEGVNLSEIQSIKLYYGGTEALQDSGKKRFAPVGYISSNTPGKTLAANPSYSIKKSEVTN
PGNQVVLKGDQKLFPGINYFWISLQMKPGTSLTSKVTADIASITLDGKKALLDVVSENGIEH
RMGVGVRHAGADNSAAFRIPGLVTTNKGTLLGVYDVRYNSSVDLQEHVDVGLSRSTDGGKTW
EKMRLPLAFGEFGGLPAGQNGVGDPSILVDTKTNNVWVVAAWTHGMGNQRAWWSSHPGMDMN
HTAQLVLAKSTDDGKTWSAPINITEQVKDPSWYFLLQGPGRGITMSDGTLVFPTQFIDSTRV
PNAGIMYSKDGGKNWKMHNYARTNTTEAQVAEVEPGVLMLNMRDNRGGSRAVAITKDLGKTW
TEHESSRKALPESVCMASLISVKAKDNVLGKDLLIFSNPNTTKGRYNTTIKISLDGGVTWSP
EHQLLLDEGNNWGYSCLSMIDKETIGILYESSVAHMTFQAVKLKDIIK
SEQ ID NO: 87:
GEPLYTEQDLAVNGREGFPNYRIPALTVTPDGDLLASYDGRPTGIDAPGPNSILQRRSTDGG
RTWGEQQVVSAGQTTAPIKGFSDPSYLVDRETGTIFNFHVYSQRQGFAGSRPGTDPADPNVL
HANVATSTDGGLTWSHRTITADITPDPGWRSRFAASGEGIQLRYGPHAGRLIQQYTIINAAG
AFQAVSVYSDDHGRTWRAGEAVGVGMDANKTVELSDGRVLLNSRDSARSGYRKVAVSTDGGH
SYGPVTIDRDLPDPTNNASIIRAFPDAPAGSARAKVLLFSNAASQTSRSQGTIRMSCDDGQT
WPVSKVFQPGSMSYSTLTALPDGTYGLLYEPGTGIRYANFNLAWLGGICAPFTIPDVALEPG
QQVTVPVAVTNQSGIAVPKPSLQLDASPDWQVQGSVEPLMPGRQAKGQVTITVPAGTTPGRY
RVGATLRTSAGNASTTFTVTVGLLDQARMSIADVDSEETAREDGRASNVIDGNPSTFWHTEW
SRADAPGYPHRISLDLGGTHTISGLQYTRRQNSANEQVADYEIYTSLNGTTWDGPVASGRFT
TSLAPQRAVFPARDARYIRLVALSEQTGHKYAAVAELEVEGQR
SEQ ID NO: 88:
MSYFRNRDIDIERNSMNRSVQERKCRYSIRKLSVGAVSMIVGAVVFGTSPVLAQEGASEQPL
ANETQLSGESSTLTDTEKSQPSSETELSGNKQEQERKDKQEEKIPRDYYARDLENVETVIEK
EDVETNASNGQRVDLSSELDKLKKLENATVHMEFKPDAKAPAFYNLFSVSSATKKDEYFTMA
VYNNTATLEGRGSDGKQFYNNYNDAPLKVKPGQWNSVTFTVEKPTAELPKGRVRLYVNGVLS
RTSLRSGNFIKDMPDVTHVQIGATKRANNTVWGSNLQIRNLTVYNRALTPEEVQKRSQLFKR
SDLEKKLPEGAALTEKTDIFESGRNGKPNKDGIKSYRIPALLKTDKGTLIAGADERRLHSSD
WGDIGMVIRRSEDNGKTWGDRVTITNLRDNPKASDPSIGSPVNIDMVLVQDPETKRIFSIYD
MFPEGKGIFGMSSQKEEAYKKIDGKTYQILYREGEKGAYTIRENGTVYTPDGKATDYRVVVD
PVKPAYSDKGDLYKGNQLLGNIYFTTNKTSPFRIAKDSYLWMSYSDDDGKTWSAPQDITPMV
KADWMKFLGVGPGTGIVLRNGPHKGRILIPVYTTNNVSHLNGSQSSRIIYSDDHGKTWHAGE
AVNDNRQVDGQKIHSSTMNNRRAQNTESTVVQLNNGDVKLFMRGLTGDLQVATSKDGGVTWE
KDIKRYPQVKDVYVQMSAIHTMHEGKEYIILSNAGGPKRENGMVHLARVEENGELTWLKHNP
IQKGEFAYNSLQELGNGEYGILYEHTEKGQNAYTLSFRKFNWDFLSKDLISPTEAKVKRTRE
MGKGVIGLEFDSEVLVNKAPTLQLANGKTARFMTQYDTKTLLFTVDSEDMGQKVTGLAEGAI
ESMHNLPVSVAGTKLSNGMNGSEAAVHEVPEYTGPLGTSGEEPAPTVEKPEYTGPLGTSGEE
PAPTVEKPEYTGPLGTAGEEAAPTVEKPEFTGGVNGTEPAVHEIAEYKGSDSLVTLTTKEDY
TYKAPLAQQALPETGNKESDLLASLGLTAFFLGLFTLGKKREQ
SEQ ID NO: 89:
MPPRTRRPFWLALATSCALAVSSPFPAHARPGDRAPAFGEQVLFDAARDPGGYACFRIPAIV
RTTDGTLLAFAEGRVLDCADDGDIDIVLRRSLDGGRTWGPLRVVNDGGGDTHGNPAPVVDRA
TGRVLLLETYNAGRTDSADCAVPCARVPHVQHSDDGGRTWSAPRDLSPEILPPDWNSWYATG
PVHGVQLTGGAHPGRLVVGVNAETWDGERSEMGVPPAGGWGRVTANHAALVVSDDGGEHWRT
GATDTWPVAADGTFRQKPSELTLTERADGALLVSGREENGTDPGHRTQALSRDGGDSFAAPF
RALPDLYAPQVQGAVLRLGNRILLSAPADPDRRRTMTVRSSRDGGATWDSADRGTVVTRDWA
GYSDLVTVDDDTVGLLYEGGKTDARDEIRFARLTADRLAPPRGPDPTTPDLAANAAPAAVLG
GAAPTTDGAVGGALAFDGADDAVRLPYDGRLALGEGDFTASLWFRYSAADGEQPLLWMGGIG
TTQPQVWLRAEPDAGRVQGLITARDGATAPRSAWVRTDRAYDDGRWHRLTLRRGGGRLTLFV
DGSAAADAADVPGSVSRNSPFGVHIGERMDGRARFTGAVDDVQVWNSALTDTEIAAGVPPAA
GRSTVLHLPLDRVDEAAADTGGSTDTGG
SEQ ID NO: 90:
MQRRVTVAMLSAALLVATTAGTAHGAPAAAPGELTSQDIATQGVGSPHYRIPALTTSVRGTL
IAAYDTRPTLGDLPGNLGVVVRRSTDGGATWESQQVVRKEAAPKGFGDPSLLVDRTTGRIFV
FYAGSVNQGFFGSGTGNDESDPNILQADYSYSDDDGVTWTHRRITKQIKNPAWAGMFAASGE
GIQVRHGAYEGRLIQQYAIRNNGANYAVSAYSDDHGATWKTGTPVGPGGDENKTVELSDGRI
MLNNRSAPYRTVAYSSDGGVTYTPFVQDTDLPDPANNGSVIRYAPDVPASHPQASWLLFSNT
DSTARKNLTVKMSCDNGRTWPIRKVVDPGSAAYSTLTRLPDGRLGLLYERADYRHITYSSFD
LKWLGGTCADITITPPATLRAGTTAEVTVRVVNRMDVTRSAGTLDLAVPAGWSTRQVAFPAV
RPGQGANIKVPVTIPAGATGDARLTVTYKADGKQASGSRSVTVTP
SEQ ID NO: 91:
MTLTTKLSALTTAGIMVVIGVPMVTQSAMASGRAPAPVAATTQPKLVTGDITSTDQSGTNLF
FGKKIVRNARGAIMKVDRTWPAAVPAPLPDVRADSSTRMLLGPVVDLAVNEHPEGVFYRIPA
LATASNGDLLASYDLRPGSAGDAPNPNSIVQRRSRDNGRTWGPQTVIHAGTPGRRKVGYSDP
SYLVDPATGRILNFHVKSYDRGFATSEVGTDPDDRHVLHAEVSTSTDNGHTWTHRDITREIT
PDPTTRTRFVASGQGIALLHGPHAGRLIAQMTVRNSVGQQAQSIYSDDHGITWHAGNPVGRM
MDENKVVELSDGTLMLNSRDAARSGRRKVAYSQDGGLTWGPVKLVDDLIDPTNNAQIIRAYP
NARAGSAKARILLFTNARNATERVNGTLSVSCDDGRTWVSHQTYMPGEVGYTTAAVQSDGAL
GVLWERDGIRYSTIPMGWLNSVCPLAPSGRPTSGKPTSGTSLPPTATPSGSLHGGASSRPTS
LPHTGD
SEQ ID NO: 92:
MGRIGKKAMAIALVSAVMVTPLNVCATVENQEQQQVTQGAEDIAVIDDAQETVAADEAQADE
AAAITVEGRETAEESSASIPEGILMEKNNVDIAEGQGYSLDQEAGAKYVKAMTQGTIILSYK
STSENGIQSLFSVGNSTAGNQDRHFHIYITNSGGIGIELRNTDGVFNYTLDRPASVRALYKG
ERVFNTVALKADAANKQCRLFANGELLATLDKDAFKFISDITGVDNVTLGGTKRQGKIAYPF
GGTIGDIKVYSNALSDEELIQATGVTTYGENIFYAGDVTESNYFRIPSLLTLSTGTVISAAD
ARYGGTHDSKSKINIAFAKSTDGGNTWSEPTLPLKFDDYIAKNIDWPRDSVGKNVQIQGSAS
YIDPVLLEDKLTKRIFLFADLMPAGIGSSNASVGSGFKEVNGKKYLKLRWHKDAGRAYDYTI
REKGVIYNDATNQPTEFRVDGEYNLYQHDTNLTCKQYDYNFSGNNLIESKTDVDVNMNIFYK
NSVFKAFPTNYLAMRYSDDEGASWSDLDIVSSFKPEVSKFLVVGPGIGKQISTGENAGRLLV
PLYSKSSAELGFMYSDDHGDNWTYVEADNLTGGATAEAQIVEMPDGSLKTYLRTGSNCIAEV
TSIDGGETWSDRVPLQGISTTSYGTQLSVINYSQPIDGKPAIILSSPNATNGRKNGKIWIGL
VNDTGNTGIDKYSVEWKYSYAVDTPQMGYSYSCLAELPDGQVGLLYEKYDSWSRNELHLKDI
LKFEKYSISELTGQASGN
SEQ ID NO: 93:
MLAPGSSRVELFKRQSSKVPFEKDGKVTERVVHSFRLPALVNVDGVMVAIADARYETSNDNS
LIDTVAKYSVDDGETWETQIAIKNSRASSVSRVVDPTVIVKGNKLYVLVGSYNSSRSYWTSH
GDARDWDILLAVGEVTKSTAGGKITASIKWGSPVSLKEFFPAEMEGMHTNQFLGGAGVAIVA
SNGNLVYPVQVTNKKKQVFSKIFYSEDDGKTWKFGEGRSAFGCSEAVALEWEGKLIINTRVD
YRRRLVYESSDMGNTWLEAVGTLSRVWGPSPKSNQPGSQSSFTAVTIEGMRVMLFTHPLNFK
GRWLRDRLNLWLTDNQRIYNVGQVSIGDENSAHSSVLYKDDKLYCLHEINSNEVYSLVFARL
VGELRIIKSVLQSWKNWDSHLSSICTPADPAASSSERGCGPAVTTVGLVGFLSHSATKTEWE
DAYRCVNASTANAERVPNGLKFAGVGGGALWPVSQQGQNQRYRFANHAFTVVASVTIHEVPS
VASPLLGASLDSSGGKKLLGLSYDEKHQWQPIYGSTPVTPTGSWETGKRYHVVLTMANKIGS
VYIDGEPLQGSGQTVVPDERTPDISHFYVGGYKRSDMPTISHVTVNNVLLYNRQLNAEEIRT
LFLSQDLIGTEAHMDSSSDTSA
SEQ ID NO: 94:
MRLSLNKLMGLGLLCALGLSIPSVLGKESFEQARRGKFTTLSTKYGLMSCRNGVAEIGGGGK
SGEASLRMFGGQDAELKLDLKDTPSREVRLSAWAERWTGQAPFEFSIVAIGPNGEKKIYDGK
DIRTGGFHTKIETSVPAGTRSLVFRLTSPENKGMKLDDLFLVPCIPMKVNPQVEMSSSAYPV
MVRIPCSPVLSLNVRTDGCLNPQFLTAVNLDFTGTTKLSDIESVAVIRGEEAPIIHHGEEPF
PKDSSQVLGTVKLAGSARPQISVKGKMELEPGDNYLWACVTMKEGATLDGRVVVRPASVVAD
NKPVRVANAAPVVQRIGVAVVRHGDFKSKFYRIPGLARSRKGTLLAVYDIRYNHSGDLPANI
DVGVSRSTDGGRTWSDVKIAIDDSKIAPSLGATRGVGDPAILVDEKTGRIWVAAIWSHRHSI
WGSKSGDNSPEACGQLVLAYSDNDGLTWSRPINITEQTKNKDWRILFNGPGNGICMKDGTLV
FAAQYWDGKGVPWSTIVYSKDRGKTWHCGTGVNQQTTEAQVIELEDGSVMINARCNWGGSRI
VGVTKDLGQTWEKHPTNRTAQLKEPVCQGSLLAVDGVPGAGRVVLFSNPNTTSGRSHMTLKA
STNDAGSWPEDKWLLYDARKGWGYSCLAPVDKNHVGVLYESQGALNFLKIPYKDVLNAKNAR
SEQ ID NO: 95:
MKKAVILFSLFCFLCAIPVVQAADTIFVRETRIPILIERQDNVLFYLRLDAKESQTLNDVVL
NLGEGVNLSEIQSIKLYYGGTEALQDSGKKRFAPVGYISSNTPGKTLAANPSYSIKKSEVTN
PGNQVVLKGDQKLFPGINYFWISLQMKPGTSLTSKVTADIASITLDGKKALLDVVSENGIEH
RMGVGVRHAGDDNSAAFRIPGLVTTNKGTLLGVYDVRYNSSVDLQEHVDVGLSRSTDGGKTW
EKMRLPLAFGEFGGLPAGQNGVGDPSILVDTKTNNVWVVAAWTHGMGNQRAWWSSHPGMDMN
HTAQLVLAKSTDDGKTWSAPINITEQVKDPSWYFLLQGPGRGITMSDGTLVFPTQFIDSTRV
PNAGIMYSKDGGKNWKMHNYARTNTTEAQVAEVEPGVLMLNMRDNRGGSRAVAITKDLGKTW
TEHESSRKALPESVCMASLISVKAKDNVLGKDLLIFSNPNTTKGRYNTTIKISLDGGVTWSP
EHQLLLDEGNNWGYSCLSMIDKETIGILYESSVAHMTFQAVKLKDIIK
SEQ ID NO: 96:
MKRNHYLFTLILLLGCSIFVKASDTVFVHQTQIPILIERQDNVLFYFRLDAKESRMMDEIVL
DFGKSVNLSDVQAVKLYYGGTEALQDKGKKRFAPVDYISSHRPGNTLAAIPSYSIKCAEALQ
PSAKVVLKSHYKLFPGINFFWISLQMKPETSLFTKISSELQSVKIDGKEAICEERSPKDIIH
RMAVGVRHAGDDGSASFRIPGLVTSNKGTLLGVYDVRYNSSVDLQEYVDVGLSRSTDGGKTW
EKMRLPLSFGEYDGLPAAQNGVGDPSILVDTQTNTIWVVAAWTHGMGNQRAWWSSHPGMDLY
QTAQLVMAKSTDDGKTWSKPINITEQVKDPSWYFLLQGPGRGITMSDGTLVFPTQFIDSTRV
PNAGIMYSKDRGKTWKMHNMARTNTTEAQVVETEPGVLMLNMRDNRGGSRAVAITKDLGKTW
TEHPSSRKALQEPVCMASLIHVEAEDNVLDKDILLFSNPNTTRGRNHITIKASLDDGLTWLP
EHQLMLDEGEGWGYSCLTMIDRETIGILYESSAAHMTFQAVKLKDLIR
SEQ ID NO: 97:
MKKQVLLIILLALPLWLKAADVSVTGLRTEQMVDPMGLDTAAPRMSWRLESSQRNVMQTAYR
ILVASSPELLAQDKGDLWDSGKVESDASVWIPYQGKRLKSNQRVYWKVRSYTNRGETEWSEP
ARWGMGPLGEIHWKGRWIGWDAAFAWDREDSHSRLSSRYLRTEFKTQAKEIKYATLHLCGLG
MYELFINGQRIGDQVLAPAPSDYRRTVLYNSFDVTKQVAGGNADNAIGVTLGNGRFYTMRQN
YKPYKIPTFGYPKLRLNLIIEYTDGSIQRINSDEKWRLTAQGPIRSNNEYDGEIYDARMELG
NWTQPGYDDSKWLKAQRVSLPYGTLRGNTAPNMKVMKTLKPAVFKQYGDRYMIDFGQNMAGW
VRINIAKAAAGDTICIRYAERVKNDGTELDVENLRHSQSTDYYICNGKENNTSWSSRFSYHG
FQYVEVTGYKNLKAEDLVAEVVYDDLEDNGTFECSNDIMNRVYRNAWWGISSNYKGVPLDCP
QRDERQPWLGDHAMGTWGESFMFNNGNTYAKWADDIREAQREDGCIPDICPAYYNYYTSEMT
WSSTFPVICDMVYEQFGNIEPIRKNYAAIKKWMHHIRSEFTTEDGVINADKYGDWCMPPESP
ELIHSQDPARKTDGALIATAYYYKVSQMLAKFARLQGLEDEAKGFEKDAAKIKDCFNARFLT
VKKGTSPVQTPHVLYPDSIFYGNNTVTANILPLAFDMVPEAYREEVEKNVITGIITRNKGHI
SSGVIGMNWMMRELTRMGRGDVAFLLASNKTYPSYGYMIEKGATAIWELWNGDTANRWMNSC
NHVMILGDLLTWYFRDLAGFNPAQPAYKQIILKPDFSIQELSYVKASHNTLYGKMISNWKKT
LTHLEWDITVPCNTTALVYLPTLDEKAVKDKDVTFVRREGNSTVWSVPSGNYHFSVSMDPSS
GKNRAGIVEDQFLYEQASFPECHGATIVELKNGDLVASFFGGTKERNPDCCIWVCRKPKGAT
EWSAPYLAADGVFSLDDPQAVLAGITAESTPADAGPVVSTFKGDKSRARRKACWNPVLFQIP
GGDLILFYKIGLKVADWSGWLVRSKDGGKTWSQREPLPKGFLGPIKNKPEYVDGRIICPSST
EGDGGWRIHFEISDDKGKTWKMVGPVEAEMSVPTALRKANAANVDDQEGGEAIKGEGEKPIY
AIQPSILRHKDGRLQVLCRTRNAQIATSWSSDNGETWSKVTLLDVPNNNSGTDAVTMKDGRH
VLIYNDFSTLPGTPKGPRTPLCVAVSDDGIHWKNVMTLEDSPISQYSYPSIIQGKDGKLHAV
YTWRRQRVAYKELDLSKLK
SEQ ID NO: 98:
MVRSTKPSLLRRFGALVAAAAMLVVLPAGVSTASAASDDADMLTVTMTRTDALGDEVYVGDT
LTYSFTNTNNTSSAFTAFPAESNLSGVLTTGTPNCRYENLAGGASYPCSTASHTITADDLTA
GSFTPRTVWKATSDRGGTQVLQDNIVSTGDTVTVKEGKRPDPATIPTDRADGEAVRLATARQ
NLGTECYRIPALAEAPNGWILAAFDQRPNTAMANGSGVKCWDAPQPNSIVQRISKDGGKSWT
PIQYVAQGKNAPERYGYSDPSYVVDKETGEIFLFFVHSYNKGFADSQLGVDESNRNVLHAVV
VSSKDNGETWSKPRDITADITKGYENEWKSRFATSGAGIQLKYGKYKGRLIQQYAVGRTTGS
NAAVSVYSDDHGKTWQAGNPVTGMLMDENKVVELSDGRVMLNSRPGAAGYRRVAISEDGGVN
YGTVKNETQLPDPNNNAHITRAFPNAPEGSAKAKVLLYSSPRANNEGRANGVVRISLDDGTT
WSSGKLYKEGSMAYSVITALSDAAGGGYGLLYEGAWVTGGGIDSHDIMYTHISMDWLGYLSA
TADDVTASVEEGASTVDVTVPVSNVGSVDYTGVTVTPADLPTGWSASPVNVGALASGTSKDV
TVTVNVPATAKKDDVAKIVLRVTGTSAANANATTGFDGSITVNVTEKSEPDPEPEPTITGVS
AVTSQAGVKVGDVFDASKVSVTAAMSDGSSKALAAGEYSLSAVDADGKAVDLAEPFAAAGVV
TVTVSVPVEGAGPLTASFTIDVAEKSVDPEPKPEPEPKPEPEKPAGPKVDVPTEQPGLSKTG
ASTAGMSIVFVLLALSGIAALSLRRRSVH
SEQ ID NO: 99:
MCNKNNTFEKNLDISHKPEPLILFNKDNNIWNSKYFRIPNIQLLNDGTILTFSDIRYNGPDD
HAYIDIASARSTDFGKTWSYNIAMKNNRIDSTYSRVMDSTTVITNTGRIILIAGSWNTNGNW
AMTTSTRRSDWSVQMIYSDDNGLTWSNKIDLTKDSSKVKNQPSNTIGWLGGVGSGIVMDDGT
IVMPAQISLRENNENNYYSLIIYSKDNGETWTMGNKVPNSNTSENMVIELDGALIMSTRYDY
SGYRAAYISHDLGTTWEIYEPLNGKILTGKGSGCQGSFIKATTSNGHRIGLISAPKNTKGEY
IRDNIAVYMIDFDDLSKGVQEICIPYPEDGNKLGGGYSCLSFKNNHLGIVYEANGNIEYQDL
TPYYSLINKQ
SEQ ID NO: 100:
MCNKNNTFEKNLDISHHPEPLILFNKDANIWNSKYFRIPNVQLLNDVTILTFSDIRYNAPDD
HAYIDIASARSTDFGKTWSYNIAMKNNRIDSTYSRAMDSTTLITNTVRIILIASSWNTNGNW
AMTTSARRSDWSVQMIYSDDNGLTWSNKIDLTKDSSKVKNQPSNTIGWLGGVGSGITMDDGT
IVMPSQISARENNENNYYSLIIYSKDNGETWTMGNKVPNSNTSENMVIELDVALIMSTRYDY
SGYRAAYISHDLGTTWEIYEPLNGKILTGKGSGCQGSFIHATTSNGKRIALISAPKNTHGEY
IRDQIAVYMIDFDDLSKGVQEICIPYPEDGNKLGGGYSCLSFKNNHLGIVYDFNGNIEYQDL
YPYYSLINKQ
SEQ ID NO: 101:
MNYKGITLILTAAMVISGGNYVLVKGSTLDSGKNNSGYEIKVNNSENLSSLGEYKDINLESS
NASNITYDLEKYKNLDEGTIVVRFNSKDSKIQSLLGISNSKTKNGYFNFYVTNSRVGFELRN
QKNEGNTQNGTENLVHMYKDVALNDGDNTVALKIEKNKGYKLFLNGKMIKEVKDTNTKFLNN
IENLDSAFIGKTNRYGQSNEYNFKGNIGFMNIYNEPLGDDYLLSKTGETKAKEEVLVEGAVK
TEPVDLFHPGFLNSSNYRIPALFKTKEGTLIASIDARRQGGADAPNNDIDTAVRRSEDGGKT
WDEGQIIMDYPDKSSVIDTTLIQDDETGRIFLLVTHFPSKYGFWNAGLGSGFKNIDGKEYLC
LYDSSGKEFTVRENVVYDKDGNKTEYTTNALGDLFRNGTKIDNINSSTAPLKAKGTSYINLV
YSDDDGKTWSEPQNINFQVKKDWMKFLGIAPGRGIQIKNGEHKGRIVVPVYYTNEKGKQSSA
VIYSDDSGKNWTIGESPNDNRKLENGKIINSKTLSDDAPQLTECQVVEMPNGQLKLFMRNLS
GYLNIATSFDGGATWDETVEKDTNVLEPYCQLSVINYSQKIDGKDAVIFSNPNARSRSNGTV
RIGLINQVGTYENGEPKYEFDWKYNKLVKPGYYAYSCLTELSNGNIGLLYEGTPSEEMSYIE
MNLKYLESGANK
SEQ ID NO: 102:
MNYKGITLILTAAMVISGGNYVLVKGSTLDSGKNNSGYEIKVNNSESLSSLGEYKDINLESS
NASNITYDLEKYKNLDEGTIVVRFNSKDSKIQSLLGISNSKTKNGYFNFYVTNSRVGFELRN
QKNEGNTQSGTENLVHMYKDVALNDGDNTVALKIEKNKGYKLFLNGKIIKEVKDTNTKFLNN
IENLDSAFIGKTNRYGQSNEYNFKGNIGFMNIYNEPLGDDYLLSKTGETKAKEEVLVEGAVK
TEPVDLFHPGFLNSSNYRIPALFKTKEGTLIASIDARRHGGADAPNNDIDTAVRRSEDGGKT
WDEGQIIMDYPDKSSVIDTTLIQDDETGRIFLLVTHFPSKYGFWNAGLGSGFKNIDGKEYLC
LYDSSGKEFTVRENVVYDKDGNKTEYTTNALGDLFKNGTKIDNINSSTAPLKAKGTSYINLV
YSDDDGKTWSEPQNINFQVKKDWMKFLGIAPGRGIQIKNGEHKGRIVVPVYYTNEKGKQSSA
VIYSDDSGKNWTIGESPNDNRKLENGKIINSKTLSDDAPQLTECQVVEMPNGQLKLFMRNLS
GYLNIATSFDGGATWDETVEKDTNVLEPYCQLSVINYSQKIDGKDAVIFSNPNARSRSNGTV
RIGLINQVGTYENGEPKYEFDWKYNKLVKPGYYAYSCLTELSNGNIGLLYEGTPSEEMSYIE
MNLKYLESGANK
SEQ ID NO: 103:
MKSKKIIATLVASLVISNMGGYLVKANPNVNHKAVIIEDRQAIIETAIPQSEMTASATSEEG
QDPASSAIDGNINTMWHTKWNGSDALPQSLSVNLGKARKVSSIAITPRTSGNNGFITKYEIH
AINNGVETLVAEGTWEENNLVKTVTFDSPIDAEEIKITAIQGVGGFASIAELNVYEIKGEVD
EIANYGNLKITKEEERLNITGDLEKFSSLDEGTIVTRFNMNDTSIQSLIGLSDGNKANNYFS
LYVSGGKVGYELRRQEGNGDFNVHHSADVTFNKGINTLALKIEKGVGAKIFLNGSLVKTVSD
PNIKFLNAINLNSGFIGKTDRANGYNEYLFRGNIDFMNIYDKPVSDNYLLRKTGETKAPSED
SLLPDDVYKTQPVELFYPGYLESRGYRIPALETTKKGTVLASIDVRNNGDHDAPNNNIDVGI
RRKEVNGEWEEGKVILDYPGKSAAIDTSLMSATIEENGIEKERIFLIVTHFPEGYGFPNTEG
GSGYKEIDGKYYFILKDAQNNEYTVREDGIVYNSEGNQTDYVMKNDKTLIQNGEEVGNALLS
NSPLKAVGTAHIEMIYSDDDGKTWSEPEDLNPGLKKEWMKFFGTAPGKGIQIKNGEHKGRLV
FPIYYTNQNNFQSSAVIYSDDFGETWKLGESPIDTASVSSETVSSGTQLTECQVVEMPNGQL
KLFMRNTGSYTKIATSFDGGATWHDEVPEDTSLREPYCQLSVINYSGKINGKDAIIFSNPDA
SSRVNGSVKVGLINENGTYDNGEPRYEFDWIYNKTVKPGSFAYSCLTELPDGNLGLFYEGEG
AGRMAYTEFDLNYLKFNASEDSPSASVQSIEVLDEDLAYNSGEEVSIKVNFNQLVSIIGDRK
ITLDIGGVDVPLNMVNYEGKSSAIFKGTIPEGINQGNYEIKLKENNTLELNTVYNKVSTFNG
LDNTGINVQIGELKTTVGNSTIKVNDEVQVGSAFEAILGIEGLNGDTEVYSAEYLFEYNVEA
FILNEITSFNDSLFVKSKEVEPGKVRILVASLGNEIEKDSDLVKVNLTPKISSELEVLGLTT
ALVGAGDGNTHDLELSSKEVKINEEASGEIVVNPVQNFEIPEINKKNVKLTWNAPITTEGLE
GYVIYKDGKKLSEVPAESTEFVVSKLNRHTIYNFKVAAKYSNGELSAKESKTIRTAR
SEQ ID NO: 104:
MYSLIKKSISTIALSTLAITSLPTYSVSSQTTEEYGARKYFINNNIENIKNIENKSFDLIQN
LNTKILEKENIETLSGTVVDFTKEATSNSTIPNGLIIEKSNINITAGKGYDLSSEMGSEYVK
ALEKGTIIVSYKSTSNNSIQSLVSIGNNTSGNRDRHFHIYITNTGEVGMELRNTDSVLKYTL
GRPAAVRSIYKNNLVFNTIAFKADPSNKQYKLFANGELLATLNTDVYKFINDITGVNNVMLG
GTVRDGVIAYPFGGTIGNVKIYNEILTDEALKAETGATTYGKNIFYAGDSTKSNYFRIPSLL
SLRSGTVVSAADARYGGTHDSKSNIDIAFSKSLDGGIIWKNPTIPLQFNDYVARNIDWPRDS
IGKNVQIQGSASFIDPVLLEDKETKRLFIFADAMPAGIGSSNASTGSGYKDIAGKKYMKLRW
HQDGSSTYNYSIRENGVIYNDVTNLPTEYKIDGDYNLYKNGIALLYKQYDYNFSGTTLLETA
TNIDVNMNVFYKDSLFKVFPTTYLDMKYSDDEGETWSNLNIVSSFKPENSKFLVLGPGVGKQ
ISKGQYKGRLIVPLYSSSYAELGFMYSDDHGQTWNYVAADNRNTGTTAEAQIVEMPDGSLKS
YLRTGSGVIAEVTSINGGETWSDRVTVPNMHTTSYGTQLSVINYAGLIDGKEAIILSAPDSS
SARRNGKIWIGLISDTGASGINKYSIEWKYCYSVDSSNMGYSYSCLTELPNGDIGLLYEKYD
SWSRNELHLKNILKYETFSINELKQPISN
SEQ ID NO: 105:
GAMADIGSNIFYAGDATKSNYFRIPSLLALDSGTVIAAADARYGGTHDAKSKINTAFAKSTD
GGKTWGQPTLPLKFDDYVAKNIDWPRDSVGKNVQIQGSASYIDPVLLEDKETHRVFLFADMM
PAGIGSSNASVGSGFKEVDGKKYLKLHWKDDAAGTYDYSVRENGTIYNDTTNSATEYSVDGE
YNLYKNGNAMLCKQYDYNFEGTKLLETQTDTDVNMNVFYKDADFKVFPTTYLAMKYSDDEGE
TWSDLQIVSTFKPEESKFLVLGPGVGKQIANGEHAGRLIVPLYSKSSAELGFMYSDDHGNNW
TYVEADQNTGGATAEAQIVEMPDGSLKTYLRTGSGYIAQVMSTDGGETWSERVPLTEIATTG
YGTQLSVINYSQPVDGKPAILLSAPNATNGRKNGKIWIGLISETGNSGKDKYSVDWKYCYSV
DTPQMGYSYSCLTELPDGEIGLLYEKYDSWSRNELHLKNILKYERFNIDELKVQP
SEQ ID NO: 106:
MTVEKSVVFKAEGEHFTDQKGNTIVGSGSGGTTKYFRIPAMCTTSKGTIVVFADARHNTASD
QSFIDTAAARSTDGGKTWNKKIAIYNDRVNSKLSRVMDPTCIVANIQGRETILVMVGKWNNN
DKTWGAYRDKAPDTDWDLVLYKSTDDGVTFSKVETNIHDIVTKNGTISAMLGGVGSGLQLND
GKLVFPVQMVRTKNITTVLNTSFIYSTDGITWSLPSGYCEGFGSENNIIEFNASLVNNIRNS
GLRRSFETKDFGKTWTEFPPMDKKVDNRNHGVQGSTITIPSGNKLVAAHSSAQNKNNDYTRS
DISLYAHNLYSGEVKLIDDFYPKVGNASGAGYSCLSYRKNVDKETLYVVYEANGSIEFQDLS
RHLPVIKSYN
SEQ ID NO: 107:
MNKKKIMSILVSAFLITNLSSNIIFADIKENVYINQYSEGNRSQPIAEKLVPRSEIQASATS
ALTGEGPEKAIDGNTSTLWHTPWAGVDIQINPQSLTLKLGKTRNISSICVTPRQEGTNGMIT
DYKIYSGDDVIAEGKWKSDSSDKYVVFDNPISTDNIRIEAISTVGDENNKHASIAEVEVYEL
ADTPVKLAESNNKVINNGNGGNYEGDISEISLLEEGTAIIRFTNNGSSLFSISNNERTNEHF
HVYINGGAIGYELRKQSGNLATGSVNKALNAGINTIAFKAEKGKGYSIYLNGEKILTSSSIT
ANFLSTLEGLNTLSLGKTDRPSGSNEYNFTGEIDFFELYSKPLADRYLKERTGETTSKDLPF
PEGAVKTEPVDIETPGELGSNNFRIPALYTTKDGTVLASIDVRKGGGHDAPNNIDTGIKRST
DGGVTWDEGKIILDYPGASSAIDTSLLQDDETGRIFLIVTHFAEGYGFGNSKTGSGYVEIEG
KRYLKLLGANDTIYTVREGVVYDSNGEATNYTVDNNNELYENGNRIGNVLLSNSPLKVMGTS
FLSLIYSDDDGQTWSDPIDLNKEVKTDWMRFLGTGPGKGHQIKTGRYAGRLLFPVYLTNASG
FQSSAVIYSDDNGATWNIGETATDGRLMDNGDRASAETITTNTSGGVGQLTECQVVEMPNGQ
LKMFMRNTGGNSGRVRIATSFDGGATWEDDVVRDENIKEPYCQLSVINYSQKIDGKDAIIFA
IPDANYPNRVNGTVRVGLITENGSYENGEPRYDIEWRYNKVVAPGTYGYSCLSEMPNGEIGL
FYEGRGSRQMSFTRMNIDYLKADLLQDVPAANIKSYTTNSENNIYDPGDKISLNVTFDQTVS
LIGDRTITADIGGKEVLLTLANSKGGSEYTFEGTVPADISNGNYTITIKGKSGLKIVNVVNK
VTDITEDRNTGLNVQVGEEVQSVDKTLLQDLVDSTSNLIKEDYTEESWILYEKALEVANKFL
VNEIAVQEEVDAAKPTLENAYK
SEQ ID NO: 108:
MSYFRNRDIDIERNSMNRSVQERKCRYSIRKLSVGAVSMIVGAVVFGTSPVLAQEGASEQPL
ANETQLSGESSTLTDTEKSQPSSETELSGNKQEQERKDKQEEKIPRDYYARDLENVETVIEK
EDVETNASNGQRVDLSSELDKLKKLENATVHMEFKPDAKAPAFYNLFSVSSATKKDEYFTMA
VYNNTATLEGRGSDGKQFYNNYNDAPLKVKPGQWNSVTFTVEKPTAELPKGRVRLYVNGVLS
RTSLRSGNFIKDMPDVTHVQIGATKRANNTVWGSNLQIRNLTVYNRALTPEEVQKRSQLFKR
SDLEKKLPEGAALTEKTDIFESGRNGKPNKDGIKSYRIPALLKTDKGTLIAGADERRLHSSD
WGDIGMVIRRSEDNGKTWGDRVTITNLRDNPKASDPSIGSPVNIDMVLVQDPETKRIFSIYD
MFPEGKGIFGMSSQKEEAYKKIDGKTYQILYREGEKGAYTIRENGTVYTPDGKATDYRVVVD
PVKPAYSDKGDLYKGNQLLGNIYFTTNKTSPFRIAKDSYLWMSYSDDDGKTWSAPQDITPMV
KADWMKFLGVGPGTGIVLRNGPHKGRILIPVYTTNNVSHLNGSQSSRIIYSDDHGKTWHAGE
AVNDNRQVDGQKIHSSTMNNRRAQNTESTVVQLNNGDVKLFMRGLTGDLQVATSKDGGVTWE
KDIKRYPQVKDVYVQMSAIHTMHEGKEYIILSNAGGPKRENGMVHLARVEENGELTWLKHNP
IQKGEFAYNSLQELGNGEYGILYEHTEKGQNAYTLSFRKFNWDFLSKDLISPTEAKVKRTRE
MGKGVIGLEFDSEVLVNKAPTLQLANGKTARFMTQYDTKTLLFTVDSEDMGQKVTGLAEGAI
ESMHNLPVSVAGTKLSNGMNGSEAAVHEVPEYTGPLGTSGEEPAPTVEKPEYTGPLGTSGEE
PAPTVEKPEYTGPLGTAGEEAAPTVEKPEFTGGVNGTEPAVHEIAEYKGSDSLVTLTTKEDY
TYKAPLAQQALPETGNKESDLLASLGLTAFFLGLFTLGKKREQ
SEQ ID NO: 109:
LKEAKEKAIEELKKAGITSDYYFDLINKAKTVEGVNALKDEILKA
SEQ ID NO: 110:
(GGP)ₙ, where n is 1-5
SEQ ID NO: 111:
(GGGGS)ₙ, where n is 1-5
SEQ ID NO: 112:
GGGGSGGGGS
SEQ ID NO: 113:
ASLPVLQKESVFQSGAHAYRIPALLYLPGQQSLLAFAEQRASKKDEHAELIVLRRGDYDAPT
HQVQWQAQEVVAQARLDGHRSMNPCPLYDAQTGTLFLFFIAIPGQVTEQQQLQTRANVTRLC
QVTSTDHGRTWSSPRDLTDAAIGPAYREWSTFAVGPGHCLQLHDRARSLVVPAYAYRKLHPI
QRPIPSAFCFLSHDHGRTWARGHFVAQDTLECQVAEVETGEQRVVTLNARSHLRARVQAQST
NDGLDFQESQLVKKLVEPPPQGCQGSVISFPSPRSGPGSPAQWLLYTHPTHSWQRADLGAYL
NPRPPAPEAWSEPVLLAKGSCAYSDLQSMGTGPDGSPLFGCLYEANDYEEIVFLMFTLKQAF
PAEYLPQGGGGSGGGGSDKTHTCPPCPAPELLGGPSVFLFPPKPKDTLMISRTPEVTCVVVD
VSHEDPEVKFNWYVDGVEVHNAKTKPREEQYNSTYRVVSVLTVLHQDWLNGKEYKCKVSNKA
LPAPIEKTISKAKGQPREPQVYTLPPSREEMTKNQVSLTCLVKGFYPSDIAVEWESNGQPEN
NYKTTPPVLDSDGSFFLTSKLTVDKSRWQQGNVFSCSVMHEALHNHYTQKSLSLSPGK
SEQ ID NO: 114:
GCATCTCTGCCTGTGCTGCAGAAAGAAAGCGTGTTCCAGTCTGGCGCCCACGCCTACAGAAT
TCCCGCTCTGCTGTATCTGCCAGGCCAGCAGTCTCTGCTGGCTTTCGCTGAACAGCGGGCCA
GCAAGAAGGATGAGCACGCCGAACTGATCGTGCTGCGGAGAGGCGATTACGACGCCCCTACA
CATCAGGTGCAGTGGCAGGCTCAAGAGGTGGTGGCTCAGGCTAGACTGGACGGCCACAGATC
TATGAACCCCTGTCCTCTGTACGATGCCCAGACCGGCACACTGTTTCTGTTCTTTATCGCTA
TCCCCGGCCAAGTGACCGAGCAGCAGCAGCTGCAGACAAGAGCCAACGTGACCAGACTGTGT
CAAGTGACCTCCACCGACCACGGCAGAACCTGGTCTAGCCCTAGAGATCTGACCGACGCCGC
CATCGGACCTGCCTATAGAGAGTGGTCCACCTTCGCCGTTGGACCTGGACACTGTCTCCAGC
TGCACGACAGGGCTAGATCTCTGGTGGTGCCTGCCTACGCCTATAGAAAGCTGCACCCCATC
CAGCGGCCTATTCCTAGCGCCTTCTGCTTTCTGAGCCACGATCACGGCAGGACATGGGCCAG
AGGACATTTCGTGGCCCAGGACACACTGGAATGCCAGGTGGCCGAAGTGGAAACCGGCGAGC
AGAGAGTCGTGACCCTGAACGCCAGATCTCACCTGAGAGCCAGAGTGCAGGCCCAGAGCACA
AACGACGGCCTGGATTTCCAAGAGAGCCAGCTGGTCAAGAAACTGGTGGAACCTCCTCCACA
GGGCTGTCAGGGAAGCGTGATCAGCTTTCCATCTCCTAGAAGCGGCCCTGGCTCTCCTGCTC
AGTGGCTGCTGTATACACACCCCACACACAGCTGGCAGAGAGCCGATCTGGGCGCCTACCTG
AATCCTAGACCTCCTGCTCCTGAGGCTTGGAGCGAACCTGTTCTGCTGGCCAAGGGCAGCTG
TGCCTACAGCGATCTGCAGTCTATGGGCACAGGCCCTGATGGCAGCCCTCTGTTTGGCTGTC
TGTACGAGGCCAACGACTACGAAGAGATCGTGTTCCTGATGTTCACCCTGAAGCAGGCCTTT
CCAGCCGAGTACCTGCCTCAAGGCGGAGGTGGAAGTGGCGGAGGCGGATCcGACAAAACTCA
CACATGCCCACCGTGCCCAGCACCTGAACTCCTGGGGGGACCGTCAGTCTTCCTCTTCCCCC
CAAAACCCAAGGACACCCTCATGATCTCCCGGACCCCTGAGGTCACATGCGTGGTGGTGGAC
GTGAGCCACGAAGACCCTGAGGTCAAGTTCAACTGGTACGTGGACGGCGTGGAGGTGCATAA
TGCCAAGACAAAGCCGCGGGAGGAGCAGTACAACAGCACGTACCGTGTGGTCAGCGTCCTCA
CCGTCCTGCACCAGGACTGGCTGAATGGCAAGGAGTACAAGTGCAAGGTCTCCAACAAAGCC
CTCCCAGCCCCCATCGAGAAAACCATCTCCAAAGCCAAAGGGCAGCCCCGAGAACCACAGGT
cTACACCCTGCCCCCATCCCGGGAGGAGATGACCAAGAACCAGGTCAGCCTGACCTGCCTGG
TCAAAGGCTTCTATCCCAGCGACATCGCCGTGGAGTGGGAGAGCAATGGGCAGCCGGAGAAC
AACTACAAGACCACGCCTCCCGTGCTGGACTCCGACGGCTCCTTCTTCCTCTACAGCAAGCT
CACCGTGGACAAGAGCAGGTGGCAGCAGGGGAACGTCTTCTCATGCTCCGTGATGCATGAGG
CTCTGCACAACCACTACACGCAGAAGAGCCTCTCCCTGTCTCCGGGTAAA
SEQ ID NO: 115:
DASLPYLQKESVFQSGAHAYRIPALLYLPGQQSLLAFAEQRASKKDEHAELIVLRRGDYDAG
THQVQWQAQEVVAQARLDGHRSMNPCPLYDEQTGTLFLFFIAIPGQVTEQQQLQTRANVTRL
CQVTSTDHGRTWSSPRDLTDAAIGPAYREWSTFAVGPGHCLQLHDRARSLVVPAYAYRKLHP
KQRPIPSAFCFLSHDHGRTWARGHFVAQDTLECQVAEVETGEQRVVTLNARSHLRARVQAQS
TNDGLDFQESQLVKKLVEPPPQGCQGSVISFPSPRSGPGSPAQWLLYTHPTHSWQRADLGAY
LNPRPPAPEAWSEPVLLAKGSAAYSDLQSMGTGPDGSPLFGCLYEANDYEEIVFLMFTLKQA
FPAEYLPQGGGGSGGGGSDKTHTCPPCPAPELLGGPSVFLFPPKPKDTLMISRTPEVTCVVV
DVSHEDPEVKFNWYVDGVEVHNAKTKPREEQYNSTYRVVSVLTVLHQDWLNGKEYKCKVSNK
ALPAPIEKTISKAKGQPREPQVYTLPPSREEMTKNQVSLTCLVKGFYPSDIAVEWESNGQPE
NNYKTTPPVLDSDGSFFLTSKLTVDKSRWQQGNVFSCSVMHEALHNHYTQKSLSLSPGK
SEQ ID NO: 116:
GATGCATCTCTGCCTTACCTGCAGAAAGAAAGCGTGTTCCAGTCTGGCGCCCACGCCTACAG
AATTCCCGCTCTGCTGTATCTGCCAGGCCAGCAGTCTCTGCTGGCTTTCGCTGAACAGCGGG
CCAGCAAGAAGGATGAGCACGCCGAACTGATCGTGCTGCGGAGAGGCGATTACGACGCCGGC
ACACATCAGGTGCAGTGGCAGGCTCAAGAGGTGGTGGCTCAGGCTAGACTGGACGGCCACAG
ATCTATGAACCCCTGTCCTCTGTACGATGAACAGACCGGCACACTGTTTCTGTTCTTTATCG
CTATCCCCGGCCAAGTGACCGAGCAGCAGCAGCTGCAGACAAGAGCCAACGTGACCAGACTG
TGTCAAGTGACCTCCACCGACCACGGCAGAACCTGGTCTAGCCCTAGAGATCTGACCGACGC
CGCCATCGGACCTGCCTATAGAGAGTGGTCCACCTTCGCCGTTGGACCTGGACACTGTCTCC
AGCTGCACGACAGGGCTAGATCTCTGGTGGTGCCTGCCTACGCCTATAGAAAGCTGCACCCC
AAACAGCGGCCTATTCCTAGCGCCTTCTGCTTTCTGAGCCACGATCACGGCAGGACATGGGC
CAGAGGACATTTCGTGGCCCAGGACACACTGGAATGCCAGGTGGCCGAAGTGGAAACCGGCG
AGCAGAGAGTCGTGACCCTGAACGCCAGATCTCACCTGAGAGCCAGAGTGCAGGCCCAGAGC
ACAAACGACGGCCTGGATTTCCAAGAGAGCCAGCTGGTCAAGAAACTGGTGGAACCTCCTCC
ACAGGGCTGTCAGGGAAGCGTGATCAGCTTTCCATCTCCTAGAAGCGGCCCTGGCTCTCCTG
CTCAGTGGCTGCTGTATACACACCCCACACACAGCTGGCAGAGAGCCGATCTGGGCGCCTAC
CTGAATCCTAGACCTCCTGCTCCTGAGGCTTGGAGCGAACCTGTTCTGCTGGCCAAGGGCAG
CGCTGCCTACAGCGATCTGCAGTCTATGGGCACAGGCCCTGATGGCAGCCCTCTGTTTGGCT
GTCTGTACGAGGCCAACGACTACGAAGAGATCGTGTTCCTGATGTTCACCCTGAAGCAGGCC
TTTCCAGCCGAGTACCTGCCTCAAGGCGGAGGTGGAAGTGGCGGAGGCGGATCCGACAAAAC
TCACACATGCCCACCGTGCCCAGCACCTGAACTCCTGGGGGGACCGTCAGTCTTCCTCTTCC
CCCCAAAACCCAAGGACACCCTCATGATCTCCCGGACCCCTGAGGTCACATGCGTGGTGGTG
GACGTGAGCCACGAAGACCCTGAGGTCAAGTTCAACTGGTACGTGGACGGCGTGGAGGTGCA
TAATGCCAAGACAAAGCCGCGGGAGGAGCAGTACAACAGCACGTACCGTGTGGTCAGCGTCC
TCACCGTCCTGCACCAGGACTGGCTGAATGGCAAGGAGTACAAGTGCAAGGTCTCCAACAAA
GCCCTCCCAGCCCCCATCGAGAAAACCATCTCCAAAGCCAAAGGGCAGCCCCGAGAACCACA
GGTCTACACCCTGCCCCCATCCCGGGAGGAGATGACCAAGAACCAGGTCAGCCTGACCTGCC
TGGTCAAAGGCTTCTATCCCAGCGACATCGCCGTGGAGTGGGAGAGCAATGGGCAGCCGGAG
AACAACTACAAGACCACGCCTCCCGTGCTGGACTCCGACGGCTCCTTCTTCCTCACTAGCAA
GCTCACCGTGGACAAGAGCAGGTGGCAGCAGGGGAACGTCTTCTCATGCTCCGTGATGCATG
AGGCTCTGCACAACCACTACACGCAGAAGAGCCTCTCCCTGTCTCCGGGTAAA
SEQ ID NO: 117:
EEVTTCSFNSPLFRQEDDRGITYRIPALLYIPPTHTFLAFAEKRSTRRDEDALHLVLRRGLR
IGQLVQWGPLKPLMEATLPGHRTMNPCPVWEQKSGCVFLFFICVRGHVTERQQIVSGRNAAR
LCFIYSQDAGCSWSEVRDLTEEVIGSELKHWATFAVGPGHGIQLQSGRLVIPAYTYRKLHPK
QRTRPHSLMIYSDDLGVTWHHGRLIRPMVTVECEVAEVTGRAGHPVLYCSARTPNRCRAEAL
STDHGEGFQRLALSRQLCEPPHGCQGSVVSFRPLEIPHRCQDSSSKDAPTIQQSSPGSSLRL
EEEAGTPSESWLLYSHPTSRKQRVDLGIYLNQTPLEAACWSRPWILHCGPCGYSDLAALEEE
GLFGCLFECGTKQECEQIAFRLFTHREILSHLQGDCTSPGRNPSQFKSNGGGGSGGGGSDKT
HTCPPCPAPELLGGPSVFLFPPKPKDTLMISRTPEVTCVVVDVSHEDPEVKFNWYVDGVEVH
NAKTKPREEQYNSTYRVVSVLTVLHQDWLNGKEYKCKVSNKALPAPIEKTISKAKGQPREPQ
VYTLPPSREEMTKNQVSLTCLVKGFYPSDIAVEWESNGQPENNYKTTPPVLDSDGSFFLYSK
LTVDKSRWQQGNVFSCSVMHEALHNHYTQKSLSLSPGK
SEQ ID NO: 118:
GAGGAAGTGACCACCTGTAGCTTCAACAGCCCTCTGTTCCGGCAAGAGGACGACCGGGGCAT
CACCTACAGAATCCCTGCTCTGCTcTACATCCCTCCTACACACACCTTTCTGGCCTTCGCCG
AGAAGCGGAGCACCAGACGAGATGAAGATGCCCTGCACCTGGTGCTGAGAAGAGGCCTGAGA
ATCGGACAGCTGGTGCAGTGGGGACCTCTGAAGCCTCTGATGGAAGCCACACTGCCCGGCCA
CAGAACCATGAATCCTTGTCCTGTGTGGGAGCAGAAAAGCGGCTGCGTGTTCCTGTTCTTCA
TCTGCGTGCGGGGCCACGTGACCGAGAGACAGCAAATCGTGTCCGGCAGAAACGCCGCCAGA
CTGTGCTTCATCTACAGCCAGGATGCCGGCTGCTCTTGGAGCGAAGTTCGGGATCTGACCGA
AGAAGTGATCGGCAGCGAGCTGAAGCACTGGGCCACATTTGCTGTTGGCCCTGGCCACGGAA
TCCAGCTGCAATCTGGCAGACTGGTCATCCCCGCCTACACCTACAGAAAGCTGCACCCCAAA
CAGCGGACCCGGCCTCACAGCCTGATGATCTACAGCGACGATCTGGGCGTGACATGGCACCA
CGGCAGACTGATCAGACCCATGGTCACCGTGGAATGCGAGGTGGCCGAAGTGACAGGCAGAG
CTGGACACCCTGTGCTGTACTGCTCTGCCAGAACACCCAACCGGTGTAGAGCCGAGGCTCTG
TCTACAGATCACGGCGAGGGCTTTCAGAGACTGGCCCTCTCTAGACAGCTGTGCGAACCTCC
TCATGGCTGTCAGGGCAGCGTGGTGTCCTTCAGACCTCTGGAAATCCCTCACCGGTGCCAGG
ACAGCAGCTCTAAGGATGCCCCTACCATCCAGCAGTCTAGCCCTGGCAGCAGCCTGAGACTG
GAAGAGGAAGCCGGAACACCTAGCGAGAGCTGGCTGCTGTACTCTCACCCCACCAGCAGAAA
GCAGAGAGTGGACCTGGGCATCTACCTGAATCAGACCCCTCTGGAAGCCGCCTGTTGGAGCA
GACCTTGGATTCTGCACTGTGGCCCTTGCGGCTACTCTGATCTGGCCGCTCTGGAAGAAGAG
GGCCTGTTCGGCTGCCTGTTTGAGTGCGGCACAAAGCAAGAGTGCGAGCAGATCGCCTTCCG
GCTGTTCACCCACAGAGAGATCCTGAGCCATCTGCAGGGCGACTGCACAAGCCCAGGCAGAA
ATCCCAGCCAGTTCAAGAGCAACGGCGGAGGTGGAAGTGGCGGAGGCGGATCcGACAAAACT
CACACATGCCCACCGTGCCCAGCACCTGAACTCCTGGGGGGACCGTCAGTCTTCCTCTTCCC
CCCAAAACCCAAGGACACCCTCATGATCTCCCGGACCCCTGAGGTCACATGCGTGGTGGTGG
ACGTGAGCCACGAAGACCCTGAGGTCAAGTTCAACTGGTACGTGGACGGCGTGGAGGTGCAT
AATGCCAAGACAAAGCCGCGGGAGGAGCAGTACAACAGCACGTACCGTGTGGTCAGCGTCCT
CACCGTCCTGCACCAGGACTGGCTGAATGGCAAGGAGTACAAGTGCAAGGTCTCCAACAAAG
CCCTCCCAGCCCCCATCGAGAAAACCATCTCCAAAGCCAAAGGGCAGCCCCGAGAACCACAG
GTcTACACCCTGCCCCCATCCCGGGAGGAGATGACCAAGAACCAGGTCAGCCTGACCTGCCT
GGTCAAAGGCTTCTATCCCAGCGACATCGCCGTGGAGTGGGAGAGCAATGGGCAGCCGGAGA
ACAACTACAAGACCACGCCTCCCGTGCTGGACTCCGACGGCTCCTTCTTCCTCtaTAGCAAG
CTCACCGTGGACAAGAGCAGGTGGCAGCAGGGGAACGTCTTCTCATGCTCCGTGATGCATGA
GGCTCTGCACAACCACTACACGCAGAAGAGCCTCTCCCTGTCTCCGGGTAAA
SEQ ID NO: 119:
TVEKSVVFKAEGEHFTDQKGNTIVGSGSGGTTKYFRIPAMCTTSKGTIVVFADARHNTASDQ
SFIDTAAARSTDGGKTWNKKIAIYNDRVNSKLSRVMDPTCIVANIQGRETILVMVGKWNNND
KTWGAYRDKAPDTDWDLVLYKSTDDGVTFSKVETNIHDIVTKNGTISAMLGGVGSGLQLNDG
KLVFPVQMVRTKNITTVLNTSFIYSTDGITWSLPSGYCEGFGSENNIIEFNASLVNNIRNSG
LRRSFETKDFGKTWTEFPPMDKKVDNRNHGVQGSTITIPSGNKLVAAHSSAQNKNNDYTRSD
ISLYAHNLYSGEVKLIDDFYPKVGNASGAGYSCLSYRKNVDKETLYVVYEANGSIEFQDLSR
HLPVIKSYNGGGGSGGGGSDKTHTCPPCPAPELLGGPSVFLFPPKPKDTLMISRTPEVTCVV
VDVSHEDPEVKFNWYVDGVEVHNAKTKPREEQYNSTYRVVSVLTVLHQDWLNGKEYKCKVSN
KALPAPIEKTISKAKGQPREPQVYTLPPSREEMTKNQVSLTCLVKGFYPSDIAVEWESNGQP
ENNYKTTPPVLDSDGSFFLTSKLTVDKSRWQQGNVFSCSVMHEALHNHYTQKSLSLSPGK
SEQ ID NO: 120:
TVEKSVVFKAEGEHFTDQKGNTIVGSGSGGTTKYFRIPAMCTTSKGTIVVFADARHNTASDQ
SFIDTAAARSTDGGKTWNKKIAIYNDRVNSKLSRVMDPTCIVANIQGRETILVMVGKWNNND
KTWGAYRDKAPDTDWDLVLYKSTDDGVTFSKVETNIHDIVTKNGTISAMLGGVGSGLQLNDG
KLVFPVQMVRTKNITTVLNTSFIYSTDGITWSLPSGYCEGFGSENNIIEFNASLVNNIRNSG
LRRSFETKDFGKTWTEFPPMDKKVDNRNHGVQGSTITIPSGNKLVAAHSSAQNKNNDYTRSD
ISLYAHNLYSGEVKLIDDFYPKVGNASGAGYSCLSYRKNVDKETLYVVYEANGSIEFQDLSR
HLPVIKSYNGGGGSGGGGSDKTHTCPPCPAPELLGGPSVFLFPPKPKDTLMISRTPEVTCVV
VDVSHEDPEVKFNWYVDGVEVHNAKTKPREEQYNSTYRVVSVLTVLHQDWLNGKEYKCKVSN
KALPAPIEKTISKAKGQPREPQVYTLPPSREEMTKNQVSLYCLVKGFYPSDIAVEWESNGQP
ENNYKTTPPVLDSDGSFFLYSKLTVDKSRWQQGNVFSCSVMHEALHNHYTQKSLSLSPGK
SEQ ID NO: 121:
ACAGTGGAAAAGTCCGTGGTGTTCAAGGCCGAGGGCGAGCACTTCACCGACCAGAAAGGCAA
TACCATCGTCGGCTCTGGCAGCGGCGGCACCACCAAGTACTTTAGAATCCCCGCCATGTGCA
CCACCAGCAAGGGCACCATTGTGGTGTTCGCCGACGCCAGACACAACACCGCCAGCGATCAG
AGCTTCATCGATACCGCTGCCGCCAGAAGTACAGACGGCGGCAAGACCTGGAACAAGAAGAT
CGCCATCTACAACGACCGCGTGAACAGCAAGCTGAGCAGAGTGATGGACCCTACCTGCATCG
TGGCCAACATCCAGGGCAGAGAAACCATCCTGGTCATGGTCGGAAAGTGGAACAACAACGAT
AAGACCTGGGGCGCCTACAGAGACAAGGCCCCTGATACCGATTGGGACCTCGTGCTGTATAA
GAGCACCGACGACGGCGTGACCTTCAGCAAGGTGGAAACAAACATCCACGACATCGTGACCA
AGAACGGCACCATCTCTGCCATGCTCGGCGGCGTTGGATCTGGCCTGCAACTGAATGATGGC
AAGCTGGTGTTCCCCGTGCAGATGGTCCGAACAAAGAACATCACCACCGTGCTGAATACCAG
CTTCATCTACTCCACCGACGGCATCACATGGTCCCTGCCTAGCGGCTACTGTGAAGGCTTTG
GCAGCGAGAACAACATCATCGAGTTCAACGCCAGCCTGGTCAACAACATCCGGAACAGCGGC
CTGCGGAGAAGCTTCGAGACAAAGGACTTCGGAAAGACGTGGACCGAGTTTCCTCCAATGGA
CAAGAAGGTGGACAACCGGAACCACGGCGTGCAGGGCAGCACAATCACAATCCCTAGCGGCA
ACAAACTGGTGGCCGCTCACTCTAGCGCCCAGAACAAGAACAACGATTACACCAGAAGCGAC
ATCAGCCTGTACGCCCACAACCTGTACTCCGGCGAAGTGAAGCTGATCGACGACTTCTACCC
CAAAGTGGGCAATGCCAGCGGAGCCGGCTACAGCTGTCTGAGCTACCGGAAAAATGTGGACA
AAGAAACCCTGTACGTGGTGTACGAGGCCAACGGCAGCATCGAGTTTCAGGACCTGAGCAGA
CATCTGCCCGTGATCAAGAGCTACAATGGCGGAGGTGGAAGTGGCGGAGGCGGATCCGACAA
AACTCACACATGCCCACCGTGCCCAGCACCTGAACTCCTGGGGGGACCGTCAGTCTTCCTCT
TCCCCCCAAAACCCAAGGACACCCTCATGATCTCCCGGACCCCTGAGGTCACATGCGTGGTG
GTGGACGTGAGCCACGAAGACCCTGAGGTCAAGTTCAACTGGTACGTGGACGGCGTGGAGGT
GCATAATGCCAAGACAAAGCCGCGGGAGGAGCAGTACAACAGCACGTACCGTGTGGTCAGCG
TCCTCACCGTCCTGCACCAGGACTGGCTGAATGGCAAGGAGTACAAGTGCAAGGTCTCCAAC
AAAGCCCTCCCAGCCCCCATCGAGAAAACCATCTCCAAAGCCAAAGGGCAGCCCCGAGAACC
ACAGGTCTACACCCTGCCCCCATCCCGGGAGGAGATGACCAAGAACCAGGTCAGCCTGACCT
GCCTGGTCAAAGGCTTCTATCCCAGCGACATCGCCGTGGAGTGGGAGAGCAATGGGCAGCCG
GAGAACAACTACAAGACCACGCCTCCCGTGCTGGACTCCGACGGCTCCTTCTTCCTCACTAG
CAAGCTCACCGTGGACAAGAGCAGGTGGCAGCAGGGGAACGTCTTCTCATGCTCCGTGATGC
ATGAGGCTCTGCACAACCACTACACGCAGAAGAGCCTCTCCCTGTCTCCGGGTAAA
SEQ ID NO: 122:
ACAGTGGAAAAGTCCGTGGTGTTCAAGGCCGAGGGCGAGCACTTCACCGACCAGAAAGGCAA
TACCATCGTCGGCTCTGGCAGCGGCGGCACCACCAAGTACTTTAGAATCCCCGCCATGTGCA
CCACCAGCAAGGGCACCATTGTGGTGTTCGCCGACGCCAGACACAACACCGCCAGCGATCAG
AGCTTCATCGATACCGCTGCCGCCAGAAGTACAGACGGCGGCAAGACCTGGAACAAGAAGAT
CGCCATCTACAACGACCGCGTGAACAGCAAGCTGAGCAGAGTGATGGACCCTACCTGCATCG
TGGCCAACATCCAGGGCAGAGAAACCATCCTGGTCATGGTCGGAAAGTGGAACAACAACGAT
AAGACCTGGGGCGCCTACAGAGACAAGGCCCCTGATACCGATTGGGACCTCGTGCTGTATAA
GAGCACCGACGACGGCGTGACCTTCAGCAAGGTGGAAACAAACATCCACGACATCGTGACCA
AGAACGGCACCATCTCTGCCATGCTCGGCGGCGTTGGATCTGGCCTGCAACTGAATGATGGC
AAGCTGGTGTTCCCCGTGCAGATGGTCCGAACAAAGAACATCACCACCGTGCTGAATACCAG
CTTCATCTACTCCACCGACGGCATCACATGGTCCCTGCCTAGCGGCTACTGTGAAGGCTTTG
GCAGCGAGAACAACATCATCGAGTTCAACGCCAGCCTGGTCAACAACATCCGGAACAGCGGC
CTGCGGAGAAGCTTCGAGACAAAGGACTTCGGAAAGACGTGGACCGAGTTTCCTCCAATGGA
CAAGAAGGTGGACAACCGGAACCACGGCGTGCAGGGCAGCACAATCACAATCCCTAGCGGCA
ACAAACTGGTGGCCGCTCACTCTAGCGCCCAGAACAAGAACAACGATTACACCAGAAGCGAC
ATCAGCCTGTACGCCCACAACCTGTACTCCGGCGAAGTGAAGCTGATCGACGACTTCTACCC
CAAAGTGGGCAATGCCAGCGGAGCCGGCTACAGCTGTCTGAGCTACCGGAAAAATGTGGACA
AAGAAACCCTGTACGTGGTGTACGAGGCCAACGGCAGCATCGAGTTTCAGGACCTGAGCAGA
CATCTGCCCGTGATCAAGAGCTACAATGGCGGAGGTGGAAGTGGCGGAGGCGGATCCGACAA
AACTCACACATGCCCACCGTGCCCAGCACCTGAACTCCTGGGGGGACCGTCAGTCTTCCTCT
TCCCCCCAAAACCCAAGGACACCCTCATGATCTCCCGGACCCCTGAGGTCACATGCGTGGTG
GTGGACGTGAGCCACGAAGACCCTGAGGTCAAGTTCAACTGGTACGTGGACGGCGTGGAGGT
GCATAATGCCAAGACAAAGCCGCGGGAGGAGCAGTACAACAGCACGTACCGTGTGGTCAGCG
TCCTCACCGTCCTGCACCAGGACTGGCTGAATGGCAAGGAGTACAAGTGCAAGGTCTCCAAC
AAAGCCCTCCCAGCCCCCATCGAGAAAACCATCTCCAAAGCCAAAGGGCAGCCCCGAGAACC
ACAGGTCTACACCCTGCCCCCATCCCGGGAGGAGATGACCAAGAACCAGGTCAGCCTGTACT
GCCTGGTCAAAGGCTTCTATCCCAGCGACATCGCCGTGGAGTGGGAGAGCAATGGGCAGCCG
GAGAACAACTACAAGACCACGCCTCCCGTGCTGGACTCCGACGGCTCCTTCTTCCTCTATAG
CAAGCTCACCGTGGACAAGAGCAGGTGGCAGCAGGGGAACGTCTTCTCATGCTCCGTGATGC
ATGAGGCTCTGCACAACCACTACACGCAGAAGAGCCTCTCCCTGTCTCCGGGTAAA
SEQ ID NO: 123:
DASLPYLQKESVFQSGAHAYRIPALLYLPGQQSLLAFAEQRASKKDEHAELIVLRRGDYDAP
THQVQWQAQEVVAQARLDGHRSMNPCPLYDAQTGTLFLFFIAIPGQVTEQQQLQTRANVTRL
CQVTSTDHGRTWSSPRDLTDAAIGPAYREWSTFAVGPGHCLQLHDRARSLVVPAYAYRKLHP
KQRPIPSAFCFLSHDHGRTWARGHFVAQDTLECQVAEVETGEQRVVTLNARSHLRARVQAQS
TNDGLDFQESQLVKKLVEPPPQGCQGSVISFPSPRSGPGSPAQWLLYTHPTHSWQRADLGAY
LNPRPPAPEAWSEPVLLAKGSAAYSDLQSMGTGPDGSPLFGCLYEANDYEEIVFLMFTLKQA
FPAEYLPQGGGGSGGGGSDKTHTCPPCPAPELLGGPSVFLFPPKPKDTLMISRTPEVTCVVV
DVSHEDPEVKFNWYVDGVEVHNAKTKPREEQYNSTYRVVSVLTVLHQDWLNGKEYKCKVSNK
ALPAPIEKTISKAKGQPREPQVYTLPPSREEMTKNQVSLTCLVKGFYPSDIAVEWESNGQPE
NNYKTTPPVLDSDGSFFLTSKLTVDKSRWQQGNVFSCSVMHEALHNHYTQKSLSLSPGK
SEQ ID NO: 124:
DIQMTQSPSSLSASVGDRVTITCRASQDVNTAVAWYQQKPGKAPKLLIYSASFLYSGVPSRF
SGSRSGTDFTLTISSLQPEDFATYYCQQHYTTPPTFGQGTKVEIKRTVAAPSVFIFPPSDEQ
LKSGTASVVCLLNNFYPREAKVQWKVDNALQSGNSQESVTEQDSKDSTYSLSSTLTLSKADY
EKHKVYACEVTHQGLSSPVTKSFNRGEC
SEQ ID NO: 125:
GACATCCAGATGACACAGAGCCCTAGCAGCCTGTCTGCCAGCGTGGGAGACAGAGTGACCAT
CACCTGTAGAGCCAGCCAGGACGTGAACACAGCCGTGGCTTGGTATCAGCAGAAGCCTGGCA
AGGCCCCTAAGCTGCTGATCTACAGCGCCAGCTTTCTGTACTCCGGCGTGCCCAGCAGATTC
AGCGGCTCTAGAAGCGGCACCGACTTCACCCTGACCATAAGCAGTCTGCAGCCCGAGGACTT
CGCCACCTACTACTGTCAGCAGCACTACACCACACCTCCAACCTTTGGCCAGGGCACCAAGG
TGGAAATCAAGCGTACGGTGGCTGCACCATCTGTCTTCATCTTCCCGCCATCTGATGAGCAG
TTGAAATCTGGAACTGCCTCTGTTGTGTGCCTGCTGAATAACTTCTATCCCAGAGAGGCCAA
AGTACAGTGGAAGGTGGATAACGCCCTCCAATCGGGTAACTCCCAGGAGAGTGTCACAGAGC
AGGACAGCAAGGACAGCACCTACAGCCTCAGCAGCACCCTGACGCTGAGCAAAGCAGACTAC
GAGAAACACAAAGTCTACGCCTGCGAAGTCACCCATCAGGGCCTGAGCTCGCCCGTCACAAA
GAGCTTCAACAGGGGAGAGTGT
SEQ ID NO: 126:
EVQLVESGGGLVQPGGSLRLSCAASGFNIKDTYIHWVRQAPGKGLEWVARIYPTNGYTRYAD
SVKGRFTISADTSKNTAYLQMNSLRAEDTAVYYCSRWGGDGFYAMDYWGQGTLVTVSSASTK
GPSVFPLAPSSKSTSGGTAALGCLVKDYFPEPVTVSWNSGALTSGVHTFPAVLQSSGLYSLS
SVVTVPSSSLGTQTYICNVNHKPSNTKVDKKVEPKSCDKTHTCPPCPAPELLGGPSVFLFPP
KPKDTLMISRTPEVTCVVVDVSHEDPEVKFNWYVDGVEVHNAKTKPREEQYNSTYRVVSVLT
VLHQDWLNGKEYKCKVSNKALPAPIEKTISKAKGQPREPQVYTLPPSREEMTKNQVSLYCLV
KGFYPSDIAVEWESNGQPENNYKTTPPVLDSDGSFFLYSKLTVDKSRWQQGNVFSCSVMHEA
LHNHYTQKSLSLSPGK
SEQ ID NO: 127:
GAGGTGCAGCTGGTTGAATCTGGCGGAGGACTGGTTCAGCCTGGCGGATCTCTGAGACTGTC
TTGTGCCGCCAGCGGCTTCAACATCAAGGACACCTACATCCACTGGGTCCGACAGGCCCCTG
GCAAAGGACTTGAATGGGTCGCCAGAATCTACCCCACCAACGGCTACACCAGATACGCCGAC
TCTGTGAAGGGCAGATTCACCATCAGCGCCGACACCAGCAAGAACACCGCCTACCTGCAGAT
GAACAGCCTGAGAGCCGAGGACACCGCCGTGTACTACTGTTCTAGATGGGGAGGCGACGGCT
TCTACGCCATGGATTATTGGGGCCAGGGCACCCTGGTCACCGTTTCTTCTGCtagcACCAAG
GGCCCATCcGTCTTCCCCCTGGCACCCTCCTCCAAGAGCACCTCTGGGGGCACAGCGGCCCT
GGGCTGCCTGGTCAAGGACTACTTCCCCGAACCGGTGACGGTGTCcTGGAACTCAGGCGCtC
TGACCAGCGGCGTGCACACCTTCCCGGCTGTCCTACAGTCCTCAGGACTCTACTCCCTCAGC
AGCGTGGTGACCGTGCCCTCCAGCAGCTTGGGCACCCAGACCTACATCTGCAACGTGAATCA
CAAGCCGAGCAACACCAAGGTGGAGAAGAAAGTTGAGCCCAAATCTTGTGAGAAAACTCACA
CATGCCCACCGTGCCCAGCACCTGAACTCCTGGGGGGACCGTCAGTCTTCCTCTTCCCCCCA
AAACCCAAGGACACCCTCATGATCTCCCGGACCCCTGAGGTCACATGCGTGGTGGTGGACGT
GAGCCACGAAGACCCTGAGGTCAAGTTCAACTGGTACGTGGACGGCGTGGAGGTGCATAATG
CCAAGACAAAGCCGCGGGAGGAGCAGTACAACAGCACGTACCGTGTGGTCAGCGTCCTCACC
GTCCTGCACCAGGACTGGCTGAATGGCAAGGAGTACAAGTGCAAGGTCTCCAACAAAGCCCT
CCCAGCCCCCATCGAGAAAACCATCTCCAAAGCCAAAGGGCAGCCCCGAGAACCACAGGTcT
ACACCCTGCCCCCATCCCGGGAGGAGATGACCAAGAACCAGGTCAGCCTGtacTGCCTGGTC
AAAGGCTTCTATCCCAGCGACATCGCCGTGGAGTGGGAGAGCAATGGGCAGCCGGAGAACAA
CTACAAGACCACGCCTCCCGTGCTGGACTCCGACGGCTCCTTCTTCCTCTATAGCAAGCTCA
CCGTGGACAAGAGCAGGTGGCAGCAGGGGAACGTCTTCTCATGCTCCGTGATGCATGAGGCT
CTGCACAACCACTACACGCAGAAGAGCCTCTCCCTGTCTCCGGGTAAA
SEQ ID NO: 128:
GACGCCTCTTTACCCTATTTACAGAAGGAGAGCGTCTTTCAGTCCGGCGCTCACGCCTATAG
GATCCCCGCTTTACTGTATTTACCCGGTCAGCAGTCTTTACTGGCTTTCGCCGAGCAGCGGG
CTTCCAAGAAGGACGAGCACGCTGAGCTGATCGTGTTACGTAGGGGAGACTACGACGCCCCC
ACCCATCAAGTTCAATGGCAAGCTCAAGAAGTGGTGGCTCAAGCTCGGCTCGATGGCCATCG
GAGCATGAACCCTTGTCCCCTCTACGACGCCCAAACCGGCACTTTATTTCTGTTCTTCATCG
CCATCCCCGGTCAAGTTACCGAGCAGCAACAGCTGCAGACCCGGGCTAACGTGACAAGGCTG
TGCCAAGTTACCTCCACCGACCACGGAAGGACTTGGTCCTCCCCTCGTGATCTGACCGATGC
CGCTATCGGCCCCGCTTACCGGGAGTGGTCCACCTTTGCCGTGGGACCCGGCCATTGTCTGC
AGCTGCATGATAGGGCTCGGTCTTTAGTGGTGCCCGCTTACGCCTACCGGAAGCTGCACCCC
AAGCAGCGGCCTATCCCCTCCGCTTTTTGTTTTTTAAGCCATGACCATGGTCGTACTTGGGC
TCGTGGCCATTTTGTGGCCCAAGATACTTTAGAGTGCCAAGTTGCCGAGGTGGAGACTGGTG
AGCAGCGGGTGGTGACTTTAAATGCCCGGTCCCATTTAAGGGCTAGGGTGCAAGCCCAGTCC
ACCAACGACGGACTGGATTTCCAAGAATCCCAGCTGGTGAAGAAGCTCGTCGAACCTCCCCC
CCAAGGTTGCCAAGGAAGCGTGATCTCCTTCCCCTCCCCTAGGAGCGGACCCGGTTCCCCCG
CTCAGTGGCTGCTCTACACCCATCCCACCCATTCTTGGCAGAGGGCTGATTTAGGCGCCTAT
TTAAACCCTCGTCCTCCCGCTCCCGAAGCTTGGAGCGAGCCCGTGCTGCTCGCTAAGGGCAG
CGCCGCCTACAGCGATTTACAGTCCATGGGAACCGGACCCGATGGCAGCCCTCTGTTCGGCT
GTTTATATGAGGCTAACGACTACGAGGAGATCGTGTTTCTCATGTTCACTTTAAAGCAAGCT
TTTCCCGCTGAGTATCTGCCCCAAGGTGGAGGCGGCAGCGGCGGCGGCGGCTCCGACAAAAC
TCACACATGCCCACCGTGCCCAGCACCTGAACTCCTGGGGGGACCGTCAGTCTTCCTCTTCC
CCCCAAAACCCAAGGACACCCTCATGATCTCCCGGACCCCTGAGGTCACATGCGTGGTGGTG
GACGTGAGCCACGAAGACCCTGAGGTCAAGTTCAACTGGTACGTGGACGGCGTGGAGGTGCA
TAATGCCAAGACAAAGCCGCGGGAGGAGCAGTACAACAGCACGTACCGTGTGGTCAGCGTCC
TCACCGTCCTGCACCAGGACTGGCTGAATGGCAAGGAGTACAAGTGCAAGGTCTCCAACAAA
GCCCTCCCAGCCCCCATCGAGAAAACCATCTCCAAAGCCAAAGGGCAGCCCCGAGAACCACA
GGTGTACACCCTGCCCCCATCCCGGGAGGAGATGACCAAGAACCAGGTCAGCCTGACCTGCC
TGGTCAAAGGCTTCTATCCCAGCGACATCGCCGTGGAGTGGGAGAGCAATGGGCAGCCGGAG
AACAACTACAAGACCACGCCTCCCGTGCTGGACTCCGACGGCTCCTTCTTCCTCACCAGCAA
GCTCACCGTGGACAAGAGCAGGTGGCAGCAGGGGAACGTCTTCTCATGCTCCGTGATGCATG
AGGCTCTGCACAACCACTACACGCAGAAGAGCCTCTCCCTGTCTCCGGGTAAATGA
SEQ ID NO: 129:
ASLPYLQKESVFQSGAHAYRIPALLYLPGQQSLLAFAEQRASKKDEHAELIVLRRGDYDAPT
HQVQWQAQEVVAQARLDGHRSMNPCPLYDAQTGTLFLFFIAIPGQVTEQQQLQTRANVTRLC
QVTSTDHGRTWSSPRDLTDAAIGPAYREWSTFAVGPGHCLQLHDRARSLVVPAYAYRKLHPK
QRPIPSAFCFLSHDHGRTWARGHFVAQDTLECQVAEVETGEQRVVTLNARSHLRARVQAQST
NDGLDFQESQLVKKLVEPPPQGCQGSVISFPSPRSGPGSPAQWLLYTHPTHSWQRADLGAYL
NPRPPAPEAWSEPVLLAKGSCAYSDLQSMGTGPDGSPLFGCLYEANDYEEIVFLMFTLKQAF
PAEYLPQGGGGSGGGGSDKTHTCPPCPAPELLGGPSVFLFPPKPKDTLMISRTPEVTCVVVD
VSHEDPEVKFNWYVDGVEVHNAKTKPREEQYNSTYRVVSVLTVLHQDWLNGKEYKCKVSNKA
LPAPIEKTISKAKGQPREPQVYTLPPSREEMTKNQVSLTCLVKGFYPSDIAVEWESNGQPEN
NYKTTPPVLDSDGSFFLYSKLTVDKSRWQQGNVFSCSVMHEALHNHYTQKSLSLSPGK
SEQ ID NO: 130:
ASLPYLQKESVFQSGAHAYRIPALLYLPGQQSLLAFAEQRASKKDEHAELIVLRRGDYDAPT
HQVQWQAQEVVAQARLDGHRSMNPCPLYDAQTGTLFLFFIAIPGQVTEQQQLQTRANVTRLC
QVTSTDHGRTWSSPRDLTDAAIGPAYREWSTFAVGPGHCLQLHDRARSLVVPAYAYRKLHPK
QRPIPSAFCFLSHDHGRTWARGHFVAQDTLECQVAEVETGEQRVVTLNARSHLRARVQAQST
NDGLDFQESQLVKKLVEPPPQGCQGSVISFPSPRSGPGSPAQWLLYTHPTHSWQRADLGAYL
NPRPPAPEAWSEPVLLAKGSAAYSDLQSMGTGPDGSPLFGCLYEANDYEEIVFLMFTLKQAF
PAEYLPQGGGGSGGGGSDKTHTCPPCPAPELLGGPSVFLFPPKPKDTLMISRTPEVTCVVVD
VSHEDPEVKFNWYVDGVEVHNAKTKPREEQYNSTYRVVSVLTVLHQDWLNGKEYKCKVSNKA
LPAPIEKTISKAKGQPREPQVYTLPPSREEMTKNQVSLTCLVKGFYPSDIAVEWESNGQPEN
NYKTTPPVLDSDGSFFLYSKLTVDKSRWQQGNVFSCSVMHEALHNHYTQKSLSLSPGK
SEQ ID NO: 131:
AASLPYLQKESVFQSGAHAYRIPALLYLPGQQSLLAFAEQRASKKDEHAELIVLRRGDYDAP
THQVQWQAQEVVAQARLDGHRSMNPCPLYDAQTGTLFLFFIAIPGQVTEQQQLQTRANVTRL
CQVTSTDHGRTWSSPRDLTDAAIGPAYREWSTFAVGPGHCLQLHDRARSLVVPAYAYRKLHP
KQRPIPSAFCFLSHDHGRTWARGHFVAQDTLECQVAEVETGEQRVVTLNARSHLRARVQAQS
TNDGLDFQESQLVKKLVEPPPQGCQGSVISFPSPRSGPGSPAQWLLYTHPTHSWQRADLGAY
LNPRPPAPEAWSEPVLLAKGSAAYSDLQSMGTGPDGSPLFGCLYEANDYEEIVFLMFTLKQA
FPAEYLPQGGGGSGGGGSDKTHTCPPCPAPELLGGPSVFLFPPKPKDTLMISRTPEVTCVVV
DVSHEDPEVKFNWYVDGVEVHNAKTKPREEQYNSTYRVVSVLTVLHQDWLNGKEYKCKVSNK
ALPAPIEKTISKAKGQPREPQVYTLPPSREEMTKNQVSLTCLVKGFYPSDIAVEWESNGQPE
NNYKTTPPVLDSDGSFFLYSKLTVDKSRWQQGNVFSCSVMHEALHNHYTQKSLSLSPGK
SEQ ID NO: 132:
DASLPYLQKESVFQSGAHAYRIPALLYLPGQQSLLAFAEQRASKKDEHAELIVLRRGDYDAP
THQVQWQAQEVVAQARLDGHRSMNPCPLYDAQTGTLFLFFIAIPGQVTEQQQLQTRANVTRL
CQVTSTDHGRTWSSPRDLTDAAIGPAYREWSTFAVGPGHCLQLHDRARSLVVPAYAYRKLHP
KQRPIPSAFCFLSHDHGRTWARGHFVAQDTLECQVAEVETGEQRVVTLNARSHLRARVQAQS
TNDGLDFQESQLVKKLVEPPPQGCQGSVISFPSPRSGPGSPAQWLLYTHPTHSWQRADLGAY
LNPRPPAPEAWSEPVLLAKGSAAYSDLQSMGTGPDGSPLFGCLYEANDYEEIVFLMFTLKQA
FPAEYLPQGGGGSGGGGSDKTHTCPPCPAPELLGGPSVFLFPPKPKDTLMISRTPEVTCVVV
DVSHEDPEVKFNWYVDGVEVHNAKTKPREEQYNSTYRVVSVLTVLHQDWLNGKEYKCKVSNK
ALPAPIEKTISKAKGQPREPQVYTLPPSREEMTKNQVSLTCLVKGFYPSDIAVEWESNGQPE
NNYKTTPPVLDSDGSFFLYSKLTVDKSRWQQGNVFSCSVMHEALHNHYTQKSLSLSPGK
SEQ ID NO: 133:
AASLPYLQKESVFQSGAHAYRIPALLYLPGQQSLLAFAEQRASKKDEHAELIVLRRGDYDAP
THQVQWQAQEVVAQARLDGHRSMNPCPLYDAQTGTLFLFFIAIPGQVTEQQQLQTRANVTRL
CQVTSTDHGRTWSSPRDLTDAAIGPAYREWSTFAVGPGHCLQLHDRARSLVVPAYAYRKLHP
KQRPIPSAFCFLSHDHGRTWARGHFVAQDTLECQVAEVETGEQRVVTLNARSHLRARVQAQS
TNDGLDFQESQLVKKLVEPPPQGCQGSVISFPSPRSGPGSPAQWLLYTHPTHSWQRADLGAY
LNPRPPAPEAWSEPVLLAKGSCAYSDLQSMGTGPDGSPLFGCLYEANDYEEIVFLMFTLKQA
FPAEYLPQGGGGSGGGGSDKTHTCPPCPAPELLGGPSVFLFPPKPKDTLMISRTPEVTCVVV
DVSHEDPEVKFNWYVDGVEVHNAKTKPREEQYNSTYRVVSVLTVLHQDWLNGKEYKCKVSNK
ALPAPIEKTISKAKGQPREPQVYTLPPSREEMTKNQVSLTCLVKGFYPSDIAVEWESNGQPE
NNYKTTPPVLDSDGSFFLYSKLTVDKSRWQQGNVFSCSVMHEALHNHYTQKSLSLSPGK
SEQ ID NO: 134:
DASLPYLQKESVFQSGAHAYRIPALLYLPGQQSLLAFAEQRASKKDEHAELIVLRRGDYDAP
THQVQWQAQEVVAQARLDGHRSMNPCPLYDAQTGTLFLFFIAIPGQVTEQQQLQTRANVTRL
CQVTSTDHGRTWSSPRDLTDAAIGPAYREWSTFAVGPGHCLQLHDRARSLVVPAYAYRKLHP
KQRPIPSAFCFLSHDHGRTWARGHFVAQDTLECQVAEVETGEQRVVTLNARSHLRARVQAQS
TNDGLDFQESQLVKKLVEPPPQGCQGSVISFPSPRSGPGSPAQWLLYTHPTHSWQRADLGAY
LNPRPPAPEAWSEPVLLAKGSCAYSDLQSMGTGPDGSPLFGCLYEANDYEEIVFLMFTLKQA
FPAEYLPQGGGGSGGGGSDKTHTCPPCPAPELLGGPSVFLFPPKPKDTLMISRTPEVTCVVV
DVSHEDPEVKFNWYVDGVEVHNAKTKPREEQYNSTYRVVSVLTVLHQDWLNGKEYKCKVSNK
ALPAPIEKTISKAKGQPREPQVYTLPPSREEMTKNQVSLTCLVKGFYPSDIAVEWESNGQPE
NNYKTTPPVLDSDGSFFLYSKLTVDKSRWQQGNVFSCSVMHEALHNHYTQKSLSLSPGK
SEQ ID NO: 135:
MASLPYLQKESVFQSGAHAYRIPALLYLPGQQSLLAFAEQRASKKDEHAELIVLRRGDYDAP
THQVQWQAQEVVAQARLDGHRSMNPCPLYDAQTGTLFLFFIAIPGQVTEQQQLQTRANVTRL
CQVTSTDHGRTWSSPRDLTDAAIGPAYREWSTFAVGPGHCLQLHDRARSLVVPAYAYRKLHP
KQRPIPSAFCELSHDHGRTWARGHEVAQDTLECQVAEVETGEQRVVTLNARSHLRARVQAQS
TNDGLDFQESQLVKKLVEPPPQGCQGSVISFPSPRSGPGSPAQWLLYTHPTHSWQRADLGAY
LNPRPPAPEAWSEPVLLAKGSCAYSDLQSMGTGPDGSPLFGCLYEANDYEEIVFLMFTLKQA
FPAEYLPQGGGGSGGGGSDKTHTCPPCPAPELLGGPSVFLFPPKPKDTLMISRTPEVTCVVV
DVSHEDPEVKFNWYVDGVEVHNAKTKPREEQYNSTYRVVSVLTVLHQDWLNGKEYKCKVSNK
ALPAPIEKTISKAKGQPREPQVYTLPPSREEMTKNQVSLTCLVKGFYPSDIAVEWESNGQPE
NNYKTTPPVLDSDGSFFLYSKLTVDKSRWQQGNVFSCSVMHEALHNHYTQKSLSLSPGK
SEQ ID NO: 136:
MASLPYLQKESVFQSGAHAYRIPALLYLPGQQSLLAFAEQRASKKDEHAELIVLRRGDYDAP
THQVQWQAQEVVAQARLDGHRSMNPCPLYDAQTGTLFLFFIAIPGQVTEQQQLQTRANVTRL
CQVTSTDHGRTWSSPRDLTDAAIGPAYREWSTFAVGPGHCLQLHDRARSLVVPAYAYRKLHP
KQRPIPSAFCFLSHDHGRTWARGHFVAQDTLECQVAEVETGEQRVVTLNARSHLRARVQAQS
TNDGLDFQESQLVKKLVEPPPQGCQGSVISFPSPRSGPGSPAQWLLYTHPTHSWQRADLGAY
LNPRPPAPEAWSEPVLLAKGSAAYSDLQSMGTGPDGSPLFGCLYEANDYEEIVFLMFTLKQA
FPAEYLPQGGGGSGGGGSDKTHTCPPCPAPELLGGPSVFLFPPKPKDTLMISRTPEVTCVVV
DVSHEDPEVKFNWYVDGVEVHNAKTKPREEQYNSTYRVVSVLTVLHQDWLNGKEYKCKVSNK
ALPAPIEKTISKAKGQPREPQVYTLPPSREEMTKNQVSLTCLVKGFYPSDIAVEWESNGQPE
NNYKTTPPVLDSDGSFFLYSKLTVDKSRWQQGNVFSCSVMHEALHNHYTQKSLSLSPGK
SEQ ID NO: 137:
DASLPYLQKESVFQSGAHAYRIPALLYLPGQQSLLAFAEQRASKKDEHAELIVLRRGDYDAG
THQVQWQAQEVVAQARLDGHRSMNPCPLYDEQTGTLFLFFIAIPGQVTEQQQLQTRANVTRL
CQVTSTDHGRTWSSPRDLTDAAIGPAYREWSTFAVGPGHCLQLHDRARSLVVPAYAYRKLHP
KQRPIPSAFCFLSHDHGRTWARGHFVAQDTLECQVAEVETGEQRVVTLNARSHLRARVQAQS
TNDGLDFQESQLVKKLVEPPPQGCQGSVISFPSPRSGPGSPAQWLLYTHPTHSWQRADLGAY
LNPRPPAPEAWSEPVLLAKGSAAYSDLQSMGTGPDGSPLFGCLYEANDYEEIVFLMFTLKQA
FPAEYLPQGGGGSGGGGSDKTHTCPPCPAPELLGGPSVFLFPPKPKDTLMISRTPEVTCVVV
DVSHEDPEVKFNWYVDGVEVHNAKTKPREEQYNSTYRVVSVLTVLHQDWLNGKEYKCKVSNK
ALPAPIEKTISKAKGQPREPQVYTLPPSREEMTKNQVSLTCLVKGFYPSDIAVEWESNGQPE
NNYKTTPPVLDSDGSFFLYSKLTVDKSRWQQGNVFSCSVMHEALHNHYTQKSLSLSPGK
SEQ ID NO: 138:
DASLPYLQDESVFQSGAHAYRIPALLYLPGQQSLLAFAEQRASKKDEHAELIVLRRGDYDAP
THQVQWQAQEVVAQARLDGHRSMNPCPLYDEQTGTLFLFFIAIPGQVTEQQQLQTRANVTRL
CQVTSTDHGRTWSSPRDLTDAAIGPAYREWSTFAVGPGHCLQLHDRARSLVVPAYAYRKLHP
KQRPIPSAFCFLSHDHGRTWARGHFVAQDTLECQVAEVETGEQRVVTLNARSHLRARVQAQS
TNDGLDFQESQLVKKLVEPPPQGCQGSVISFPSPRSGPGSPAQWLLYTHPTHSWQRADLGAY
LNPRPPAPEAWSEPVLLAKGSAAYSDLQSMGTGPDGSPLFGCLYEANDYEEIRFIMFTLKQA
FPAEYLPQGGGGSGGGGSDKTHTCPPCPAPELLGGPSVFLFPPKPKDTLMISRTPEVTCVVV
DVSHEDPEVKFNWYVDGVEVHNAKTKPREEQYNSTYRVVSVLTVLHQDWLNGKEYKCKVSNK
ALPAPIEKTISKAKGQPREPQVYTLPPSREEMTKNQVSLTCLVKGFYPSDIAVEWESNGQPE
NNYKTTPPVLDSDGSFFLYSKLTVDKSRWQQGNVFSCSVMHEALHNHYTQKSLSLSPGK
SEQ ID NO: 139:
DASLPYLQKESVFQSGAHAYRIPALLYLPGQQSLLAFAEQRASKKDEHAELIVLRRGDYDAN
THQVQWQAQEVVAQARLDGHRSMNPCPLYDAQTGTLFLFFIAIPGQVTEQQQLQTRANVTRL
CQVTSTDHGRTWSSPRDLTDAAIGPAYREWSTFAVGPGHCLQLHDRARSLVVPAYAYRKLHP
KQRPIPSAFCFLSHDHGRTWARGHFVAQDTLECQVAEVETGEQRVVTLNARSHLRARVQAQS
TNDGLDFQESQLVKKLVEPPPQGCQGSVISFPSPRSGPGSPAQWLLYTHPTHSWQRADLGAY
LNPRPPAPEAWSEPVLLAKGSAAYSDLQSMGTGPDGSPLFGCLYEANDYEEIVFLMFTLKQA
FPAEYLPQGGGGSGGGGSDKTHTCPPCPAPELLGGPSVFLFPPKPKDTLMISRTPEVTCVVV
DVSHEDPEVKFNWYVDGVEVHNAKTKPREEQYNSTYRVVSVLTVLHQDWLNGKEYKCKVSNK
ALPAPIEKTISKAKGQPREPQVYTLPPSREEMTKNQVSLTCLVKGFYPSDIAVEWESNGQPE
NNYKTTPPVLDSDGSFFLYSKLTVDKSRWQQGNVFSCSVMHEALHNHYTQKSLSLSPGK
SEQ ID NO: 140:
DASLPYLQKESVFQSGAHAYRIPALLYLPGQQSLLAFAEQRASKKDEHAELIVLRRGDYDAP
THQVQWQAQEVVAQARLDGHRSMNPCPLYDAQTGTLFLFFIAIPGQVTEQQQLQTRANVTRL
CQVTSTDHGRTWSSPRDLTDAAIGPAYREWSTFAVGPGHCLQLHDRARSLVVPAYAYRKLHP
KQRPIPSAFCFLSHDHGRTWARGHFVAQDTLECQVAEVETGEQRVVTLNARSHLRARVQAQS
TNDGLDFQESQLVKKLVEPPPAGCQGSVISFPSPRSGPGSPAQWLLYTHPTHRKQRADLGAY
LNPRPPAPEAWSEPVLLAKGSAAYSDLQSMGTGPDGSPLFGCLYEANDYEEIVFLMFTLKQA
FPAEYLPQGGGGSGGGGSDKTHTCPPCPAPELLGGPSVFLFPPKPKDTLMISRTPEVTCVVV
DVSHEDPEVKFNWYVDGVEVHNAKTKPREEQYNSTYRVVSVLTVLHQDWLNGKEYKCKVSNK
ALPAPIEKTISKAKGQPREPQVYTLPPSREEMTKNQVSLTCLVKGFYPSDIAVEWESNGQPE
NNYKTTPPVLDSDGSFFLYSKLTVDKSRWQQGNVFSCSVMHEALHNHYTQKSLSLSPGK
SEQ ID NO: 141:
DASLPYLQKESVFQSGAHAYRIPALLYLPGQQSLLAFAEQRASKKDEHAELIVLRRGDYDAS
THQVQWQAQEVVAQARLDGHRSMNPCPLYDAQTGTLFLFFIAIPGQVTEQQQLQTRANVTRL
CQVTSTDHGRTWSSPRDLTDAAIGPAYREWSTFAVGPGHCLQLHDRARSLVVPAYAYRKLHP
KQRPIPSAFCFLSHDHGRTWARGHFVAQDTLECQVAEVETGEQRVVTLNARSHLRARVQAQS
TNDGLDFQESQLVKKLVEPPPAGCQGSVISFPSPRSGPGSPAQWLLYTHPTHRKQRADLGAY
LNPRPPAPEAWSEPVLLAKGSAAYSDLQSMGTGPDGSPLFGCLYEANDYEEIVFLMFTLKQA
FPAEYLPQGGGGSGGGGSDKTHTCPPCPAPELLGGPSVFLFPPKPKDTLMISRTPEVTCVVV
DVSHEDPEVKFNWYVDGVEVHNAKTKPREEQYNSTYRVVSVLTVLHQDWLNGKEYKCKVSNK
ALPAPIEKTISKAKGQPREPQVYTLPPSREEMTKNQVSLTCLVKGFYPSDIAVEWESNGQPE
NNYKTTPPVLDSDGSFFLYSKLTVDKSRWQQGNVFSCSVMHEALHNHYTQKSLSLSPGK
SEQ ID NO: 142:
DASLPYLQKESVFQSGAHAYRIPALLYLPGQQSLLAFAEQRASKKDEHAELIVLRRGDYDAT
THQVQWQAQEVVAQARLDGHRSMNPCPLYDAQTGTLFLFFIAIPGQVTEQQQLQTRANVTRL
CQVTSTDHGRTWSSPRDLTDAAIGPAYREWSTFAVGPGHCLQLHDRARSLVVPAYAYRKLHP
KQRPIPSAFCFLSHDHGRTWARGHFVAQDTLECQVAEVETGEQRVVTLNARSHLRARVQAQS
TNDGLDFQESQLVKKLVEPPPAGCQGSVISFPSPRSGPGSPAQWLLYTHPTHRKQRADLGAY
LNPRPPAPEAWSEPVLLAKGSAAYSDLQSMGTGPDGSPLFGCLYEANDYEEIVFLMFTLKQA
FPAEYLPQGGGGSGGGGSDKTHTCPPCPAPELLGGPSVFLFPPKPKDTLMISRTPEVTCVVV
DVSHEDPEVKFNWYVDGVEVHNAKTKPREEQYNSTYRVVSVLTVLHQDWLNGKEYKCKVSNK
ALPAPIEKTISKAKGQPREPQVYTLPPSREEMTKNQVSLTCLVKGFYPSDIAVEWESNGQPE
NNYKTTPPVLDSDGSFFLYSKLTVDKSRWQQGNVFSCSVMHEALHNHYTQKSLSLSPGK
SEQ ID NO: 143:
DASLPYLQKESVFQSGAHAYRIPALLYLPGQQSLLAFAEQRASKKDEHAELIVLRRGDYDAN
THQVQWQAQEVVAQARLDGHRSMNPCPLYDAQTGTLFLFFIAIPGQVTEQQQLQTRANVTRL
CQVTSTDHGRTWSSPRDLTDAAIGPAYREWSTFAVGPGHCLQLHDRARSLVVPAYAYRKLHP
KQRPIPSAFCFLSHDHGRTWARGHFVAQDTLECQVAEVETGEQRVVTLNARSHLRARVQAQS
TNDGLDFQESQLVKKLVEPPPAGCQGSVISFPSPRSGPGSPAQWLLYTHPTHRKQRADLGAY
LNPRPPAPEAWSEPVLLAKGSAAYSDLQSMGTGPDGSPLFGCLYEANDYEEIVFLMFTLKQA
FPAEYLPQGGGGSGGGGSDKTHTCPPCPAPELLGGPSVFLFPPKPKDTLMISRTPEVTCVVV
DVSHEDPEVKFNWYVDGVEVHNAKTKPREEQYNSTYRVVSVLTVLHQDWLNGKEYKCKVSNK
ALPAPIEKTISKAKGQPREPQVYTLPPSREEMTKNQVSLTCLVKGFYPSDIAVEWESNGQPE
NNYKTTPPVLDSDGSFFLYSKLTVDKSRWQQGNVFSCSVMHEALHNHYTQKSLSLSPGK
SEQ ID NO: 144:
ASLPYLQKESVFQSGAHAYRIPALLYLPGQQSLLAFAEQRASKKDEHAELIVLRRGDYDAPT
HQVQWQAQEVVAQARLDGHRSMNPCPLYDAQTGTLFLFFIAIPGQVTEQQQLQTRANVTRLC
QVTSTDHGRTWSSPRDLTDAAIGPAYREWSTFAVGPGHCLQLHDRARSLVVPAYAYRKLHPK
QRPIPSAFCFLSHDHGRTWARGHFVAQDTLECQVAEVETGEQRVVTLNARSHLRARVQAQST
NDGLDFQESQLVKKLVEPPPQGCQGSVISFPSPRSGPGSPAQWLLYTHPTHSWQRADLGAYL
NPRPPAPEAWSEPVLLAKGSCAYSDLQSMGTGPDGSPLFGCLYEANDYEEIVFLMFTLKQAF
PAEYLPQEPKSSDKTHTCPPCPAPELLGGPSVFLFPPKPKDTLMISRTPEVTCVVVDVSHED
PEVKFNWYVDGVEVHNAKTKPREEQYNSTYRVVSVLTVLHQDWLNGKEYKCKVSNKALPAPI
EKTISKAKGQPREPQVYTLPPSREEMTKNQVSLTCLVKGFYPSDIAVEWESNGQPENNYKTT
PPVLDSDGSFFLYSKLTVDKSRWQQGNVFSCSVMHEALHNHYTQKSLSLSPGK
SEQ ID NO: 145:
ASLPYLQKESVFQSGAHAYRIPALLYLPGQQSLLAFAEQRASKKDEHAELIVLRRGDYDAPT
HQVQWQAQEVVAQARLDGHRSMNPCPLYDAQTGTLFLFFIAIPGQVTEQQQLQTRANVTRLC
QVTSTDHGRTWSSPRDLTDAAIGPAYREWSTFAVGPGHCLQLHDRARSLVVPAYAYRKLHPK
QRPIPSAFCFLSHDHGRTWARGHFVAQDTLECQVAEVETGEQRVVTLNARSHLRARVQAQST
NDGLDFQESQLVKKLVEPPPQGCQGSVISFPSPRSGPGSPAQWLLYTHPTHSWQRADLGAYL
NPRPPAPEAWSEPVLLAKGSAAYSDLQSMGTGPDGSPLFGCLYEANDYEEIVFLMFTLKQAF
PAEYLPQEPKSSDKTHTCPPCPAPELLGGPSVFLFPPKPKDTLMISRTPEVTCVVVDVSHED
PEVKFNWYVDGVEVHNAKTKPREEQYNSTYRVVSVLTVLHQDWLNGKEYKCKVSNKALPAPI
EKTISKAKGQPREPQVYTLPPSREEMTKNQVSLTCLVKGFYPSDIAVEWESNGQPENNYKTT
PPVLDSDGSFFLYSKLTVDKSRWQQGNVFSCSVMHEALHNHYTQKSLSLSPGK
SEQ ID NO: 146:
AASLPYLQKESVFQSGAHAYRIPALLYLPGQQSLLAFAEQRASKKDEHAELIVLRRGDYDAP
THQVQWQAQEVVAQARLDGHRSMNPCPLYDAQTGTLFLFFIAIPGQVTEQQQLQTRANVTRL
CQVTSTDHGRTWSSPRDLTDAAIGPAYREWSTFAVGPGHCLQLHDRARSLVVPAYAYRKLHP
KQRPIPSAFCFLSHDHGRTWARGHFVAQDTLECQVAEVETGEQRVVTLNARSHLRARVQAQS
TNDGLDFQESQLVKKLVEPPPQGCQGSVISFPSPRSGPGSPAQWLLYTHPTHSWQRADLGAY
LNPRPPAPEAWSEPVLLAKGSAAYSDLQSMGTGPDGSPLFGCLYEANDYEEIVFLMFTLKQA
FPAEYLPQEPKSSDKTHTCPPCPAPELLGGPSVFLFPPKPKDTLMISRTPEVTCVVVDVSHE
DPEVKFNWYVDGVEVHNAKTKPREEQYNSTYRVVSVLTVLHQDWLNGKEYKCKVSNKALPAP
IEKTISKAKGQPREPQVYTLPPSREEMTKNQVSLTCLVKGFYPSDIAVEWESNGQPENNYKT
TPPVLDSDGSFFLYSKLTVDKSRWQQGNVFSCSVMHEALHNHYTQKSLSLSPGK
SEQ ID NO: 147:
DASLPYLQKESVFQSGAHAYRIPALLYLPGQQSLLAFAEQRASKKDEHAELIVLRRGDYDAP
THQVQWQAQEVVAQARLDGHRSMNPCPLYDAQTGTLFLFFIAIPGQVTEQQQLQTRANVTRL
CQVTSTDHGRTWSSPRDLTDAAIGPAYREWSTFAVGPGHCLQLHDRARSLVVPAYAYRKLHP
KQRPIPSAFCFLSHDHGRTWARGHFVAQDTLECQVAEVETGEQRVVTLNARSHLRARVQAQS
TNDGLDFQESQLVKKLVEPPPQGCQGSVISFPSPRSGPGSPAQWLLYTHPTHSWQRADLGAY
LNPRPPAPEAWSEPVLLAKGSAAYSDLQSMGTGPDGSPLFGCLYEANDYEEIVFLMFTLKQA
FPAEYLPQEPKSSDKTHTCPPCPAPELLGGPSVFLFPPKPKDTLMISRTPEVTCVVVDVSHE
DPEVKFNWYVDGVEVHNAKTKPREEQYNSTYRVVSVLTVLHQDWLNGKEYKCKVSNKALPAP
IEKTISKAKGQPREPQVYTLPPSREEMTKNQVSLTCLVKGFYPSDIAVEWESNGQPENNYKT
TPPVLDSDGSFFLYSKLTVDKSRWQQGNVFSCSVMHEALHNHYTQKSLSLSPGK
SEQ ID NO: 148:
AASLPYLQKESVFQSGAHAYRIPALLYLPGQQSLLAFAEQRASKKDEHAELIVLRRGDYDAP
THQVQWQAQEVVAQARLDGHRSMNPCPLYDAQTGTLFLFFIAIPGQVTEQQQLQTRANVTRL
CQVTSTDHGRTWSSPRDLTDAAIGPAYREWSTFAVGPGHCLQLHDRARSLVVPAYAYRKLHP
KQRPIPSAFCFLSHDHGRTWARGHFVAQDTLECQVAEVETGEQRVVTLNARSHLRARVQAQS
TNDGLDFQESQLVKKLVEPPPQGCQGSVISFPSPRSGPGSPAQWLLYTHPTHSWQRADLGAY
LNPRPPAPEAWSEPVLLAKGSCAYSDLQSMGTGPDGSPLFGCLYEANDYEEIVFLMFTLKQA
FPAEYLPQEPKSSDKTHTCPPCPAPELLGGPSVFLFPPKPKDTLMISRTPEVTCVVVDVSHE
DPEVKFNWYVDGVEVHNAKTKPREEQYNSTYRVVSVLTVLHQDWLNGKEYKCKVSNKALPAP
IEKTISKAKGQPREPQVYTLPPSREEMTKNQVSLTCLVKGFYPSDIAVEWESNGQPENNYKT
TPPVLDSDGSFFLYSKLTVDKSRWQQGNVFSCSVMHEALHNHYTQKSLSLSPGK
SEQ ID NO: 149:
DASLPYLQKESVFQSGAHAYRIPALLYLPGQQSLLAFAEQRASKKDEHAELIVLRRGDYDAP
THQVQWQAQEVVAQARLDGHRSMNPCPLYDAQTGTLFLFFIAIPGQVTEQQQLQTRANVTRL
CQVTSTDHGRTWSSPRDLTDAAIGPAYREWSTFAVGPGHCLQLHDRARSLVVPAYAYRKLHP
KQRPIPSAFCFLSHDHGRTWARGHFVAQDTLECQVAEVETGEQRVVTLNARSHLRARVQAQS
TNDGLDFQESQLVKKLVEPPPQGCQGSVISFPSPRSGPGSPAQWLLYTHPTHSWQRADLGAY
LNPRPPAPEAWSEPVLLAKGSCAYSDLQSMGTGPDGSPLFGCLYEANDYEEIVFLMFTLKQA
FPAEYLPQEPKSSDKTHTCPPCPAPELLGGPSVFLFPPKPKDTLMISRTPEVTCVVVDVSHE
DPEVKFNWYVDGVEVHNAKTKPREEQYNSTYRVVSVLTVLHQDWLNGKEYKCKVSNKALPAP
IEKTISKAKGQPREPQVYTLPPSREEMTKNQVSLTCLVKGFYPSDIAVEWESNGQPENNYKT
TPPVLDSDGSFFLYSKLTVDKSRWQQGNVFSCSVMHEALHNHYTQKSLSLSPGK
SEQ ID NO: 150:
MASLPYLQKESVFQSGAHAYRIPALLYLPGQQSLLAFAEQRASKKDEHAELIVLRRGDYDAP
THQVQWQAQEVVAQARLDGHRSMNPCPLYDAQTGTLFLFFIAIPGQVTEQQQLQTRANVTRL
CQVTSTDHGRTWSSPRDLTDAAIGPAYREWSTFAVGPGHCLQLHDRARSLVVPAYAYRKLHP
KQRPIPSAFCFLSHDHGRTWARGHFVAQDTLECQVAEVETGEQRVVTLNARSHLRARVQAQS
TNDGLDFQESQLVKKLVEPPPQGCQGSVISFPSPRSGPGSPAQWLLYTHPTHSWQRADLGAY
LNPRPPAPEAWSEPVLLAKGSCAYSDLQSMGTGPDGSPLFGCLYEANDYEEIVFLMFTLKQA
FPAEYLPQEPKSSDKTHTCPPCPAPELLGGPSVFLFPPKPKDTLMISRTPEVTCVVVDVSHE
DPEVKFNWYVDGVEVHNAKTKPREEQYNSTYRVVSVLTVLHQDWLNGKEYKCKVSNKALPAP
IEKTISKAKGQPREPQVYTLPPSREEMTKNQVSLTCLVKGFYPSDIAVEWESNGQPENNYKT
TPPVLDSDGSFFLYSKLTVDKSRWQQGNVFSCSVMHEALHNHYTQKSLSLSPGK
SEQ ID NO: 151:
MASLPYLQKESVFQSGAHAYRIPALLYLPGQQSLLAFAEQRASKKDEHAELIVLRRGDYDAP
THQVQWQAQEVVAQARLDGHRSMNPCPLYDAQTGTLFLFFIAIPGQVTEQQQLQTRANVTRL
CQVTSTDHGRTWSSPRDLTDAAIGPAYREWSTFAVGPGHCLQLHDRARSLVVPAYAYRKLHP
KQRPIPSAFCFLSHDHGRTWARGHFVAQDTLECQVAEVETGEQRVVTLNARSHLRARVQAQS
TNDGLDFQESQLVKKLVEPPPQGCQGSVISFPSPRSGPGSPAQWLLYTHPTHSWQRADLGAY
LNPRPPAPEAWSEPVLLAKGSAAYSDLQSMGTGPDGSPLFGCLYEANDYEEIVFLMFTLKQA
FPAEYLPQEPKSSDKTHTCPPCPAPELLGGPSVFLFPPKPKDTLMISRTPEVTCVVVDVSHE
DPEVKFNWYVDGVEVHNAKTKPREEQYNSTYRVVSVLTVLHQDWLNGKEYKCKVSNKALPAP
IEKTISKAKGQPREPQVYTLPPSREEMTKNQVSLTCLVKGFYPSDIAVEWESNGQPENNYKT
TPPVLDSDGSFFLYSKLTVDKSRWQQGNVFSCSVMHEALHNHYTQKSLSLSPGK
SEQ ID NO: 152:
DASLPYLQKESVFQSGAHAYRIPALLYLPGQQSLLAFAEQRASKKDEHAELIVLRRGDYDAG
THQVQWQAQEVVAQARLDGHRSMNPCPLYDEQTGTLFLFFIAIPGQVTEQQQLQTRANVTRL
CQVTSTDHGRTWSSPRDLTDAAIGPAYREWSTFAVGPGHCLQLHDRARSLVVPAYAYRKLHP
KQRPIPSAFCFLSHDHGRTWARGHFVAQDTLECQVAEVETGEQRVVTLNARSHLRARVQAQS
TNDGLDFQESQLVKKLVEPPPQGCQGSVISFPSPRSGPGSPAQWLLYTHPTHSWQRADLGAY
LNPRPPAPEAWSEPVLLAKGSAAYSDLQSMGTGPDGSPLFGCLYEANDYEEIVFLMFTLKQA
FPAEYLPQEPKSSDKTHTCPPCPAPELLGGPSVFLFPPKPKDTLMISRTPEVTCVVVDVSHE
DPEVKFNWYVDGVEVHNAKTKPREEQYNSTYRVVSVLTVLHQDWLNGKEYKCKVSNKALPAP
IEKTISKAKGQPREPQVYTLPPSREEMTKNQVSLTCLVKGFYPSDIAVEWESNGQPENNYKT
TPPVLDSDGSFFLYSKLTVDKSRWQQGNVFSCSVMHEALHNHYTQKSLSLSPGK
SEQ ID NO: 153:
DASLPYLQDESVFQSGAHAYRIPALLYLPGQQSLLAFAEQRASKKDEHAELIVLRRGDYDAP
THQVQWQAQEVVAQARLDGHRSMNPCPLYDEQTGTLFLFFIAIPGQVTEQQQLQTRANVTRL
CQVTSTDHGRTWSSPRDLTDAAIGPAYREWSTFAVGPGHCLQLHDRARSLVVPAYAYRKLHP
KQRPIPSAFCFLSHDHGRTWARGHFVAQDTLECQVAEVETGEQRVVTLNARSHLRARVQAQS
TNDGLDFQESQLVKKLVEPPPQGCQGSVISFPSPRSGPGSPAQWLLYTHPTHSWQRADLGAY
LNPRPPAPEAWSEPVLLAKGSAAYSDLQSMGTGPDGSPLFGCLYEANDYEEIRFIMFTLKQA
FPAEYLPQEPKSSDKTHTCPPCPAPELLGGPSVFLFPPKPKDTLMISRTPEVTCVVVDVSHE
DPEVKFNWYVDGVEVHNAKTKPREEQYNSTYRVVSVLTVLHQDWLNGKEYKCKVSNKALPAP
IEKTISKAKGQPREPQVYTLPPSREEMTKNQVSLTCLVKGFYPSDIAVEWESNGQPENNYKT
TPPVLDSDGSFFLYSKLTVDKSRWQQGNVFSCSVMHEALHNHYTQKSLSLSPGK
SEQ ID NO: 154:
DASLPYLQKESVFQSGAHAYRIPALLYLPGQQSLLAFAEQRASKKDEHAELIVLRRGDYDAN
THQVQWQAQEVVAQARLDGHRSMNPCPLYDAQTGTLFLFFIAIPGQVTEQQQLQTRANVTRL
CQVTSTDHGRTWSSPRDLTDAAIGPAYREWSTFAVGPGHCLQLHDRARSLVVPAYAYRKLHP
KQRPIPSAFCFLSHDHGRTWARGHFVAQDTLECQVAEVETGEQRVVTLNARSHLRARVQAQS
TNDGLDFQESQLVKKLVEPPPQGCQGSVISFPSPRSGPGSPAQWLLYTHPTHSWQRADLGAY
LNPRPPAPEAWSEPVLLAKGSAAYSDLQSMGTGPDGSPLFGCLYEANDYEEIVFLMFTLKQA
FPAEYLPQEPKSSDKTHTCPPCPAPELLGGPSVFLFPPKPKDTLMISRTPEVTCVVVDVSHE
DPEVKFNWYVDGVEVHNAKTKPREEQYNSTYRVVSVLTVLHQDWLNGKEYKCKVSNKALPAP
IEKTISKAKGQPREPQVYTLPPSREEMTKNQVSLTCLVKGFYPSDIAVEWESNGQPENNYKT
TPPVLDSDGSFFLYSKLTVDKSRWQQGNVFSCSVMHEALHNHYTQKSLSLSPGK
SEQ ID NO: 155:
DASLPYLQKESVFQSGAHAYRIPALLYLPGQQSLLAFAEQRASKKDEHAELIVLRRGDYDAP
THQVQWQAQEVVAQARLDGHRSMNPCPLYDAQTGTLFLFFIAIPGQVTEQQQLQTRANVTRL
CQVTSTDHGRTWSSPRDLTDAAIGPAYREWSTFAVGPGHCLQLHDRARSLVVPAYAYRKLHP
KQRPIPSAFCFLSHDHGRTWARGHFVAQDTLECQVAEVETGEQRVVTLNARSHLRARVQAQS
TNDGLDFQESQLVKKLVEPPPAGCQGSVISFPSPRSGPGSPAQWLLYTHPTHRKQRADLGAY
LNPRPPAPEAWSEPVLLAKGSAAYSDLQSMGTGPDGSPLFGCLYEANDYEEIVFLMFTLKQA
FPAEYLPQEPKSSDKTHTCPPCPAPELLGGPSVFLFPPKPKDTLMISRTPEVTCVVVDVSHE
DPEVKFNWYVDGVEVHNAKTKPREEQYNSTYRVVSVLTVLHQDWLNGKEYKCKVSNKALPAP
IEKTISKAKGQPREPQVYTLPPSREEMTKNQVSLTCLVKGFYPSDIAVEWESNGQPENNYKT
TPPVLDSDGSFFLYSKLTVDKSRWQQGNVFSCSVMHEALHNHYTQKSLSLSPGK
SEQ ID NO: 156:
DASLPYLQKESVFQSGAHAYRIPALLYLPGQQSLLAFAEQRASKKDEHAELIVLRRGDYDAS
THQVQWQAQEVVAQARLDGHRSMNPCPLYDAQTGTLFLFFIAIPGQVTEQQQLQTRANVTRL
CQVTSTDHGRTWSSPRDLTDAAIGPAYREWSTFAVGPGHCLQLHDRARSLVVPAYAYRKLHP
KQRPIPSAFCFLSHDHGRTWARGHFVAQDTLECQVAEVETGEQRVVTLNARSHLRARVQAQS
TNDGLDFQESQLVKKLVEPPPAGCQGSVISFPSPRSGPGSPAQWLLYTHPTHRKQRADLGAY
LNPRPPAPEAWSEPVLLAKGSAAYSDLQSMGTGPDGSPLFGCLYEANDYEEIVFLMFTLKQA
FPAEYLPQEPKSSDKTHTCPPCPAPELLGGPSVFLFPPKPKDTLMISRTPEVTCVVVDVSHE
DPEVKFNWYVDGVEVHNAKTKPREEQYNSTYRVVSVLTVLHQDWLNGKEYKCKVSNKALPAP
IEKTISKAKGQPREPQVYTLPPSREEMTKNQVSLTCLVKGFYPSDIAVEWESNGQPENNYKT
TPPVLDSDGSFFLYSKLTVDKSRWQQGNVFSCSVMHEALHNHYTQKSLSLSPGK
SEQ ID NO: 157:
DASLPYLQKESVFQSGAHAYRIPALLYLPGQQSLLAFAEQRASKKDEHAELIVLRRGDYDAT
THQVQWQAQEVVAQARLDGHRSMNPCPLYDAQTGTLFLFFIAIPGQVTEQQQLQTRANVTRL
CQVTSTDHGRTWSSPRDLTDAAIGPAYREWSTFAVGPGHCLQLHDRARSLVVPAYAYRKLHP
KQRPIPSAFCFLSHDHGRTWARGHFVAQDTLECQVAEVETGEQRVVTLNARSHLRARVQAQS
TNDGLDFQESQLVKKLVEPPPAGCQGSVISFPSPRSGPGSPAQWLLYTHPTHRKQRADLGAY
LNPRPPAPEAWSEPVLLAKGSAAYSDLQSMGTGPDGSPLFGCLYEANDYEEIVFLMFTLKQA
FPAEYLPQEPKSSDKTHTCPPCPAPELLGGPSVFLFPPKPKDTLMISRTPEVTCVVVDVSHE
DPEVKFNWYVDGVEVHNAKTKPREEQYNSTYRVVSVLTVLHQDWLNGKEYKCKVSNKALPAP
IEKTISKAKGQPREPQVYTLPPSREEMTKNQVSLTCLVKGFYPSDIAVEWESNGQPENNYKT
TPPVLDSDGSFFLYSKLTVDKSRWQQGNVFSCSVMHEALHNHYTQKSLSLSPGK
SEQ ID NO: 158:
DASLPYLQKESVFQSGAHAYRIPALLYLPGQQSLLAFAEQRASKKDEHAELIVLRRGDYDAN
THQVQWQAQEVVAQARLDGHRSMNPCPLYDAQTGTLFLFFIAIPGQVTEQQQLQTRANVTRL
CQVTSTDHGRTWSSPRDLTDAAIGPAYREWSTFAVGPGHCLQLHDRARSLVVPAYAYRKLHP
KQRPIPSAFCFLSHDHGRTWARGHFVAQDTLECQVAEVETGEQRVVTLNARSHLRARVQAQS
TNDGLDFQESQLVKKLVEPPPAGCQGSVISFPSPRSGPGSPAQWLLYTHPTHRKQRADLGAY
LNPRPPAPEAWSEPVLLAKGSAAYSDLQSMGTGPDGSPLFGCLYEANDYEEIVFLMFTLKQA
FPAEYLPQEPKSSDKTHTCPPCPAPELLGGPSVFLFPPKPKDTLMISRTPEVTCVVVDVSHE
DPEVKFNWYVDGVEVHNAKTKPREEQYNSTYRVVSVLTVLHQDWLNGKEYKCKVSNKALPAP
IEKTISKAKGQPREPQVYTLPPSREEMTKNQVSLTCLVKGFYPSDIAVEWESNGQPENNYKT
TPPVLDSDGSFFLYSKLTVDKSRWQQGNVFSCSVMHEALHNHYTQKSLSLSPGK
SEQ ID NO: 167:
EPKSCDKTHTCPPCPAPELLGGPSVFLFPPKPKDTLMISRTPEVTCVVVDVSHEDPEVKFNW
YVDGVEVHNAKTKPREEQYNSTYRVVSVLTVLHQDWLNGKEYKCKVSNKALPAPIEKTISKA
KGQPREPQVYTLPPSREEMTKNQVSLTCLVKGFYPSDIAVEWESNGQPENNYKTTPPVLDSD
GSFFLTSKLTVDKSRWQQGNVFSCSVMHEALHNHYTQKSLSLSPGK
SEQ ID NO: 168:
DKTHTCPPCPAPELLGGPSVFLFPPKPKDTLMISRTPEVTCVVVDVSHEDPEVKFNWYVDGV
EVHNAKTKPREEQYNSTYRVVSVLTVLHQDWLNGKEYKCKVSNKALPAPIEKTISKAKGQPR
EPQVYTLPPSREEMTKNQVSLYCLVKGFYPSDIAVEWESNGQPENNYKTTPPVLDSDGSFFL
YSKLTVDKSRWQQGNVFSCSVMHEALHNHYTQKSLSLSPGK
SEQ ID NO: 169:
DASLPYLQKESVFQSGAHAYRIPALLYLPGQQSLLAFAEQRASKKDEHAELIVLRRGDYDAG
THQVQWQAQEVVAQARLDGHRSMNPCPLYDEQTGTLFLFFIAIPGQVTEQQQLQTRANVTRL
CQVTSTDHGRTWSSPRDLTDAAIGPAYREWSTFAVGPGHCLQLHDRARSLVVPAYAYRKLHP
KQRPIPSAFCFLSHDHGRTWARGHFVAQDTLECQVAEVETGEQRVVTLNARSHLRARVQAQS
TNDGLDFQESQLVKKLVEPPPQGCQGSVISFPSPRSGPGSPAQWLLYTHPTHARQRADLGAY
LNPRPPAPEAWSEPVLLAKGSAAYSDLQSMGTGPDGSPLFGCLYEANDYEEIVFLMFTLKQA
FPAEYLPQ
SEQ ID NO: 170:
DASLPYLQKESVFQSGAHAYRIPALLYLPGQQSLLAFAEQRASKKDEHAELIVLRRGDYDAG
THQVQWQAQEVVAQARLDGHRSMNPCPLYDEQTGTLFLFFIAIPGQVTEQQQLQTRANVTRL
CYVTSTDHGRTWSSPRDLTDAAIGPAYREWSTFAVGPGHCLQLHDRARSLVVPAYAYRKLHP
KQRPIPSAFCFLSHDHGRTWARGHFVAQDTLECQVAEVETGEQRVVTLNARSHLRARVQAQS
TNDGLDFQESQLVKKLVEPPPTGCQGSVISFPSPRSGPGSPAQWLLYTHPTHSWQRADLGAY
LNPRPPAPEAWSEPVLLAKGSAAYSDLQSMGTGPDGSPLFGCLYEANDYEEIVFLMFTLKQA
FPAEYLPQ
SEQ ID NO: 171:
DASLPYLQKESVFQSGAHAYRIPALLYLPGQQSLLAFAEQRASKKDEHAELIVLRRGDYDAG
THQVQWQAQEVVAQARLDGHRSMNPCPLYDEQTGTLFLFFIAIPGQVTEQQQLQTRANVTRL
CYVTSTDHGRTWSSPRDLTDAAIGPAYREWSTFAVGPGHCLQLHDRARSLVVPAYAYRKLHP
KQRPIPSAFCFLSHDHGRTWARGHFVAQDTLECQVAEVETGEQRVVTLNARSHLRFRVQAQS
TNDGLDFQESQLVKKLVEPPPTGCQGSVISFPSPRSGPGSPAQWLLYTHPTHSWQRADLGAY
LNPRPPAPEAWSEPVLLAKGSAAYSDLQSMGTGPDGSPLFGCLYEANDYEEIVFLMFTLKQA
FPAEYLPQ
SEQ ID NO: 175:
DASLPYLQDESVFQSGAHAYRIPALLYLPGQQSLLAFAEQRASKKDEHAELIVLRRGDYDAP
THQVQWQAQEVVAQARLDGHRSMNPCPLYDEQTGTLFLFFIAIPGQVTEQQQLQTRANVTRL
CQVTSTDHGRTWSSPRDLTDAAIGPAYREWSTFAVGPGHCLQLHDRARSLVVPAYAYRKLHP
KQRPIPSAFCFLSHDHGRTWARGHFVAQDTLANQVAEVETGEQRVVTLNARSHLRARVQAQS
TNDGLDFQESQLVKKLVEPPPQGCQGSVISFPSPRSGPGSPAQWLLYTHPTHSWQRADLGAY
LNPRPPAPEAWSEPVLLAKGSAAYSDLQSMGTGPDGSPLFGCLYEANDYEEIRFRMETLKQA
FPAEYLPQGGGGSGGGGSDKTHTCPPCPAPELLGGPSVFLFPPKPKDTLMISRTPEVTCVVV
DVSHEDPEVKFNWYVDGVEVHNAKTKPREEQYNSTYRVVSVLTVLHQDWLNGKEYKCKVSNK
ALPAPIEKTISKAKGQPREPQVYTLPPSREEMTKNQVSLTCLVKGFYPSDIAVEWESNGQPE
NNYKTTPPVLDSDGSFFLTSKLTVDKSRWQQGNVFSCSVMHEALHNHYTQKSLSLSPGK
SEQ ID NO: 176:
GATGCATCTCTGCCTTACCTGCAGGATGAAAGCGTGTTCCAGTCTGGCGCCCACGCCTACAG
AATTCCCGCTCTGCTGTATCTGCCAGGCCAGCAGTCTCTGCTGGCTTTCGCTGAACAGCGGG
CCAGCAAGAAGGATGAGCACGCCGAACTGATCGTGCTGCGGAGAGGCGATTACGACGCCcct
ACACATCAGGTGCAGTGGCAGGCTCAAGAGGTGGTGGCTCAGGCTAGACTGGACGGCCACAG
ATCTATGAACCCCTGTCCTCTGTACGATGAACAGACCGGCACACTGTTTCTGTTCTTTATCG
CTATCCCCGGCCAAGTGACCGAGCAGCAGCAGCTGCAGACAAGAGCCAACGTGACCAGACTG
TGTCAAGTGACCTCCACCGACCACGGCAGAACCTGGTCTAGCCCTAGAGATCTGACCGACGC
CGCCATCGGACCTGCCTATAGAGAGTGGTCCACCTTCGCCGTTGGACCTGGACACTGTCTCC
AGCTGCACGACAGGGCTAGATCTCTGGTGGTGCCTGCCTACGCCTATAGAAAGCTGCACCCC
AAACAGCGGCCTATTCCTAGCGCCTTCTGCTTTCTGAGCCACGATCACGGCAGGACATGGGC
CAGAGGACATTTCGTGGCCCAGGACACACTGGCGAATCAGGTGGCCGAAGTGGAAACCGGCG
AGCAGAGAGTCGTGACCCTGAACGCCAGATCTCACCTGAGAGCCAGAGTGCAGGCCCAGAGC
ACAAACGACGGCCTGGATTTCCAAGAGAGCCAGCTGGTCAAGAAACTGGTGGAACCTCCTCC
ACAGGGCTGTCAGGGAAGCGTGATCAGCTTTCCATCTCCTAGAAGCGGCCCTGGCTCTCCTG
CTCAGTGGCTGCTGTATACACACCCCACACACAGCTGGCAGAGAGCCGATCTGGGCGCCTAC
CTGAATCCTAGACCTCCTGCTCCTGAGGCTTGGAGCGAACCTGTTCTGCTGGCCAAGGGCAG
CGCTGCCTACAGCGATCTGCAGTCTATGGGCACAGGCCCTGATGGCAGCCCTCTGTTTGGCT
GTCTGTACGAGGCCAACGACTACGAAGAGATCCGTTTCCGTATGTTCACCCTGAAGCAGGCC
TTTCCAGCCGAGTACCTGCCTCAAGGCGGAGGTGGAAGTGGCGGAGGCGGATCcGACAAAAC
TCACACATGCCCACCGTGCCCAGCACCTGAACTCCTGGGGGGACCGTCAGTCTTCCTCTTCC
CCCCAAAACCCAAGGACACCCTCATGATCTCCCGGACCCCTGAGGTCACATGCGTGGTGGTG
GACGTGAGCCACGAAGACCCTGAGGTCAAGTTCAACTGGTACGTGGACGGCGTGGAGGTGCA
TAATGCCAAGACAAAGCCGCGGGAGGAGCAGTACAACAGCACGTACCGTGTGGTCAGCGTCC
TCACCGTCCTGCACCAGGACTGGCTGAATGGCAAGGAGTACAAGTGCAAGGTCTCCAACAAA
GCCCTCCCAGCCCCCATCGAGAAAACCATCTCCAAAGCCAAAGGGCAGCCCCGAGAACCACA
GGTcTACACCCTGCCCCCATCCCGGGAGGAGATGACCAAGAACCAGGTCAGCCTGACCTGCC
TGGTCAAAGGCTTCTATCCCAGCGACATCGCCGTGGAGTGGGAGAGCAATGGGCAGCCGGAG
AACAACTACAAGACCACGCCTCCCGTGCTGGACTCCGACGGCTCCTTCTTCCTCacTAGCAA
GCTCACCGTGGACAAGAGCAGGTGGCAGCAGGGGAACGTCTTCTCATGCTCCGTGATGCATG
AGGCTCTGCACAACCACTACACGCAGAAGAGCCTCTCCCTGTCTCCGGGTAAA
SEQ ID NO: 177:
DASLPYLQKESVFQSGAHAYRIPALLYLPGQQSLLAFAEQRASKKDEHAELIVLRRGDYDAG
THQVQWQAQEVVAQARLDGHRSMNPCPLYDEQTGTLFLFFIAIPGQVTEQQQLQTRANVTRL
CQVTSTDHGRTWSSPRDLTDAAIGPAYREWSTFAVGPGHCLQLHDRARSLVVPAYAYRKLHP
KQRPIPSAFCFLSHDHGRTWARGHFVAQDTLECQVAEVETGEQRVVTLNARSHLRARVQAQS
TNDGLDFQESQLVKKLVEPPPQGCQGSVISFPSPRSGPGSPAQWLLYTHPTHARQRADLGAY
LNPRPPAPEAWSEPVLLAKGSAAYSDLQSMGTGPDGSPLFGCLYEANDYEEIVFLMFTLKQA
FPAEYLPQGGGGSGGGGSDKTHTCPPCPAPELLGGPSVFLFPPKPKDTLMISRTPEVTCVVV
DVSHEDPEVKFNWYVDGVEVHNAKTKPREEQYNSTYRVVSVLTVLHQDWLNGKEYKCKVSNK
ALPAPIEKTISKAKGQPREPQVYTLPPSREEMTKNQVSLTCLVKGFYPSDIAVEWESNGQPE
NNYKTTPPVLDSDGSFFLTSKLTVDKSRWQQGNVFSCSVMHEALHNHYTQKSLSLSPGK
SEQ ID NO: 178:
DASLPYLQKESVFQSGAHAYRIPALLYLPGQQSLLAFAEQRASKKDEHAELIVLRRGDYDAG
THQVQWQAQEVVAQARLDGHRSMNPCPLYDEQTGTLFLFFIAIPGQVTEQQQLQTRANVTRL
CQVTSTDHGRTWSSPRDLTDAAIGPAYREWSTFAVGPGHCLQLHDRARSLVVPAYAYRKLHP
KQRPIPSAFCFLSHDHGRTWARGHFVAQDTLECQVAEVETGEQRVVTLNARSHLRARVQAQS
TNDGLDFQESQLVKKLVEPPPQGCQGSVISFPSPRSGPGSPAQWLLYTHPTHARQRADLGAY
LNPRPPAPEAWSEPVLLAKGSAAYSDLQSMGTGPDGSPLFGCLYEANDYEEIVFLMFTLKQA
FPAEYLPQEPKSSDKTHTCPPCPAPELLGGPSVFLFPPKPKDTLMISRTPEVTCVVVDVSHE
DPEVKFNWYVDGVEVHNAKTKPREEQYNSTYRVVSVLTVLHQDWLNGKEYKCKVSNKALPAP
IEKTISKAKGQPREPQVYTLPPSREEMTKNQVSLTCLVKGFYPSDIAVEWESNGQPENNYKT
TPPVLDSDGSFFLTSKLTVDKSRWQQGNVFSCSVMHEALHNHYTQKSLSLSPGK
SEQ ID NO: 179:
DASLPYLQKESVFQSGAHAYRIPALLYLPGQQSLLAFAEQRASKKDEHAELIVLRRGDYDAG
THQVQWQAQEVVAQARLDGHRSMNPCPLYDEQTGTLFLFFIAIPGQVTEQQQLQTRANVTRL
CQVTSTDHGRTWSSPRDLTDAAIGPAYREWSTFAVGPGHCLQLHDRARSLVVPAYAYRKLHP
KQRPIPSAFCFLSHDHGRTWARGHFVAQDTLECQVAEVETGEQRVVTLNARSHLRARVQAQS
TNDGLDFQESQLVKKLVEPPPQGCQGSVISFPSPRSGPGSPAQWLLYTHPTHARQRADLGAY
LNPRPPAPEAWSEPVLLAKGSAAYSDLQSMGTGPDGSPLFGCLYEANDYEEIVFLMFTLKQA
FPAEYLPQGGGGSGGGGSDKTHTCPPCPAPELLGGPSVFLFPPKPKDTLMISRTPEVTCVVV
DVSHEDPEVKFNWYVDGVEVHNAKTKPREEQYNSTYRVVSVLTVLHQDWLNGKEYKCKVSNK
ALPAPIEKTISKAKGQPREPQVYTLPPSREEMTKNQVSLTCLVKGFYPSDIAVEWESNGQPE
NNYKTTPPVLDSDGSFFLYSKLTVDKSRWQQGNVFSCSVMHEALHNHYTQKSLSLSPGK
SEQ ID NO: 180:
DASLPYLQKESVFQSGAHAYRIPALLYLPGQQSLLAFAEQRASKKDEHAELIVLRRGDYDAG
THQVQWQAQEVVAQARLDGHRSMNPCPLYDEQTGTLFLFFIAIPGQVTEQQQLQTRANVTRL
CQVTSTDHGRTWSSPRDLTDAAIGPAYREWSTFAVGPGHCLQLHDRARSLVVPAYAYRKLHP
KQRPIPSAFCFLSHDHGRTWARGHFVAQDTLECQVAEVETGEQRVVTLNARSHLRARVQAQS
TNDGLDFQESQLVKKLVEPPPQGCQGSVISFPSPRSGPGSPAQWLLYTHPTHARQRADLGAY
LNPRPPAPEAWSEPVLLAKGSAAYSDLQSMGTGPDGSPLFGCLYEANDYEEIVFLMFTLKQA
FPAEYLPQEPKSSDKTHTCPPCPAPELLGGPSVFLFPPKPKDTLMISRTPEVTCVVVDVSHE
DPEVKFNWYVDGVEVHNAKTKPREEQYNSTYRVVSVLTVLHQDWLNGKEYKCKVSNKALPAP
IEKTISKAKGQPREPQVYTLPPSREEMTKNQVSLTCLVKGFYPSDIAVEWESNGQPENNYKT
TPPVLDSDGSFFLYSKLTVDKSRWQQGNVFSCSVMHEALHNHYTQKSLSLSPGK
SEQ ID NO: 181:
DASLPYLQKESVFQSGAHAYRIPALLYLPGQQSLLAFAEQRASKKDEHAELIVLRRGDYDAG
THQVQWQAQEVVAQARLDGHRSMNPCPLYDEQTGTLFLFFIAIPGQVTEQQQLQTRANVTRL
CYVTSTDHGRTWSSPRDLTDAAIGPAYREWSTFAVGPGHCLQLHDRARSLVVPAYAYRKLHP
KQRPIPSAFCFLSHDHGRTWARGHFVAQDTLECQVAEVETGEQRVVTLNARSHLRARVQAQS
TNDGLDFQESQLVKKLVEPPPTGCQGSVISFPSPRSGPGSPAQWLLYTHPTHSWQRADLGAY
LNPRPPAPEAWSEPVLLAKGSAAYSDLQSMGTGPDGSPLFGCLYEANDYEEIVFLMFTLKQA
FPAEYLPQGGGGSGGGGSDKTHTCPPCPAPELLGGPSVFLFPPKPKDTLMISRTPEVTCVVV
DVSHEDPEVKFNWYVDGVEVHNAKTKPREEQYNSTYRVVSVLTVLHQDWLNGKEYKCKVSNK
ALPAPIEKTISKAKGQPREPQVYTLPPSREEMTKNQVSLTCLVKGFYPSDIAVEWESNGQPE
NNYKTTPPVLDSDGSFFLTSKLTVDKSRWQQGNVFSCSVMHEALHNHYTQKSLSLSPGK
SEQ ID NO: 182:
DASLPYLQKESVFQSGAHAYRIPALLYLPGQQSLLAFAEQRASKKDEHAELIVLRRGDYDAG
THQVQWQAQEVVAQARLDGHRSMNPCPLYDEQTGTLFLFFIAIPGQVTEQQQLQTRANVTRL
CYVTSTDHGRTWSSPRDLTDAAIGPAYREWSTFAVGPGHCLQLHDRARSLVVPAYAYRKLHP
KQRPIPSAFCFLSHDHGRTWARGHFVAQDTLECQVAEVETGEQRVVTLNARSHLRARVQAQS
TNDGLDFQESQLVKKLVEPPPTGCQGSVISFPSPRSGPGSPAQWLLYTHPTHSWQRADLGAY
LNPRPPAPEAWSEPVLLAKGSAAYSDLQSMGTGPDGSPLFGCLYEANDYEEIVFLMFTLKQA
FPAEYLPQEPKSSDKTHTCPPCPAPELLGGPSVFLFPPKPKDTLMISRTPEVTCVVVDVSHE
DPEVKFNWYVDGVEVHNAKTKPREEQYNSTYRVVSVLTVLHQDWLNGKEYKCKVSNKALPAP
IEKTISKAKGQPREPQVYTLPPSREEMTKNQVSLTCLVKGFYPSDIAVEWESNGQPENNYKT
TPPVLDSDGSFFLTSKLTVDKSRWQQGNVFSCSVMHEALHNHYTQKSLSLSPGK
SEQ ID NO: 183:
DASLPYLQKESVFQSGAHAYRIPALLYLPGQQSLLAFAEQRASKKDEHAELIVLRRGDYDAG
THQVQWQAQEVVAQARLDGHRSMNPCPLYDEQTGTLFLFFIAIPGQVTEQQQLQTRANVTRL
CYVTSTDHGRTWSSPRDLTDAAIGPAYREWSTFAVGPGHCLQLHDRARSLVVPAYAYRKLHP
KQRPIPSAFCFLSHDHGRTWARGHFVAQDTLECQVAEVETGEQRVVTLNARSHLRARVQAQS
TNDGLDFQESQLVKKLVEPPPTGCQGSVISFPSPRSGPGSPAQWLLYTHPTHSWQRADLGAY
LNPRPPAPEAWSEPVLLAKGSAAYSDLQSMGTGPDGSPLFGCLYEANDYEEIVFLMFTLKQA
FPAEYLPQGGGGSGGGGSDKTHTCPPCPAPELLGGPSVFLFPPKPKDTLMISRTPEVTCVVV
DVSHEDPEVKFNWYVDGVEVHNAKTKPREEQYNSTYRVVSVLTVLHQDWLNGKEYKCKVSNK
ALPAPIEKTISKAKGQPREPQVYTLPPSREEMTKNQVSLTCLVKGFYPSDIAVEWESNGQPE
NNYKTTPPVLDSDGSFFLYSKLTVDKSRWQQGNVFSCSVMHEALHNHYTQKSLSLSPGK
SEQ ID NO: 184:
DASLPYLQKESVFQSGAHAYRIPALLYLPGQQSLLAFAEQRASKKDEHAELIVLRRGDYDAG
THQVQWQAQEVVAQARLDGHRSMNPCPLYDEQTGTLFLFFIAIPGQVTEQQQLQTRANVTRL
CYVTSTDHGRTWSSPRDLTDAAIGPAYREWSTFAVGPGHCLQLHDRARSLVVPAYAYRKLHP
KQRPIPSAFCFLSHDHGRTWARGHFVAQDTLECQVAEVETGEQRVVTLNARSHLRARVQAQS
TNDGLDFQESQLVKKLVEPPPTGCQGSVISFPSPRSGPGSPAQWLLYTHPTHSWQRADLGAY
LNPRPPAPEAWSEPVLLAKGSAAYSDLQSMGTGPDGSPLFGCLYEANDYEEIVFLMFTLKQA
FPAEYLPQEPKSSDKTHTCPPCPAPELLGGPSVFLFPPKPKDTLMISRTPEVTCVVVDVSHE
DPEVKFNWYVDGVEVHNAKTKPREEQYNSTYRVVSVLTVLHQDWLNGKEYKCKVSNKALPAP
IEKTISKAKGQPREPQVYTLPPSREEMTKNQVSLTCLVKGFYPSDIAVEWESNGQPENNYKT
TPPVLDSDGSFFLYSKLTVDKSRWQQGNVFSCSVMHEALHNHYTQKSLSLSPGK
SEQ ID NO: 185:
DASLPYLQKESVFQSGAHAYRIPALLYLPGQQSLLAFAEQRASKKDEHAELIVLRRGDYDAG
THQVQWQAQEVVAQARLDGHRSMNPCPLYDEQTGTLFLFFIAIPGQVTEQQQLQTRANVTRL
CYVTSTDHGRTWSSPRDLTDAAIGPAYREWSTFAVGPGHCLQLHDRARSLVVPAYAYRKLHP
KQRPIPSAFCFLSHDHGRTWARGHFVAQDTLECQVAEVETGEQRVVTLNARSHLRFRVQAQS
TNDGLDFQESQLVKKLVEPPPTGCQGSVISFPSPRSGPGSPAQWLLYTHPTHSWQRADLGAY
LNPRPPAPEAWSEPVLLAKGSAAYSDLQSMGTGPDGSPLFGCLYEANDYEEIVFLMFTLKQA
FPAEYLPQGGGGSGGGGSDKTHTCPPCPAPELLGGPSVFLFPPKPKDTLMISRTPEVTCVVV
DVSHEDPEVKFNWYVDGVEVHNAKTKPREEQYNSTYRVVSVLTVLHQDWLNGKEYKCKVSNK
ALPAPIEKTISKAKGQPREPQVYTLPPSREEMTKNQVSLTCLVKGFYPSDIAVEWESNGQPE
NNYKTTPPVLDSDGSFFLTSKLTVDKSRWQQGNVFSCSVMHEALHNHYTQKSLSLSPGK
SEQ ID NO: 186:
DASLPYLQKESVFQSGAHAYRIPALLYLPGQQSLLAFAEQRASKKDEHAELIVLRRGDYDAG
THQVQWQAQEVVAQARLDGHRSMNPCPLYDEQTGTLFLFFIAIPGQVTEQQQLQTRANVTRL
CYVTSTDHGRTWSSPRDLTDAAIGPAYREWSTFAVGPGHCLQLHDRARSLVVPAYAYRKLHP
KQRPIPSAFCFLSHDHGRTWARGHFVAQDTLECQVAEVETGEQRVVTLNARSHLRFRVQAQS
TNDGLDFQESQLVKKLVEPPPTGCQGSVISFPSPRSGPGSPAQWLLYTHPTHSWQRADLGAY
LNPRPPAPEAWSEPVLLAKGSAAYSDLQSMGTGPDGSPLFGCLYEANDYEEIVFLMFTLKQA
FPAEYLPQEPKSSDKTHTCPPCPAPELLGGPSVFLFPPKPKDTLMISRTPEVTCVVVDVSHE
DPEVKFNWYVDGVEVHNAKTKPREEQYNSTYRVVSVLTVLHQDWLNGKEYKCKVSNKALPAP
IEKTISKAKGQPREPQVYTLPPSREEMTKNQVSLTCLVKGFYPSDIAVEWESNGQPENNYKT
TPPVLDSDGSFFLTSKLTVDKSRWQQGNVFSCSVMHEALHNHYTQKSLSLSPGK
SEQ ID NO: 187:
DASLPYLQKESVFQSGAHAYRIPALLYLPGQQSLLAFAEQRASKKDEHAELIVLRRGDYDAG
THQVQWQAQEVVAQARLDGHRSMNPCPLYDEQTGTLFLFFIAIPGQVTEQQQLQTRANVTRL
CYVTSTDHGRTWSSPRDLTDAAIGPAYREWSTFAVGPGHCLQLHDRARSLVVPAYAYRKLHP
KQRPIPSAFCFLSHDHGRTWARGHFVAQDTLECQVAEVETGEQRVVTLNARSHLRFRVQAQS
TNDGLDFQESQLVKKLVEPPPTGCQGSVISFPSPRSGPGSPAQWLLYTHPTHSWQRADLGAY
LNPRPPAPEAWSEPVLLAKGSAAYSDLQSMGTGPDGSPLFGCLYEANDYEEIVFLMFTLKQA
FPAEYLPQGGGGSGGGGSDKTHTCPPCPAPELLGGPSVFLFPPKPKDTLMISRTPEVTCVVV
DVSHEDPEVKFNWYVDGVEVHNAKTKPREEQYNSTYRVVSVLTVLHQDWLNGKEYKCKVSNK
ALPAPIEKTISKAKGQPREPQVYTLPPSREEMTKNQVSLTCLVKGFYPSDIAVEWESNGQPE
NNYKTTPPVLDSDGSFFLYSKLTVDKSRWQQGNVFSCSVMHEALHNHYTQKSLSLSPGK
SEQ ID NO: 188:
DASLPYLQKESVFQSGAHAYRIPALLYLPGQQSLLAFAEQRASKKDEHAELIVLRRGDYDAG
THQVQWQAQEVVAQARLDGHRSMNPCPLYDEQTGTLFLFFIAIPGQVTEQQQLQTRANVTRL
CYVTSTDHGRTWSSPRDLTDAAIGPAYREWSTFAVGPGHCLQLHDRARSLVVPAYAYRKLHP
KQRPIPSAFCFLSHDHGRTWARGHFVAQDTLECQVAEVETGEQRVVTLNARSHLRFRVQAQS
TNDGLDFQESQLVKKLVEPPPTGCQGSVISFPSPRSGPGSPAQWLLYTHPTHSWQRADLGAY
LNPRPPAPEAWSEPVLLAKGSAAYSDLQSMGTGPDGSPLFGCLYEANDYEEIVFLMFTLKQA
FPAEYLPQEPKSSDKTHTCPPCPAPELLGGPSVFLFPPKPKDTLMISRTPEVTCVVVDVSHE
DPEVKFNWYVDGVEVHNAKTKPREEQYNSTYRVVSVLTVLHQDWLNGKEYKCKVSNKALPAP
IEKTISKAKGQPREPQVYTLPPSREEMTKNQVSLTCLVKGFYPSDIAVEWESNGQPENNYKT
TPPVLDSDGSFFLYSKLTVDKSRWQQGNVFSCSVMHEALHNHYTQKSLSLSPGK
SEQ ID NO: 189:
DASLPYLQKESVFQSGAHAYRIPALLYLPGQQSLLAFAEQRASKKDEHAELIVLRRGDYDAG
THQVQWQAQEVVAQARLDGHRSMNPCPLYDEQTGTLFLFFIAIPGQVTEQQQLQTRANVTRL
CYVTSTDHGRTWSSPRDLTDAAIGPAYREWSTFAVGPGHCLQLHDRARSLVVPAYAYRKLHP
KQRPIPSAFCFLSHDHGRTWARGHFVAQDTLECQVAEVETGEQRVVTLNARSHLRARVQAQS
TNDGLDFQESQLVKKLVEPPPTGCQGSVISFPSPRSGPGSPAQWLLYTHPTHSWQRADLGAY
LNPRPPAPEAWSEPVLLAKGSAAYSDLQSMGTGPDGSPLFGCLYEANDYEEIVFLMFTLKQA
FPAEYLPQEPKSSDKTHTCPPCPAPELLGGPSVFLFPPKPKDTLMISRTPEVTCVVVDVSHE
DPEVKENWYVDGVEVHNAKTKPREEQYNSTYRVVSVLTVLHQDWLNGKEYKCKVSNKALPAP
IEKTISKAKGQPREPQVYTLPPSREEMTKNQVSLYCLVKGFYPSDIAVEWESNGQPENNYKT
TPPVLDSDGSFFLYSKLTVDKSRWQQGNVFSCSVMHEALHNHYTOKSLSLSPGK
SEQ ID NO: 190:
gatGCATCTCTGCCTTACCTGCAGAAAGAAAGCGTGTTCCAGTCTGGCGCCCACGCCTACAG
AATTCCCGCTCTGCTGTATCTGCCAGGCCAGCAGTCTCTGCTGGCTTTCGCTGAACAGCGGG
CCAGCAAGAAGGATGAGCACGCCGAACTGATCGTGCTGCGGAGAGGCGATTACGACGCCggc
ACACATCAGGTGCAGTGGCAGGCTCAAGAGGTGGTGGCTCAGGCTAGACTGGACGGCCACAG
ATCTATGAACCCCTGTCCTCTGTACGATgaaCAGACCGGCACACTGTTTCTGTTCTTTATCG
CTATCCCCGGCCAAGTGACCGAGCAGCAGCAGCTGCAGACAAGAGCCAACGTGACCAGACTG
TGTtacGTGACCTCCACCGACCACGGCAGAACCTGGTCTAGCCCTAGAGATCTGACCGACGC
CGCCATCGGACCTGCCTATAGAGAGTGGTCCACCTTCGCCGTTGGACCTGGACACTGTCTCC
AGCTGCACGACAGGGCTAGATCTCTGGTGGTGCCTGCCTACGCCTATAGAAAGCTGCACCCC
AAACAGCGGCCTATTCCTAGCGCCTTCTGCTTTCTGAGCCACGATCACGGCAGGACATGGGC
CAGAGGACATTTCGTGGCCCAGGACACACTGGAATGCCAGGTGGCCGAAGTGGAAACCGGCG
AGCAGAGAGTCGTGACCCTGAACGCCAGATCTCACCTGAGAGCCAGAGTGCAGGCCCAGAGC
ACAAACGACGGCCTGGATTTCCAAGAGAGCCAGCTGGTCAAGAAACTGGTGGAACCTCCTCC
AaccGGCTGTCAGGGAAGCGTGATCAGCTTTCCATCTCCTAGAAGCGGCCCTGGCTCTCCTG
CTCAGTGGCTGCTGTATACACACCCCACACACAGCTGGCAGAGAGCCGATCTGGGCGCCTAC
CTGAATCCTAGACCTCCTGCTCCTGAGGCTTGGAGCGAACCTGTTCTGCTGGCCAAGGGCAG
CgctGCCTACAGCGATCTGCAGTCTATGGGCACAGGCCCTGATGGCAGCCCTCTGTTTGGCT
GTCTGTACGAGGCCAACGACTACGAAGAGATCGTGTTCCTGATGTTCACCCTGAAGCAGGCC
TTTCCAGCCGAGTACCTGCCTCAAGAGCCCAAATCTTCTGACAAAACTCACACATGCCCACC
GTGCCCAGCACCTGAACTCCTGGGGGGACCGTCAGTCTTCCTCTTCCCCCCAAAACCCAAGG
ACACCCTCATGATCTCCCGGACCCCTGAGGTCACATGCGTGGTGGTGGACGTGAGCCACGAA
GACCCTGAGGTCAAGTTCAACTGGTACGTGGACGGCGTGGAGGTGCATAATGCCAAGACAAA
GCCGCGGGAGGAGCAGTACAACAGCACGTACCGTGTGGTCAGCGTCCTCACCGTCCTGCACC
AGGACTGGCTGAATGGCAAGGAGTACAAGTGCAAGGTCTCCAACAAAGCCCTCCCAGCCCCC
ATCGAGAAAACCATCTCCAAAGCCAAAGGGCAGCCCCGAGAACCACAGGTcTACACCCTGCC
CCCATCCCGGGAGGAGATGACCAAGAACCAGGTCAGCCTGtaCTGCCTGGTCAAAGGCTTCT
ATCCCAGCGACATCGCCGTGGAGTGGGAGAGCAATGGGCAGCCGGAGAACAACTACAAGACC
ACGCCTCCCGTGCTGGACTCCGACGGCTCCTTCTTCCTCtatAGCAAGCTCACCGTGGACAA
GAGCAGGTGGCAGCAGGGGAACGTCTTCTCATGCTCCGTGATGCATGAGGCTCTGCACAACC
ACTACACGCAGAAGAGCCTCTCCCTGTCTCCGGGTAAA
SEQ ID NO: 191:
DASLPYLQKESVFQSGAHAYRIPALLYLPGQQSLLAFAEQRASKKDEHAELIVLRRGDYDAG
THQVQWQAQEVVAQARLDGHRSMNPCPLYDEQTGTLFLFFIAIPGQVTEQQQLQTRANVTRL
CYVTSTDHGRTWSSPRDLTDAAIGPAYREWSTFAVGPGHCLQLHDRARSLVVPAYAYRKLHP
KQRPIPSAFCFLSHDHGRTWARGHFVAQDTLECQVAEVETGEQRVVTLNARSHLRFRVQAQS
TNDGLDFQESQLVKKLVEPPPTGCQGSVISFPSPRSGPGSPAQWLLYTHPTHSWQRADLGAY
LNPRPPAPEAWSEPVLLAKGSAAYSDLQSMGTGPDGSPLFGCLYEANDYEEIVFLMFTLKQA
FPAEYLPQEPKSSDKTHTCPPCPAPELLGGPSVFLFPPKPKDTLMISRTPEVTCVVVDVSHE
DPEVKFNWYVDGVEVHNAKTKPREEQYNSTYRVVSVLTVLHQDWLNGKEYKCKVSNKALPAP
IEKTISKAKGQPREPQVYTLPPSREEMTKNQVSLYCLVKGFYPSDIAVEWESNGQPENNYKT
TPPVLDSDGSFFLYSKLTVDKSRWQQGNVFSCSVMHEALHNHYTQKSLSLSPGK
SEQ ID NO: 192:
gatGCATCTCTGCCTTACCTGCAGAAAGAAAGCGTGTTCCAGTCTGGCGCCCACGCCTACAG
AATTCCCGCTCTGCTGTATCTGCCAGGCCAGCAGTCTCTGCTGGCTTTCGCTGAACAGCGGG
CCAGCAAGAAGGATGAGCACGCCGAACTGATCGTGCTGCGGAGAGGCGATTACGACGCCggc
ACACATCAGGTGCAGTGGCAGGCTCAAGAGGTGGTGGCTCAGGCTAGACTGGACGGCCACAG
ATCTATGAACCCCTGTCCTCTGTACGATgaaCAGACCGGCACACTGTTTCTGTTCTTTATCG
CTATCCCCGGCCAAGTGACCGAGCAGCAGCAGCTGCAGACAAGAGCCAACGTGACCAGACTG
TGTtacGTGACCTCCACCGACCACGGCAGAACCTGGTCTAGCCCTAGAGATCTGACCGACGC
CGCCATCGGACCTGCCTATAGAGAGTGGTCCACCTTCGCCGTTGGACCTGGACACTGTCTCC
AGCTGCACGACAGGGCTAGATCTCTGGTGGTGCCTGCCTACGCCTATAGAAAGCTGCACCCC
AAACAGCGGCCTATTCCTAGCGCCTTCTGCTTTCTGAGCCACGATCACGGCAGGACATGGGC
CAGAGGACATTTCGTGGCCCAGGACACACTGGAATGCCAGGTGGCCGAAGTGGAAACCGGCG
AGCAGAGAGTCGTGACCCTGAACGCCAGATCTCACCTGAGAttCAGAGTGCAGGCCCAGAGC
ACAAACGACGGCCTGGATTTCCAAGAGAGCCAGCTGGTCAAGAAACTGGTGGAACCTCCTCC
AaccGGCTGTCAGGGAAGCGTGATCAGCTTTCCATCTCCTAGAAGCGGCCCTGGCTCTCCTG
CTCAGTGGCTGCTGTATACACACCCCACACACAGCTGGCAGAGAGCCGATCTGGGCGCCTAC
CTGAATCCTAGACCTCCTGCTCCTGAGGCTTGGAGCGAACCTGTTCTGCTGGCCAAGGGCAG
CgctGCCTACAGCGATCTGCAGTCTATGGGCACAGGCCCTGATGGCAGCCCTCTGTTTGGCT
GTCTGTACGAGGCCAACGACTACGAAGAGATCGTGTTCCTGATGTTCACCCTGAAGCAGGCC
TTTCCAGCCGAGTACCTGCCTCAAGAGCCCAAATCTTCTGACAAAACTCACACATGCCCACC
GTGCCCAGCACCTGAACTCCTGGGGGGACCGTCAGTCTTCCTCTTCCCCCCAAAACCCAAGG
ACACCCTCATGATCTCCCGGACCCCTGAGGTCACATGCGTGGTGGTGGACGTGAGCCACGAA
GACCCTGAGGTCAAGTTCAACTGGTACGTGGACGGCGTGGAGGTGCATAATGCCAAGACAAA
GCCGCGGGAGGAGCAGTACAACAGCACGTACCGTGTGGTCAGCGTCCTCACCGTCCTGCACC
AGGACTGGCTGAATGGCAAGGAGTACAAGTGCAAGGTCTCCAACAAAGCCCTCCCAGCCCCC
ATCGAGAAAACCATCTCCAAAGCCAAAGGGCAGCCCCGAGAACCACAGGTcTACACCCTGCC
CCCATCCCGGGAGGAGATGACCAAGAACCAGGTCAGCCTGtaCTGCCTGGTCAAAGGCTTCT
ATCCCAGCGACATCGCCGTGGAGTGGGAGAGCAATGGGCAGCCGGAGAACAACTACAAGACC
ACGCCTCCCGTGCTGGACTCCGACGGCTCCTTCTTCCTCtatAGCAAGCTCACCGTGGACAA
GAGCAGGTGGCAGCAGGGGAACGTCTTCTCATGCTCCGTGATGCATGAGGCTCTGCACAACC
ACTACACGCAGAAGAGCCTCTCCCTGTCTCCGGGTAAA
SEQ ID NO: 193:
gatGCATCTCTGCCTTACCTGCAGAAAGAAAGCGTGTTCCAGTCTGGCGCCCACGCCTACAG
AATTCCCGCTCTGCTGTATCTGCCAGGCCAGCAGTCTCTGCTGGCTTTCGCTGAACAGCGGG
CCAGCAAGAAGGATGAGCACGCCGAACTGATCGTGCTGCGGAGAGGCGATTACGACGCCggc
ACACATCAGGTGCAGTGGCAGGCTCAAGAGGTGGTGGCTCAGGCTAGACTGGACGGCCACAG
ATCTATGAACCCCTGTCCTCTGTACGATgaaCAGACCGGCACACTGTTTCTGTTCTTTATCG
CTATCCCCGGCCAAGTGACCGAGCAGCAGCAGCTGCAGACAAGAGCCAACGTGACCAGACTG
TGTCAAGTGACCTCCACCGACCACGGCAGAACCTGGTCTAGCCCTAGAGATCTGACCGACGC
CGCCATCGGACCTGCCTATAGAGAGTGGTCCACCTTCGCCGTTGGACCTGGACACTGTCTCC
AGCTGCACGACAGGGCTAGATCTCTGGTGGTGCCTGCCTACGCCTATAGAAAGCTGCACCCC
AAACAGCGGCCTATTCCTAGCGCCTTCTGCTTTCTGAGCCACGATCACGGCAGGACATGGGC
CAGAGGACATTTCGTGGCCCAGGACACACTGGAATGCCAGGTGGCCGAAGTGGAAACCGGCG
AGCAGAGAGTCGTGACCCTGAACGCCAGATCTCACCTGAGAGCCAGAGTGCAGGCCCAGAGC
ACAAACGACGGCCTGGATTTCCAAGAGAGCCAGCTGGTCAAGAAACTGGTGGAACCTCCTCC
ACAGGGCTGTCAGGGAAGCGTGATCAGCTTTCCATCTCCTAGAAGCGGCCCTGGCTCTCCTG
CTCAGTGGCTGCTGTATACACACCCCACACACAGCTGGCAGAGAGCCGATCTGGGCGCCTAC
CTGAATCCTAGACCTCCTGCTCCTGAGGCTTGGAGCGAACCTGTTCTGCTGGCCAAGGGCAG
CgctGCCTACAGCGATCTGCAGTCTATGGGCACAGGCCCTGATGGCAGCCCTCTGTTTGGCT
GTCTGTACGAGGCCAACGACTACGAAGAGATCGTGTTCCTGATGTTCACCCTGAAGCAGGCC
TTTCCAGCCGAGTACCTGCCTCAAGAGCCCAAATCTTCTGACAAAACTCACACATGCCCACC
GTGCCCAGCACCTGAACTCCTGGGGGGACCGTCAGTCTTCCTCTTCCCCCCAAAACCCAAGG
ACACCCTCATGATCTCCCGGACCCCTGAGGTCACATGCGTGGTGGTGGACGTGAGCCACGAA
GACCCTGAGGTCAAGTTCAACTGGTACGTGGACGGCGTGGAGGTGCATAATGCCAAGACAAA
GCCGCGGGAGGAGCAGTACAACAGCACGTACCGTGTGGTCAGCGTCCTCACCGTCCTGCACC
AGGACTGGCTGAATGGCAAGGAGTACAAGTGCAAGGTCTCCAACAAAGCCCTCCCAGCCCCC
ATCGAGAAAACCATCTCCAAAGCCAAAGGGCAGCCCCGAGAACCACAGGTcTACACCCTGCC
CCCATCCCGGGAGGAGATGACCAAGAACCAGGTCAGCCTGACCTGCCTGGTCAAAGGCTTCT
ATCCCAGCGACATCGCCGTGGAGTGGGAGAGCAATGGGCAGCCGGAGAACAACTACAAGACC
ACGCCTCCCGTGCTGGACTCCGACGGCTCCTTCTTCCTCtatAGCAAGCTCACCGTGGACAA
GAGCAGGTGGCAGCAGGGGAACGTCTTCTCATGCTCCGTGATGCATGAGGCTCTGCACAACC
ACTACACGCAGAAGAGCCTCTCCCTGTCTCCGGGTAAA
SEQ ID NO: 194:
gatGCATCTCTGCCTTACCTGCAGAAAGAAAGCGTGTTCCAGTCTGGCGCCCACGCCTACAG
AATTCCCGCTCTGCTGTATCTGCCAGGCCAGCAGTCTCTGCTGGCTTTCGCTGAACAGCGGG
CCAGCAAGAAGGATGAGCACGCCGAACTGATCGTGCTGCGGAGAGGCGATTACGACGCCggc
ACACATCAGGTGCAGTGGCAGGCTCAAGAGGTGGTGGCTCAGGCTAGACTGGACGGCCACAG
ATCTATGAACCCCTGTCCTCTGTACGATgaaCAGACCGGCACACTGTTTCTGTTCTTTATCG
CTATCCCCGGCCAAGTGACCGAGCAGCAGCAGCTGCAGACAAGAGCCAACGTGACCAGACTG
TGTtacGTGACCTCCACCGACCACGGCAGAACCTGGTCTAGCCCTAGAGATCTGACCGACGC
CGCCATCGGACCTGCCTATAGAGAGTGGTCCACCTTCGCCGTTGGACCTGGACACTGTCTCC
AGCTGCACGACAGGGCTAGATCTCTGGTGGTGCCTGCCTACGCCTATAGAAAGCTGCACCCC
AAACAGCGGCCTATTCCTAGCGCCTTCTGCTTTCTGAGCCACGATCACGGCAGGACATGGGC
CAGAGGACATTTCGTGGCCCAGGACACACTGGAATGCCAGGTGGCCGAAGTGGAAACCGGCG
AGCAGAGAGTCGTGACCCTGAACGCCAGATCTCACCTGAGAGCCAGAGTGCAGGCCCAGAGC
ACAAACGACGGCCTGGATTTCCAAGAGAGCCAGCTGGTCAAGAAACTGGTGGAACCTCCTCC
AaccGGCTGTCAGGGAAGCGTGATCAGCTTTCCATCTCCTAGAAGCGGCCCTGGCTCTCCTG
CTCAGTGGCTGCTGTATACACACCCCACACACAGCTGGCAGAGAGCCGATCTGGGCGCCTAC
CTGAATCCTAGACCTCCTGCTCCTGAGGCTTGGAGCGAACCTGTTCTGCTGGCCAAGGGCAG
CgctGCCTACAGCGATCTGCAGTCTATGGGCACAGGCCCTGATGGCAGCCCTCTGTTTGGCT
GTCTGTACGAGGCCAACGACTACGAAGAGATCGTGTTCCTGATGTTCACCCTGAAGCAGGCC
TTTCCAGCCGAGTACCTGCCTCAAGAGCCCAAATCTTCTGACAAAACTCACACATGCCCACC
GTGCCCAGCACCTGAACTCCTGGGGGGACCGTCAGTCTTCCTCTTCCCCCCAAAACCCAAGG
ACACCCTCATGATCTCCCGGACCCCTGAGGTCACATGCGTGGTGGTGGACGTGAGCCACGAA
GACCCTGAGGTCAAGTTCAACTGGTACGTGGACGGCGTGGAGGTGCATAATGCCAAGACAAA
GCCGCGGGAGGAGCAGTACAACAGCACGTACCGTGTGGTCAGCGTCCTCACCGTCCTGCACC
AGGACTGGCTGAATGGCAAGGAGTACAAGTGCAAGGTCTCCAACAAAGCCCTCCCAGCCCCC
ATCGAGAAAACCATCTCCAAAGCCAAAGGGCAGCCCCGAGAACCACAGGTcTACACCCTGCC
CCCATCCCGGGAGGAGATGACCAAGAACCAGGTCAGCCTGACCTGCCTGGTCAAAGGCTTCT
ATCCCAGCGACATCGCCGTGGAGTGGGAGAGCAATGGGCAGCCGGAGAACAACTACAAGACC
ACGCCTCCCGTGCTGGACTCCGACGGCTCCTTCTTCCTCtatAGCAAGCTCACCGTGGACAA
GAGCAGGTGGCAGCAGGGGAACGTCTTCTCATGCTCCGTGATGCATGAGGCTCTGCACAACC
ACTACACGCAGAAGAGCCTCTCCCTGTCTCCGGGTAAA
SEQ ID NO: 195:
GATGCATCTCTGCCTTACCTGCAGAAAGAAAGCGTGTTCCAGTCTGGCGCCCACGCCTACAG
AATTCCCGCTCTGCTGTATCTGCCAGGCCAGCAGTCTCTGCTGGCTTTCGCTGAACAGCGGG
CCAGCAAGAAGGATGAGCACGCCGAACTGATCGTGCTGCGGAGAGGCGATTACGACGCCGGC
ACACATCAGGTGCAGTGGCAGGCTCAAGAGGTGGTGGCTCAGGCTAGACTGGACGGCCACAG
ATCTATGAACCCCTGTCCTCTGTACGATGAACAGACCGGCACACTGTTTCTGTTCTTTATCG
CTATCCCCGGCCAAGTGACCGAGCAGCAGCAGCTGCAGACAAGAGCCAACGTGACCAGACTG
TGTTACGTGACCTCCACCGACCACGGCAGAACCTGGTCTAGCCCTAGAGATCTGACCGACGC
CGCCATCGGACCTGCCTATAGAGAGTGGTCCACCTTCGCCGTTGGACCTGGACACTGTCTCC
AGCTGCACGACAGGGCTAGATCTCTGGTGGTGCCTGCCTACGCCTATAGAAAGCTGCACCCC
AAACAGCGGCCTATTCCTAGCGCCTTCTGCTTTCTGAGCCACGATCACGGCAGGACATGGGC
CAGAGGACATTTCGTGGCCCAGGACACACTGGAATGCCAGGTGGCCGAAGTGGAAACCGGCG
AGCAGAGAGTCGTGACCCTGAACGCCAGATCTCACCTGAGATTCAGAGTGCAGGCCCAGAGC
ACAAACGACGGCCTGGATTTCCAAGAGAGCCAGCTGGTCAAGAAACTGGTGGAACCTCCTCC
AACCGGCTGTCAGGGAAGCGTGATCAGCTTTCCATCTCCTAGAAGCGGCCCTGGCTCTCCTG
CTCAGTGGCTGCTGTATACACACCCCACACACAGCTGGCAGAGAGCCGATCTGGGCGCCTAC
CTGAATCCTAGACCTCCTGCTCCTGAGGCTTGGAGCGAACCTGTTCTGCTGGCCAAGGGCAG
CGCTGCCTACAGCGATCTGCAGTCTATGGGCACAGGCCCTGATGGCAGCCCTCTGTTTGGCT
GTCTGTACGAGGCCAACGACTACGAAGAGATCGTGTTCCTGATGTTCACCCTGAAGCAGGCC
TTTCCAGCCGAGTACCTGCCTCAAGAGCCCAAATCTTCTGACAAAACTCACACATGCCCACC
GTGCCCAGCACCTGAACTCCTGGGGGGACCGTCAGTCTTCCTCTTCCCCCCAAAACCCAAGG
ACACCCTCATGATCTCCCGGACCCCTGAGGTCACATGCGTGGTGGTGGACGTGAGCCACGAA
GACCCTGAGGTCAAGTTCAACTGGTACGTGGACGGCGTGGAGGTGCATAATGCCAAGACAAA
GCCGCGGGAGGAGCAGTACAACAGCACGTACCGTGTGGTCAGCGTCCTCACCGTCCTGCACC
AGGACTGGCTGAATGGCAAGGAGTACAAGTGCAAGGTCTCCAACAAAGCCCTCCCAGCCCCC
ATCGAGAAAACCATCTCCAAAGCCAAAGGGCAGCCCCGAGAACCACAGGTCTACACCCTGCC
CCCATCCCGGGAGGAGATGACCAAGAACCAGGTCAGCCTGACCTGCCTGGTCAAAGGCTTCT
ATCCCAGCGACATCGCCGTGGAGTGGGAGAGCAATGGGCAGCCGGAGAACAACTACAAGACC
ACGCCTCCCGTGCTGGACTCCGACGGCTCCTTCTTCCTCTATAGCAAGCTCACCGTGGACAA
GAGCAGGTGGCAGCAGGGGAACGTCTTCTCATGCTCCGTGATGCATGAGGCTCTGCACAACC
ACTACACGCAGAAGAGCCTCTCCCTGTCTCCGGGTAAA
SEQ ID NO: 196:
DASLPYLQKESVFQSGAHAYRIPALLYLPGQQSLLAFAEQRASKKDEHAELIVLRRGDYDAG
THQVQWQAQEVVAQARLDGHRSMNPCPLYDEQTGTLFLFFIAIPGQVTEQQQLQTRANVTRL
CYVTSTDHGRTWSSPRDLTDAAIGPAYREWSTFAVGPGHCLQLHDRARSLVVPAYAYRKLHP
KQRPIPSAFCFLSHDHGRTWARGHFVAQDTLECQVAEVETGEQRVVTLNARSHLRARVQAQS
TNDGLDFQESQLVKKLVEPPPQGCQGSVISFPSPRSGPGSPAQWLLYTHPTHSWQRADLGAY
LNPRPPAPEAWSEPVLLAKGSAAYSDLQSMGTGPDGSPLFGCLYEANDYEEIVFLMFTLKQA
FPAEYLPQ
SEQ ID NO: 197:
DASLPYLQKESVFQSGAHAYRIPALLYLPGQQSLLAFAEQRASKKDEHAELIVLRRGDYDAG
THQVQWQAQEVVAQARLDGHRSMNPCPLYDEQTGTLFLFFIAIPGQVTEQQQLQTRANVTRL
CYVTSTDHGRTWSSPRDLTDAAIGPAYREWSTFAVGPGHCLQLHDRARSLVVPAYAYRKLHP
KQRPIPSAFCFLSHDHGRTWARGHFVAQDTLECQVAEVETGEQRVVTLNARSHLRARVQAQS
TNDGLDFQESQLVKKLVEPPPQGCQGSVISFPSPRSGPGSPAQWLLYTHPTHSWQRADLGAY
LNPRPPAPEAWSEPVLLAKGSAAYSDLQSMGTGPDGSPLFGCLYEANDYEEIVFLMFTLKQA
FPAEYLPQEPKSSDKTHTCPPCPAPELLGGPSVFLFPPKPKDTLMISRTPEVTCVVVDVSHE
DPEVKFNWYVDGVEVHNAKTKPREEQYNSTYRVVSVLTVLHQDWLNGKEYKCKVSNKALPAP
IEKTISKAKGQPREPQVYTLPPSREEMTKNQVSLTCLVKGFYPSDIAVEWESNGQPENNYKT
TPPVLDSDGSFFLTSKLTVDKSRWQQGNVFSCSVMHEALHNHYTOKSLSLSPGK
SEQ ID NO: 198:
DASLPYLQKESVFQSGAHAYRIPALLYLPGQQSLLAFAEQRASKKDEHAELIVLRRGDYDAG
THQVQWQAQEVVAQARLDGHRSMNPCPLYDEQTGTLFLFFIAIPGQVTEQQQLQTRANVTRL
CYVTSTDHGRTWSSPRDLTDAAIGPAYREWSTFAVGPGHCLQLHDRARSLVVPAYAYRKLHP
KQRPIPSAFCFLSHDHGRTWARGHFVAQDTLECQVAEVETGEQRVVTLNARSHLRARVQAQS
TNDGLDFQESQLVKKLVEPPPQGCQGSVISFPSPRSGPGSPAQWLLYTHPTHSWQRADLGAY
LNPRPPAPEAWSEPVLLAKGSAAYSDLQSMGTGPDGSPLFGCLYEANDYEEIVFLMFTLKQA
FPAEYLPQEPKSSDKTHTCPPCPAPELLGGPSVFLFPPKPKDTLMISRTPEVTCVVVDVSHE
DPEVKFNWYVDGVEVHNAKTKPREEQYNSTYRVVSVLTVLHQDWLNGKEYKCKVSNKALPAP
IEKTISKAKGQPREPQVYTLPPSREEMTKNQVSLTCLVKGFYPSDIAVEWESNGQPENNYKT
TPPVLDSDGSFFLYSKLTVDKSRWQQGNVFSCSVMHEALHNHYTQKSLSLSPGK
SEQ ID NO: 199:
DASLPYLQKESVFQSGAHAYRIPALLYLPGQQSLLAFAEQRASKKDEHAELIVLRRGDYDAG
THQVQWQAQEVVAQARLDGHRSMNPCPLYDEQTGTLFLFFIAIPGQVTEQQQLQTRANVTRL
CYVTSTDHGRTWSSPRDLTDAAIGPAYREWSTFAVGPGHCLQLHDRARSLVVPAYAYRKLHP
KQRPIPSAFCFLSHDHGRTWARGHFVAQDTLECQVAEVETGEQRVVTLNARSHLRARVQAQS
TNDGLDFQESQLVKKLVEPPPQGCQGSVISFPSPRSGPGSPAQWLLYTHPTHSWQRADLGAY
LNPRPPAPEAWSEPVLLAKGSAAYSDLQSMGTGPDGSPLFGCLYEANDYEEIVFLMFTLKQA
FPAEYLPQEPKSSDKTHTCPPCPAPELLGGPSVFLFPPKPKDTLMISRTPEVTCVVVDVSHE
DPEVKFNWYVDGVEVHNAKTKPREEQYNSTYRVVSVLTVLHQDWLNGKEYKCKVSNKALPAP
IEKTISKAKGQPREPQVYTLPPSREEMTKNQVSLTCLVKGFYPSDIAVEWESNGQPENNYKT
TPPVLDSDGSFFLYSKLTVDKSRWQQGNVFSCSVMHEALHNHYTQKSLSLSPGKGGGGSGGG
GSGGGGSEVQLVESGGGLVQPGGSLRLSCAASGFNIKDTYIHWVRQAPGKGLEWVARIYPTN
GYTRYADSVKGRFTISADTSKNTAYLQMNSLRAEDTAVYYCSRWGGDGFYAMDYWGQGTLVT
VSSGGGGSGGGGSGGGGSDIQMTQSPSSLSASVGDRVTITCRASQDVNTAVAWYQQKPGKAP
KLLIYSASFLYSGVPSRFSGSRSGTDFTLTISSLQPEDFATYYCQQHYTTPPTFGQGTKVEI
K
SEQ ID NO: 200:
DASLPYLQKESVFQSGAHAYRIPALLYLPGQQSLLAFAEQRASKKDEHAELIVLRRGDYDAG
THQVQWQAQEVVAQARLDGHRSMNPCPLYDEQTGTLFLFFIAIPGQVTEQQQLQTRANVTRL
CYVTSTDHGRTWSSPRDLTDAAIGPAYREWSTFAVGPGHCLQLHDRARSLVVPAYAYRKLHP
KQRPIPSAFCFLSHDHGRTWARGHFVAQDTLECQVAEVETGEQRVVTLNARSHLRARVQAQS
TNDGLDFQESQLVKKLVEPPPQGCQGSVISFPSPRSGPGSPAQWLLYTHPTHSWQRADLGAY
LNPRPPAPEAWSEPVLLAKGSAAYSDLQSMGTGPDGSPLFGCLYEANDYEEIVFLMFTLKQA
FPAEYLPQGGGGSGGGGSDKTHTCPPCPAPELLGGPSVFLFPPKPKDTLMISRTPEVTCVVV
DVSHEDPEVKFNWYVDGVEVHNAKTKPREEQYNSTYRVVSVLTVLHQDWLNGKEYKCKVSNK
ALPAPIEKTISKAKGQPREPQVYTLPPSREEMTKNQVSLTCLVKGFYPSDIAVEWESNGQPE
NNYKTTPPVLDSDGSFFLTSKLTVDKSRWQQGNVFSCSVMHEALHNHYTQKSLSLSPGK
SEQ ID NO: 201:
GATGCATCTCTGCCTTACCTGCAGAAAGAAAGCGTGTTCCAGTCTGGCGCCCACGCCTACAG
AATTCCCGCTCTGCTGTATCTGCCAGGCCAGCAGTCTCTGCTGGCTTTCGCTGAACAGCGGG
CCAGCAAGAAGGATGAGCACGCCGAACTGATCGTGCTGCGGAGAGGCGATTACGACGCCGGC
ACACATCAGGTGCAGTGGCAGGCTCAAGAGGTGGTGGCTCAGGCTAGACTGGACGGCCACAG
ATCTATGAACCCCTGTCCTCTGTACGATGAACAGACCGGCACACTGTTTCTGTTCTTTATCG
CTATCCCCGGCCAAGTGACCGAGCAGCAGCAGCTGCAGACAAGAGCCAACGTGACCAGACTG
TGTTACGTGACCTCCACCGACCACGGCAGAACCTGGTCTAGCCCTAGAGATCTGACCGACGC
CGCCATCGGACCTGCCTATAGAGAGTGGTCCACCTTCGCCGTTGGACCTGGACACTGTCTCC
AGCTGCACGACAGGGCTAGATCTCTGGTGGTGCCTGCCTACGCCTATAGAAAGCTGCACCCC
AAACAGCGGCCTATTCCTAGCGCCTTCTGCTTTCTGAGCCACGATCACGGCAGGACATGGGC
CAGAGGACATTTCGTGGCCCAGGACACACTGGAATGCCAGGTGGCCGAAGTGGAAACCGGCG
AGCAGAGAGTCGTGACCCTGAACGCCAGATCTCACCTGAGAGCCAGAGTGCAGGCCCAGAGC
ACAAACGACGGCCTGGATTTCCAAGAGAGCCAGCTGGTCAAGAAACTGGTGGAACCTCCTCC
ACAGGGCTGTCAGGGAAGCGTGATCAGCTTTCCATCTCCTAGAAGCGGCCCTGGCTCTCCTG
CTCAGTGGCTGCTGTATACACACCCCACACACAGCTGGCAGAGAGCCGATCTGGGCGCCTAC
CTGAATCCTAGACCTCCTGCTCCTGAGGCTTGGAGCGAACCTGTTCTGCTGGCCAAGGGCAG
CGCTGCCTACAGCGATCTGCAGTCTATGGGCACAGGCCCTGATGGCAGCCCTCTGTTTGGCT
GTCTGTACGAGGCCAACGACTACGAAGAGATCGTGTTCCTGATGTTCACCCTGAAGCAGGCC
TTTCCAGCCGAGTACCTGCCTCAAGAGCCCAAATCTTCTGACAAAACTCACACATGCCCACC
GTGCCCAGCACCTGAACTCCTGGGGGGACCGTCAGTCTTCCTCTTCCCCCCAAAACCCAAGG
ACACCCTCATGATCTCCCGGACCCCTGAGGTCACATGCGTGGTGGTGGACGTGAGCCACGAA
GACCCTGAGGTCAAGTTCAACTGGTACGTGGACGGCGTGGAGGTGCATAATGCCAAGACAAA
GCCGCGGGAGGAGCAGTACAACAGCACGTACCGTGTGGTCAGCGTCCTCACCGTCCTGCACC
AGGACTGGCTGAATGGCAAGGAGTACAAGTGCAAGGTCTCCAACAAAGCCCTCCCAGCCCCC
ATCGAGAAAACCATCTCCAAAGCCAAAGGGCAGCCCCGAGAACCACAGGTCTACACCCTGCC
CCCATCCCGGGAGGAGATGACCAAGAACCAGGTCAGCCTGACCTGCCTGGTCAAAGGCTTCT
ATCCCAGCGACATCGCCGTGGAGTGGGAGAGCAATGGGCAGCCGGAGAACAACTACAAGACC
ACGCCTCCCGTGCTGGACTCCGACGGCTCCTTCTTCCTCTATAGCAAGCTCACCGTGGACAA
GAGCAGGTGGCAGCAGGGGAACGTCTTCTCATGCTCCGTGATGCATGAGGCTCTGCACAACC
ACTACACGCAGAAGAGCCTCTCCCTGTCTCCGGGTAAA

What is claimed is:

1. A pharmaceutical composition comprising a sialidase conjugated to a serum half-life enhancer that increases the serum half-life of the sialidase when administered to a subject.

2. The pharmaceutical composition of claim 1, wherein the sialidase is not conjugated to a cancer antigen targeting agent that binds a cancer antigen associated with a cancerous cell.

3. The pharmaceutical composition of claims 1-2, wherein the sialidase is a functional fragment of a full-length sialidase that exhibits at least 50% of the activity of the full-length sialidase.

4. The pharmaceutical composition of any one of claims 1-3, wherein the sialidase is a variant that exhibits at least 50% of the activity of the wild-type sialidase.

5. The pharmaceutical composition of any one of claims 1-4, wherein the sialidase and the serum half-life enhancer are covalently linked together in a fusion protein.

6. The pharmaceutical composition of any one of claims 1-4, wherein the sialidase and serum half-life enhancer are chemically conjugated together.

7. The pharmaceutical composition of claims 1-6, wherein the serum half-life enhancer is selected from the group consisting of an Fc domain, transferrin, albumin, XTEN, a homo-amino acid polymer (HAP), a proline-alanine-serine polymer (PAS), an elastin-like peptide (ELP), albumin binding domain, CTP fusion, GLK fusion, and a polyethylene glycol.

8. The pharmaceutical composition of claims 1-7, wherein the serum half-life enhancer is an Fc domain.

9. The pharmaceutical composition of claims 1-7, wherein the serum half-life enhancer is not an Fc domain or polyethylene glycol.

10. The pharmaceutical composition of claims 1-9, wherein the sialidase comprises one or more mutations relative to a template, wild-type sialidase.

11. The pharmaceutical composition of claims 1-10, wherein the sialidase comprises:
(a) a substitution or deletion of a methionine residue at a position corresponding to position 1 of wild-type human Neu2 (M1);
(b) a substitution of a valine residue at a position corresponding to position 6 of wild-type human Neu2 (V6);
(c) a substitution of an isoleucine residue at a position corresponding to position 187 of wild-type human Neu2 (I187); or
(d) a substitution of a cysteine residue at a position corresponding to position 332 of wild-type human Neu2 (C332);
or a combination of any of the foregoing substitutions.

12. The pharmaceutical compositions of claim 11, wherein, in the sialidase:
(a) the methionine residue at a position corresponding to position 1 of wild-type human Neu2 is deleted (ΔM1), is substituted by alanine (M1A), or is substituted by aspartic acid (M1D);
(b) the valine residue at a position corresponding to position 6 of wild-type human Neu2 is substituted by tyrosine (V6Y);
(c) the isoleucine residue at a position corresponding to position 187 of wild-type human Neu2 is substituted by lysine (I187K); or
(d) the cysteine residue at a position corresponding to position 332 of wild-type human Neu2 is substituted by alanine (C332A);
or the sialidase comprises a combination of any of the foregoing substitutions.

13. The pharmaceutical composition of claims 1-11, wherein the sialidase comprises:
(a) a substitution or deletion of a methionine residue at a position corresponding to position 1 of wild-type human Neu2 (M1);
(b) a substitution of a valine residue at a position corresponding to position 6 of wild-type human Neu2 (V6);
(c) a substitution of an proline residue at a position corresponding to position 62 of wild-type human Neu2 (P62);
(d) a substitution of an alanine residue at a position corresponding to position 93 of wild-type human Neu2 (A93);
(e) a substitution of an isoleucine residue at a position corresponding to position 187 of wild-type human Neu2 (I187);
(f) a substitution of a glutamine residue at a position corresponding to position 126 of wild-type human Neu2 (Q126);
(g) a substitution of an alanine residue at a position corresponding to position 242 of wild-type human Neu2 (A242);
(h) a substitution of a glutamine residue at a position corresponding to position 270 of wild-type human Neu2 (Q270);
(i) a substitution of a serine residue at a position corresponding to position 301 of wild-type human Neu2 (S301);
(j) a substitution of a tryptophan residue at a position corresponding to position 302 of wild-type human Neu2 (W302);
(k) a substitution of a cysteine residue at a position corresponding to position 332 of wild-type human Neu2 (C332);
(l) or a combination of any of the foregoing substitutions.

14. The pharmaceutical composition of any preceding claim, wherein the sialidase comprises a combination of substitutions selected from the group consisting of:
(a) M1D, V6Y, P62G, A93E, I187K, C332A;
(b) M1D, V6Y, P62G, A93E, I187K, S301A, W302R, C332A;
(c) M1D, V6Y, P62G, A93E, Q126Y, I187K, A242F, Q270T, C332A;
(d) M1D, V6Y, P62G, A93E, Q126Y, I187K, C332A; and
(e) A93E, Q126Y, I187K, A242F, Q270T, C332A.

15. The pharmaceutical composition of claim 14, wherein the sialidase conjugated to a serum half-life enhancer comprises an amino acid sequence selected from the group consisting of SEQ ID NOs: 115, 152, 180, 184, and 188, or an amino acid sequence that has at least 85%, 90%, 95%, 96%, 97%, 98%, or 99% to an amino acid sequence selected from the group consisting of SEQ ID NOs: 115, 152, 180, 184, and 188.

16. The pharmaceutical composition of any preceding claim, wherein the sialidase comprises:
(a) a substitution of a proline residue at a position corresponding to position 5 of wild-type human Neu2 (PS);
(b) a substitution of a lysine residue at a position corresponding to position 9 of wild-type human Neu2 (K9);
(c) a substitution of a lysine residue at a position corresponding to position 44 of wild-type human Neu2 (K44);
(d) a substitution of a lysine residue at a position corresponding to position 45 of wild-type human Neu2 (K45);
(e) a substitution of a leucine residue at a position corresponding to position 54 of wild-type human Neu2 (L54);
(f) a substitution of a proline residue at a position corresponding to position 62 of wild-type human Neu2 (P62);
(g) a substitution of a glutamine residue at a position corresponding to position 69 of wild-type human Neu2 (Q69);
(h) a substitution of an arginine residue at a position corresponding to position 78 of wild-type human Neu2 (R78);
(i) a substitution of an aspartic acid residue at a position corresponding to position 80 of wild-type human Neu2 (D80);
(j) a substitution of an alanine residue at a position corresponding to position 93 of wild-type human Neu2 (A93);
(k) a substitution of a glycine residue at a position corresponding to position 107 of wild-type human Neu2 (G107);
(l) a substitution of a glutamine residue at a position corresponding to position 108 of wild-type human Neu2 (Q108);
(m) a substitution of a glutamine residue at a position corresponding to position 112 of wild-type human Neu2 (Q112);
(n) a substitution of a cysteine residue at a position corresponding to position 125 of wild-type human Neu2 (C125);
(o) a substitution of a glutamine residue at a position corresponding to position 126 of wild-type human Neu2 (Q126);
(p) a substitution of an alanine residue at a position corresponding to position 150 of wild-type human Neu2 (A150);
(q) a substitution of a cysteine residue at a position corresponding to position 164 of wild-type human Neu2 (C164);
(r) a substitution of an arginine residue at a position corresponding to position 170 of wild-type human Neu2 (R170);
(s) a substitution of an alanine residue at a position corresponding to position 171 of wild-type human Neu2 (A171);
(t) a substitution of a glutamine residue at a position corresponding to position 188 of wild-type human Neu2 (Q188);
(u) a substitution of an arginine residue at a position corresponding to position 189 of wild-type human Neu2 (R189);
(v) a substitution of an alanine residue at a position corresponding to position 213 of wild-type human Neu2 (A213);
(w) a substitution of a leucine residue at a position corresponding to position 217 of wild-type human Neu2 (L217);
(x) a substitution of a glutamic acid residue at a position corresponding to position 225 of wild-type human Neu2 (E225);
(y) a substitution of a histidine residue at a position corresponding to position 239 of wild-type human Neu2 (H239);
(z) a substitution of a leucine residue at a position corresponding to position 240 of wild-type human Neu2 (L240);
(aa) a substitution of an arginine residue at a position corresponding to position 241 of wild-type human Neu2 (R241);
(bb) a substitution of an alanine residue at a position corresponding to position 242 of wild-type human Neu2 (A242);
(cc) a substitution of a valine residue at a position corresponding to position 244 of wild-type human Neu2 (V244);
(dd) a substitution of a threonine residue at a position corresponding to position 249 of wild-type human Neu2 (T249);
(ee) a substitution of an aspartic acid residue at a position corresponding to position 251 of wild-type human Neu2 (D251);
(ff) a substitution of a glutamic acid residue at a position corresponding to position 257 of wild-type human Neu2 (E257);
(gg) a substitution of a serine residue at a position corresponding to position 258 of wild-type human Neu2 (S258);
(hh) a substitution of a leucine residue at a position corresponding to position 260 of wild-type human Neu2 (L260);
(ii) a substitution of a valine residue at a position corresponding to position 265 of wild-type human Neu2 (V265);
(jj) a substitution of a glutamine residue at a position corresponding to position 270 of wild-type human Neu2 (Q270);
(kk) a substitution of a tryptophan residue at a position corresponding to position 292 of wild-type human Neu2 (W292);
(ll) a substitution of a serine residue at a position corresponding to position 301 of wild-type human Neu2 (S301);
(mm) a substitution of a tryptophan residue at a position corresponding to position 302 of wild-type human Neu2 (W302);
(nn) a substitution of a cysteine residue at a position corresponding to position 332 of wild-type human Neu2 (C332);
(oo) a substitution of a valine residue at a position corresponding to position 363 of wild-type human Neu2 (V363); or
(pp) a substitution of a leucine residue at a position corresponding to position 365 of wild-type human Neu2 (L365);
or a combination of any of the foregoing substitutions.

17. The pharmaceutical composition of claims 1-16, wherein the sialidase is selected from the group consisting of a bacterial sialidase, a viral sialidase, and a mammalian sialidase.

18. The pharmaceutical composition of claim 17, wherein the mammalian sialidase is a human sialidase.

19. The pharmaceutical composition of claim 18, wherein the human sialidase is selected from the group consisting of neu1, neu2, neu3, and neu4.

20. The pharmaceutical composition of claim 19, wherein the human sialidase is neu2.

21. The pharmaceutical composition of any one of claims 1-20, comprising from about 0.01 mg/kg to about 100 mg/kg of the sialidase.

22. The pharmaceutical composition of any one of claims 1-21 wherein the pharmaceutical composition comprises a second therapeutic agent.

23. The pharmaceutical composition of claim 22, wherein the second therapeutic agent is selected from the group consisting of an anti-inflammatory agent, anti-angiogenic agent, anti-fibrotic agent, or an anti-proliferative compound (e.g., a cytotoxic agent or a checkpoint inhibitor).

24. The pharmaceutical composition of any one of claims 1-23, wherein the pharmaceutical composition further comprises a stabilizing amount of a sialidase stabilizing agent.

25. The pharmaceutical composition of claim 24, wherein the sialidase stabilizing agent is a cation.

26. The pharmaceutical composition of claim 25, wherein the cation is selected from the group consisting of calcium and magnesium.

27. The pharmaceutical composition of any one of claims 1-26, wherein the pharmaceutical composition is disposed in a sterile container (e.g., bottle or vial).

28. The pharmaceutical composition of claim 27, wherein the pharmaceutical composition is lyophilized in the sterile container.

29. The pharmaceutical composition of claim 28, wherein the pharmaceutical composition is present as a solution in the sterile container.

30. The pharmaceutical composition of any one of claims 27-29, wherein the sterile container is sealed with a septum.

31. The pharmaceutical composition of any one of claims 27-30, wherein the sterile container has a label disposed thereon identifying the pharmaceutical composition contained in the container.

32. A method of treating a sialic acid-related disorder in a subject in need thereof, the method comprising administering to the subject a pharmaceutical composition comprising an effective amount of a sialidase and a serum half-life enhancer that increases the serum half-life of the sialidase when administered to a subject, thereby to treat the disorder.

33. The method of claim 32, wherein the sialic acid-related disorder is cancer.

34. The method of claim 33, wherein the sialidase is not conjugated to a cancer antigen targeting agent that binds a cancer antigen associated with a cancerous cell.

35. The method of any one of claims 32-34, wherein the sialidase is a functional fragment of a full-length sialidase that exhibits at least 50% of the activity of the full-length sialidase.

36. The method of any one of claims 32-35, wherein the sialidase is a variant that exhibits at least 50% of the activity of the wild-type sialidase.

37. The method of any one of claims 32-36, wherein the sialidase and the serum half-life enhancer are covalently linked together in a fusion protein.

38. The method of any one of claims 32-36, wherein the sialidase and serum half-life enhancer are chemically conjugated together.

39. The method of any one of claims 32-37, wherein the serum half-life enhancer is selected from the group consisting of an Fc domain, transferrin, albumin, XTEN, a homo-amino acid polymer (HAP), a proline-alanine-serine polymer (PAS), an elastin-like peptide (ELP), and a polyethylene glycol.

40. The method of any one of claims 32-39, wherein the serum half-life enhancer is an Fc domain.

41. The method of any one of claims 32-39, wherein the serum half-life enhancer is not an Fc domain or polyethylene glycol.

42. The method of any one of claims 32-41, wherein the sialidase comprises one or more mutations relative to a template, wild-type sialidase.

43. The method of any one of claims 32-42, wherein the sialidase comprises:
(a) a substitution or deletion of a methionine residue at a position corresponding to position 1 of wild-type human Neu2 (M1);
(b) a substitution of a valine residue at a position corresponding to position 6 of wild-type human Neu2 (V6);
(c) a substitution of an isoleucine residue at a position corresponding to position 187 of wild-type human Neu2 (I187); or
(d) a substitution of a cysteine residue at a position corresponding to position 332 of wild-type human Neu2 (C332);
or a combination of any of the foregoing substitutions.

44. The method of claim 43, wherein, in the sialidase:
(a) the methionine residue at a position corresponding to position 1 of wild-type human Neu2 is deleted (ΔM1), is substituted by alanine (M1A), or is substituted by aspartic acid (M1D);
(b) the valine residue at a position corresponding to position 6 of wild-type human Neu2 is substituted by tyrosine (V6Y);
(c) the isoleucine residue at a position corresponding to position 187 of wild-type human Neu2 is substituted by lysine (I187K); or
(d) the cysteine residue at a position corresponding to position 332 of wild-type human Neu2 is substituted by alanine (C332A);
or the sialidase comprises a combination of any of the foregoing substitutions.

45. The method of any one of claims 32-44, wherein the sialidase comprises:
(a) a substitution or deletion of a methionine residue at a position corresponding to position 1 of wild-type human Neu2 (M1);
(b) a substitution of a valine residue at a position corresponding to position 6 of wild-type human Neu2 (V6);
(c) a substitution of an proline residue at a position corresponding to position 62 of wild-type human Neu2 (P62);
(d) a substitution of an alanine residue at a position corresponding to position 93 of wild-type human Neu2 (A93);
(e) a substitution of an isoleucine residue at a position corresponding to position 187 of wild-type human Neu2 (I187);
(f) a substitution of a glutamine residue at a position corresponding to position 126 of wild-type human Neu2 (Q126);
(g) a substitution of an alanine residue at a position corresponding to position 242 of wild-type human Neu2 (A242);
(h) a substitution of a glutamine residue at a position corresponding to position 270 of wild-type human Neu2 (Q270);
(i) a substitution of a serine residue at a position corresponding to position 301 of wild-type human Neu2 (S301);
(j) a substitution of a tryptophan residue at a position corresponding to position 302 of wild-type human Neu2 (W302);
(k) a substitution of a cysteine residue at a position corresponding to position 332 of wild-type human Neu2 (C332);
(l) or a combination of any of the foregoing substitutions.

46. The method of any one of claims 32-45, wherein the sialidase comprises a combination of substitutions selected from the group consisting of:
(a) M1D, V6Y, P62G, A93E, I187K, C332A;
(b) M1D, V6Y, P62G, A93E, I187K, S301A, W302R, C332A;
(c) M1D, V6Y, P62G, A93E, Q126Y, I187K, A242F, Q270T, C332A;
(d) M1D, V6Y, P62G, A93E, Q126Y, I187K, C332A; and
(e) A93E, Q126Y, I187K, A242F, Q270T, C332A.

47. The method of claim 46, wherein the sialidase conjugated to a serum half-life enhancer comprises an amino acid sequence selected from the group consisting of SEQ ID NOs: 115, 152, 180, 184, and 188, or an amino acid sequence that has at least 85%, 90%, 95%, 96%, 97%, 98%, or 99% to an amino acid sequence selected from the group consisting of SEQ ID NOs: 115, 152, 180, 184, and 188.

48. The method of any one of claims 32-47, wherein the sialidase comprises:
(a) a substitution of a proline residue at a position corresponding to position 5 of wild-type human Neu2 (P5);
(b) a substitution of a lysine residue at a position corresponding to position 9 of wild-type human Neu2 (K9);
(c) a substitution of a lysine residue at a position corresponding to position 44 of wild-type human Neu2 (K44);
(d) a substitution of a lysine residue at a position corresponding to position 45 of wild-type human Neu2 (K45);
(e) a substitution of a leucine residue at a position corresponding to position 54 of wild-type human Neu2 (L54);
(f) a substitution of a proline residue at a position corresponding to position 62 of wild-type human Neu2 (P62);
(g) a substitution of a glutamine residue at a position corresponding to position 69 of wild-type human Neu2 (Q69);
(h) a substitution of an arginine residue at a position corresponding to position 78 of wild-type human Neu2 (R78);
(i) a substitution of an aspartic acid residue at a position corresponding to position 80 of wild-type human Neu2 (D80);
(j) a substitution of an alanine residue at a position corresponding to position 93 of wild-type human Neu2 (A93);
(k) a substitution of a glycine residue at a position corresponding to position 107 of wild-type human Neu2 (G107);
(l) a substitution of a glutamine residue at a position corresponding to position 108 of wild-type human Neu2 (Q108);
(m) a substitution of a glutamine residue at a position corresponding to position 112 of wild-type human Neu2 (Q112);
(n) a substitution of a cysteine residue at a position corresponding to position 125 of wild-type human Neu2 (C125);
(o) a substitution of a glutamine residue at a position corresponding to position 126 of wild-type human Neu2 (Q126);
(p) a substitution of an alanine residue at a position corresponding to position 150 of wild-type human Neu2 (A150);
(q) a substitution of a cysteine residue at a position corresponding to position 164 of wild-type human Neu2 (C164);
(r) a substitution of an arginine residue at a position corresponding to position 170 of wild-type human Neu2 (R170);
(s) a substitution of an alanine residue at a position corresponding to position 171 of wild-type human Neu2 (A171);
(t) a substitution of a glutamine residue at a position corresponding to position 188 of wild-type human Neu2 (Q188);
(u) a substitution of an arginine residue at a position corresponding to position 189 of wild-type human Neu2 (R189);
(v) a substitution of an alanine residue at a position corresponding to position 213 of wild-type human Neu2 (A213);
(w) a substitution of a leucine residue at a position corresponding to position 217 of wild-type human Neu2 (L217);
(x) a substitution of a glutamic acid residue at a position corresponding to position 225 of wild-type human Neu2 (E225);
(y) a substitution of a histidine residue at a position corresponding to position 239 of wild-type human Neu2 (H239);
(z) a substitution of a leucine residue at a position corresponding to position 240 of wild-type human Neu2 (L240);
(aa) a substitution of an arginine residue at a position corresponding to position 241 of wild-type human Neu2 (R241);
(bb) a substitution of an alanine residue at a position corresponding to position 242 of wild-type human Neu2 (A242);
(cc) a substitution of a valine residue at a position corresponding to position 244 of wild-type human Neu2 (V244);
(dd) a substitution of a threonine residue at a position corresponding to position 249 of wild-type human Neu2 (T249);
(ee) a substitution of an aspartic acid residue at a position corresponding to position 251 of wild-type human Neu2 (D251);
(ff) a substitution of a glutamic acid residue at a position corresponding to position 257 of wild-type human Neu2 (E257);
(gg) a substitution of a serine residue at a position corresponding to position 258 of wild-type human Neu2 (S258);
(hh) a substitution of a leucine residue at a position corresponding to position 260 of wild-type human Neu2 (L260);
(ii) a substitution of a valine residue at a position corresponding to position 265 of wild-type human Neu2 (V265);
(jj) a substitution of a glutamine residue at a position corresponding to position 270 of wild-type human Neu2 (Q270);
(kk) a substitution of a tryptophan residue at a position corresponding to position 292 of wild-type human Neu2 (W292);
(ll) a substitution of a serine residue at a position corresponding to position 301 of wild-type human Neu2 (S301);
(mm) a substitution of a tryptophan residue at a position corresponding to position 302 of wild-type human Neu2 (W302);
(nn) a substitution of a cysteine residue at a position corresponding to position 332 of wild-type human Neu2 (C332);
(oo) a substitution of a valine residue at a position corresponding to position 363 of wild-type human Neu2 (V363); or
(pp) a substitution of a leucine residue at a position corresponding to position 365 of wild-type human Neu2 (L365);
or a combination of any of the foregoing substitutions.

49. The method of any one of claims 32-48, wherein the sialidase is selected from the group consisting of a bacterial sialidase, a viral sialidase, and a mammalian sialidase.

50. The method of claim 49, wherein the mammalian sialidase is a human sialidase.

51. The method of claim 50, wherein the human sialidase is selected from the group consisting of neu1, neu2, neu3, and neu4.

52. The method of claim 51, wherein the human sialidase is neu2.

53. The method of any one of claims 32-52, wherein from about 0.01 mg/kg to about 100 mg/kg of the sialidase is administered to the subject.

54. The method of any one of claims 32-53, wherein the cancer is a solid tumor, soft tissue tumor, hematopoietic tumor or metastatic lesion.

55. The method of claim 54, wherein the solid tumor is a sarcoma, adenocarcinoma, or carcinoma.

56. The method of claim 54 or 55, wherein the solid tumor is a head and neck (e.g., pharynx), thyroid, lung (e.g., small cell or non-small cell lung carcinoma (NSCLC)), breast, lymphoid, gastrointestinal (e.g., oral, esophageal, stomach, liver, pancreas, small intestine, colon and rectum, anal canal), genital or genitourinary tract (e.g., renal, urothelial, bladder, ovarian, uterine, cervical, endometrial, prostate, testicular), CNS (e.g., neural or glial cell, e.g., neuroblastoma or glioma), or skin (e.g., melanoma) tumor.

57. The method of claim 56, wherein the hematopoietic tumor is a leukemia, acute leukemia, acute lymphoblastic leukemia (ALL), B-cell, T-cell or FAB ALL, acute myeloid leukemia (AML), chronic myelocytic leukemia (CML), chronic lymphocytic leukemia (CLL), e.g., transformed CLL, diffuse large B-cell lymphoma (DLBCL), follicular lymphoma, hairy cell leukemia, myelodyplastic syndrome (MDS), lymphoma, Hodgkin's disease, malignant lymphoma, non-Hodgkin's lymphoma, Burkitt's lymphoma, multiple myeloma, or Richter's Syndrome (Richter's Transformation).

58. The method of claim 56, wherein the cancer is breast cancer.

59. The method of any one of claim 56, wherein the cancer is lymphoma.

60. The method of any one of claims 32-59, wherein administration of the pharmaceutical composition increases expression of granzyme B, IFNγ, IL-10, IL-6, or IL-17A in the subject.

61. The method of any one of claims 32-60, wherein the pharmaceutical composition is administered to the subject in combination with another therapeutic agent.

62. The method of claim 61, wherein the therapeutic agent is selected from the group consisting of an anti-inflammatory agent, anti-angiogenic agent, anti-fibrotic agent, or an anti-proliferative compound (e.g., a cytotoxic agent or a checkpoint inhibitor).

63. The method of any one of claims 32-62, wherein the pharmaceutical composition further comprises a stabilizing amount of a sialidase stabilizing agent.

64. The method of claim 63, wherein the sialidase stabilizing agent is a cation.

65. The method of claim 64, wherein the cation is selected from the group consisting of calcium and magnesium.

66. The method of any one of claim 65, wherein the pharmaceutical composition, prior to administration, is disposed in a sterile container (e.g., bottle or vial).

67. A method of treating cancer in a subject in need thereof, the method comprising administering an effective amount of the pharmaceutical composition of any one of claims 1-31 to the subject.

68. A method of removing sialic acid from a cell in a subject, the method comprising administering to the subject an effective amount of the pharmaceutical composition of any one of claims 1-31 thereby to remove sialic acid from the cell.

69. The method of claim 68, wherein the cell is a tumor cell, dendritic cell (DC) or monocyte.

70. The method of claim 69, wherein the cell is a monocyte, and the method results in increased expression of an MHC-11 molecule on the monocyte.

71. A method of increasing phagocytosis of a tumor cell in a subject, the method comprising administering to the subject an effective amount of the pharmaceutical composition of any one of claims 1-31 in an amount effective to remove sialic acid from the tumor cell, thereby increasing phagocytosis of the tumor cell.

72. A method of activating a dendritic cell (DC) in a subject, the method comprising administering to the subject an amount of the pharmaceutical composition of any one of claims 1-31 effective to remove sialic acid from a tumor cell in the subject, thereby to activate the DC in the subject.

73. A method of reducing Siglec-15 binding activity, thereby increasing anti-tumor activity in a tumor microenvironment of a patient, the method comprising administering to the subject an effective amount of the pharmaceutical composition of any one of claims 1-31, thereby increasing anti-tumor activity (e.g., T cell activity) in the subject.

74. A method of expressing a recombinant sialidase, the method comprising:
(a) providing a cell comprising a nucleic acid encoding the recombinant sialidase; and
(b) expressing the recombinant sialidase in the presence of a stabilizing agent.

75. The method of claim 74, wherein the method further comprises purifying the recombinant sialidase produced in step (b).

76. The method of claim 75, wherein the purification is performed in the presence of a stabilizing agent.

77. The method of any one of claims 74-76, wherein the stabilizing agent is a cation.

78. The method of claim 77, wherein the cation is selected from the group consisting of calcium and magnesium.
